(19) United States
(12) Patent Application Publication
(10) Pub. No.: US 2025/0387425 A1
(43) Pub. Date: Dec. 25, 2025

(54) NOVEL PERSONAL NEOANTIGEN VACCINES AND MARKERS

(71) Applicant: ANDA BIOLOGY MEDICINE DEVELOPMENT (SHENZHEN) CO., LTD., Shenzhen, Guangdong (CN)

(72) Inventors: Landian HU, Shenzhen, Guangdong (CN); Xiangyin KONG, Shenzhen, Guangdong (CN); Yuchao ZHANG, Shenzhen, Guangdong (CN); Rongjing WANG, Shenzhen, Guangdong (CN); Zhenchuan WU, Shenzhen, Guangdong (CN)

(21) Appl. No.: 18/727,112

(22) PCT Filed: Jan. 9, 2023
(86) PCT No.: PCT/CN2023/071209

§ 371 Date: Jul. 8, 2024

(30) Foreign Application Priority Data
Jan. 7, 2022 (WO) PCT/CN2022/070855

(51) Int. Cl. A61K 31/7105; A61K 31/137; A61K 31/352; A61K 31/5386; A61K 31/7084; A61K 40/11; A61K 40/42; A61P 35/00; C12N 5/0783; C12Q 1/6886

(52) U.S. Cl. CPC A61K 31/7105; A61K 31/137; A61K 31/352; A61K 31/5386; A61K 31/7084; A61K 40/11; A61K 40/4201; A61P 35/00; C12N 5/0636; C12Q 1/6886; A61K 2239/39; C12Q 2600/106; C12Q 2600/118; C12Q 2600/158

(57) ABSTRACT

The invention provides personal neoantigen vaccines, and uses thereof. The invention also provides markers MX1 and PPP1R15A, and uses thereof. The invention also provides sets of biomarkers, and uses thereof.

### FIELD OF THE INVENTION

The present disclosure generally relates to novel personal neoantigen vaccines, and uses thereof. The present disclosure also generally relates to novel markers MX1 and PPP1R15A, and uses thereof.

### BACKGROUND

Neoantigen vaccines, synthesized antigens, was designed for providing sufficient tumor-specific antigens to stimulate T cell-mediated immunity and eliminate the tumor cells. The proof of concept for effectiveness of neoantigen vaccine or combined immune checkpoint inhibition (ICI) has been established in limited number of patients in melanoma, non-small cell lung cancer, and urothelial carcinoma of the bladder¹⁻⁴. Clinical use of neoantigen vaccine await widespread rollout and FDA authorization. Pancreatic ductal adenocarcinoma (PDAC) is an intractable malignancy with worst prognosis. The patient even with resection surgery is prone to experience recurrence and has a poor prognostic outcome in the late stages. In resectable pancreatic cancer, with low tumor burden and sufficient immune status, adjuvant neoantigen vaccine therapy might achieve better results. In addition, in radical resection-recurrence/no recurrence setting, it is rationale to evaluate the effect of neoantigen vaccines or combination of vaccines and ICIs and the response mechanism for preventing tumor recurrence and improving the prognosis.

The difficulty faced in conventional tumor vaccine therapy is the lack of markers that can detect specific changes in immune cells and correlate with the dose effect of the vaccine. Therefore, there exists great needs for treatments and markers for tumor vaccine therapy.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “a method” means one method or more than one method.

The present disclosure provides novel methods and compositions for enhancing cell-mediated immunity, stimulating and/or expanding T cells, potentiating immunogenicity, treating a condition that would benefit from upregulation of immune response, and promoting clonal expansion of T cells. The present disclosure also provides methods of using novel biomarkers of MX1 and PPP1R15A.

In one aspect, the present disclosure provides a method of enhancing cell-mediated immunity in a subject in need thereof, comprising administering to the subject an effective amount of a MX1 agonist, thereby enhancing cell-mediated immunity in the subject.

In certain embodiments, the cell-mediated immunity is T cell-mediated immunity.

In one aspect, the present disclosure provides a method of stimulating and/or expanding T cells in a subject in need thereof, comprising administering to the subject an effective amount of a MX1 agonist, thereby stimulating and/or expanding T cells in the subject.

In one aspect, the present disclosure provides a method of potentiating immunogenicity of an immunogenic composition in a subject, comprising administering to the subject an effective amount of a MX1 agonist in combination with the immunogenic composition.

In certain embodiments, the immunogenic composition is a vaccine or a composition for CAR-T treatment.

In certain embodiments, the vaccine is a tumor vaccine.

In certain embodiments, the subject is suffering from a condition that would benefit from upregulation of immune response.

In certain embodiments, the condition is tumor, infectious disease, cardiovascular disease or inflammatory disease.

In certain embodiments, the condition is tumor or infectious disease.

In certain embodiments, the subject is receiving a therapy whose efficacy can be potentiated by enhanced cell-mediated immunity.

In certain embodiments, the therapy is an anti-tumor therapy or anti-infectious therapy.

In certain embodiments, the anti-tumor therapy is selected from the group consisting of chemotherapy, targeted therapy, immunotherapy, cell therapy (e.g. CAR-T therapy), and tumor vaccine.

In one aspect, the present disclosure provides a method of treating a condition that would benefit from upregulation of immune response in a subject in need thereof, comprising administering to the subject an effective amount of a MX1 agonist.

In certain embodiments, the condition is tumor, infectious disease, cardiovascular disease or inflammatory disease.

In certain embodiments, the condition is tumor or infectious disease.

In certain embodiments, the MX1 agonist is administered in combination with a therapy that treats the condition.

In certain embodiments, the therapy is an anti-tumor therapy or anti-infectious therapy.

In certain embodiments, the anti-tumor therapy is selected from the group consisting of chemotherapy, targeted therapy, immunotherapy, cell therapy (e.g. CAR-T therapy), tumor vaccine and CAR-T therapy.

In certain embodiments, the MX1 agonist is selected from the group consisting of full-length mRNA of MX1, and small molecule compounds of DMXAA, ADU-S100, 2′,3′-cGAMP sodium and cGAMP.

In one aspect, the present disclosure provides a method of promoting clonal expansion of T cells, comprising treating the T cells with an effective amount of a MX1 agonist under suitable conditions, thereby allowing clonal expansion of the T cells.

In certain embodiments, the T cells are memory T cells.

In one aspect, the present disclosure provides a method of promoting T cell activation or promoting cytotoxicity of T cells, comprising treating the T cells with an effective amount of a MX1 agonist under suitable conditions.

In certain embodiments, the T cells are cultured with the MX1 agonist in combination with a second agent for stimulating the T cells.

In certain embodiments, the MX1 agonist is selected from the group consisting of full-length mRNA of MX1, and small molecule compounds of DMXAA, ADU-S100, 2′,3′-cGAMP sodium and cGAMP.

In one aspect, the present disclosure provides a composition comprising the T cells prepared using the method provided herein.

In one aspect, the present disclosure provides a method of treating a condition that would benefit from upregulation of immune response in a subject in need thereof, comprising administering to the subject the composition provided herein.

In one aspect, the present disclosure provides a method of evaluating activation state, or activity or cytotoxicity of T cells, comprising detecting expression level of MX1 in the T cells, wherein an increase in the expression level of MX1 relative to a control T cell is indicative of cytotoxicity of the T cells.

In certain embodiments, the T cells are CAR-T cells, or TCR-T cells.

In one aspect, the present disclosure provides a method of preparing a population of T cells for cell therapy, the method comprising:
a) identifying the population of T cells having increased expression level of MX1 relative to a control T cell; and
b) selectively enriching the identified T cells for cell therapy.

In one aspect, the present disclosure provides a method of converting a first population of inactive T cells to a second population of active T cells, the method comprising:
a) providing the first population of inactive T cells, wherein the inactive T cells do not show an increase in the expression level of MX1 relative to a control T cell;
b) treating the first population of inactive T cells with an effective amount of a MX1 agonist under suitable conditions; and
c) detecting expression level of MX1 in the population of T cells obtained in step b), wherein an increase in the expression level of MX1 relative to a control T cell is indicative of successful conversion to the second population of active T cells.

In one aspect, the present disclosure provides a method of preparing a population of T cells for cell therapy, the method comprising:
a) identifying the population of T cells as inactive if it does not show an increase in the expression level of MX1 relative to a control T cell; and
b) treating the identified inactive population of T cells with an effective amount of a MX1 agonist under suitable conditions, thereby obtaining a population of activated T cells.

In certain embodiments, the MX1 agonist is selected from the group consisting of full-length mRNA of MX1, and small molecule compounds of DMXAA, ADU-S100, 2′,3′-cGAMP sodium and cGAMP.

In one aspect, the present disclosure provides a composition comprising the population of T cells prepared or converted by the method provided herein.

In one aspect, the present disclosure provides a method of treating a condition that would benefit from upregulation of immune response in a subject in need thereof, comprising administering to the subject the population of T cells prepared or converted by the method provided herein.

In one aspect, the present disclosure provides a method of reducing cell-mediated immunity in a subject in need thereof, comprising administering to the subject an effective amount of a MX1 antagonist, thereby reducing cell-mediated immunity in the subject.

In certain embodiments, the cell-mediated immunity is T cell-mediated immunity.

In one aspect, the present disclosure provides a method of deactivating T cells in a subject in need thereof, comprising administering to the subject an effective amount of a MX1 antagonist, thereby deactivating T cells in the subject.

In certain embodiments, the subject has been determined to have T cells having an increase in the expression level of MX1 relative to a control T cell.

In certain embodiments, the subject is suffering from a condition characterized in excessive cell-mediated immunity.

In one aspect, the present disclosure provides a method of treating a condition that would benefit from downregulation of immune response in a subject in need thereof, comprising administering to the subject an effective amount of a MX1 antagonist.

In certain embodiments, the condition is an inflammatory disease, an autoimmune disease, an allergic disease, or a T cell cancer.

In certain embodiments, the condition is where the subject has received or is contemplated to receive an allogeneic or xenogeneic transplant.

In certain embodiments, the MX1 antagonist is selected from the group consisting of CCCP and H-151.

In certain embodiments, the control T cell is CD8+ T cell.

In one aspect, the present disclosure provides a method of assessing responsiveness of a subject to a tumor neoantigen vaccine, comprising:
a) determining expression level of one or more genes in one or more immune cells from a sample obtained from the subject; wherein the one or more genes are selected from the group consisting of: AC011815.2, ANKRD29, AP3M2, ARL5B, ATF4, ATP5F1B, C15orf54, CHCHD2, COL4A3BP, COPE, CTTNBP2, DDOST, DERL1, DNPH1, EGF, FERMT3, FOSL2, GCH1, GK, GLA, LMAN2, LRRK1, MAP9, MN1, MTDH, NFIL3, NUAK1, PDE4D, PIM1, PRKACA, PRMT1, SERTAD2, SLC16A6, SYT17, TMED10, TMEM258, TRDV1, WDFY3, ZBTB43 and ZDHHC7, or are any combination thereof, b) comparing the expression level of the one or more genes determined in step a) with a reference level to determine difference from the reference level; and
c) assessing the responsiveness of the subject to the tumor neoantigen vaccine based on the difference determined in step b).

In certain embodiments, the one or more immune cells are selected from the group consisting of: CD8+ T cells, CD4+ T cells, monocytes, B cells, NK cells and macrophages.

In certain embodiments, the one or more immune cells are CD8+ T cells and the one or more genes are selected from the group consisting of: AC011815.2, EGF, GCH1, NUAK1 and SERTAD2, or are any combination thereof.

In certain embodiments, the one or more immune cells are CD4+ T cells and the one or more genes are selected from the group consisting of: ANKRD29, C15orf54, FOSL2, GCH1, PRKACA, SERTAD2, SLC16A6, TRDV1, WDFY3 and ZBTB43, or are any combination thereof.

In certain embodiments, the one or more immune cells are monocytes and the one or more genes are selected from the group consisting of: AP3M2, ARL5B, ATP5F1B, CHCHD2, COPE, GLA, MN1 and MTDH, or are any combination thereof.

In certain embodiments, the one or more immune cells are B cells and the one or more genes are selected from the group consisting of: ATF4, ATP5F1B, DDOST, DERL1, DNPH1, LMAN2, MAP9, PDE4D, PIM1, TMEM258 and ZDHHC7, or are any combination thereof.

In certain embodiments, the one or more immune cells are NK cells and the one or more genes are selected from the group consisting of: COL4A3BP, FERMT3 and NFIL3, or are any combination thereof.

In certain embodiments, the one or more immune cells are macrophages and the one or more genes are selected from the group consisting of: COPE, CTTNBP2, GK, LRRK1, MTDH, PRMT1, SYT17 and TMED10, or are any combination thereof.

In one aspect, the present disclosure provides a method of assessing responsiveness of a subject to a tumor neoantigen vaccine during priming phase, comprising:
a) determining expression level of one or more genes in one or more immune cells from a sample obtained from the subject following the at least one priming dose of tumor neoantigen vaccine; wherein the one or more genes are selected from the group consisting of: AC011815.2, ATF4, C15orf54, DDOST, DERL1, DNPH1, EGF, FERMT3, FOSL2, GCH1, GK, LMAN2, MAP9, NUAK1, PDE4D, PRKACA, SERTAD2, SLC16A6, TMED10, TMEM258, WDFY3, ZBTB43 and ZDHHC7, or are any combination thereof;
b) comparing the expression level of the one or more genes determined in step a) with a reference level to determine difference from the reference level; and
c) assessing the responsiveness of the subject to the at least one priming dose of tumor neoantigen vaccine, based on the difference determined in step b).

In certain embodiments, the one or more immune cells are selected from the group consisting of: CD8+ T cells, CD4+ T cells, monocytes, B cells, NK cells and macrophages. In certain embodiments, the one or more immune cells are selected from the group consisting of: CD8+ T cells, CD4+ T cells, B cells, NK cells and macrophages.

In certain embodiments, the one or more immune cells are CD8+ T cells and the one or more genes are selected from the group consisting of: AC011815.2, EGF, GCH1, NUAK1 and SERTAD2, or are any combination thereof.

In certain embodiments, the one or more immune cells are CD4+ T cells and the one or more genes are selected from the group consisting of: C15orf54, FOSL2, GCH1, PRKACA, SERTAD2, SLC16A6, WDFY3 and ZBTB43, or are any combination thereof.

In certain embodiments, the one or more immune cells are B cells and the one or more genes are selected from the group consisting of: ATF4, DDOST, DERL1, DNPH1, LMAN2, MAP9, PDE4D, TMEM258 and ZDHHC7, or are any combination thereof.

In certain embodiments, the one or more immune cells are NK cells and the gene is FERMT3.

In certain embodiments, the one or more immune cells are macrophages and the one or more genes are selected from the group consisting of: GK and TMED10, or are any combination thereof.

In one aspect, the present disclosure provides a method of assessing responsiveness of a subject to a tumor neoantigen vaccine during boosting phase, comprising:
a) determining expression level of one or more genes in one or more immune cells from a sample obtained from the subject during the boosting phase;
wherein the one or more genes are selected from the group consisting of: AC011815.2, ANKRD29, AP3M2, ARL5B, ATP5F1B, C15orf54, CHCHD2, COL4A3BP, COPE, CTTNBP2, EGF, FERMT3, FOSL2, GK, GLA, LRRK1, MAP9, MN1, MTDH, NFIL3, NUAK1, PIM1, PRMT1, SERTAD2, SLC16A6, SYT17, TMED10, TRDV1 and ZDHHC7, or are any combination thereof, b) comparing the expression level of the one or more genes determined in step a) with a reference expression level to determine difference from the reference level; and
c) assessing the responsiveness of the subject to the at least one boosting dose of tumor neoantigen vaccine, based on the difference determined in step b).

In certain embodiments, the one or more immune cells are selected from the group consisting of: CD8+ T cells, CD4+ T cells, monocytes, B cells, NK cells and macrophages.

In certain embodiments, the one or more immune cells are CD8+ T cells and the one or more genes are selected from the group consisting of: AC011815.2, EGF, NUAK1 and SERTAD2, or are any combination thereof.

In certain embodiments, the one or more immune cells are CD4+ T cells and the one or more genes are selected from the group consisting of: ANKRD29, C15orf54, FOSL2, SLC16A6 and TRDV1, or are any combination thereof.

In certain embodiments, the one or more immune cells are monocytes and the one or more genes are selected from the group consisting of: AP3M2, ARL5B, ATP5F1B, CHCHD2, COPE, GLA, MN1 and MTDH, or are any combination thereof.

In certain embodiments, the one or more immune cells are B cells and the one or more genes are selected from the group consisting of: ATP5F1B, MAP9, PIM1 and ZDHHC7, or are any combination thereof.

In certain embodiments, the one or more immune cells are NK cells and the one or more genes are selected from the group consisting of: COL4A3BP, FERMT3 and NFIL3, or are any combination thereof.

In certain embodiments, the one or more immune cells are macrophages and the one or more genes are selected from the group consisting of: COPE, CTTNBP2, GK, LRRK1, MTDH, PRMT1, SYT17 and TMED10, or are any combination thereof.

In certain embodiments, the expression level of a given gene is represented by percentage of a given type of immune cells that express the given gene.

In certain embodiments, the reference expression level is the expression level of the one or more genes in the one or more immune cells from a sample obtained from the subject before receiving first dose of the tumor neoantigen vaccine.

In certain embodiments, the subject is determined as having a good response to the tumor neoantigen vaccine when the difference reaches or exceeds a predetermined threshold, or the subject is determined as having an insufficient response to the tumor neoantigen vaccine when the difference is below a predetermined threshold.

In one aspect, the present disclosure provides a method of predicting the risk of tumor relapse in a subject before or after receiving at least one dose of tumor neoantigen vaccine, comprising:
a) determining expression level of one or more genes in one or more immune cells from a sample obtained from the subject; wherein the one or more genes are selected from the group consisting of: ABCA13, AC022726.2, AC105094.2, AC243829.2, AL021807.1, AL391807.1, AL662907.3, APOBEC3C, ATP1B3, ATP6V1B2, C1QBP, CBX5, CCNE2, CD63, CEBPA, CHP1, CLN8, CMBL, CNIH4, COL4A3BP, CPNE3, DACH1, DNAJC12, DNAJC3, DOC2B, EEA1, ENAM, ERG, FAM43A, FBXO43, FIGN, GCA, GNG4, GNLY, GOLIM4, GPM6B, GPR171, GPRC5D, HHEX, HID1, ILF2, IRF2BP2, KIF22, LACC1, LIPA, MANEA, MECOM, MID1IP1, MX2, MYOM2, NABP1, NEFH, NFE4, NLN, OXCT2, P3H2, PI3, PIM1, PLAU, PRSS3, PSMA3, RAB10, RAB20, RASGRP2, RBMS3, RPL23, RPS6KA5, RPS8, RUNX1, SAMD3, 11-Sep, SERTAD2, SIGLEC6, SIT1, SOCS1, SSR1, ST6GALNAC1, SYNE2, TRAV16, TREM2, TXNDC15, TXNDC17, UAP1, UFM1, UGT2B17, XPNPEP1, ZBTB16, ZNF608 and STAT1, or are any combination thereof;
b) comparing the expression level of the one or more genes determined in step a) with a reference level to determine difference from the reference level; and
c) assessing the risk of tumor relapse in the subject based on the difference determined in step b).

In certain embodiments, the one or more genes are selected from the group consisting of: AC022726.2, AC105094.2, AC243829.2, AL021807.1, AL391807.1, ATP1B3, CMBL, DACH1, DNAJC12, DOC2B, EEA1, ERG, FBXO43, FIGN, GPRC5D, HID1, NFE4, OXCT2, P3H2, PI3, PLAU, PRSS3, RASGRP2, RPL23, RPS8, ST6GALNAC1, SYNE2, TRAV16, TREM2 and ZNF608, or are any combination thereof.

In certain embodiments, the expression level of the one or more genes are determined in one or more immune cells from a sample obtained from the subject after receiving the at least one dose of tumor neoantigen vaccine, wherein the one or more genes are selected from the group consisting of: AC022726.2, AC105094.2, AC243829.2, AL021807.1, AL391807.1, ATP1B3, ATP6V1B2, CHP1, CLN8, CMBL, CNIH4, CPNE3, DACH1, DNAJC12, DOC2B, EEA1, ERG, FAM43A, FBXO43, FIGN, GNG4, GPM6B, GPRC5D, HHEX, HID1, IRF2BP2, KIF22, LACC1, MECOM, MID1IP1, MYOM2, NABP1, NFE4, OXCT2, P3H2, PI3, PLAU, PRSS3, RAB10, RASGRP2, RBMS3, RPL23, RPS8, RUNX1, SAMD3, SERTAD2, SIGLEC6, SIT1, SOCS1, ST6GALNAC1, SYNE2, TRAV16, TREM2, TXNDC17, UGT2B17, XPNPEP1 and ZNF608, or are any combination thereof.

In certain embodiments, the expression level of the one or more genes are determined in one or more immune cells from a sample obtained from the subject before receiving the at least one dose of tumor neoantigen vaccine, wherein the one or more genes are selected from the group consisting of: ABCA13, AC022726.2, AC105094.2, AC243829.2, AL021807.1, AL391807.1, AL662907.3, ATP1B3, CBX5, CMBL, DACH1, DNAJC12, DNAJC3, DOC2B, ENAM, ERG, FBXO43, FIGN, GOLIM4, GPRC5D, HID1, ILF2, NEFH, NFE4, NLN, OXCT2, P3H2, PI3, PLAU, PRSS3, PSMA3, RASGRP2, RPL23, RPS6KA5, RPS8, 11-Sep, ST6GALNAC1, SYNE2, TRAV16, TREM2, TXNDC15, UAP1, UFM1, ZBTB16 and ZNF608, or are any combination thereof.

In certain embodiments, the one or more immune cells are selected from the group consisting of: CD8+ T cells, CD4+ T cells, monocytes, B cells, NK cells and macrophages.

In certain embodiments, the one or more immune cells are CD8+ T cells and the one or more genes are selected from the group consisting of: CHP1, EEA1, RPS8, RUNX1, UGT2B17, XPNPEP1, ZNF608 and STAT1, or are any combination thereof.

The method of any one of claims 72 to 76, wherein the one or more immune cells are CD4+ T cells and the one or more genes are selected from the group consisting of: AL662907.3, CD63, COL4A3BP, CPNE3, EEA1, GPM6B, LIPA, PSMA3, RAB10, RAB20, RBMS3, RPS8, SAMD3, SERTAD2, SOCS1, TXNDC15 and STAT1, or are any combination thereof.

In certain embodiments, the one or more immune cells are monocytes and the one or more genes are selected from the group consisting of: ABCA13, ATP1B3, DNAJC3, ENAM, FIGN, GNG4, ILF2, KIF22, MANEA, NEFH, NLN, P3H2, PLAU, SIGLEC6, SSR1, TRAV16, UFM1 and ZBTB16, or are any combination thereof.

In certain embodiments, the one or more immune cells are B cells and the one or more genes are selected from the group consisting of: AC105094.2, AC243829.2, AL021807.1, AL391807.1, ATP6V1B2, C1QBP, CMBL, CNIH4, DOC2B, FAM43A, GCA, GNLY, HID1, LACC1, MID1IP1, MX2, PI3, PIM1, 11-Sep, SIT1 and SYNE2, or are any combination thereof.

In certain embodiments, the one or more immune cells are NK cells and the one or more genes are selected from the group consisting of: AC022726.2, APOBEC3C, DACH1, ERG, IRF2BP2, MYOM2, NFE4, PRSS3, RPL23, RPS6KA5 and TREM2, or are any combination thereof.

In certain embodiments, the one or more immune cells are macrophages and the one or more genes are selected from the group consisting of: CBX5, CCNE2, CEBPA, CLN8, DNAJC12, FBXO43, FIGN, GOLIM4, GPR171, GPRC5D, HHEX, MECOM, NABP1, OXCT2, RASGRP2, ST6GALNAC1, TXNDC17 and UAP1, or are any combination thereof.

In certain embodiments, the expression level of a given gene is represented by percentage of a given type of immune cells that express the given gene.

In certain embodiments, the reference expression level is expression level of the corresponding gene in the corresponding immune cells that is representative for a relapse subject.

In certain embodiments, the reference expression level is a standard or average expression level determined from a representative population of relapse subjects.

In certain embodiments, the subject is determined as having low risk of tumor relapse when the difference reaches or exceeds a predetermined threshold, or the subject is determined as having high risk of tumor relapse when the difference is below a predetermined threshold.

In certain embodiments, the reference expression level is expression level of the corresponding gene in the corresponding immune cells that is representative for a non-relapse subject.

In certain embodiments, the reference expression level is a standard or average expression level determined from a representative population of non-relapse subjects.

In certain embodiments, the subject is determined as having high risk of tumor relapse when the difference reaches or exceeds a predetermined threshold, or the subject is determined as having low risk of tumor relapse when the difference is below a predetermined threshold.

In one aspect, the present disclosure provides a method of assessing therapeutic efficacy in a subject having been treated with an anti-tumor therapy in combination with at least one dose of tumor neoantigen vaccine, comprising:
a) determining the level of one or more genes in one or more immune cells from a sample obtained from the subject after the treatment; wherein the one or more genes are selected from the group consisting of: AC092490.1, ALDH1L2, LILRB5, PALD1, PKP2 and TRAV35, or are any combination thereof;
b) comparing the level of the one or more genes determined in step a) with a reference level to determine difference from the reference level; and
c) assessing the therapeutic efficacy in the subject based on the difference determined in step b).

In certain embodiments, the one or more immune cells are selected from the group consisting of: CD8+ T cells, CD4+ T cells, monocytes, B cells, NK cells and macrophages.

In certain embodiments, the one or more immune cells are CD4+ T cells and the one or more genes is LILRB5.

In certain embodiments, the one or more immune cells are monocytes and the one or more genes are selected from the group consisting of: ALDH1L2 and PKP2, or are any combination thereof.

In certain embodiments, the one or more immune cells are B cells and the one or more genes are selected from the group consisting of: AC092490.1, PALD1 and TRAV35, or are any combination thereof.

In certain embodiments, the expression level of a given gene is represented by percentage of a given type of immune cells that express the given gene.

In certain embodiments, the reference expression level is expression level of the corresponding gene in the corresponding immune cells from a sample obtained from the subject before receiving the anti-tumor therapy.

In certain embodiments, the anti-tumor therapy comprises a PD-1 antagonist.

In certain embodiments, the subject has shown tumor relapse after tumor neoantigen vaccination.

In certain embodiments, the subject has received tumor resection surgery before receiving first dose of the tumor neoantigen vaccine, optionally the subject had no chemotherapy before the resection surgery.

In certain embodiments, tumor tissue, adjacent tissue and/or a peripheral blood sample of the subject have been analysed to identify one or more tumor-specific mutations in the subject.

In certain embodiments, the tumor neoantigen vaccine is prepared based on the identified tumor-specific mutations.

In certain embodiments, the subject has been diagnosed to have pancreatic cancer, optionally pancreatic ductal adenocarcinoma.

In certain embodiments, the sample comprises or is derived from peripheral blood mononuclear cells (PBMCs), a blood sample, or tumor infiltrating immune cells.

In certain embodiments, the level of the one or more genes is measured via an amplification assay, a hybridization assay, sequencing methods (e.g. single-cell sequencing), or an immunoassay (e.g. flow cytometry or immunohistochemistry).

In one aspect, the present disclosure provides a kit for assessing responsiveness of a subject to a tumor neoantigen vaccine, comprising one or more reagents for detecting expression level of one or more genes in one or more immune cells from a sample obtained from the subject; wherein the one or more genes are selected from the group consisting of: AC011815.2, ANKRD29, AP3M2, ARL5B, ATF4, ATP5F1B, C15orf54, CHCHD2, COL4A3BP, COPE, CTTNBP2, DDOST, DERL1, DNPH1, EGF, FERMT3, FOSL2, GCH1, GK, GLA, LMAN2, LRRK1, MAP9, MN1, MTDH, NFIL3, NUAK1, PDE4D, PIM1, PRKACA, PRMT1, SERTAD2, SLC16A6, SYT17, TMED10, TMEM258, TRDV1, WDFY3, ZBTB43 and ZDHHC7, or are any combination thereof.

In one aspect, the present disclosure provides a kit for assessing responsiveness of a subject to at least one priming dose of tumor neoantigen vaccine, comprising one or more reagents for detecting expression level of one or more genes in one or more immune cells from a sample obtained from the subject following the at least one priming dose of tumor neoantigen vaccine; wherein the one or more genes are selected from the group consisting of: AC011815.2, ATF4, C15orf54, DDOST, DERL1, DNPH1, EGF, FERMT3, FOSL2, GCH1, GK, LMAN2, MAP9, NUAK1, PDE4D, PRKACA, SERTAD2, SLC16A6, TMED10, TMEM258, WDFY3, ZBTB43 and ZDHHC7, or are any combination thereof.

In one aspect, the present disclosure provides a kit for assessing responsiveness of a subject to at least one boosting dose of tumor neoantigen vaccine, comprising one or more reagents for detecting expression level of one or more genes in one or more immune cells from a sample obtained from the subject during the boosting phase; wherein the one or more genes are selected from the group consisting of: AC011815.2, ANKRD29, AP3M2, ARL5B, ATP5F1B, C15orf54, CHCHD2, COL4A3BP, COPE, CTTNBP2, EGF, FERMT3, FOSL2, GK, GLA, LRRK1, MAP9, MN1, MTDH, NFIL3, NUAK1, PIM1, PRMT1, SERTAD2, SLC16A6, SYT17, TMED10, TRDV1 and ZDHHC7, or are any combination thereof.

In one aspect, the present disclosure provides a kit for predicting the risk of tumor relapse in a subject before or after receiving at least one dose of tumor neoantigen vaccine, comprising one or more reagents for detecting expression level of one or more genes in one or more immune cells from a sample obtained from the subject; wherein the one or more genes are selected from the group consisting of: ABCA13, AC022726.2, AC105094.2, AC243829.2, AL021807.1, AL391807.1, AL662907.3, APOBEC3C, ATP1B3, ATP6V1B2, C1QBP, CBX5, CCNE2, CD63, CEBPA, CHP1, CLN8, CMBL, CNIH4, COL4A3BP, CPNE3, DACH1, DNAJC12, DNAJC3, DOC2B, EEA1, ENAM, ERG, FAM43A, FBXO43, FIGN, GCA, GNG4, GNLY, GOLIM4, GPM6B, GPR171, GPRC5D, HHEX, HID1, ILF2, IRF2BP2, KIF22, LACC1, LIPA, MANEA, MECOM, MID1IP1, MX2, MYOM2, NABP1, NEFH, NFE4, NLN, OXCT2, P3H2, PI3, PIM1, PLAU, PRSS3, PSMA3, RAB10, RAB20, RASGRP2, RBMS3, RPL23, RPS6KA5, RPS8, RUNX1, SAMD3, 11-Sep, SERTAD2, SIGLEC6, SIT1, SOCS1, SSR1, ST6GALNAC1, SYNE2, TRAV16, TREM2, TXNDC15, TXNDC17, UAP1, UFM1, UGT2B17, XPNPEP1, ZBTB16, ZNF608 and STAT1, or are any combination thereof.

In one aspect, the present disclosure provides a kit for assessing therapeutic efficacy in a subject having been treated with an anti-tumor therapy in combination with at least one dose of tumor neoantigen vaccine, comprising one or more reagents for detecting the level of one or more genes in one or more immune cells from a sample obtained from the subject after the treatment; wherein the one or more genes are selected from the group consisting of: AC092490.1, ALDH1L2, LILRB5, PALD1, PKP2 and TRAV35, or are any combination thereof.

In one aspect, the present disclosure provides a method of enhancing cell-mediated immunity in a subject in need thereof, comprising administering to the subject an effective amount of a PPP1R15A agonist, thereby enhancing cell-mediated immunity in the subject.

In certain embodiments, the cell-mediated immunity is T cell-mediated immunity.

In one aspect, the present disclosure provides a method of stimulating and/or expanding T cells in a subject in need thereof, comprising administering to the subject an effective amount of a PPP1R15A agonist, thereby stimulating and/or expanding T cells in the subject.

In one aspect, the present disclosure provides a method of potentiating immunogenicity of an immunogenic composition in a subject, comprising administering to the subject an effective amount of a PPP1R15A agonist in combination with the immunogenic composition.

In certain embodiments, the immunogenic composition is a vaccine or a composition for CAR-T treatment.

In certain embodiments, the vaccine is a tumor vaccine.

In certain embodiments, the subject is suffering from a condition that would benefit from upregulation of immune response.

In certain embodiments, the subject is determined to have reduced expression level of PPP1R15A.

In certain embodiments, the condition is tumor, infectious disease, cardiovascular disease or inflammatory disease.

In certain embodiments, the condition is tumor or infectious disease.

In certain embodiments, the tumor is selected from the group consisting of brain cancer, renal cell carcinoma, ovarian cancer, gastric cancer, bladder cancer, breast cancer, ovarian cancer, prostate cancer, colon cancer, lung cancer, squamous cell carcinoma of head and neck, colorectal cancer, melanoma, and myeloma.

In certain embodiments, the subject is receiving a therapy whose efficacy can be potentiated by enhanced cell-mediated immunity.

In certain embodiments, the therapy is an anti-tumor therapy or anti-infectious therapy.

In certain embodiments, the anti-tumor therapy is selected from the group consisting of chemotherapy, targeted therapy, immunotherapy, cell therapy (e.g. CAR-T therapy), and tumor vaccine.

In one aspect, the present disclosure provides a method of treating a condition that would benefit from upregulation of immune response in a subject in need thereof, comprising administering to the subject an effective amount of a PPP1R15A agonist.

In certain embodiments, the condition is tumor, infectious disease, cardiovascular disease or inflammatory disease.

In certain embodiments, the condition is tumor or infectious disease.

In certain embodiments, the tumor is selected from the group consisting of brain cancer, renal cell carcinoma, ovarian cancer, gastric cancer, bladder cancer, breast cancer, ovarian cancer, prostate cancer, colon cancer, lung cancer, squamous cell carcinoma of head and neck, colorectal cancer, melanoma, and myeloma.

In certain embodiments, the subject is diagnosed as having reduced expression level of PPP1R15A.

In certain embodiments, the PPP1R15A agonist is administered in combination with a therapy that treats the condition.

In certain embodiments, the therapy is an anti-tumor therapy or anti-infectious therapy.

In certain embodiments, the anti-tumor therapy is selected from the group consisting of chemotherapy, targeted therapy, immunotherapy, cell therapy (e.g. CAR-T therapy), tumor vaccine and CAR-T therapy.

In certain embodiments, the PPP1R15A agonist is selected from the group consisting of full-length mRNA of PPP1R15A, and a synthetic cannabinoid (WIN 55,212-2 mesylate [WIN]).

In one aspect, the present disclosure provides a method of promoting clonal expansion of T cells, comprising treating the T cells with an effective amount of a PPP1R15A agonist under suitable conditions, thereby allowing clonal expansion of the T cells.

In certain embodiments, the T cells are memory T cells.

In one aspect, the present disclosure provides a method of promoting T cell activation or promoting cytotoxicity of T cells, comprising treating the T cells with an effective amount of a PPP1R15A agonist under suitable conditions.

In certain embodiments, the T cells are cultured with the PPP1R15A agonist in combination with a second agent for stimulating the T cells.

In certain embodiments, the PPP1R15A agonist is selected from the group consisting of full-length mRNA of PPP1R15A, and a synthetic cannabinoid (WIN 55,212-2 mesylate [WIN]).

In one aspect, the present disclosure provides a composition comprising the T cells prepared using the method provided herein.

In one aspect, the present disclosure provides a method of treating a condition that would benefit from upregulation of immune response in a subject in need thereof, comprising administering to the subject the composition provided herein.

In one aspect, the present disclosure provides a method of evaluating activation state, or activity or cytotoxicity of T cells, comprising detecting expression level of PPP1R15A in the T cells, wherein an increase in the expression level of PPP1R15A relative to a control T cell is indicative of cytotoxicity of the T cells.

In certain embodiments, the T cells are CAR-T cells, or TCR-T cells.

In one aspect, the present disclosure provides a method of preparing a population of T cells for cell therapy, the method comprising:
a) identifying the population of T cells having increased expression level of PPP1R15A relative to a control T cell; and
b) selectively enriching the identified T cells for cell therapy.

In one aspect, the present disclosure provides a method of converting a first population of inactive T cells to a second population of active T cells, the method comprising:
a) providing the first population of inactive T cells, wherein the inactive T cells do not show an increase in the expression level of PPP1R15A relative to a control T cell;
b) treating the first population of inactive T cells with an effective amount of a PPP1R15A agonist under suitable conditions; and
c) detecting expression level of PPP1R15A in the population of T cells obtained in step b), wherein an increase in the expression level of PPP1R15A relative to a control T cell is indicative of successful conversion to the second population of active T cells.

In one aspect, the present disclosure provides a method of preparing a population of T cells for cell therapy, the method comprising:
a) identifying the population of T cells as inactive if it does not show an increase in the expression level of PPP1R15A relative to a control T cell; and
b) treating the identified inactive population of T cells with an effective amount of a PPP1R15A agonist under suitable conditions, thereby obtaining a population of activated T cells.

In certain embodiments, the PPP1R15A agonist is selected from the group consisting of full-length mRNA of PPP1R15A, and a synthetic cannabinoid (WIN 55,212-2 mesylate [WIN]).

In one aspect, the present disclosure provides a composition comprising the population of T cells prepared or converted by the method provided herein.

In one aspect, the present disclosure provides a method of treating a condition that would benefit from upregulation of immune response in a subject in need thereof, comprising administering to the subject the population of T cells prepared or converted by the method provided herein.

In one aspect, the present disclosure provides method of reducing cell-mediated immunity in a subject in need thereof, comprising administering to the subject an effective amount of a PPP1R15A antagonist, thereby reducing cell-mediated immunity in the subject.

In certain embodiments, the cell-mediated immunity is T cell-mediated immunity.

In one aspect, the present disclosure provides a method of deactivating T cells in a subject in need thereof, comprising administering to the subject an effective amount of a PPP1R15A antagonist, thereby deactivating T cells in the subject.

In certain embodiments, the subject has been determined to have T cells having an increase in the expression level of PPP1R15A relative to a control T cell.

In certain embodiments, the subject is suffering from a condition characterized in excessive cell-mediated immunity.

In one aspect, the present disclosure provides a method of treating a condition that would benefit from downregulation of immune response in a subject in need thereof, comprising administering to the subject an effective amount of a PPP1R15A antagonist.

In certain embodiments, the condition is an autoimmune disease, organ or tissue graft rejection, a graft-versus-host disease, an inflammatory disease, an infectious disease, or a cancer.

In certain embodiments, the condition is where the subject is diagnosed as having increased expression level of PPP1R15A.

In certain embodiments, the PPP1R15A antagonist is selected from the group consisting of Guanabenz and Sephin1.

In certain embodiments, the control T cell is CD8+ T cell.

In one aspect, the present disclosure provides a method of predicting the risk of developing a disease or condition associated with downregulation of immune response in a subject, comprising
a) determining the expression level of PPP1R15A from a sample obtained from the subject;
b) comparing the level determined in step a) with a reference level to determine difference from the reference level, and
c) predicting the risk of developing the disease or condition associated with downregulation of immune response based on the difference determined in step b).

In certain embodiments, the subject is predicated as having the risk of developing the disease or condition associated with downregulation of immune response, when the difference indicates a reduction in expression level of PPP1R15A relative to a reference level.

In certain embodiments, the disease or condition is tumor, infectious disease, cardiovascular disease or inflammatory disease.

In certain embodiments, the tumor is selected from the group consisting of brain cancer, renal cell carcinoma, ovarian cancer, gastric cancer, bladder cancer, breast cancer, ovarian cancer, prostate cancer, colon cancer, lung cancer, squamous cell carcinoma of head and neck, colorectal cancer, melanoma, and myeloma.

In one aspect, the present disclosure provides a method of predicting risk of developing a disease or condition associated with upregulation of immune response in a subject, comprising
a) determining the level of PPP1R15A in the T cells from a sample obtained from the subject;
b) comparing the level of PPP1R15A determined in step a) with a reference level to determine difference from the reference level; and
c) determining the risk of developing the disease or condition in the subject based on the difference determined in step b).

In certain embodiments, the subject is determined as having a risk of developing the disease or condition associated with upregulation of immune response when the difference reaches or exceeds a first predetermined threshold.

In certain embodiments, the disease or condition is an autoimmune disease, organ or tissue graft rejection, a graft-versus-host disease, an inflammatory disease, an infectious disease, or a cancer.

In one aspect, the present disclosure provides a method of predicting likelihood of responsiveness of a subject in need thereof to the treatment of PPP1R15A agonist, comprising:
a) determining the level of PPP1R15A in the T cells from a sample obtained from the subject;
b) comparing the level of PPP1R15A determined in step a) with a reference level to determine difference from the reference level; and
c) determining the likelihood of responsiveness in the subject based on the difference determined in step b).

In certain embodiments, the subject is determined as likely to be responsive to the treatment of PPP1R15A agonist when the difference reaches or exceeds a first predetermined threshold.

### BRIEF DESCRIPTION OF FIGURES

FIG. 1A shows clinical treatment and event timeline for the 12 patients who received at least 7 doses of vaccines from surgery until the time of death or end of follow-up.

FIG. 1B shows Kaplan-Meier curves showing the relapse-free survival of 12 patients during neoantigen vaccine treatment and patients who were treated with chemotherapy after the surgery in the ICGC PACA-AU project, PACA-CA project and the Changhai Hospital (historical controls).

FIG. 1C shows Kaplan-Meier curves showing the overall survival of 12 patients during neoantigen vaccine treatment and patients who were treated with chemotherapy after the surgery in the ICGC PACA-AU project, PACA-CA project and the Changhai Hospital (historical controls).

FIG. 1D shows serum CA19-9 levels were examined before the surgery, before the vaccination, during the vaccination and follow-up. Levels of CA19-9 were reported as U/mL. The y-axis was log 2 transformed values. The black horizontal dashed line indicates the upper limit of the normal reference (37 U/mL) and the red horizontal dashed line indicates the level of 90.65 U/mL (2.45 times of 37 U/mL [2.45 times elevated CA19-9 values shows recurrence with 90% sensitivity and 83.33% specificity]).

FIG. 1E shows serum CA72-4 levels were examined before the surgery, before the vaccination, during the vaccination and follow-up. Levels of CA72-4 were reported as U/mL. The y-axis was log 2 transformed values. The black horizontal dashed line indicates the upper limit of the normal reference (9.8 U/mL) and the red horizontal dashed line indicates the level of 14.7 U/mL (1.5 times of 9.8 U/mL).

FIG. 2A shows the diversity of expression of TCR genes in single-cell 3′ library transcriptome sequencing. The changes of the diversity during the treatment in the CD4+, CD8+ and other T cells respectively. The higher the Shannon index, the higher the expression diversity.

FIG. 2B shows the comparison of the diversity of TCR clones in single-cell TCR sequencing data.

FIG. 2C shows comparison of cell proportions of T cell subtypes in the different days during vaccine treatment in the single-cell RNA sequencing data. Top panel, Changes of cell proportions of CD8+, CD4+ and CD4/CD8 low T cells during the treatment. Bottom panel, changes of cell proportions of effector T (Teff), exhausted T (Tex), T helper 1 (Th1), T helper 9 (Th9), memory T (Tmem) and regulatory T (Treg) cells during the treatment. Percent value were transformed by the hyperbolic arcsine function. The values in the y axis indicate the relative changes compared to the pre-vaccine. The proportion of cells in the pre-vaccination for all patients are all normalized to 0 (horizontal dashed line). The circle dots represent patients with tumor relapse and triangles represent patients with non-relapse. The red background box represents that the relative cell proportion of the patients on that day are significantly greater than those in pre-vaccine, and the blue background box represents that the cell proportion of relapsed and non-relapsed patients is significantly different. All p-values were calculated using LMM (see Method) and were corrected for the multiple comparison using the Benjamini-Hochberg adjustment. The P-value<0.05 was considered as the significance for all the test.

FIG. 2D shows types and percentages of changes in the number of TCR clones that had the same sequence with the TCR identified in the patients' tumors during the treatment.

FIG. 2E shows differences in percentage of 4-1BB+ and CD69+ cell populations in CD8+ (top) and CD4+ (bottom) T cells using flow cytometric. * indicates the significant differences by using the LMM method.

FIG. 2F shows function enrichment analysis of significantly changed genes in CD4+, CD8+ and CD4/CD8 low T cells comparing the gene expression of pre-vaccine, priming and booster phases.

FIG. 2G shows function enrichment analysis of significantly differently expressed genes between patients with tumor relapse and without relapse. All enrichment analyses were performed using the annotated genes from the hallmark gene sets and ontology gene sets in the MSigDB database.

FIG. 2H shows changes in average expression levels of modules for IFN-γ response pathway genes and G2M checkpoint genes in CD4+, CD8+ and CD4/CD8low T cells.

FIG. 2I shows the significant difference in the percent of cells that positively expressed STAT1 between patients with tumor relapse and without relapse after the neoantigen vaccination. The proportion of cells in the pre-vaccination for all patients are all normalized to 0 (horizontal dashed line). The lines indicate the average values and the vertical lines is the error bars, SEM.

FIG. 3A shows IFN-γ secretion induced by four neoantigen peptides of the patient No. 4 using the ELISpot assay.

FIG. 3B shows the effect of tumor removal by the four neoantigen peptides of the patient P4 against autologous tumor and the blank control.

FIG. 3C shows Uniform Manifold Approximation and Projection (UMAP) plot showing the cell populations and cells of different groups under the stimulation of different neoantigen peptides in the CD8+ T cells using the scRNA-seq data.

FIG. 3D shows the markers used to annotate the cell types for central memory cells (CM) and Tumor reactive cells (T-Reactive) and expression levels of marker genes (MX1 and STAT1) in those two subpopulation.

FIG. 3E shows the percentage of cell populations in the CD8+ T cells after the in vitro stimulation of those four neoantigen peptides using the single-cell transcriptome sequencing. Red stars indicate the major subtypes (CM and T-Reactive) in the stimulation of PCNAT-4-2 and PCNAT-4-3 peptides.

FIG. 3F shows the comparison of TCR clones of PCNAT-4-2 and PCNAT-4-3 stimulation to those in blank control using the single-cell TCR sequencing. If the TCR clone did not found in the blank control, the cells with that TCR were defined as ‘different’, otherwise, the cells were classified into ‘Decays’ or ‘Expands’ according the occurrence frequency of that TCR compared to the blank. Y axis indicates the number of cells contain above types of TCR clones.

FIG. 3G shows the expression levels of MX1 between pre- and post-vaccination in CD8+ T cells in the patient P5 and P9.

FIG. 3H shows the up-regulation of average proportion of MX1+ cells in CD8+ T cells in the blood of patients after the neoantigen vaccines treatment (P<0.05, LMM test, see Method). The proportion of cells in the pre-vaccination for all patients are all normalized to 0 (horizontal dashed line). The circle dots represent patients with tumor relapse and triangles represent patients with non-relapse.

FIG. 3I shows correlation between MX1 expression and gene expression related to cytotoxicity and IFN-γ in relapsed and non-relapsed patients.

FIG. 3J shows qPCR assay detecting the expression levels of MX1 after knockdown by siRNA. NC stands for negative control oligonucleotides. Student's t test was used. Bars, mean; error bars, SEM; ** indicates P<0.01. ns is non-significant.

FIG. 3K shows the percent of tumor removal along with the time for the inhibition of MX1 by siRNA and negative control oligonucleotides in PBMC cells. ** indicates P<0.01.

FIG. 4A shows the significant differences of genes that are involved in activation of T cells in CD4+, CD8+ and CD4/CD8low T cells comparing the boosting and priming phases in patients treated with adjuvant anti-PD1 and with only neoantigen vaccines. Circles with red border indicates genes that are only significant changed in patients with adjuvant anti-PD1.

FIG. 4B shows function enrichment analysis of significantly changed genes in CD8+ T cells comparing the gene expression of between boosting and priming phases in patients treated with adjuvant anti-PD1 and with only neoantigen vaccines.

FIG. 4C shows changes in average expression levels of modules for response to molecule of bacterial origin function genes and cellular response to biotic stimulus function genes in CD8+ T cells for the combined anti-PD1 and only neoantigen vaccine patients.

FIG. 4D shows survival analysis of genes related to the effect of combination of neoantigen vaccines and anti-PD1. Gene hazard ratios of genes in each gene set (columns) are shown by treatment arms (rows) in the clinical trial. Vertical lines mark hazard ratio=1. Vertical jitter distinguishes overlapping dots. Hazard ratio<1 (left of vertical lines) indicates greater PFS. Mean hazard ratio and one-sided P values are shown from a one-sample z-test on hazard ratios for genes in the signature, highlighted with associated data in red when P<0.01.

FIG. 5A shows stacked bar plot showing the percentage of different immune cells in each stage of vaccination in each patient. The percentage was calculated using the single-cell transcriptome sequencing and the types of immune cells were defined according to the gene expression of known makers. The total percentage was normalized to 1 for each sample.

FIG. 5B shows the changes of the relative percent of megakaryocyte, B cell, Monocyte, and NK cells after the first neoantigen vaccination. The proportion of cells in the pre-vaccination for all patients are all normalized to 0 (horizontal dashed line). Pattern ‘up’ patients were defined as having at least 3 time points with a ratio of cell population greater than 20% of the maximum value; Pattern ‘down’ patients were defined as having at least 3 time points with a ratio of cell population less than 20% of the minimum value; others were defined as ‘flat’. * indicates the significant difference (P<0.05) between the priming, booster phase and the pre-vaccination using the LMM method (see Supplementary Method).

FIG. 6A shows the percentage of TCR clones, which were also identified in the tumors, in the peripheral blood of 12 patients. The TCR clones in tumors were identified by MiXCR using the RNA bulk sequencing data of tumors. The TCR clones in PBMCs were identified by single-cell TCR sequencing.

FIG. 6B shows the heatmap of the ssGSEA scores of immune cells in the tumors of 12 patients. The patients were divided into 3 groups (high, median and low infiltration) based on the average scores of immune cells.

FIG. 7 shows identification of differences in immune cell populations between pre- and post-vaccination using the flow cytometric analysis. Red is for non-relapse patients and blue is for relapse patients. * indicates the significant difference (P<0.05).

FIG. 8 shows differential expression in genes that are related with activation of T cells for priming versus pre-vaccine phases (top) and boosting versus pre-vaccine phases (bottom) in the blood of patients. Differential expression was performed among CD4+, CD8+ and CD4/CD8low T cells respectively. A significant differently expressed gene was shown as a dot in the plot. For each gene, the average log fold change and the percentage of cells that express the gene above background are compared between the 2 phases. For example, a delta percent of +0.1 indicates that 10% more cells in the priming phase express the gene above background than those in the pre-vaccine. The top 20 changed genes for each function (Cytokine, Cytotoxic, IFN response and Proliferation) were labeled by their gene names.

FIG. 9A shows gene Set Enrichment Analysis (GSEA) of the interferon gamma response pathway for the significantly changed genes in CD8+ T cells.

FIG. 9B shows changes in average expression levels of modules for IFN-γ response pathway genes and G2M checkpoint genes in T cells for the relapse and non-relapse patients.

FIG. 10A shows differential expression in genes that are related with activation of T cells (top) and antigen-presenting cells (bottom) for non-relapse versus relapse patients. Differential expression was performed among pre-vaccine, priming and boosting phases and CD4+, CD8+ and CD4/CD8low T cells respectively. A significant differently expressed gene was shown as a dot in the plot. For each gene, the average log fold change and the percentage of cells that express the gene above background are compared between the 2 phases. For example, a delta percent of +0.1 indicates that 10% more cells in the priming phase express the gene above background than those in the pre-vaccine. The top 20 changed genes for each T cell subpopulation were labeled by their gene names.

FIG. 10B shows differential expression of GSVA scores for STAT1+ T cells between relapse and non-relapse patients.

FIG. 11 shows Ex vivo IFN-γ ELISPOT of PBMCs for each single neoantigen peptide used in the vaccines of patients with triplicate wells per time point. Normalized spot count was calculated using the number of spot-forming count in stimulation of peptides minus the number in the corresponding blank control.

FIG. 12A shows the intersect of marker genes of TReactive and CM.

FIG. 12B shows the heatmap of the number of significantly changed genes respectively overlapping with the marker genes of TReactive and CM in the priming and booster phases compared with pre-vaccination.

FIG. 12C shows changes in average expression levels of modules for TReactive gene signature and CM gene signature in CD4+, CD8+ and CD4/CD8low T cells during the vaccination.

FIG. 12D shows comparison of average expression levels of modules for TReactive gene signature and CM gene signature between relapse and non-relapse patients during the vaccination.

FIG. 13A shows UMAP plot showing the cell populations and expression levels of the IFN-γ response pathway related genes under the stimulation of the neoantigen peptides of patient P4 in the CD8+ T cells using the scRNA-seq data.

FIG. 13B shows the up-regulation of average proportion of corresponding genes above in CD8+ T cells in patients after the neoantigen vaccines treatment (P<0.05, LMM test, see Method). The proportion of cells in the pre-vaccination for all patients are all normalized to 0 (horizontal dashed line). The circle dots represent patients with tumor relapse and triangles represent patients with non-relapse.

FIG. 14A shows the percentage of cell populations in the CD4+ (left) and CD4/CD8 low T cells (right) after the in vitro stimulation of the neoantigen peptides.

FIGS. 14B-14C show UMAP plot showing the cell populations, cells of different groups and expression levels of marker genes for the increased cell population under the stimulation of PCNAT-4-2 and PCNAT-4-3 in the CD4+ (B) and CD4/CD8 low T cells (C) using the scRNA-seq data.

FIGS. 15A-15B shows the average proportion of marker genes identified from the in vitro stimulation of the neoantigen peptides in CD4+ (A) and CD4/CD8 low T cells (B) in the blood of patients during the neoantigen vaccines treatment. The proportion of cells in the pre-vaccination for all patients are all normalized to 0 (horizontal dashed line). The circle dots represent patients with tumor relapse and triangles represent patients with non-relapse.

FIG. 16A shows the heat map showing the genes with top 50 correlation coefficient with MX1 in immune cells. Left annotation are cell types of immune cells and involved gene functions including antigen presentation, immune check point, ligand/receptor, molecular function of MHC class I/II protein complex, T cell activation and molecular function of Toll-like receptors respectively.

FIG. 16B shows the correlation of CD40 and CD226 with MX1 in CD8+ T cells.

FIG. 16C shows the changes of proportion of CD40+ and CD226+ cells in CD8+ T cells during the vaccination.

FIG. 16D shows the gene interaction network in different types of immune cells for the genes that are correlated with the MX1 in CD8+ T cells.

FIG. 17 shows the sequences of MX1 and PPP1R15A.

FIG. 18A shows overall experimental design. Mice were first separated into the normal group and Sephin1 group and injected with solvent or Sephin1 for two weeks. Then, B16F1 cells were injected. PBMCs were collected on Days 0 and 15 after tumor injection, and immune cells were isolated from tumor tissues on Day 15 and subjected to single-cell sequencing.

FIG. 18B shows tumor growth curves of the normal group and Sephin1 group (n=8). The tumor volume in the Sephin1 group was significantly higher than that in the normal group. Multiple t tests was used without adjustments, and each row was analyzed individually. Bars: mean; error bars: SEM; *: p<0.05.

FIG. 18C shows tumor weights of the two groups (n=8) on Day 15. The tumor weight of the Sephin1 group was significantly higher. Unpaired, two-tailed t test was used. Bars: mean; error bars: SEM; **: p<0.01.

FIG. 18D shows SCENIC analysis based on the single-cell sequencing data for different samples. The Atf3 regulon had higher activity in the Sephin1 group in all three sample types. The number of genes in each regulon is shown in brackets.

FIG. 18E shows tumor images from the normal (top) and Sephin1 (bottom) groups.

FIG. 18F shows cell type annotation of all 12 samples. Sixteen cell types were identified.

FIG. 18G shows cell distribution of all 6 sample types (2 samples of each type).

FIG. 18H shows distribution of major cell types in different sample types. Day 0_Blood-PBMCs in blood samples collected on Day 0. Day 15_Blood-PBMCs in blood samples collected on Day 15. Day 15_Tumor-Cd45+ immune cells in tumor tissues collected on Day 15. Chi square test was used to evaluate the distribution of normal/Sephin1 groups. *: p<0.05; **: p<0.01; ***: p<0.001; ****: p<0.0001.

FIG. 19A shows a UMAP plot showing the detailed cell type annotation of lymphocytes NK-NK cells. NKT-NKT cells. Exhausted_Cd8-exhausted Cd8+ T cells. Cyt_Cd8-cytotoxic Cd8+ T cells. Naïve_Cd8-naïve Cd8+ T cells. Effector_Cd4-effector Cd4+ T cells. Treg_Cd4-regulatory T cells. Naïve_Cd4 naïve Cd4+ T cells.

FIG. 19B shows gene markers of different types of lymphocytes. Klrb1-c NK cells. Cd3d-T cells. Cd4-Cd4+ T cells. Cd8a-Cd8+ T cells. Gzmb-cytotoxic T cells. Foxp3-regulatory T cells. Pdcd1, Havcr2, Lag3-exhausted T cells. Cd44-Sell(Cd62L)+-naïve T cells. Cd44+Sell(Cd62L)-effector T cells.

FIG. 19C shows distribution of lymphocyte types. The percentages of NK cells and exhausted Cd8+ T cells in the Sephin1 group were significantly reduced compared with those in the normal group. The percentages of NKT cells and cytotoxic Cd8+ T cells were significantly reduced in the blood and tumor tissue on Day 15 but not in the blood on Day 0. Chi square test was used to evaluate the distribution of normal/Sephin1 groups. *: p<0.05; **: p<0.01; ***: p<0.001; ****: p<0.0001.

FIG. 19D shows percentage changes of Cd4+ T cells, Cd8+ T cells, NK cells, regulatory T cells, exhausted Cd8+ T cells, and activated Cd8+ T cells in all immune cells determined by FACS (n=8). Wilcoxon test was used in each cell type without adjustment and p values was marked in the graph.

FIG. 20A shows GSEA of Cd8+ T cells in tumor tissue. Right: pathways that were upregulated in the Sephin1 group. Left: pathways that were downregulated in the Sephin1 group. Pink/blue: significant up/downregulated pathways in the Sephin1 group. Gray: nonsignificant pathways in the Sephin1 group.

FIG. 20B shows expression scores for cytotoxicity-related genes in Cd8+ T cells. The expression score was significantly lower in the tumor tissue in the Sephin1 group. The p values was calculated by Wilcoxon test and adjusted by Bonferroni-Holm method.

FIG. 20C shows expression scores for genes related to the positive regulation of Cd8+ cell cytotoxicity. Expression scores were significantly downregulated in the Sephin1 group in the blood samples collected on Day 0 and Day 15 and tumor tissues. The p values was calculated by Wilcoxon test and adjusted by Bonferroni-Holm method.

FIG. 20D shows expression scores for genes related to the positive regulation of NK-cell activity. In the blood and tumor tissue samples collected on Day 15, all the expression scores in the Sephin1 group were significantly lower than those in the normal group. The p values was calculated by Wilcoxon test and adjusted by Bonferroni-Holm method.

FIG. 20E shows expression scores for genes related to NK-cell activity. For the blood and tumor tissue samples collected on Day 15, the scores were significantly lower in the Sephin1 group. The p values was calculated by Wilcoxon test and adjusted by Bonferroni-Holm method.

FIG. 20F shows SCENIC analysis of different lymphocyte subtypes between normal and Sephin1 group. The activity of the Atf3 regulon was upregulated in exhausted Cd8+ T cells, cytotoxic Cd8+ T cells, NK cells and NKT cells but downregulated in regulatory T cells.

FIG. 21A shows distribution of different TCR clonotypes based on clonotype frequency: hyperexpanded, large, medium and small (from high to low). Clonotypes in the Sephin1 group tended to have a lower frequency.

FIG. 21B shows distribution of cells belonging to different TCR types. The percentage of cells with a higher TCR frequency, including the hyperexpanded and large TCR types, was significantly lower in the Sephin1 group for all three sample types. Chi square test was used to evaluate the distribution of normal/Sephin1 groups. *: p<0.05; **: p<0.01; ***: p<0.001; ****: p<0.0001.

FIG. 21C shows UMAP plot of the distribution of all four TCR types.

FIG. 21D shows UMAP plot of the TCR types split by group. All the cells belonging to the hyperexpanded TCR type were in the normal group.

FIG. 21E shows distribution of the four TCR types in different cell types. In addition to T cells, macrophages also exhibited populations in the hyperexpanded, large and small types.

FIG. 21F shows genes specifically expressed in different TCR types. The hyperexpanded type had higher expression activity, and the expression levels of cytotoxicity-related genes, such as Gzmb and Gzmk, were also significantly higher.

FIG. 21G shows GSVA of all four TCR types. Enrichment analysis was performed based on the biological process database for GO. The top 5 most highly enriched pathways for each cell type are displayed.

FIG. 21H shows GSEA of the hyperexpanded type. NES-normalized enrichment score. Significance was calculated as −log₁₀(P).

FIG. 22A shows subtypes of macrophages named by the specifically highly expressed genes of each cluster. Nine subtypes were identified.

FIG. 22B shows gene markers of each macrophage subtype.

FIG. 22C shows distribution of macrophage subtypes. Chil3+ and Hcar2+ macrophages mainly existed in the Sephin1 group. Chi square test was used to evaluate the distribution of normal/Sephin1 groups. *: p<0.05; **: p<0.01; ***: p<0.001; ****: p<0.0001.

FIG. 22D shows distribution of macrophage subtypes in different tissues. Chil3+, Fn1+ and Ace+ macrophages mainly existed in the blood, and the other subtypes mainly existed in tumor tissues.

FIG. 22E shows characteristics of the M1-M2 polarization pattern of all macrophages. The M1_to_M2 score was calculated by subtracting the M2 expression score from the M1 expression score. A higher M1_to_M2 score indicated that the cells tended to exhibit M1 polarization. Macrophages in the blood and tumor tissues collected on Day 15 in the Sephin1 group tended to exhibit M2 polarization over M1 polarization. The p values was calculated by Wilcoxon test and adjusted by Bonferroni-Holm method.

FIG. 22F shows M1_to_M2 scores of macrophage subtypes. Subtypes that mainly existed in the blood, including Ace+, Chil3+ and Fn1+ macrophages, were not significantly different between the normal and Sephin1 groups. Subtypes in tumor tissues tended to exhibit M2 polarization in the Sephin1 group, except for Retnla+ and Hcar2+ macrophages, which each contained a small number of cells. The p values was calculated by Wilcoxon test and adjusted by Bonferroni-Holm method.

FIG. 23A shows distribution of TCR+ macrophages (TCR_Mac) and other conventional macrophages (Conventional_Mac).

FIG. 23B shows expression of gene markers of T cells and macrophages in TCR+ macrophages.

FIG. 23C shows GSEA of differentially expressed genes between TCR+ macrophages and conventional macrophages in tumors. Upregulated genes were enriched in pathways related to T-cell activities.

FIG. 23D shows distribution of different TCR types in macrophage subtypes. TCR+ macrophages were mostly enriched in Fscn1+ macrophages.

FIG. 23E shows M1_to_M2 scores of conventional macrophages and TCR+ macrophages in tumor samples. TCR+ macrophages tended to be more M1 polarized than conventional macrophages in the normal group and were more significantly affected by Sephin1. The p values was calculated by Wilcoxon test and adjusted by Bonferroni-Holm method.

FIG. 23F shows distribution of different TCR types in macrophages. The percentages of highly proliferated TCR types, i.e., the hyperexpanded and large TCR types, were downregulated in the Sephin1 group. Chi square test was used to evaluate the distribution of normal/Sephin1 groups. *: p<0.05; **: p<0.01; ***: p<0.001; ****: p<0.0001.

FIGS. 23G-23H show distribution of macrophages which had shared TCRs with T cells in tumor microenvironment. Macrophages in the Sephin1 group had lower percentage of shared TCRs with Cd8+ T cells.

FIG. 24A shows differentiated communication strengths between the normal and Sephin1 groups of different tissues. Red: upregulated in the Sephin1 group. Blue: down-regulated in the Sephin1 group. All three tissue types had the downregulation tendency in the Sephin1 group, especially in tumor.

FIG. 24B shows mostly differentiated communication pathways between Cd8+ T cells and NK cells, which were downregulated in the Sephin1 group of all three tissue types, including communications of NK-NK, Cd8-Cd8, NK-Cd8 and Cd8-NK.

FIG. 24C shows ligand-receptor pairs of mostly downregulated pathways in the tumor tissue, including MHC-I, LCK and SELPLG pathways.

FIG. 24D shows mostly differentiated communication pathways between Cd4+ T cells and macrophages, which were upregulated in the Sephin1 group of all three tissue types.

FIG. 24E shows ligand-receptor pairs of mostly upregulated pathways in the tumor tissue, including FN1, GALECTIN, SPP1, MHC-I, THBS, TGFb, APP, THY1, TNF and CSF pathways.

FIG. 25A shows original Seurat clusters of all samples.

FIG. 25B shows cell-type specific regulators calculated based on the Regulon Specificity Score (RSS).

FIG. 25C shows UMAP plot of all samples split by sample type.

FIG. 25D shows GSVA of differentially expressed genes between samples.

FIG. 25E shows enriched genes from Atf3 regulon in the Sephin1 group either in the blood of day 0 and day 15, or in the tumor tissue of day 15.

FIG. 26A shows percentages of different cell types in different sample types.

FIG. 26B shows gene markers used for cell type annotation. Ptprc-immune cells. Cd3d, Cd4, Cd8a-T cells. Cd79a-B cells. Klrb1c-NK cells. S100a8-granulocytes. Kit-mast cells. Cd200r3-basophils. Cd68, Csf1r, Adgre1-macrophages. H2-DMb1, Cd83, Siglech-DCs.

FIG. 27A shows heatmap of gene expression generated using AddModuleScore.

FIG. 27B shows expression patterns of two cell cycle-related genes in lymphocyte subtypes.

FIG. 27C shows expression score of regulatory differentiation-related genes in the Cd4+ T cells calculated by AddModuleScore. The p values was calculated by Wilcoxon test and adjusted by Bonferroni-Holm method.

FIG. 27D shows GSEA of the most differentially expressed genes between the normal and Sephin1 groups in NK cells. Genes in pathways related to cytokine signaling and antigen processing were downregulated. Genes related to the cellular response to stress were upregulated, which showed activation of the ISR process.

FIG. 27E shows expression level of regulatory differentiation-related genes mentioned in FIG. 27C.

FIGS. 28A-28G show FACS results of antitumor-related lymphocytes in the mouse tumor tissue, including Cd4+ and Cd8+ T cells (A & B), NK cells (C), Cd4+ regular T cells (E), exhausted Cd8+ T cells (F) and active Cd8+ T cells (G).

FIG. 29A shows FACS results for IFNG and CFSE. Top: percentage of IFNG+ cells in Cd8+ T cells. Bottom: percentage of proliferated cells among Cd8+ T cells. Sephin1: Cd8+ T cells supplemented with 20 μg/mL Sephin1 and 0.2% DMSO. Control-DMSO: Cd8+ T cells treated with 0.2% DMSO. Control: Cd8+ T cells without Sephin1 or DMSO treatment.

FIG. 29B shows statistical analysis of the results for the Sephin1, Control-DMSO and Control groups. Cd8+ T cells treated with Sephin1 had significantly lower expression of IFNG and a lower proliferative ability (n=3). Multiple t tests was used without adjustments. Bars: mean; error bars: SEM; **: p<0.01; ***: p<0.001.

FIG. 30A shows distribution of unique clonotypes. The percentage of nonunique clonotypes in the tumor microenvironment was lower than that in the blood, and the tumors in the Sephin1 group had more unique TCR clonotypes than those in the normal group.

FIG. 30B shows distribution of the clonal proportion in different sample types. Clonotypes in separate samples were ranked by clone number and placed into different bins. Tissues in the Sephin1 group were more enriched in low-ranking clonotypes.

FIG. 30C shows diversity analysis of TCR clonotypes in different sample types. The Shannon index, inverse Simpson index, Chao1 estimator and abundance-based coverage estimator (ACE) were used for analysis. Samples in the Sephin1 group had higher TCR richness, and PBMCs had higher TCR richness than immune cells in tumors.

FIG. 30D shows distribution of different TCR clonotypes between samples and groups. Different samples had various TCR clonotype distribution patterns.

FIG. 31A shows Seurat cluster distribution of macrophages.

FIG. 31B shows expression patterns of M1- and M2-related genes in different sample types. Macrophages among immune cells in tumors in the Sephin1 group were more likely to express M2-related genes.

FIG. 31C shows GSVA of differentially expressed genes in macrophage subtypes.

FIG. 31D shows GSEA of macrophages among immune cells in tumors between the normal and Sephin1 groups. Genes in pathways related to T-cell activation were downregulated in the Sephin1 group.

FIG. 32A shows most highly expressed genes in macrophage subtypes. The top 10 genes in each subgroup are shown.

FIG. 32B shows expression level of M1 and M2 related genes in TCR+ macrophages and conventional macrophages in the tumor tissue.

FIG. 32C shows SCENIC analysis of macrophages among sample types. The Atf3 regulon was upregulated in the Sephin1 group in Day 0 Blood and Day 15_Tumor samples.

FIG. 32D shows GSVA of different TCR types in TCR+ macrophages. The hyperexpanded cluster was more enriched in pathways related to T cell positive regulation, mitotic chromosome condensation, cholesterol biosynthetic process, microtubule-based movement and double-strand break repair via homologous recombination.

FIG. 32E shows distribution of TCR+ and conventional macrophages in the normal group and Sephin1 group.

FIG. 33A shows percentage of macrophages in all myeloid cells and TCR+ macrophages in all macrophages in the tumor microenvironment determined by FACS (n=8). Wilcoxon test was used in each cell type without adjustment and p values was marked in the graph.

FIGS. 33B-33C show FACS results of macrophages and TCR+ macrophages.

FIGS. 33D-33E show FACS results of TCR+ macrophages in the spleen tissue of normal mice.

FIG. 33F shows distribution of macrophages having shared TCRs with T cells in different samples.

FIG. 34A shows ligand-receptor pairs of mostly downregulated pathways in the Sephin1 group in the blood of day 0 and day 15 between Cd8+ T cells and NK cells.

FIG. 34B shows ligand-receptor pairs of mostly upregulated pathways in the Sephin1 group in the blood of day 0 and day 15 between Cd4+ T cells and macrophages.

FIG. 34C shows tumor growth of 4T1 cells in female BALB/c mice (n=8). Multiple t tests was used without adjustments, and each row was analyzed individually. Bars: mean; error bars: SEM; *: p<0.05. The tumor growth rate in the Sephin1 group was higher than that in the normal group. However, the result was not as significant as that for B16F1 cells in male C57BL/6 mice.

FIGS. 35A-35F show immunofluorescence results of ligand-receptor expression in macrophages of day 15 tumor tissue. (A) Merged results of DAPI, F4/80, CD44, FN1 and SPP1, 50 μm. (B) Merged and separated results of each molecule. 20 μm.

FIG. 36 shows genes associated with significant differential changes in immune response in peripheral blood of pancreatic cancer patients treated with personalized neoantigen vaccines.

FIG. 37 shows percentage changes relative to baselines for CD69+ T, CD28+ T, B cells and NK cells in blood of patients during the vaccination using flow cytometric analysis. P6, P9, P11, P12, and P10 used the results of the flow cytometric analysis of the blood samples (B1) before vaccine treatment as the baseline, while P1, P2, P4, P7 and P8 used the results of the flow analysis of their respective earliest blood samples as the baseline because of the missing results of B1 samples. Because P5 had only one flow cytometric result from a blood sample, it was excluded from this analysis. CD69 and CD28 are the markers for activation of T cells. CD19 is the marker for B cells. Lymphocytes were sorted by low side scatter (SSC) and CD45+ in flow cytometric analysis. NK cells were sorted by CD19− and CD3− in lymphocytes. The mean±SE % of CD69+ CD8+ T cells increased from 20.4±1.4 to 34.6±6.3; CD69+ CD4+ T cells increased from 18.3±3.4 to 34±3.5; CD28+ CD8+ T cells increased from 39.9±8.6 to 56.5±10.7; CD28+ CD4+ T cells increased from 86.7±4.7 to 96.5±1.2; B cells increased from 3.8±1 to 9.6±2.2; NK cells increased from 20.7±2.7 to 25.9±3.

FIG. 38A-38B shows Dynamics of the proportion of immune cells in peripheral blood during neoantigen peptide vaccines treatment. A, time points for neoantigen vaccination and blood collection for single-cell sequencing. Blood samples are obtained a few minutes before each of administration of the vaccines. B, Dot plot showing the level of significance and direction of differences comparing each time point (column) to pre-vaccines (as the reference) in immune cells as labeled (rows): Monocyte, Macrophage, B cells, NK, T cells (rows 1-5) and their subtypes (rows 6-34). Row labels denote the positively expressed gene markers of each subtype. Red/blue dot indicates higher/lower levels of cell percent change relative to B1 (pre-vaccine); darker intensity reflects larger change; size of dot reflects strength of change; white background indicates p<0.05. In the single cell transcriptome data, we classified PBMCs into several immune cell types and their subtypes according to the genes specifically expressed and calculated the proportion of each.

FIG. 39 shows bar plots showing the percent of clonally expanded T cells compared to pre-vaccine and the percent of VRD-T cells and GD-T cells in each T cell subtype. The bottom bar plot gives the average expression levels of CD8 and CD4 genes for each subtype.

FIG. 40 shows cell abundance (%) of T-cell clonotypes with and without clonal expansion (top and bottom panels) after the vaccination at pre (shaped circle), priming (shaped Square) and boosting (shaped triangle) vaccination in blood samples of patient P6. Filled color indicates which neoantigen peptide is specifically recognized by the T cell clonotypes. We constructed a total of 5 peptide-MHC(HLA-A*11:01) tetramers corresponding to 2 TCR recognition epitopes (YVECGKAFK and KYVECGKAFK) of neoantigen-peptide-85 and 3 recognition epitopes (TTSCPECDK, TSCPECDKTSLK and GTTSCPECDK) of neoantigen-peptide-89 in P6 patients. No tetramer-positive T-cell clones targeting neoantigen-peptide-85-epitopel were detected.

FIG. 41 shows bar plots showing the percent of clonally expanded B cells compared to pre-vaccine and the percent of clonal (>1 cells) B cells in each B cell subtype. The bottom bar plot shows the average expression levels of TCL1A, AIM2 and IGHA1 genes for each subtype. Clonal expansion was classified according to the time of occurrence as 1) transient expansion, where the percentage of cells at priming was higher than pre-vaccine but lower than pre-vaccine at boosting, 2) priming expansion, where the percentage of cells at both priming and boosting was higher than pre-vaccine, and 3) boosting expansion, where the percentage of cells at priming was lower than pre-vaccine but higher than pre-vaccine at boosting.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “neoantigen” or “neoantigenic” means a class of tumor antigens that arises from a tumor-specific mutation(s) which alters the amino acid sequence of genome encoded proteins.

As used herein, the terms “prevent”, “preventing”, “prevention”, “prophylactic treatment”, and the like, refer to reducing the probability of developing a disease or condition in a subject, who does not have, but is at risk of or susceptible to developing a disease or condition.

“Treating” or “treatment” of a condition as used herein includes alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, the term “subject” refers to a human or any non-human animal or mammal (e.g., mouse, rat, rabbit, dog, cat, cattle, swine, sheep, horse or primate). In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The terms “administer”, “administering” or “administration” include any method of delivery of a pharmaceutical composition or agent into a subject's system or to a particular region in or on a subject. In certain embodiments, the agent is delivered orally, or parenterally. In certain embodiments, the agent is delivered by injection or infusion, or delivered topically including transmucosally. In certain embodiments, the agent is delivered by inhalation. In certain embodiments of the invention, an agent is administered by parenteral delivery, including, intravenous, intramuscular, subcutaneous, intramedullary injections, as well as intrathecal, direct intraventricular, intraperitoneal, intranasal, or intraocular injections. In one embodiment, the agent may be administered by injecting directly to a tumor. In some embodiments, the agent may be administered by intravenous injection or intravenous infusion. In certain embodiments, the agent can be administered by continuous infusion. In certain embodiments, administration is not oral. In certain embodiments, administration is systemic. In certain embodiments, administration is local. In some embodiments, one or more routes of administration may be combined, such as, intravenous and intratumoral, or intravenous and peroral, or intravenous and oral, or intravenous and topical, or intravenous and transdermal or transmucosal. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, continuous infusion, etc.

The term “therapeutically effective amount” or “effective amount” means the amount of a pharmaceutical agent that that produces some desired local or systemic therapeutic effect at a reasonable benefit/risk ratio applicable to any treatment. When administered for preventing a disease, the amount is sufficient to avoid or delay onset of the disease. A therapeutically effective amount or an effective amount need not be curative or prevent a disease or condition from ever occurring. In certain embodiments, a therapeutically-effective amount of a pharmaceutical agent will depend on its therapeutic index, solubility, and the like.

The term “level” with respect to a biomarker (such as MX1 and PPP1R15A and other biomarkers provided herein) refers to the amount or quantity of the biomarker of interest present in a sample. Such amount or quantity may be expressed in the absolute terms, i.e., the total quantity of the biomarker in the sample, or in the relative terms, i.e., the concentration or percentage of the biomarker in the sample. Level of a biomarker can be measured at DNA level (for example, as represented by the amount or quantity or copy number of the gene in a chromosomal region), at RNA level (for example as mRNA amount or quantity), or at protein level (for example as protein or protein complex amount or quantity).

The term “expression level” with respect to a biomarker (such as MX1 and PPP1R15A and other biomarkers provided herein) refers to the amount or quantity of the expressed biomarker, such as at mRNA level or at protein level.

The terms “determining”, “measuring” and “detecting” can be used interchangeably and refer to both quantitative and semi-quantitative determinations. Level (such as an expression level) of a biomarker (such as MX1 and PPP1R15A and other biomarkers provided herein) at DNA or RNA level can be measured by any methods known in the art, for example, without limitation, an amplification assay, a hybridization assay, or a sequencing assay. Expression level of a biomarker at protein level can be measured by any methods known in the art, for example, without limitation, immunoassays.

A nucleic acid amplification assay involves copying a target nucleic acid (e.g. DNA or RNA), thereby increasing the number of copies of the amplified nucleic acid sequence. Amplification may be exponential or linear. Exemplary nucleic acid amplification methods include, but are not limited to, amplification using the polymerase chain reaction (PCR), reverse transcriptase polymerase chain reaction (RT-PCR), quantitative real-time PCR (qRT-PCR), quantitative PCR, such as TaqMan®, nested PCR, and the like.

A nucleic acid hybridization assays use probes to hybridize to the target nucleic acid, thereby allowing detection of the target nucleic acid. Non-limiting examples of hybridization assay include Northern blotting, Southern blotting, in situ hybridization, microarray analysis, and multiplexed hybridization-based assays.

Sequencing methods allow determination of the nucleic acid sequence of the target nucleic acid, and can also permit enumeration of the sequenced target nucleic acid, thereby measures the level of the target nucleic acid. Examples of sequence methods include, without limitation, RNA sequencing, pyrosequencing, high throughput sequencing, and single-cell sequencing.

Immunoassays typically involves using antibodies that specifically bind to the biomarker polypeptide or protein (such as MX1 and PPP1R15A and other biomarkers provided herein) to detect or measure the presence or level of the target polypeptide or protein. Such antibodies can be obtained using methods known in the art, or can be obtained from commercial sources. Examples of immunoassays include, without limitation, Western blotting, enzyme-linked immunosorbent assay (ELISA), enzyme immunoassay (EIA), radioimmunoassay (RIA), sandwich assays, competitive assays, immunofluorescent staining and imaging, immunohistochemistry (IHC), and fluorescent activating cell sorting (FACS).

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Similarly, ranges delimited by “at least” are understood to include the lower value provided and all higher numbers.

As used herein, “about” is understood to include within three standard deviations of the mean or within standard ranges of tolerance in the specific art. In certain embodiments, about is understood a variation of no more than 0.5.

The articles “a” and “an” are used herein to refer to one or more than one (i.e. to at least one) of the grammatical object of the article. By way of example, “an element” means one element or more than one element.

The term “including” is used herein to mean, and is used interchangeably with, the phrase “including but not limited to”. Similarly, “such as” is used herein to mean, and is used interchangeably, with the phrase “such as but not limited to”.

The term “or” is used inclusively herein to mean, and is used interchangeably with, the term “and/or,” unless context clearly indicates otherwise.

### Part I: MX1 and PPP1R15A

### I. General

The present invention is at least partially based on the discovery of roles of MX1 and PPP1R15A in immune system and cell-based immunity. Accordingly, methods of use are provided herein involving modulation of MX1 or PPP1R15A.

MX1 and PPP1R15A are identified using single-cell sequencing (sc-Seq) for improving immune response, after administration of neo-antigen tumor vaccines. The advantage of using single-cell sequencing (sc-Seq) as an efficacy monitor is that it is not limited to known immune cell markers and can help obtain novel markers that are specific and directly related to the vaccines.

MX1 is MX dynamin like GTPase 1. The term “MX1” as used herein refers to MX1 gene and MX1 gene products such as mRNA of MX1 gene and protein encoded by MX1 gene. It is intended to include fragments, variants and derivatives thereof. Human MX1 gene is located in the chromosome 21 (21: 41,420,329 to 41,459,214, 21q22.3 according to Genome Reference Consortium Human Build 38 patch release 13). It has a Gene ID of 4599 in NCBI database (the sequence is incorporated herein as SEQ ID NOs: 1-4).

MX1 encodes a guanosine triphosphate (GTP)-metabolizing protein that participates in the cellular antiviral response. The encoded protein is induced by type I and type II interferons and antagonizes the replication process of several different RNA and DNA viruses. There is a related gene located adjacent to this gene on chromosome 21, and there are multiple pseudogenes located in a cluster on chromosome 4. Alternative splicing results in multiple transcript variants.

PPP1R15A is protein phosphatase 1 regulatory subunit 15A, as used herein refers to PPP1R15A gene and PPP1R15A gene products such as mRNA of PPP1R15A gene and protein encoded by PPP1R15A gene. It is intended to include fragments, variants and derivatives thereof. Human PPP1R15A gene is located in the chromosome 19 (19: 48872421 to 48876058, 19q13.33 according to Genome Reference Consortium Human Build 38 patch release 13). It has a Gene ID of 23645 in NCBI database (the sequence is incorporated herein as SEQ ID NO: 5)

PPP1R15A is a member of a group of genes whose transcript levels are increased following stressful growth arrest conditions and treatment with DNA-damaging agents. The induction of PPP1R15A by ionizing radiation occurs in certain cell lines regardless of p53 status, and its protein response is correlated with apoptosis following ionizing radiation.

In one aspect, methods of use involving administering MX1 agonists or PPP1R15A agonists are provided.

As used herein, the term “agonist” as used herein refers to an agent that increases (e.g., agonizes, increases, elevates, improves, or enhances) the biological effect of a target molecule (e.g., MX1 or PPP1R15A). The activation effects can be exerted through, e.g., increasing the amount of the target molecule, or enhancing the activity of the target molecule, or enhancing the activity of the signaling pathway of the target molecule, for example, by activating or increasing other molecules in the signaling pathway (e.g. upstream signaling molecules or downstream signaling molecules). Such agonists can include any compound, protein, or nucleic acid or any derivatives thereof that provides the agonistic effect.

The MX1 agonists can increase the expression and/or function of MX1, which function includes the ability of MX1 to bind to its targets. The types of agents capable of acting as MX1 agonists are known to those skilled in the art. For example, the agonists can increase MX1 expression at the nucleic acid (e.g., mRNA) level or protein level. For another example, the MX1 agonists can be an agent that activates MX1 or MX1 targets. In some embodiments, the MX1 agonist is a nucleic acid molecule, a protein molecule, a compound or the like. The nucleic acid molecule may be selected from an mRNA encoding MX1, an activating oligonucleotide targeting MX1, an agent for increasing expression of MX1, or an agent that activates the signal pathway of MX1. Specifically, the MX1 agonist can be mRNA encoding MX1, a gene expression vector that is capable of expressing MX1, or a MX1 protein or agonistic fragment or the like.

In certain embodiments, the MX1 agonist is selected from the group consisting of full-length mRNA of MX1, and small molecule compounds including DMXAA, ADU-S100, 2′,3′-cGAMP sodium and cGAMP. The chemical structures of the small molecule compounds are shown below.

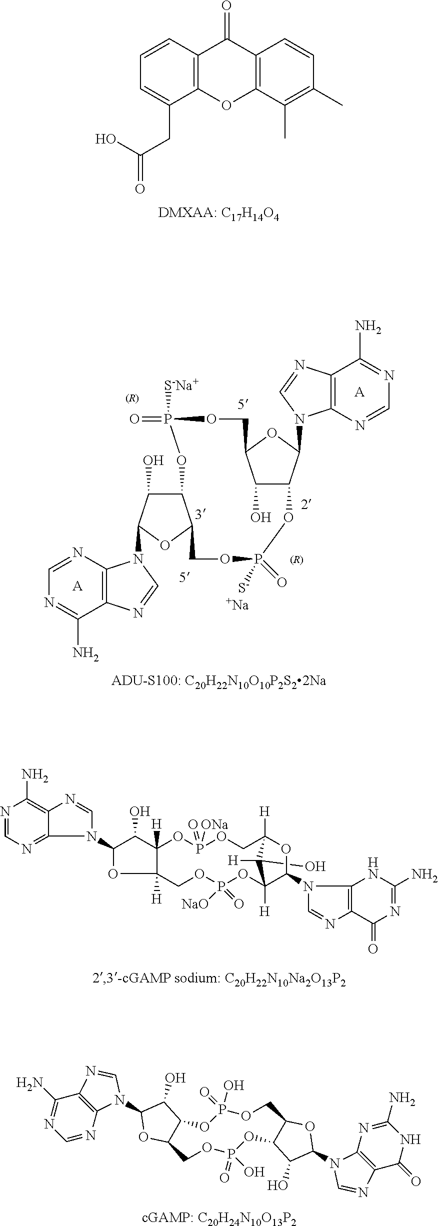

The PPP1R15A agonists can increase the expression and/or function of PPP1R15A, which function includes the ability of PPP1R15A to enhance the dephosphorylation of downstream eIF2α. The types of agents capable of acting as PPP1R15A agonists are known to those skilled in the art. For example, the agonists can increase PPP1R15A expression at the nucleic acid (e.g., mRNA) level or protein level. For another example, the PPP1R15A agonists can be an agent that activates PPP1R15A or its target eIF2α. In some embodiments, the PPP1R15A agonist is a nucleic acid molecule, a protein molecule, a compound or the like. The nucleic acid molecule may be selected from an mRNA encoding PPP1R15A, an activating oligonucleotide targeting PPP1R15A, an agent for increasing expression of PPP1R15A, or an agent that activates the signal pathway of PPP1R15A. Specifically, the PPP1R15A agonist can be mRNA encoding PPP1R15A, a gene expression vector that is capable of expressing PPP1R15A, or a PPP1R15A protein or agonistic fragment or the like.

In another aspect, methods of use involving administering MX1 antagonists or PPP1R15A antagonists are also provided.

The term “antagonists” as used herein refers to an agent that inhibits (e.g., antagonizes, reduces, decreases, blocks, reverses, or alters) the biological effect of a target molecule (e.g., MX1 or PPP1R15A). The inhibition effects can be exerted through, e.g., reducing the amount of the target molecule, or suppressing the activity of the target molecule, or suppressing the activity of the signaling pathway of the target molecule, for example, by interfering with or decreasing other molecules in the signaling pathway (e.g. upstream signaling molecules or downstream signaling molecules). Such antagonists can include any compound, protein, or nucleic acid or any derivatives thereof that provides the antagonistic effect.

The MX1 antagonists can either partially inhibit, i.e., reducing, the expression and/or function of MX1, or completely inhibit, i.e., eliminating, the expression and/or function of MX1, which function includes the ability of MX1 to bind to its targets. The types of agents capable of acting as MX1 antagonists are known to those skilled in the art. For example, the antagonists can be inhibitors, blockers and the like. For another example, the antagonists can inhibit MX1 expression at the nucleic acid (e.g., mRNA) level or protein level. For further examples, the antagonists can be an agent that competes with MX1 for binding to its targets. In some embodiments, the MX1 antagonist is a nucleic acid molecule, a protein molecule, a compound or the like. The nucleic acid molecule may be selected from an interfering RNA against MX1, an antisense oligonucleotide against MX1, an agent for knocking out or knocking down expression of MX1. Specifically, the MX1 antagonist can be siRNA, miRNA, shRNA, a gene knock-out vector or a gene expression vector that is capable of expressing siRNA, shRNA, interfering RNA or the like. The protein molecule may be selected from anti-MX1 antibodies, which may be monoclonal antibodies or polyclonal antibodies. As one example, the agent capable of competing with MX1 for binding to its targets can be CCCP or H-151.

In certain embodiments, the MX1 antagonist is selected from the group consisting of CCCP and H-151. The chemical structures of the compounds are provided below.

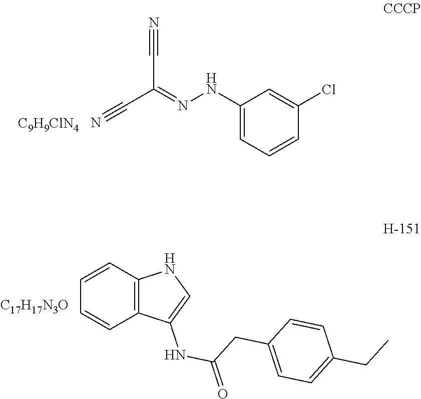 [SMILES: "C9H9ClN4" ; N#CC(C#N)=NNc1cccc(Cl)c1 ; "C17H17N3O" ; CCc1ccc(CC(=O)Nc2c[nH]c3ccccc23)cc1]

The PPP1R15A antagonists can either partially inhibit, i.e., reducing, the expression and/or function of PPP1R15A, or completely inhibit, i.e., eliminating, the expression and/or function of PPP1R15A, which function includes the ability of PPP1R15A to enhance the dephosphorylation of downstream eIF2α. The types of agents capable of acting as PPP1R15A antagonists are known to those skilled in the art. For example, the antagonists can be inhibitors, blockers and the like. For another example, the antagonists can inhibit PPP1R15A expression at the nucleic acid (e.g., mRNA) level or protein level. For further examples, the antagonists can be an agent that competes with PPP1R15A for binding to eIF2α. In some embodiments, the PPP1R15A antagonist is a nucleic acid molecule, a protein molecule, a compound or the like. The nucleic acid molecule may be selected from an interfering RNA against PPP1R15A, an antisense oligonucleotide against PPP1R15A, an agent for knocking out or knocking down expression of PPP1R15A. Specifically, the PPP1R15A antagonist can be siRNA, miRNA, shRNA, a gene knock-out vector or a gene expression vector that is capable of expressing siRNA, shRNA, interfering RNA or the like. The protein molecule may be selected from anti-PPP1R15A antibodies, which may be monoclonal antibodies or polyclonal antibodies. As one example, the agent capable of competing with PPP1R15A for binding to eIF2α can be Guanabenz or Sephin1.

Guanabenz is an alpha agonist of the alpha-2 adrenergic receptor, and has a chemical structure shown below:

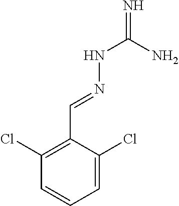 [SMILES: N=C(N)N/N=C/c1c(Cl)cccc1Cl]

Sephin 1 is an inhibitor of the regulatory subunit PPP1R15A of protein phosphatase 1, and has a chemical structure shown below:

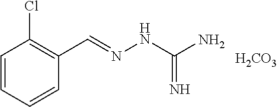

### I. Methods for In Vivo Use to Increase Immune Response

The present disclosure provides methods and compositions for enhancing cell-mediated immunity, stimulating and/or expanding T cells, potentiating immunogenicity, and treating a condition that would benefit from upregulation of immune response in a subject. In certain embodiments, the methods comprise administering to the subject an effective amount of a MX1 agonist. In certain embodiments, the methods comprise administering to the subject an effective amount of a PPP1R15A agonist.

It is unexpectedly found that MX1 agonist or PPP1R15A agonist can promote immune response in vivo, and accordingly are useful for enhancing or improving immunity (e.g. cell-based immunity) in subjects in need thereof.

In certain embodiments, the subject in need thereof can be a subject suffering from a condition that would benefit from upregulation of immune response, for example, that would benefit from induction of sustained immune responses, or from stimulation of anti-tumor immunity, or from inhibiting an immunoinhibitory receptor signaling.

In certain embodiments, the condition is tumor, infectious disease, cardiovascular disease or inflammatory disease. In certain embodiments, the condition is tumor, or infectious disease.

In certain embodiments, the subject is receiving a therapy whose efficacy can be potentiated by enhanced cell-mediated immunity. In certain embodiments, the therapy is an anti-tumor therapy or anti-infectious therapy. In certain embodiments, the anti-tumor therapy is selected from the group consisting of chemotherapy, targeted therapy, immunotherapy, cell therapy (e.g. CAR-T therapy), and tumor vaccine.

### A) Methods of Enhancing Cell-Mediated Immunity

In one aspect, the present disclosure provides a method of enhancing cell-mediated immunity in a subject in need thereof.

As used herein, the term “cell-mediated immunity” can be immunity mediated by any immune cells, for example, T cell, natural killer (NK) cell, macrophage, and so on. In certain embodiments, the cell-mediated immunity is T cell-mediated immunity.

T cell-mediated immunity can be determined using any suitable methods known in the art, including without limitation, T cell mediated cytotoxicity to a target cell (e.g. a cancer cell), T cell mediated induction of a local inflammatory response, or T cell proliferation.

In certain embodiments, the methods comprise administering to the subject an effective amount of a MX1 agonist, thereby enhancing cell-mediated immunity in the subject.

In certain embodiments, the methods comprise administering to the subject an effective amount of a PPP1R15A agonist, thereby enhancing cell-mediated immunity in the subject.

B) Methods of Stimulating and/or Expanding T Cells

In one aspect, the present disclosure provides a method of stimulating and/or expanding T cells in a subject in need thereof.

The term “stimulation” or “stimulating” with respect to T cells, refers to a primary response induced by binding of a stimulatory domain or stimulatory molecule (e.g., a TCR/CD3 complex) with its cognate ligand (e.g. MHC molecule loaded with peptide), thereby mediating signal transduction event such as T-cell response via the TCR/CD3 complex. T cell stimulation can mediate T cell proliferation, activation, differentiation, and the like.

The term “expansion” or “expanding” with respect to T cells, refers to increasing the number of T cells or promote T cell proliferation. Generally, T cells may be expanded by contacting with an agent that stimulates a CD3/TCR complex associated signal (e.g. an anti-CD3 antibody) and a ligand that stimulates a co-stimulatory molecule (e.g. an anti-CD28 antibody) on the surface of the T cells.

In certain embodiments, the methods comprise administering to the subject an effective amount of a MX1 agonist, thereby stimulating and/or expanding T cells in the subject.

In certain embodiments, the methods comprise administering to the subject an effective amount of a PPP1R15A agonist, thereby stimulating and/or expanding T cells in the subject.

### C) Methods of Potentiating Immunogenicity of an Immunogenic Composition

In one aspect, the present disclosure provides a method of potentiating immunogenicity of an immunogenic composition in a subject in need thereof.

The term “immunogenic composition” as used herein include any suitable composition that is intended to induce an immune response in a subject. The intended immune response can be either prophylactic or therapeutic. Examples of immunogenic composition include, without limitation, a vaccine, and a cell therapy such as chimeric antigen receptor (CAR)-T treatment. In certain embodiments, the vaccine is a tumor vaccine. In certain embodiments, the tumor vaccine comprises neo-antigens.

The term “potentiating immunogenicity” as used herein, means enhancing the intended immune response of the immunogenic composition.

In certain embodiments, the methods comprise administering to the subject an effective amount of a MX1 agonist, thereby potentiating immunogenicity of the immunogenic composition in the subject.

In certain embodiments, the methods comprise administering to the subject an effective amount of a PPP1R15A agonist, thereby potentiating immunogenicity of the immunogenic composition in the subject.

### D) Methods of Treating a Cell

In another aspect, the present disclosure provides a method of promoting clonal expansion of T cells, or promoting T cell activation or promoting cytotoxicity of T cells.

In certain embodiments, the methods comprise administering to the subject an effective amount of a MX1 agonist, thereby treating the condition in the subject.

In certain embodiments, the methods comprise administering to the subject an effective amount of a PPP1R15A agonist, thereby treating the condition in the subject.

In certain embodiments, the condition is tumor, infectious disease, cardiovascular disease or inflammatory disease. In certain embodiments, the condition is tumor, or infectious disease.

In certain embodiments, the methods further comprise administering in combination with a therapy that treats the condition. In certain embodiments, the therapy administered in combination is an anti-tumor therapy or anti-infectious therapy.

A therapy administered prior to or after another agent (e.g. the MX1 agonist, or PPP1R15A agonist provided herein) is considered to be administered “in combination” with that agent as the phrase is used herein, even if the therapy and the other agent are administered via different routes. Where possible, a therapy administered in combination with the agents (e.g. the MX1 agonist, or PPP1R15A agonist) disclosed herein are administered according to the schedule listed in the product information sheet of the therapy, or according to the Physicians' Desk Reference 2003 (Physicians' Desk Reference, 57th Ed; Medical Economics Company; ISBN: 1563634457; 57th edition (November 2002)) or protocols well known in the art.

### II. Methods for In Vitro Use

The present disclosure provides methods and compositions for promoting clonal expansion of T cells, or promoting T cell activation or promoting cytotoxicity of T cells. In certain embodiments, the methods comprise treating the T cells with an effective amount of a MX1 agonist under suitable conditions. In certain embodiments, the methods comprise treating the T cells with an effective amount of a PPP1R15A agonist under suitable conditions.

It is unexpectedly found that the composition provided herein (i.e. MX1 agonist, or PPP1R15A agonist) can promote T cell activation and/or T cell proliferation in vitro, and accordingly are useful for treating and/or preparing T cells useful for cell therapy.

### A) Methods of Promoting Clonal Expansion of T Cells

In one aspect, the present disclosure provides a method of promoting clonal expansion of cells, such as immune cells, and in particular, T cells.

The term “clonal expansion”, as used herein, refers to the proliferation of a cell having a specific combinatorial antigen receptor sequence, which sequence may be productively rearranged and expressed, for example where the proliferation is in response to antigenic stimulation. The expanded cell clone can have a shared combination of germline V, D, and J regions, and junctional nucleotides. In certain embodiments, the expanded cell clone may have combinatorial antigen receptors that have identical germline regions and substantially identical junctional nucleotides, e.g. differing by not more than 1, not more than 2, not more than 3 nucleotides.

In certain embodiments, the methods comprise treating the T cells with an effective amount of a MX1 agonist under suitable conditions, thereby promoting clonal expansion of the cells, for example, the T cells. In certain embodiments, the methods comprise treating the T cells with an effective amount of a PPP1R15A agonist under suitable conditions, thereby promoting clonal expansion of the cells, for example, the T cells.

In certain embodiments, the cells are lymphocytes expressing immunoglobulin, including pre-B cells, B-cells, e.g. memory B cells, and plasma cells. In certain embodiments, the cells are lymphocytes expressing T cell receptors, including thymocytes, NK cells, pre-T cells and T cells, where many subsets of T cells are known in the art, e.g. Th1, Th2, Th17, CTL, Treg, etc.

In certain embodiments, the cells are T cells. In certain embodiments, the T cells are memory T cells. Memory T cells express a specific T cell receptor and are antigen specific.

In certain embodiments, the T cells are CAR-T cells, or TCR-T cells.

### B) Methods of Promoting T Cell Activation or Promoting Cytotoxicity of T Cells

In another aspect, the present disclosure provides a method of promoting T cell activation or promoting cytotoxicity of T cells.

In certain embodiments, the methods comprise treating the T cells with an effective amount of a MX1 agonist under suitable conditions, thereby promoting T cell activation or promoting cytotoxicity of T cells. In certain embodiments, the T cells are cultured with the MX1 agonist in combination with a second agent for stimulating the T cells.

In certain embodiments, the methods comprise treating the T cells with an effective amount of a PPP1R15A agonist under suitable conditions, thereby promoting T cell activation or promoting cytotoxicity of T cells. In certain embodiments, the T cells are cultured with the PPP1R15A agonist in combination with a second agent for stimulating the T cells.

### C) Composition Comprising the T Cells Prepared

In another aspect, the present disclosure provides a composition comprising the T cells prepared using any embodiments of the methods provided herein.

In another aspect, the present disclosure provides a method of treating a condition that would benefit from upregulation of immune response in a subject in need thereof, comprising administering to the subject the composition provided herein comprising the T cells prepared using the methods described above.

### III. Methods for Detection

The present disclosure provides methods and compositions for detecting expression level of MX1 in T cells. It is unexpectedly found that level of MX1 is relevant to activation state, or activity or cytotoxicity of T cells. Accordingly, MX1 is useful as a biomarker for evaluation of T cell status.

The present disclosure provides methods and compositions for detecting expression level of PPP1R15A in T cells. It is unexpectedly found that level of PPP1R15A is relevant to activation state, or activity or cytotoxicity of T cells. Accordingly, PPP1R15A is useful as a biomarker for evaluation of T cell status.

In certain embodiments, the level of the biomarker as detected is compared with a level of the biomarker in a control cell, or is compared with a control level. In certain embodiments, the control cell is a control T cell.

In certain embodiments, the term “control T cell” refers to a T cell expressing normal or baseline level of the biomarkers (i.e. MX1 or PPP1R15A), for example, CD8+ T cells from the healthy cell or tissue sample.

In certain embodiments, the term “control level” of a biomarker described herein (i.e. MX1 or PPP1R15A) can be normal or baseline level of the biomarker, for example, a level of the biomarker in the healthy cell or tissue sample, or an average level of the biomarker in a control cell population.

In certain embodiments, the control level can be a typical level, a measured level, or a range of the level of the corresponding biomarker that would normally be observed in one or more healthy cell or tissue samples, or in one or more control cell or tissue samples. In certain embodiments, the reference level can be an average level of the corresponding biomarker in a control cell population. For example, it can be an empirical level of the biomarker that is considered to be representative of a control sample. In certain embodiments, the reference level of the biomarkers described herein is obtained using the same or comparable measurement method or assay as used in the measurement of the level of the biomarker provided herein.

### A). Methods for Evaluating Activation State, or Activity or Cytotoxicity of T Cells

In another aspect, the present disclosure provides a method of evaluating activation state, or activity or cytotoxicity of T cells.

In certain embodiments, the methods comprise detecting expression level of MX1 in the T cells, wherein an increase in the expression level of MX1 relative to a control T cell or relative to a control level is indicative of cytotoxicity of the T cells.

In certain embodiments, the methods comprise detecting expression level of PPP1R15A in the T cells, wherein an increase in the expression level of PPP1R15A relative to a control T cell or relative to a control level is indicative of cytotoxicity of the T cells. In certain embodiments, the control T cell is a CD8+ T cell.

### B). Methods for Preparing a Population of T Cells for Cell Therapy

In another aspect, the present disclosure provides a method of evaluating activation state, or activity or cytotoxicity of T cells.

In certain embodiments, the methods comprise the steps of: a) identifying the population of T cells having increased expression level of MX1 relative to a control T cell; and b) selectively enriching the identified T cells for cell therapy.

In certain embodiments, the methods comprise the steps of: a) identifying the population of T cells having increased expression level of PPP1R15A relative to a control T cell; and b) selectively enriching the identified T cells for cell therapy.

### C) Methods of Converting Inactive T Cells to Active T Cells, Composition Comprising the Converted T Cells, and Methods of Use

In another aspect, the present disclosure provides a method of converting a first population of inactive T cells to a second population of active T cells.

In certain embodiments, the methods comprises the steps of: a) providing the first population of inactive T cells, wherein the inactive T cells do not show an increase in the expression level of MX1 relative to a control T cell; b) treating the first population of inactive T cells with an effective amount of a MX1 agonist under suitable conditions; and c) detecting expression level of MX1 in the population of T cells obtained in step b), wherein an increase in the expression level of MX1 relative to a control T cell is indicative of successful conversion to the second population of active T cells.

In certain embodiments, the methods comprises the steps of: a) providing the first population of inactive T cells, wherein the inactive T cells do not show an increase in the expression level of PPP1R15A relative to a control T cell; b) treating the first population of inactive T cells with an effective amount of a PPP1R15A agonist under suitable conditions; and c) detecting expression level of PPP1R15A in the population of T cells obtained in step b), wherein an increase in the expression level of PPP1R15A relative to a control T cell is indicative of successful conversion to the second population of active T cells.

In another aspect, the present disclosure provides a composition comprising the population of T cells prepared or converted using any embodiments of the methods of converting a first population of inactive T cells to a second population of active T cells as provided herein.

In another aspect, the present disclosure provides a methods of treating a condition that would benefit from upregulation of immune response in a subject in need thereof, comprising administering to the subject the population of T cells prepared or converted by a method provided herein.

### D) Methods of Preparing a Population of T Cells for Cell Therapy and Composition Comprising the Prepared Population of T Cells

In another aspect, the present disclosure provides a method of preparing a population of T cells for cell therapy.

In certain embodiments, the methods comprises the steps of: a) identifying the population of T cells as inactive if it does not show an increase in the expression level of MX1 relative to a control T cell; and b) treating the identified inactive population of T cells with an effective amount of a MX1 agonist under suitable conditions, thereby obtaining a population of activated T cells.

In certain embodiments, the methods comprises the steps of: a) identifying the population of T cells as inactive if it does not show an increase in the expression level of PPP1R15A relative to a control T cell; and b) treating the identified inactive population of T cells with an effective amount of a PPP1R15A agonist under suitable conditions, thereby obtaining a population of activated T cells.

In another aspect, the present disclosure provides a composition comprising the population of T cells activated or prepared using any embodiments of the methods of preparing a population of T cells for cell therapy as provided herein.

In another aspect, the present disclosure provides a methods of treating a condition that would benefit from upregulation of immune response in a subject in need thereof, comprising administering to the subject the population of T cells prepared or activated by a method provided herein.

### IV. Methods for Reducing Undesired Immune Condition

In another aspect, the present disclosure further provides methods and compositions for reducing cell-mediated immunity, deactivating T cells, and treating a condition that would benefit from downregulation of immune response in a subject. In certain embodiments, the methods comprise administering to the subject an effective amount of a MX1 antagonist. In certain embodiments, the methods comprise administering to the subject an effective amount of a PPP1R15A antagonist.

It is unexpectedly found that MX1 or PPP1R15A are elevated in activated immune cells, in particular activated T cells, and accordingly, it is expected that reducing or antagonizing MX1 or PPP1R15A could be useful for reducing unwanted or undesired immunity (e.g. cell-based immunity) and treating conditions or diseases associated with such unwanted or undesired immune/inflammatory conditions in subjects in need thereof.

In another aspect, the present disclosure provides compositions of reducing cell-mediated immunity in a subject in need thereof, comprising an MX1 antagonist. Examples of MX1 antagonist include CCCP and H-151.

In certain embodiments, the subject has been determined to have T cells having an increase in the expression level of MX1 relative to a control T cell. Expression level of MX1 can be determined using any suitable methods known in the art as well as those described in the present disclosure.

In another aspect, the present disclosure provides compositions of reducing cell-mediated immunity in a subject in need thereof, comprising a PPP1R15A antagonist. Examples of PPP1R15A antagonist include Guanabenz and Sephin1.

In certain embodiments, the subject has been determined to have T cells having an increase in the expression level of PPP1R15A relative to a control T cell. Expression level of PPP1R15A can be determined using any suitable methods known in the art as well as those described in the present disclosure.

In certain embodiments, the subject is suffering from a condition characterized in excessive cell-mediated immunity.

In certain embodiments, the condition is an inflammatory disease, an autoimmune disease, an allergic disease, or a T cell cancer. In certain embodiments, the condition is lupus erythematosus, rheumatoid arthritis, inflammatory bowel disease (IBD), or GVHD.

In certain embodiments, the condition is where the subject has received or is contemplated to receive an allogeneic or xenogeneic transplant.

### A) Methods for Reducing Cell-Mediated Immunity

In another aspect, the present disclosure provides methods of reducing cell-mediated immunity in a subject in need thereof.

In certain embodiments, the methods comprise administering to the subject an effective amount of a MX1 antagonist, thereby reducing cell-mediated immunity in the subject.

In certain embodiments, the methods comprise administering to the subject an effective amount of a PPP1R15A antagonist, thereby reducing cell-mediated immunity in the subject.

In certain embodiments, the cell-mediated immunity is T cell-mediated immunity.

### B) Methods for Deactivating T Cells

In another aspect, the present disclosure provides methods of deactivating T cells in a subject in need thereof.

In certain embodiments, the methods comprise administering to the subject an effective amount of a MX1 antagonist, thereby deactivating T cells in the subject.

In certain embodiments, the methods comprise administering to the subject an effective amount of a PPP1R15A antagonist, thereby deactivating T cells in the subject.

### C) Methods for Treating a Condition

In another aspect, the present disclosure provides methods of treating a condition that would benefit from downregulation of immune response in a subject in need thereof.

In certain embodiments, the methods comprise administering to the subject an effective amount of a MX1 antagonist.

In certain embodiments, the methods comprise administering to the subject an effective amount of a PPP1R15A antagonist.

In certain embodiments, the condition that would benefit from downregulation of immune response is autoimmune disease, graft rejection or inflammatory condition. In certain embodiments, autoimmune disease is selected from the group consisting of lupus erythematosus, rheumatoid arthritis, inflammatory bowel disease (IBD), and GVHD.

### Part II: Biomarkers for Efficacy of Tumor Neoantigen Vaccine

In another aspect, the present disclosure provides methods of assessing responsiveness of a subject to a tumor neoantigen vaccine, in particular, to assess responsiveness of the subject to at least one priming dose of the tumor neoantigen vaccine, or to assess responsiveness of the subject to at least one boosting dose of the tumor neoantigen vaccine.

The term “responsiveness” to a tumor neoantigen vaccine as used in the present disclosure, refers to the immune response generated following administration of the tumor neoantigen vaccine. Tumor neoantigen vaccine is expected to activate the immune system, in particular, to induce anti-tumor immune response. Such immune response could entail changes in expression levels of certain genes in different immune cells. Characterization of differential expression of these markers can provide for indication of the level of immune responses induced by the tumor neoantigen vaccine.

In another aspect, the present disclosure provides methods of predicting the risk of tumor relapse in a subject before or after receiving at least one dose of tumor neoantigen vaccine.

In further another aspect, the present disclosure provides methods of assessing therapeutic efficacy in a subject having been treated with an anti-tumor therapy in combination with at least one dose of tumor neoantigen vaccine.

### Tumor Neoantigen Vaccine

Tumor neoantigen vaccines can be synthesized antigens (e.g. peptide antigens or polynucleotides encoding such peptide antigens) that are designed for inducing anti-tumor immune response in a subject. Tumor neoantigen vaccines can be personalized and prepared based on the tumor neoantigens identified in the subject.

In certain embodiments, the subject has been diagnosed to have cancer. In certain embodiments, the cancer is resectable. In certain embodiments, the subject has received tumor resection surgery. In certain embodiments, the subject had no chemotherapy before the resection surgery.

In certain embodiments, the tumor tissue, adjacent tissue and/or a peripheral blood sample of the subject have been analysed to identify one or more tumor-specific mutations in the subject.

In certain embodiments, the tumor neoantigen vaccine is prepared based on the identified tumor-specific mutations.

In certain embodiments, the subject has been diagnosed to have pancreatic cancer, optionally pancreatic ductal adenocarcinoma.

### 1. Biomarkers for Assessing Responsiveness to Neoantigen Vaccines

In particular, the present inventors unexpectedly identified several genes whose changes in certain immune cells correlate with the efficacy of the tumor neoantigen vaccine, and hence are useful as biomarkers for assessing responsiveness of a subject to tumor neoantigen vaccines.

In certain embodiments, the present disclosure provides methods of assessing responsiveness of a subject to a tumor neoantigen vaccine. In certain embodiments, such methods comprise a) determining expression level of one or more genes in one or more immune cells from a sample obtained from the subject receiving the tumor neoantigen vaccine.

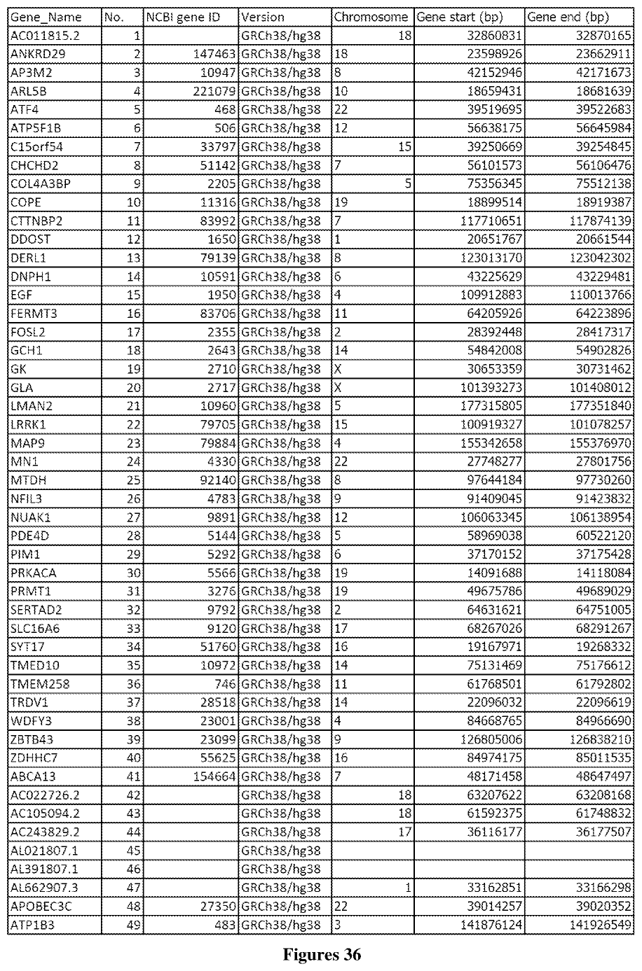

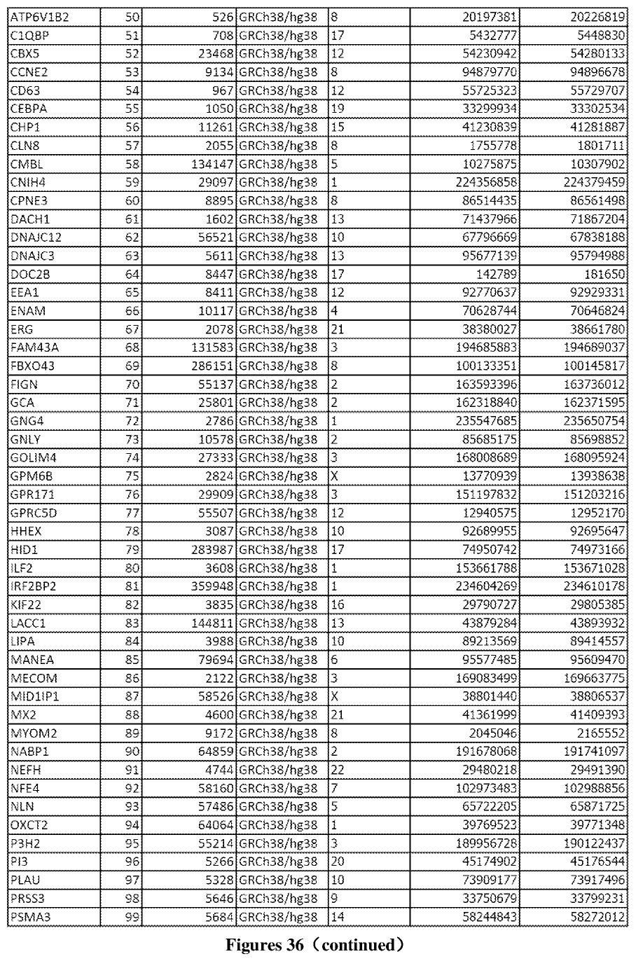

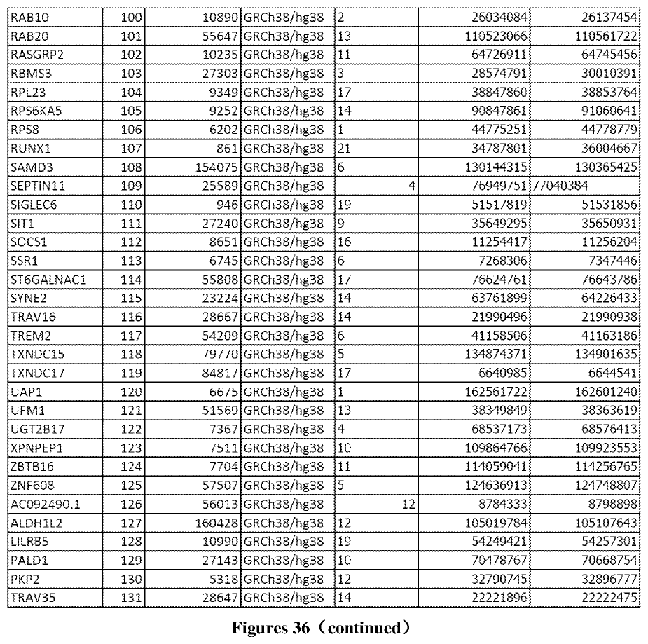

In certain embodiments, the one or more genes are selected from the group consisting of: AC011815.2, ANKRD29, AP3M2, ARL5B, ATF4, ATP5F1B, C15orf54, CHCHD2, COL4A3BP, COPE, CTTNBP2, DDOST, DERL1, DNPH1, EGF, FERMT3, FOSL2, GCH1, GK, GLA, LMAN2, LRRK1, MAP9, MN1, MTDH, NFIL3, NUAK1, PDE4D, PIM1, PRKACA, PRMT1, SERTAD2, SLC16A6, SYT17, TMED10, TMEM258, TRDV1, WDFY3, ZBTB43 and ZDHHC7, or are any combination thereof. The biomarkers are provided in FIG. 36.

In certain embodiments, the one or more immune cells are selected from the group consisting of: CD8+ T cells, CD4+ T cells, monocytes, B cells, NK cells and macrophages.

In certain embodiments, the one or more immune cells are CD8+ T cells and the one or more genes are selected from the group consisting of: AC011815.2, EGF, GCH1, NUAK1 and SERTAD2, or are any combination thereof.

In certain embodiments, the one or more immune cells are CD4+ T cells and the one or more genes are selected from the group consisting of: ANKRD29, C15orf54, FOSL2, GCH1, PRKACA, SERTAD2, SLC16A6, TRDV1, WDFY3 and ZBTB43, or are any combination thereof.

In certain embodiments, the one or more immune cells are monocytes and the one or more genes are selected from the group consisting of: AP3M2, ARL5B, ATP5F1B, CHCHD2, COPE, GLA, MN1 and MTDH, or are any combination thereof.

In certain embodiments, the one or more immune cells are B cells and the one or more genes are selected from the group consisting of: ATF4, ATP5F1B, DDOST, DERL1, DNPH1, LMAN2, MAP9, PDE4D, PIM1, TMEM258 and ZDHHC7, or are any combination thereof.

In certain embodiments, the one or more immune cells are NK cells and the one or more genes are selected from the group consisting of: COL4A3BP, FERMT3 and NFIL3, or are any combination thereof.

In certain embodiments, the one or more immune cells are macrophages and the one or more genes are selected from the group consisting of: COPE, CTTNBP2, GK, LRRK1, MTDH, PRMT1, SYT17 and TMED10, or are any combination thereof.

In particular, the present inventors have unexpectedly found that the biomarkers can be different at different vaccination stages. In general, repeated doses of vaccines are believed to induce strong and long-lasting protective immunity. The first vaccination doses are believed to prime the immune system, for example by activating naïve T cells which then undergo proliferation, contraction and differentiation to develop into primary memory T cells. Subsequent vaccination doses are believed to boost the immune system, for example by restimulate the primary memory T cells.

In certain embodiments, the subject receives multiple doses of tumor neoantigen vaccines. As used herein, the first several doses of the tumor neoantigen vaccine are referred to as priming doses, which are administered close in time to each other. In certain embodiments, the subject receives one, two, three, four or five or more priming doses of the tumor neoantigen vaccine. In certain embodiments, the priming doses are administered within 20 days, within 22 days, within 25 days, within 30 days, within 40 days, or within 45 days. In certain embodiments, the priming doses are administered on day 1, day 4, day 8, day 15, and/or day 22. The period during which priming doses are administered are priming phase of the vaccination. In certain embodiments, the priming phase is no longer than 20 days, 22 days, 25 days, 30 days, 40 days, or 45 days.

After the priming phase, the subject can receive additional doses of the tumor neoantigen vaccine, which are referred to as boosting doses. In certain embodiments, the subject receives one, two, or more boosting doses of the tumor neoantigen vaccine. In certain embodiments, the boosting doses are administered on week 12 and/or week 20. The period after the priming phase are boosting phase of the vaccination, during which boosting doses are administered. In certain embodiments, the boosting phase starts 28 days, 30 days, 35 days, 40 days, 45 days, 50 days, 55 days or 60 days after the final priming dose.

### A) Biomarkers for Priming Phase

In certain embodiments, the present disclosure provides methods of assessing responsiveness of a subject to a tumor neoantigen vaccine during the priming phase, comprising a) determining expression level of one or more genes in one or more immune cells from a sample obtained from the subject following the at least one priming dose of tumor neoantigen vaccine.

In particular, the present inventors unexpectedly identified several genes whose changes in certain immune cells correlate with the efficacy of the tumor neoantigen vaccine during priming phase, and hence are useful as biomarkers for assessing responsiveness of a subject to tumor neoantigen vaccines during the priming phase.

In certain embodiments, the one or more genes are selected from the group consisting of: AC011815.2, ATF4, C15orf54, DDOST, DERL1, DNPH1, EGF, FERMT3, FOSL2, GCH1, GK, LMAN2, MAP9, NUAK1, PDE4D, PRKACA, SERTAD2, SLC16A6, TMED10, TMEM258, WDFY3, ZBTB43 and ZDHHC7, or are any combination thereof.

In certain embodiments, the one or more immune cells are selected from the group consisting of: CD8+ T cells, CD4+ T cells, monocytes, B cells, NK cells and macrophages. In certain embodiments, the one or more immune cells are selected from the group consisting of: CD8+ T cells, CD4+ T cells, B cells, NK cells and macrophages.

In certain embodiments, the one or more immune cells are CD8+ T cells and the one or more genes are selected from the group consisting of: AC011815.2, EGF, GCH1, NUAK1 and SERTAD2, or are any combination thereof.

In certain embodiments, the one or more immune cells are CD4+ T cells and the one or more genes are selected from the group consisting of: C15orf54, FOSL2, GCH1, PRKACA, SERTAD2, SLC16A6, WDFY3 and ZBTB43, or are any combination thereof.

In certain embodiments, the one or more immune cells are B cells and the one or more genes are selected from the group consisting of: ATF4, DDOST, DERL1, DNPH1, LMAN2, MAP9, PDE4D, TMEM258 and ZDHHC7, or are any combination thereof.

In certain embodiments, the one or more immune cells are NK cells and the gene is FERMT3, or any combination thereof.

In certain embodiments, the one or more immune cells are macrophages and the one or more genes are selected from the group consisting of: GK and TMED10, or are any combination thereof.

### B) Biomarkers for Boosting Phase

In certain embodiments, the present disclosure provides methods of assessing responsiveness of a subject to a tumor neoantigen vaccine during the boosting phase, comprising a) determining expression level of one or more genes in one or more immune cells from a sample obtained from the subject during the boosting phase. In certain embodiments, the subject has completed all priming doses of the tumor neoantigen vaccine.

In particular, the present inventors unexpectedly identified several genes whose changes in certain immune cells correlate with the efficacy of the tumor neoantigen vaccine during boosting phase, and hence are useful as biomarkers for assessing responsiveness of a subject to tumor neoantigen vaccines during the boosting phase.

In certain embodiments, the one or more genes are selected from the group consisting of: AC011815.2, ANKRD29, AP3M2, ARL5B, ATP5F1B, C15orf54, CHCHD2, COL4A3BP, COPE, CTTNBP2, EGF, FERMT3, FOSL2, GK, GLA, LRRK1, MAP9, MN1, MTDH, NFIL3, NUAK1, PIM1, PRMT1, SERTAD2, SLC16A6, SYT17, TMED10, TRDV1 and ZDHHC7, or are any combination thereof.

In certain embodiments, the one or more immune cells are selected from the group consisting of: CD8+ T cells, CD4+ T cells, monocytes, B cells, NK cells and macrophages.

In certain embodiments, the one or more immune cells are CD8+ T cells and the one or more genes are selected from the group consisting of: AC011815.2, EGF, NUAK1 and SERTAD2, or are any combination thereof.

In certain embodiments, the one or more immune cells are CD4+ T cells and the one or more genes are selected from the group consisting of: ANKRD29, C15orf54, FOSL2, SLC16A6 and TRDV1, or are any combination thereof.

In certain embodiments, the one or more immune cells are monocytes and the one or more genes are selected from the group consisting of: AP3M2, ARL5B, ATP5F1B, CHCHD2, COPE, GLA, MN1 and MTDH, or are any combination thereof.

In certain embodiments, the one or more immune cells are B cells and the one or more genes are selected from the group consisting of: ATP5F1B, MAP9, PIM1 and ZDHHC7, or are any combination thereof.

In certain embodiments, the one or more immune cells are NK cells and the one or more genes are selected from the group consisting of: COL4A3BP, FERMT3 and NFIL3, or are any combination thereof.

In certain embodiments, the one or more immune cells are macrophages and the one or more genes are selected from the group consisting of: COPE, CTTNBP2, GK, LRRK1, MTDH, PRMT1, SYT17 and TMED10, or are any combination thereof.

### C) Expression Level of Biomarkers

In certain embodiments, the expression level of a given gene provided herein (i.e. the biomarkers) is determined in the given immune cell in the sample. In certain embodiments, the sample comprises or is derived from peripheral blood mononuclear cells (PBMCs), a blood sample, or tumor infiltrating immune cells.

In certain embodiments, the expression level of a given gene is represented by percentage of a given type of immune cells that express the given gene.

In certain embodiments, the expression level of a given gene is represented by the average level of the given gene expressed in the given cell type.

Any suitable methods can be used for such determination, for example, those as described in section 1.5.12 in Example 1. In certain embodiments, the expression level are determined by sequencing, for example, single cell RNA sequencing.

In certain embodiments, the reference expression level is the expression level of the one or more genes in the one or more immune cells from a sample obtained from the subject before receiving first dose of the tumor neoantigen vaccine.

In certain embodiments, the methods of assessing responsiveness to neoantigen vaccines further comprise step b): comparing the expression level of the one or more genes determined in step a) with a reference expression level to determine difference from the reference level.

In certain embodiments, the difference is determined as change in percentage of the given type of immune cell that express the given gene in the respective sample obtained at the respective time point from the subject, for example, before and after the vaccination, before vaccination and during priming phase, or before vaccination and during boosting phase.

In certain embodiments, the methods of assessing responsiveness to neoantigen vaccines further comprise step c): assessing the responsiveness of the subject to the at least one boosting dose of tumor neoantigen vaccine, based on the difference determined in step b).

In certain embodiments, the subject is determined as having a good response to the tumor neoantigen vaccine when the difference reaches or exceeds a predetermined threshold, or the subject is determined as having an insufficient response to the tumor neoantigen vaccine when the difference is below a predetermined threshold.

In certain embodiments, the threshold is 8%, 9%, 10%, 11%, 12%, 13%, 14%, 15%, 16%, 17%, 18%, 19%, or 20%.

### 2. Biomarkers for Predicting Risk of Tumor Relapse

In another aspect, the present inventors unexpectedly identified several genes whose changes in certain immune cells correlate with the tumor relapse of the tumor neoantigen vaccine, and hence are useful as biomarkers for predicting risk of tumor relapse in a subject receiving a tumor neoantigen vaccine.

In certain embodiments, tumor relapse can be indicated by tumor reoccurrence in the subject. In certain embodiments, the subject had complete resection of tumor tissue before receiving the tumor neoantigen vaccine, and reoccurrence of tumor can be indicative of tumor relapse. In certain embodiments, tumor relapse can be indicated by abnormal increase of level of serum tumor markers such as CA19-9 or CA72-4.

In certain embodiments, the present disclosure provides methods of predicting the risk of tumor relapse in a subject before or after receiving at least one dose of tumor neoantigen vaccine. In certain embodiments, such methods comprise a) determining expression level of one or more genes in one or more immune cells from a sample obtained from the subject receiving the tumor neoantigen vaccine.

### A) Biomarkers for Tumor Relapse

In certain embodiments, the one or more genes are selected from the group consisting of: ABCA13, AC022726.2, AC105094.2, AC243829.2, AL021807.1, AL391807.1, AL662907.3, APOBEC3C, ATP1B3, ATP6V1B2, C1QBP, CBX5, CCNE2, CD63, CEBPA, CHP1, CLN8, CMBL, CNIH4, COL4A3BP, CPNE3, DACH1, DNAJC12, DNAJC3, DOC2B, EEA1, ENAM, ERG, FAM43A, FBXO43, FIGN, GCA, GNG4, GNLY, GOLIM4, GPM6B, GPR171, GPRC5D, HHEX, HID1, ILF2, IRF2BP2, KIF22, LACC1, LIPA, MANEA, MECOM, MID1IP1, MX2, MYOM2, NABP1, NEFH, NFE4, NLN, OXCT2, P3H2, PI3, PIM1, PLAU, PRSS3, PSMA3, RAB10, RAB20, RASGRP2, RBMS3, RPL23, RPS6KA5, RPS8, RUNX1, SAMD3, 11-Sep, SERTAD2, SIGLEC6, SIT1, SOCS1, SSR1, ST6GALNAC1, SYNE2, TRAV16, TREM2, TXNDC15, TXNDC17, UAP1, UFM1, UGT2B17, XPNPEP1, ZBTB16, ZNF608 and STAT1, or are any combination thereof. The biomarkers are provided in FIG. 36.

In certain embodiments, the one or more genes are selected from the group consisting of: AC022726.2, AC105094.2, AC243829.2, AL021807.1, AL391807.1, ATP1B3, CMBL, DACH1, DNAJC12, DOC2B, EEA1, ERG, FBXO43, FIGN, GPRC5D, HID1, NFE4, OXCT2, P3H2, PI3, PLAU, PRSS3, RASGRP2, RPL23, RPS8, ST6GALNAC1, SYNE2, TRAV16, TREM2 and ZNF608, or are any combination thereof.

In certain embodiments, the expression level of the one or more genes are determined in one or more immune cells from a sample obtained from the subject after receiving the at least one dose of tumor neoantigen vaccine, wherein the one or more genes are selected from the group consisting of: AC022726.2, AC105094.2, AC243829.2, AL021807.1, AL391807.1, ATP1B3, ATP6V1B2, CHP1, CLN8, CMBL, CNIH4, CPNE3, DACH1, DNAJC12, DOC2B, EEA1, ERG, FAM43A, FBXO43, FIGN, GNG4, GPM6B, GPRC5D, HHEX, HID1, IRF2BP2, KIF22, LACC1, MECOM, MID1IP1, MYOM2, NABP1, NFE4, OXCT2, P3H2, PI3, PLAU, PRSS3, RAB10, RASGRP2, RBMS3, RPL23, RPS8, RUNX1, SAMD3, SERTAD2, SIGLEC6, SIT1, SOCS1, ST6GALNAC1, SYNE2, TRAV16, TREM2, TXNDC17, UGT2B17, XPNPEP1 and ZNF608, or are any combination thereof.

In certain embodiments, the expression level of the one or more genes are determined in one or more immune cells from a sample obtained from the subject before receiving the at least one dose of tumor neoantigen vaccine, wherein the one or more genes are selected from the group consisting of: ABCA13, AC022726.2, AC105094.2, AC243829.2, AL021807.1, AL391807.1, AL662907.3, ATP1B3, CBX5, CMBL, DACH1, DNAJC12, DNAJC3, DOC2B, ENAM, ERG, FBXO43, FIGN, GOLIM4, GPRC5D, HID1, ILF2, NEFH, NFE4, NLN, OXCT2, P3H2, PI3, PLAU, PRSS3, PSMA3, RASGRP2, RPL23, RPS6KA5, RPS8, 11-Sep, ST6GALNAC1, SYNE2, TRAV16, TREM2, TXNDC15, UAP1, UFM1, ZBTB16 and ZNF608, or are any combination thereof.

In certain embodiments, the one or more immune cells are selected from the group consisting of: CD8+ T cells, CD4+ T cells, monocytes, B cells, NK cells and macrophages.

In certain embodiments, the one or more immune cells are CD8+ T cells and the one or more genes are selected from the group consisting of: CHP1, EEA1, RPS8, RUNX1, UGT2B17, XPNPEP1, ZNF608 and STAT1, or are any combination thereof.

In certain embodiments, the one or more immune cells are CD4+ T cells and the one or more genes are selected from the group consisting of: AL662907.3, CD63, COL4A3BP, CPNE3, EEA1, GPM6B, LIPA, PSMA3, RAB10, RAB20, RBMS3, RPS8, SAMD3, SERTAD2, SOCS1, TXNDC15 and STAT1, or are any combination thereof.

In certain embodiments, the one or more immune cells are monocytes and the one or more genes are selected from the group consisting of: ABCA13, ATP1B3, DNAJC3, ENAM, FIGN, GNG4, ILF2, KIF22, MANEA, NEFH, NLN, P3H2, PLAU, SIGLEC6, SSR1, TRAV16, UFM1 and ZBTB16, or are any combination thereof.

In certain embodiments, the one or more immune cells are B cells and the one or more genes are selected from the group consisting of: AC105094.2, AC243829.2, AL021807.1, AL391807.1, ATP6V1B2, C1QBP, CMBL, CNIH4, DOC2B, FAM43A, GCA, GNLY, HID1, LACC1, MID1IP1, MX2, PI3, PIM1, 11-Sep, SIT1 and SYNE2, or are any combination thereof.

In certain embodiments, the one or more immune cells are NK cells and the one or more genes are selected from the group consisting of: AC022726.2, APOBEC3C, DACH1, ERG, IRF2BP2, MYOM2, NFE4, PRSS3, RPL23, RPS6KA5 and TREM2, or are any combination thereof.

In certain embodiments, the one or more immune cells are macrophages and the one or more genes are selected from the group consisting of: CBX5, CCNE2, CEBPA, CLN8, DNAJC12, FBXO43, FIGN, GOLIM4, GPR171, GPRC5D, HHEX, MECOM, NABP1, OXCT2, RASGRP2, ST6GALNAC1, TXNDC17 and UAP1, or are any combination thereof.

### B) Expression Level of Biomarkers

In certain embodiments, the expression level of a given gene provided herein (i.e. the biomarkers) is determined in the given immune cell in the sample. In certain embodiments, the sample comprises or is derived from peripheral blood mononuclear cells (PBMCs), a blood sample, or tumor infiltrating immune cells.

In certain embodiments, the expression level of a given gene is represented by percentage of a given type of immune cells that express the given gene.

In certain embodiments, the expression level of a given gene is represented by the average level of the given gene expressed in the given cell type.

Any suitable methods can be used for such determination, for example, those as described in section 1.5.12 in Example 1. In certain embodiments, the expression level are determined by sequencing, for example, single cell RNA sequencing.

In certain embodiments, the methods of predicting the risk of tumor relapse in a subject after receiving the neoantigen vaccines further comprise step b): comparing the expression level of the one or more genes determined in step a) with a reference expression level to determine difference from the reference level.

In certain embodiments, the difference is determined as difference in percentage of the given type of immune cell that express the given gene in the respective sample obtained at the respective time point from the subject, relative to the reference level.

In certain embodiments, the methods of predicting the risk of tumor relapse in a subject after receiving the neoantigen vaccines further comprise step c): assessing the responsiveness of the subject to the at least one boosting dose of tumor neoantigen vaccine, based on the difference determined in step b).

In certain embodiments, the reference expression level is a standard or average expression level determined from a representative population of relapse subjects. In certain embodiments, the reference expression level is expression level of the corresponding gene in the corresponding immune cells that is representative for a relapse subject. In such embodiments, the subject is determined as having low risk of tumor relapse when the difference reaches or exceeds a predetermined threshold, or the subject is determined as having high risk of tumor relapse when the difference is below a predetermined threshold.

In certain embodiments, the reference expression level is expression level of the corresponding gene in the corresponding immune cells that is representative for a non-relapse subject. In certain embodiments, the reference expression level is a standard or average expression level determined from a representative population of non-relapse subjects. In such embodiments, the subject is determined as having high risk of tumor relapse when the difference reaches or exceeds a predetermined threshold, or the subject is determined as having low risk of tumor relapse when the difference is below a predetermined threshold.

In certain embodiments, the threshold is 20%, 30%, 40%, 50%, 60%, 70%, or 80%.

### 3. Biomarkers for Efficacy of Combination of Anti-Tumor Therapy and Tumor Neoantigen Vaccine

In another aspect, the present inventors unexpectedly identified several genes whose changes in certain immune cells correlate with the therapeutic efficacy in a subject having been treated with an anti-tumor therapy in combination with at least one dose of tumor neoantigen vaccine, and hence are useful as biomarkers for assessing such therapeutic efficacy.

In certain embodiments, the subject has shown tumor relapse after tumor neoantigen vaccination. In certain embodiments, the relapsed subject received anti-tumor therapy. In certain embodiments, the anti-tumor therapy is immunotherapy (such as anti-PD-1 therapy). In certain embodiments, the anti-tumor therapy comprises a PD-1 antagonist. In certain embodiments, the PD-1 antagonist is an anti-PD-1 antibody or an anti-PD-L1 antibody.

In certain embodiments, the present disclosure provides methods of assessing therapeutic efficacy in a subject having been treated with an anti-tumor therapy in combination with at least one dose of tumor neoantigen vaccine. In certain embodiments, such methods comprise determining the level of one or more genes in one or more immune cells from a sample obtained from the subject after the anti-tumor treatment.

### A) Biomarkers for Efficacy of Combination of Anti-Tumor Therapy and Tumor Neoantigen Vaccine

In certain embodiments, the one or more genes are selected from the group consisting of: AC092490.1, ALDH1L2, LILRB5, PALD1, PKP2 and TRAV35, or are any combination thereof. The biomarkers are provided in FIG. 36.

In certain embodiments, the one or more immune cells are selected from the group consisting of: CD8+ T cells, CD4+ T cells, monocytes, B cells, NK cells and macrophages. In certain embodiments, the one or more immune cells are selected from the group consisting of: CD4+ T cells, monocytes and B cells.

In certain embodiments, the one or more immune cells are CD4+ T cells and the one or more genes is LILRB5.

In certain embodiments, the one or more immune cells are monocytes and the one or more genes are selected from the group consisting of: ALDH1L2 and PKP2, or are any combination thereof.

In certain embodiments, the one or more immune cells are B cells and the one or more genes are selected from the group consisting of: AC092490.1, PALD1 and TRAV35, or are any combination thereof.

### B) Expression Level of Biomarkers

In certain embodiments, the expression level of a given gene provided herein (i.e. the biomarkers) is determined in the given immune cell in the sample. In certain embodiments, the sample comprises or is derived from peripheral blood mononuclear cells (PBMCs), a blood sample, or tumor infiltrating immune cells.

In certain embodiments, the expression level of a given gene is represented by percentage of a given type of immune cells that express the given gene.

In certain embodiments, the expression level of a given gene is represented by the average level of the given gene expressed in the given cell type.

Any suitable methods can be used for such determination, for example, those as described in section 1.5.12 in Example 1. In certain embodiments, the expression level are determined by sequencing, for example, single cell RNA sequencing.

In certain embodiments, the methods of assessing therapeutic efficacy in a subject having been treated with an anti-tumor therapy in combination with at least one dose of tumor neoantigen vaccine, further comprise step b): comparing the level of the one or more genes determined in step a) with a reference level to determine difference from the reference level. In certain embodiments, the methods further comprise assessing the therapeutic efficacy in the subject based on the difference determined in step b).

In certain embodiments, the reference expression level is expression level of the corresponding gene in the corresponding immune cells from a sample obtained from the subject before receiving the anti-tumor therapy.

In certain embodiments, the threshold is 8%, 9%, 10%, 11%, 12%, 13%, 14%, 15%, 16%, 17%, 18%, 19%, or 20%.

### 4. Kits

In another aspect, the present disclosure further provides kits for assessing responsiveness of a subject to a tumor neoantigen vaccine, comprising one or more reagents for detecting expression level of one or more genes in one or more immune cells from a sample obtained from the subject; wherein the one or more genes are selected from the group consisting of: AC011815.2, ANKRD29, AP3M2, ARL5B, ATF4, ATP5F1B, C15orf54, CHCHD2, COL4A3BP, COPE, CTTNBP2, DDOST, DERL1, DNPH1, EGF, FERMT3, FOSL2, GCH1, GK, GLA, LMAN2, LRRK1, MAP9, MN1, MTDH, NFIL3, NUAK1, PDE4D, PIM1, PRKACA, PRMT1, SERTAD2, SLC16A6, SYT17, TMED10, TMEM258, TRDV1, WDFY3, ZBTB43 and ZDHHC7, or are any combination thereof.

In another aspect, the present disclosure further provides kits for assessing responsiveness of a subject to a tumor neoantigen vaccine during priming phase, comprising one or more reagents for detecting expression level of one or more genes in one or more immune cells from a sample obtained from the subject following the at least one priming dose of tumor neoantigen vaccine; wherein the one or more genes are selected from the group consisting of: AC011815.2, ATF4, C15orf54, DDOST, DERL1, DNPH1, EGF, FERMT3, FOSL2, GCH1, GK, LMAN2, MAP9, NUAK1, PDE4D, PRKACA, SERTAD2, SLC16A6, TMED10, TMEM258, WDFY3, ZBTB43 and ZDHHC7, or are any combination thereof.

In another aspect, the present disclosure further provides kits for assessing responsiveness of a subject to tumor neoantigen vaccine during boosting phase, comprising one or more reagents for detecting expression level of one or more genes in one or more immune cells from a sample obtained from the subject during the boosting phase; wherein the one or more genes are selected from the group consisting of: AC011815.2, ANKRD29, AP3M2, ARL5B, ATP5F1B, C15orf54, CHCHD2, COL4A3BP, COPE, CTTNBP2, EGF, FERMT3, FOSL2, GK, GLA, LRRK1, MAP9, MN1, MTDH, NFIL3, NUAK1, PIM1, PRMT1, SERTAD2, SLC16A6, SYT17, TMED10, TRDV1 and ZDHHC7, or are any combination thereof.

In another aspect, the present disclosure further provides kits for predicting the risk of tumor relapse in a subject before or after receiving at least one dose of tumor neoantigen vaccine, comprising one or more reagents for detecting expression level of one or more genes in one or more immune cells from a sample obtained from the subject; wherein the one or more genes are selected from the group consisting of: ABCA13, AC022726.2, AC105094.2, AC243829.2, AL021807.1, AL391807.1, AL662907.3, APOBEC3C, ATP1B3, ATP6V1B2, C1QBP, CBX5, CCNE2, CD63, CEBPA, CHP1, CLN8, CMBL, CNIH4, COL4A3BP, CPNE3, DACH1, DNAJC12, DNAJC3, DOC2B, EEA1, ENAM, ERG, FAM43A, FBXO43, FIGN, GCA, GNG4, GNLY, GOLIM4, GPM6B, GPR171, GPRC5D, HHEX, HID1, ILF2, IRF2BP2, KIF22, LACC1, LIPA, MANEA, MECOM, MID1IP1, MX2, MYOM2, NABP1, NEFH, NFE4, NLN, OXCT2, P3H2, PI3, PIM1, PLAU, PRSS3, PSMA3, RAB10, RAB20, RASGRP2, RBMS3, RPL23, RPS6KA5, RPS8, RUNX1, SAMD3, 11-Sep, SERTAD2, SIGLEC6, SIT1, SOCS1, SSR1, ST6GALNAC1, SYNE2, TRAV16, TREM2, TXNDC15, TXNDC17, UAP1, UFM1, UGT2B17, XPNPEP1, ZBTB16, ZNF608 and STAT1, or are any combination thereof.

In another aspect, the present disclosure further provides kits for assessing therapeutic efficacy in a subject having been treated with an anti-tumor therapy in combination with at least one dose of tumor neoantigen vaccine, comprising one or more reagents for detecting the level of one or more genes in one or more immune cells from a sample obtained from the subject after the treatment; wherein the one or more genes are selected from the group consisting of: AC092490.1, ALDH1L2, LILRB5, PALD1, PKP2 and TRAV35, or are any combination thereof.

The measurement or detection can be at RNA level, DNA level and/or protein level. Suitable reagents for detecting target RNA, target DNA or target proteins can be used.

In certain embodiments, the detection reagents comprise primers or probes that can hybridize to the polynucleotide of the gene of interest (e.g., the biomarkers for assessing responsiveness to neoantigen vaccines as disclosed herein, the biomarkers for Priming Phase as disclosed herein, the biomarkers for Boosting Phase as disclosed herein, the biomarkers for tumor relapse as disclosed herein). In some embodiments, the primers, and/or the probes may or may not be detectably labeled. In certain embodiments, the kits may further comprise other reagents to perform the methods described herein. In such applications the kits may include any or all of the following: suitable buffers, reagents for isolating nucleic acid, reagents for amplifying the nucleic acid (e.g. polymerase, dNTP mix), reagents for hybridizing the nucleic acid, reagents for sequencing the nucleic acid, reagents for quantifying the nucleic acid (e.g. intercalating agents, detection probes), reagents for isolating the protein, and reagents for detecting the protein (e.g. secondary antibody). Typically, the reagents useful in any of the methods provided herein are contained in a carrier or compartmentalized container. The carrier can be a container or support, in the form of, e.g., bag, box, tube, rack, and is optionally compartmentalized.

The term “primer” as used herein refers to oligonucleotides that can specifically hybridize to a target polynucleotide sequence, due to the sequence complementarity of at least part of the primer within a sequence of the target polynucleotide sequence. A primer can have a length of at least 8 nucleotides, typically 8 to 70 nucleotides, usually of 18 to 26 nucleotides. For proper hybridization to the target sequence, a primer can have at least 75%, at least 80%, at least 85%, at least 90%, or at least 95% sequence complementarity to the hybridized portion of the target polynucleotide sequence. Primers are useful in nucleic acid amplification reactions in which the primer is extended to produce a new strand of the polynucleotide. Primers can be readily designed by a skilled artisan using common knowledge known in the art, such that they can specifically anneal to the nucleotide sequence of the target nucleotide sequence of the at least one biomarker provided herein. Usually, the 3′ nucleotide of the primer is designed to be complementary to the target sequence at the corresponding nucleotide position, to provide optimal primer extension by a polymerase.

The term “probe” as used herein refers to oligonucleotides or analogs thereof that can specifically hybridize to a target polynucleotide sequence, due to the sequence complementarity of at least part of the probe within a sequence of the target polynucleotide sequence. Exemplary probes can be, for example DNA probes, RNA probes, or protein nucleic acid (PNA) probes. A probe can have a length of at least 8 nucleotides, typically 8 to 70 nucleotides, usually of 18 to 26 nucleotides. For proper hybridization to the target sequence, a probe can have at least 75%, at least 80%, at least 85%, at least 90%, or at least 95% sequence complementarity to hybridized portion of the target polynucleotide sequence.

In certain embodiments, the primes or probes provided herein comprise a polynucleotide sequence hybridizable to the polynucleotide of the gene of interest (e.g., the biomarkers for assessing responsiveness to neoantigen vaccines as disclosed herein, the biomarkers for Priming Phase as disclosed herein, the biomarkers for Boosting Phase as disclosed herein, the biomarkers for tumor relapse as disclosed herein). In certain embodiments, the primes or probes provided herein comprise a polynucleotide sequence having at least 60%, 65%, 70%, 75%, 80%, 85%, 90%, 95%, 97%, 98%, 99% or 100% complementarity to a portion within the polynucleotide of the gene of interest (e.g., the biomarkers for assessing responsiveness to neoantigen vaccines as disclosed herein, the biomarkers for Priming Phase as disclosed herein, the biomarkers for Boosting Phase as disclosed herein, the biomarkers for tumor relapse as disclosed herein).

In addition, the kits may include instructional materials containing directions (i.e., protocols) for the practice of the methods provided herein. While the instructional materials typically comprise written or printed materials they are not limited to such.

In certain embodiments, the kits can further comprise a computer program product stored on a computer readable medium. When computer program product is executed by a computer, it performs the step of assessing responsiveness of a subject to a tumor neoantigen vaccine, for predicting the risk of tumor relapse in a subject before or after receiving at least one dose of tumor neoantigen vaccine, for assessing therapeutic efficacy in a subject having been treated with an anti-tumor therapy in combination with at least one dose of tumor neoantigen vaccine, based on the methods disclosed herein. Any medium capable of storing such computer executable instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The computer programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network.

In some embodiments, the present disclosure provides oligonucleotide probes attached to a solid support, such as an array slide or chip, e.g., as described in Eds., Bowtell and Sambrook DNA Microarrays: A Molecular Cloning Manual (2003) Cold Spring Harbor Laboratory Press. Construction of such devices are well known in the art.

### EXAMPLES

### Example 1

### 1.1 Methods

### 1.1.1 Trial Design

We conducted a prospective, open label, single-arm phase Ib trial at a single medical center in China. Authors designed this trial. Personalized neoantigen vaccines were supplied to Changhai Hospital by ANDA Biopharmaceutical Development. An independent data and safety monitoring committee was established to review all the trial data and to ensure the ethical conduct of the trial. This trial was approved by the institutional review boards at Changhai Hospital, Shanghai, China. All participants provided written informed consent.

### Patients and Procedures

Eligible patients were 20 to 75 years of age, had pathologically confirmed pancreatic ductal adenocarcinoma, no chemotherapy before the resection surgery and had undergone complete macroscopic (R0 [no cancer cells within 1 mm of all resection margins]) resection (Table 1). Key exclusion criteria were radiographically confirmed recurrence or metastasis within 180 postoperative days, poor postoperative recovery, clinically significant organ dysfunction, unstable angina pectoris, symptomatic congestive heart failure, severe arrhythmias, myocardial infarction in the past 6 months, prolonged QT interval (>450 ms) and previous malignant tumors other than pancreatic cancer.

TABLE 1
Inclusion, exclusion and exit criteria in this clinical trial.

| | | |
|---|---|---|
| Inclusion | 1) | Pathologic diagnosis of pancreatic ductal adenocarcinoma |
| Criteria | 2) | Aged ≥20 and ≤75 |
| | 3) | Male or not pregnant women |
| | 4) | Undergone radical resection (R0 status of resection margins [no cancer cells |
| | | within 1 mm of all resection margins]) |
| | 5) | Radiographically confirmed free of recurrence or metastasis |
| | 6) | No serious underlying disease, Eastern Cooperative Oncology Group (ECOG) |
| | | performance status 0 or 1 |
| | 7) | No chemotherapy before resection surgery |
| | 8) | No significant cardiac, lung, liver, kidney, and bone marrow insufficiency |
| | 9) | No HIV or syphilis infection |
| | 10) | Signing informed consent |
| Exclusion | 1) | Poor postoperative situation |
| criteria | 2) | Obvious organ dysfunction |
| | 3) | Radiographically confirmed recurrence or metastasis within 180 days after the |
| | | surgery |
| | 4) | Unstable angina pectoris, symptomatic congestive heart failure, severe |
| | | arrhythmias, myocardial infarction in the past 6 months, and prolonged QT |
| | | interval (>450 ms) |
| | 5) | Previous malignant tumors other than pancreatic cancer |
| | 6) | Cannot be follow up |
| | 7) | Participating in other clinical trials |
| Exit | 1) | Missed within one month after surgery or not follow-up as required |
| criteria | 2) | Patient's own willingness to withdraw |
| | 3) | Concurrent disease or severe adverse events |
| | 4) | Protocol violations |
| | 5) | Administrative reasons |

After enrollment, all patients received tumor resection surgery and parts of tumor tissue, adjacent tissue and peripheral blood samples were supplied to pathology department for pathological examination and to a third-party company for DNA and RNA sequencing to identify the tumor-specific mutations. Personalized neoantigen peptides were designed according those mutations and patients' HLA types and then were synthetized. Clinical samples are described in the Methods section in the Supplementary Appendix. Before the personalized vaccines were prepared, all patients received gemcitabine, abraxane or S-1 adjuvant chemotherapy. After chemotherapy, patients were assessed for continuing participation according inclusion/exclusion criteria. Then all eligible patients were administered with 5 doses of priming vaccination within one month and 2 doses of boosting vaccination. If tumor recurrence or abnormal elevation of serum CA19-9 or CA72-4 level was found during treatment, the patient was also treated with conventional chemotherapy or PD-1/PD-L1 antibody.

Personalized vaccines consisted of 8-25 distinct peptides (with 27 amino acids) that were grouped into 2-4 pools and 0.5 mg of poly-ICLC as the adjuvant for each pool. Vaccines were administered subcutaneously on days 1, 4, 8, 15, and 22 (priming phase) and weeks 12 and 20 (boosting phase). The dose was 0.3 mg/peptide for each patient (Table 2) and injection sites were nonrotating extremities. Details of manufacturing and procedure are described in the Methods section in the Supplementary Appendix.

TABLE 2
Clinical dosage of the neoantigen vaccines for patients

| Patient | Total number of | Total peptide dosage | Total adjuvant dosage |
|---|---|---|---|
| ID | peptides | (mg) | (mg) |
| P1 | 8 | 2.4 mg | 1 mg |
| P2 | 15 | 4.5 mg | 2 mg |
| P3 | 11 | 3.3 mg | 1 mg |
| P4 | 19 | 5.7 mg | 2 mg |
| P5 | 22 | 6.6 mg | 2 mg |
| P6 | 23 | 6.9 mg | 2 mg |
| P7 | 14 | 4.2 mg | 2 mg |
| P8 | 10 | 3.0 mg | 2 mg |
| P9 | 15 | 4.5 mg | 2 mg |
| P10 | 20 | 6.0 mg | 2 mg |
| P11 | 25 | 7.5 mg | 2 mg |
| P12 | 13 | 3.9 mg | 1.5 mg |

### 1.1.2 End Points and Assessments

The primary endpoint was safety, assessed by the rate of grade 3 or worse adverse events (graded according to National Cancer Institute Common Terminology Criteria for Adverse Events, version 5.0). Adverse events were assessed throughout the vaccination for their incidence, grades and relatedness to the vaccines until 2 years after the surgery. Safety evaluations also included clinical laboratory examinations, electrocardiogram, abdominal ultrasound, temperature, heart rate, blood pressure, respiratory rate and the physical appearance of the skin and five senses.

The key secondary end points were serum CA19-9 or CA72-4 levels after treatment, overall survival (OS) and recurrence-free survival (RFS). We assessed the rate of patients without the abnormal elevation of the serum CA19-9 or CA72-4 levels during the vaccination and post-treatment follow-up (abnormality defined as CA19-9≥90.65 U/mL or CA27-4≥14.7 U/mL). OS was calculated from the date of surgery until the date of death (any cause). RFS was calculated from the date of surgery until the date of the first tumor recurrence (confirmed by imaging). Data of patients without events at the time of analysis were censored on the date of last follow-up.

The exploratory end points were immunologic correlates of response in peripheral blood after and during the vaccination. Ex vivo ELISpot was performed to detect the IFN-γ responses of peptides in the stimulation of PBMCs. Ten-color flow cytometry and single-cell transcriptome sequencing in all 12 patients were performed to evaluate the immunologic correlates. Single-cell T/B-cell repertoire (TCR/BCR) sequencing in 10 patients were performed to profile the expansion of T/B-cell clonotypes. Vaccine-related immunologic responses were assessed by comparing to the pre-vaccination (baseline). Details of the sequencing and analysis are described in the Methods section in the Supplementary Appendix.

### 1.1.3 Statistical Analysis

All the analyses were performed in patients who had completed 7 doses of vaccines. Adverse events and side effects were summarized descriptively. We assumed that the treatment was not safe if the probability was 50% or more that the risk of grade 3 or worse adverse effects was more than 25%. The probability of the risk of grade 3 or worse AEs were calculated by the exact binomial test (one-sided). The increase of serum CA19-9 or CA72-4 levels were summarized descriptively. Kaplan-Meier method was used to analyze the recurrence-free survival and overall survival (Further details are provided in the Supplementary Methods section). The follow-up range includes all follow-up times until July 2022. We performed immunologic analyses on available biospecimens and correlative data were analyzed as described in the Methods section in Supplementary Appendix. Reported P values are two-sided, and the significance level was set at 0.05 for all analyses unless otherwise noted. R software (version 3.6.1) was used for all statistical analyses and plotting.

### 1.2 Result

1.2.1 Prolonged Survival of Pancreatic Cancer Patients Receiving Personalized Neoantigen Immunotherapy after Surgery

From Feb. 9, 2018 through Apr. 4, 2020, we enrolled 14 patients, all of whom received at least one dose of vaccines, at the Department of Hepatobiliary Pancreatic Surgery of Changhai Hospital. Of these patients, 1 withdrew informed consent, 1 refused to undergo the evaluations specified in the protocol and finally 12 were eligible for inclusion in the study (Table 3). They were Han Chinese, with a mean age of 59.2 years. Among them, 11 and 10 patients had somatic mutations in TP53 and KRAS respectively, which are prevalent in PDAC.

TABLE 3
Demographic and Clinical Characteristics of the Patients at
Baseline, According to Relapse and Non-Relapse Groups. *

| | | | Patients with | | Patients without | |
|---|---|---|---|---|---|---|
| | All patients | | relapse | | relapse | |
| Characteristic | (N = 12) | | (N = 4) | | (N = 8) | |
| Sex - no. (%) | | | | | | |
| female | 5 | (41.7) | 0 | (0.0) | 5 | (62.5) |
| male | 7 | (58.3) | 4 | (100.0) | 3 | (37.5) |
| Age at surgery - yr | | | | | | |
| mean (SD) | 59.25 | (10.10) | 53.00 | (5.48) | 62.38 | (10.68) |
| median (IQR) | 60.5 | (51.5-68.25) | 51 | (49.75-54.25) | 65.5 | (59.5-69.25) |
| Height(cm) - median | 168.00 | (163.75-170.25) | 169.00 | (167.75-170.50) | 166.50 | (159.50-170.25) |
| (IQR) | | | | | | |
| Weight(kg) - median | 60.00 | (57.00-65.25) | 66.50 | (63.75-73.00) | 58.00 | (56.50-60.00) |
| (IQR) | | | | | | |
| Hypertension - no. (%) | | | | | | |
| No | 6 | (50.0) | 3 | (75.0) | 3 | (37.5) |
| Yes | 6 | (50.0) | 1 | (25.0) | 5 | (62.5) |
| Diabetes - no. (%) | | | | | | |
| No | 9 | (75.0) | 4 | (100.0) | 5 | (62.5) |
| Yes | 3 | (25.0) | 0 | (0.0) | 3 | (37.5) |
| Surgery - no. (%) | | | | | | |
| RAMPS | 6 | (50.0) | 2 | (50.0) | 4 | (50.0) |
| Whipple | 6 | (50.0) | 2 | (50.0) | 4 | (50.0) |
| Without vascular resection - | 12 | (100) | 4 | (100) | 8 | (100) |
| no. (%) | | | | | | |
| Cholecystectomy - | | | | | | |
| no. (%) | | | | | | |
| No | 3 | (25.0) | 1 | (25.0) | 2 | (25.0) |
| Yes | 9 | (75.0) | 3 | (75.0) | 6 | (75.0) |
| R0 status of surgical | 12 | (100) | 4 | (100) | 8 | (100) |
| margins - no. (%)† | | | | | | |
| Tumor location - | | | | | | |
| no. (%) | | | | | | |
| Body/tail | 6 | (50.0) | 2 | (50.0) | 4 | (50.0) |
| Head/Uncinate | 6 | (50.0) | 2 | (50.0) | 4 | (50.0) |
| Maximum tumor size (mm) - | 30.00 | (25.00-30.50) | 25.00 | (4.08) | 31.25 | (6.58) |
| median (IQR) | | | | | | |
| Ratio of positive lymph | 0.02 | (0.00-0.06) | 0.04 | (0.00-0.13) | 0.02 | (0.00-0.04) |
| nodes - median (IQR) | | | | | | |
| Differentiated grade - | | | | | | |
| no. (%)‡ | | | | | | |
| G2 | 10 | (83.3) | 3 | (75.0) | 7 | (87.5) |
| G3 | 2 | (16.7) | 1 | (25.0) | 1 | (12.5) |
| T stage - no. (%)‡ | | | | | | |
| T1 | 2 | (16.7) | 1 | (25.0) | 1 | (12.5) |
| T2 | 9 | (75.0) | 3 | (75.0) | 6 | (75.0) |
| T3 | 1 | (8.3) | 0 | (0.0) | 1 | (12.5) |
| N stage - no. (%)‡ | | | | | | |
| N0 | 6 | (50.0) | 2 | (50.0) | 4 | (50.0) |
| N1 | 5 | (41.7) | 1 | (25.0) | 4 | (50.0) |
| N2 | 1 | (8.3) | 1 | (25.0) | 0 | (0.0) |
| M0 stage - no. (%)‡ | 12 | (100) | 4 | (100) | 8 | (100) |
| Tumor stage - no. (%)‡ | | | | | | |
| stage IA | 1 | (8.3) | 1 | (25.0) | 0 | (0.0) |
| stage IB | 4 | (33.3) | 1 | (25.0) | 3 | (37.5) |
| stage IIA | 1 | (8.3) | 0 | (0.0) | 1 | (12.5) |
| stage IIB | 5 | (41.7) | 1 | (25.0) | 4 | (50.0) |
| stage III | 1 | (8.3) | 1 | (25.0) | 0 | (0.0) |
| Chemotherapy - no. (%) | | | | | | |
| Gemcitabine + S-1 | 5 | (41.7) | 2 | (50.0) | 3 | (37.5) |
| Gemcitabine + | 1 | (8.3) | 0 | (0.0) | 1 | (12.5) |
| Abraxane | | | | | | |
| S-1 | 6 | (50.0) | 2 | (50.0) | 4 | (50.0) |
| Hospital stay (days) - | 11.92 | (3.42) | 11.75 | (3.30) | 12.00 | (3.70) |
| mean (SD) | | | | | | |

* Within 2.5 years after the surgery (including 1 year after 7 doses of vaccination), 4 patients had tumor recurrence or abnormal increase of serum CA19-9 or CA27-4 levels (CA19-9, ≥90.65 U/mL, 2.45 times of 37 U/mL [the upper limit of the normal reference]; CA27-4, ≥14.7 U/mL) and they were designated as the relapse group. The relapse patients were also subsequently treated with conventional chemotherapy or PD-1/PD-L1 antibody. In contrast, the other 8 patients were designated as the non-relapse group. Percentages may not total 100 because of rounding.
†A surgical margin of R0 indicates that no cancer cells were present within 1 mm of all resection margins.

‡ Tumor stages (Grade [G], tumor [T], nodal status [N] and metastasis [M]) were evaluated according to the criteria of the American Joint Committee on Cancer and Union for International Cancer Control, 7th edition.

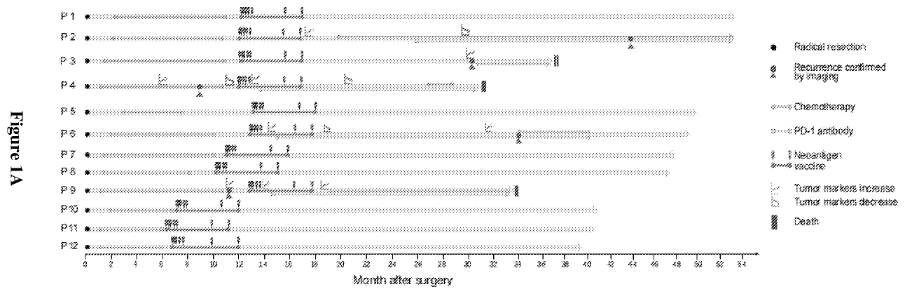

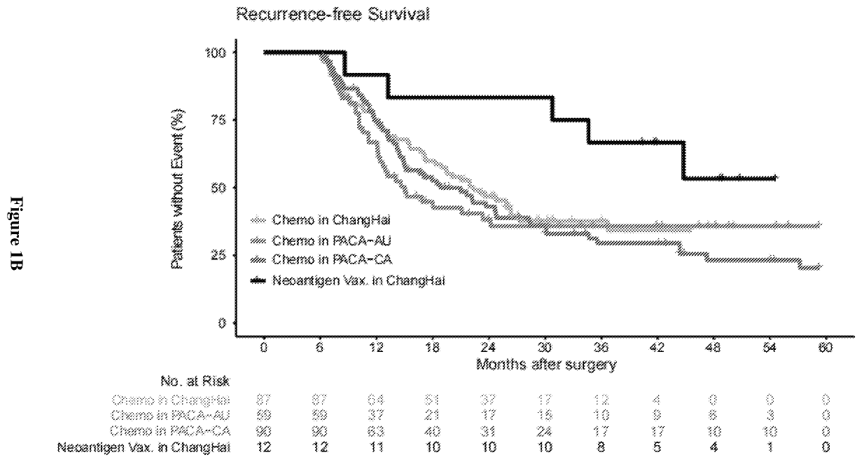

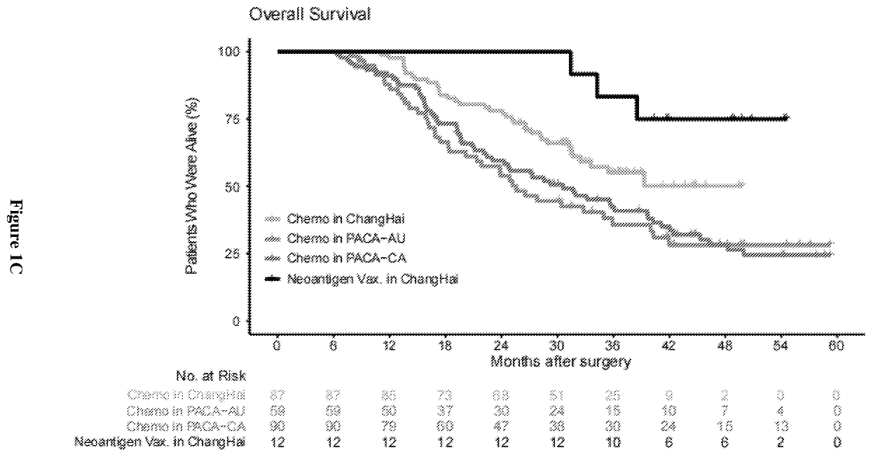

At a median of 44.25 months (range, 31˜53.5) of postoperative follow-up, 3 of the 12 patients died after tumor recurrence; nevertheless, their deaths were unrelated to the vaccines. The OS rate at 2 and 3 years were 100% (95% CI, 74˜100) and 83% (95% CI, 52˜98) respectively. The RFS rate at 2 and 3 years were 83% (95% CI, 52˜98) and 67% (95% CI, 35˜90) respectively (FIG. 1A). The mean OS of the 12 patients was 44.6 months (range, 31.4˜54.6), and the mean RFS was 38.3 months (range, 8.6˜54.6). The mean OS and RFS of enrolled patients were greater in numeric than those of patients treated with adjuvant chemotherapy in the historical control in the same hospital or other public data of PDAC (FIG. 1BC).

At a median of 31 months (range, 18.5˜58.5) of postoperative follow-up, serum CA19-9 levels of 9 (75%, 95% CI, 42.8˜94.5) patients (2 [P3 and P6] with tumor recurrence confirmed by imaging at end) were stable and mostly below the upper limit of the normal reference (37 U/mL). Among those 9 patients, P6 had 2 abnormal elevations of CA72-4 levels respectively after priming and boosting vaccination. Two (16.7%, 95% CI, 2.1˜48.4) patients (with tumor recurrence confirmed by imaging) experienced an increase of serum CA19-9 levels and a drop to normal following boosting vaccination though extremely high levels at end (FIGS. 1A and 1B).

TABLE 4
Statistics of adverse events in the 12 patients enrolled in the
clinical trial of neoantigen vaccines during the vaccination.

| | | | | | | | |
|---|---|---|---|---|---|---|---|
| | Hyperuricemia | 1 | 0.93 | 1/12 | 1 | 0 | 0 |
| General disorders and | Fever ^{#} | 4 | 3.70 | 4/12 | 4 | 0 | 0 |
| administration site | Fatigue ^{#} | 1 | 0.93 | 1/12 | 1 | 0 | 0 |
| conditions | | | | | | | |
| Respiratory, thoracic, | Sore throat ^{#} | 1 | 0.93 | 1/12 | 1 | 0 | 0 |
| and mediastinal | Rhinorrhoea ^{#} | 1 | 0.93 | 1/12 | 1 | 0 | 0 |
| diseases | | | | | | | |
| Others | Urinary leucocyte increased | 3 | 2.78 | 3/12 | 3 | 0 | 0 |
| | CRP increased ^{#} | 2 | 1.85 | 2/12 | 2 | 0 | 0 |
| | Urinary nitrite increased | 1 | 0.93 | 1/12 | 1 | 0 | 0 |
| | Dry mouth ^{#} | 1 | 0.93 | 1/12 | 1 | 0 | 0 |
| Gastrointestinal | Flatulence | 1 | 0.93 | 1/12 | 1 | 0 | 0 |
| disorders | Bloating | 1 | 0.93 | 1/12 | 1 | 0 | 0 |
| | Diarrhea | 1 | 0.93 | 1/12 | 1 | 0 | 0 |
| | Vomiting | 1 | 0.93 | 1/12 | 1 | 0 | 0 |
| Psychiatric disorders | Insomnia ^{#} | 1 | 0.93 | 1/12 | 1 | 0 | 0 |
| Nervous system | Hypersomnia ^{#} | 1 | 0.93 | 1/12 | 1 | 0 | 0 |
| disorders | Headache ^{#} | 1 | 0.93 | 1/12 | 1 | 0 | 0 |
| | Dizziness ^{#} | 1 | 0.93 | 1/12 | 1 | 0 | 0 |
| Total | Total | 108 | | | 95 | 10 | 3 |

Note:
^{#} possibly related to the vaccines.

### 1.2.2 Activation of T Cells and Up-Regulated of IFN-γ Response Pathway in the Activated T Cells During the Neoantigen Vaccination by Using the Single-Cell Sequencing

PBMCs of patient were collected on the day of the vaccine administration, as well as at the 7th, 15th and 23rd weeks to perform single-cell RNA sequencing (scRNA-seq). Among immune cells, T cells accounted for the highest proportion followed by NK cells and Monocyte cells (FIG. 5A). There were significant increases of B cells, megakaryocyte and monocyte cells while significant decrease of NK cells during the vaccination (FIG. 5B).

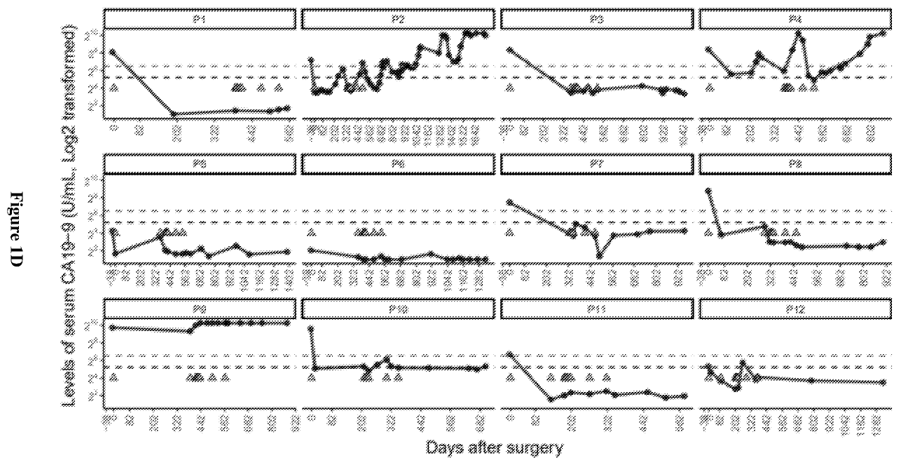

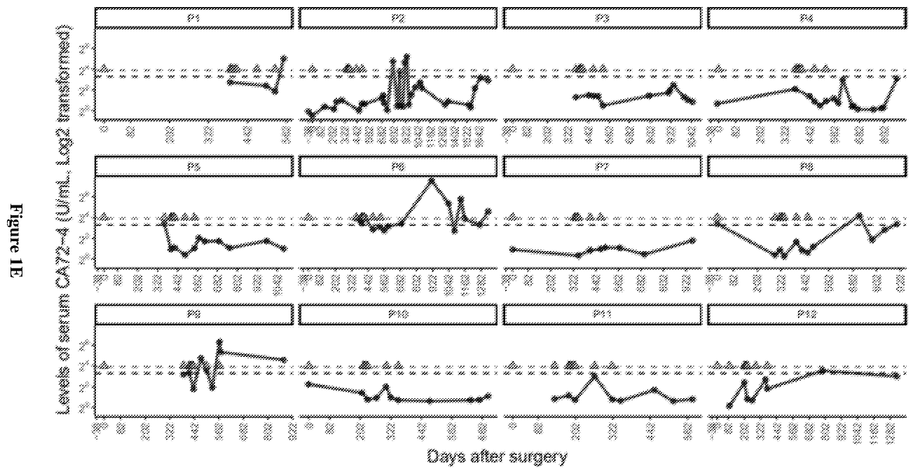

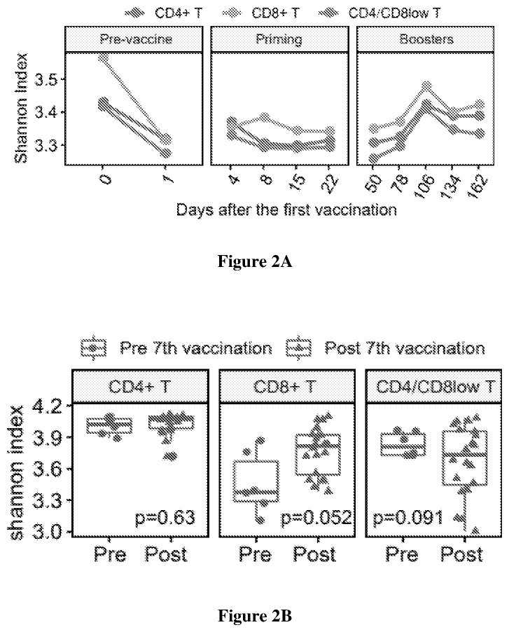

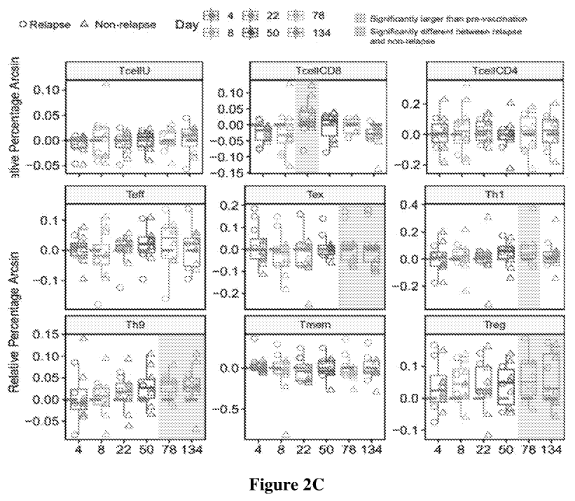

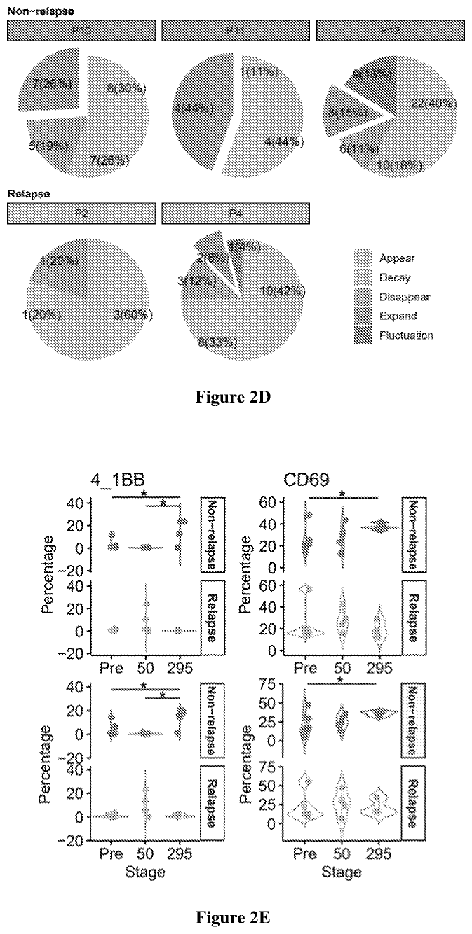

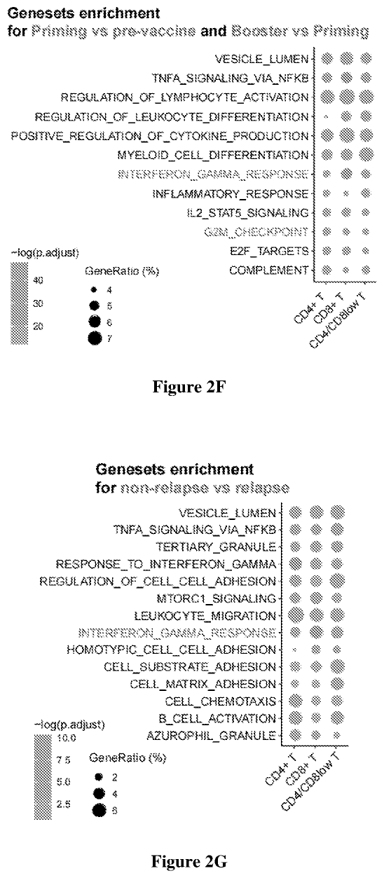

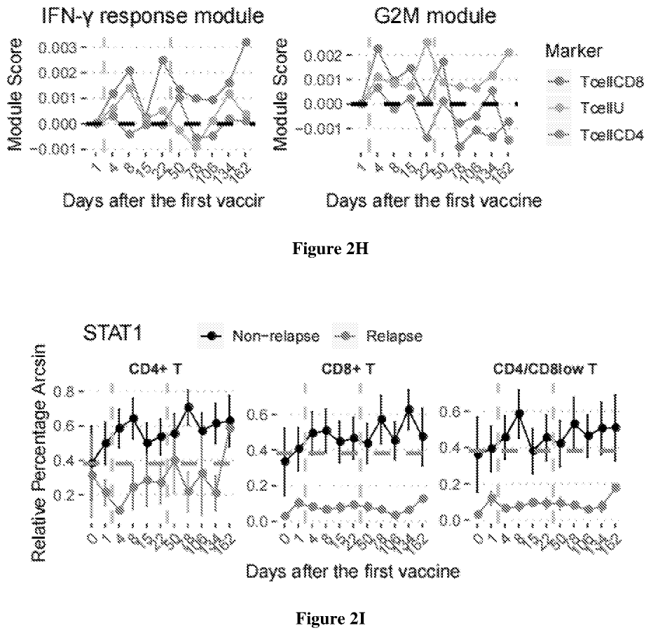

The diversity of TCR gene expression significantly increased after vaccination especially in the booster phase (FIG. 2A). Single-cell TCR sequencing (scTCR-seq) confirmed the concurrently significant increase of TCR clone diversity in the CD8+ T cells (FIG. 2B). CD8+ T cells increased slightly at 22 days (i.e. at the end of the priming phase). Effector T, Th1, Th9 and Treg cells have significant increases at the boosting phase and relapsed patients exhibited more exhausted T cells (FIG. 2C).

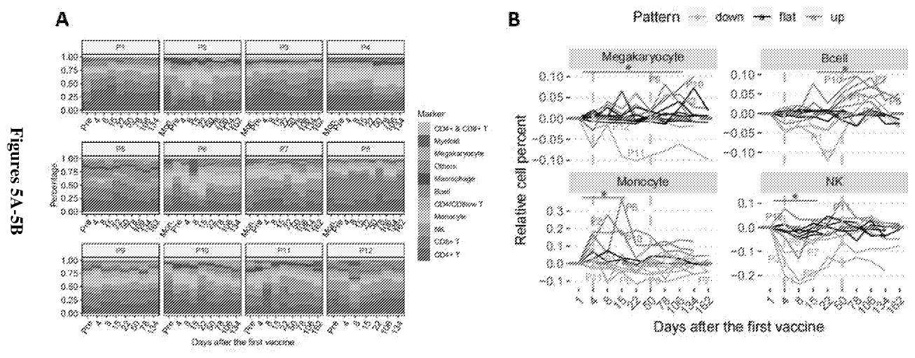

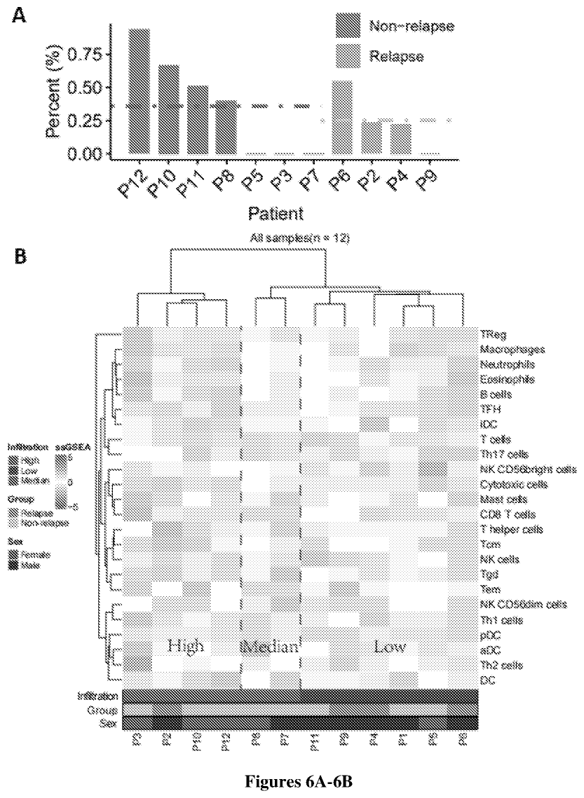

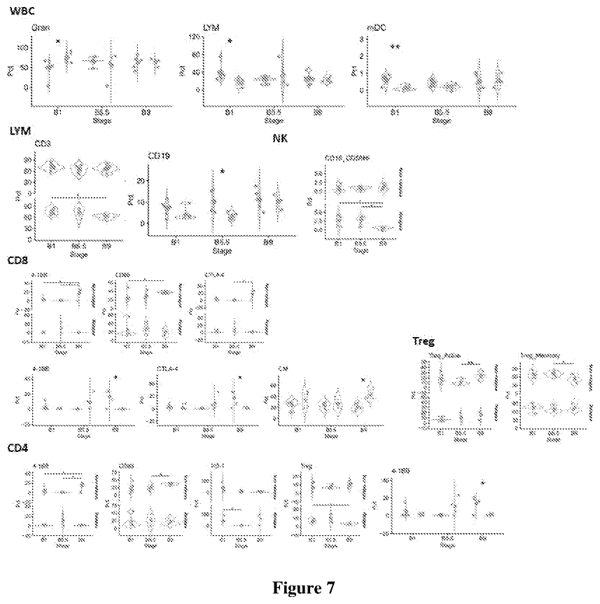

Tumor-infiltrating T cells were identified by comparing the TCRs in the tumor with the TCR clones in the blood from the scTCR-seq. Infiltrating T cells were enriched and amplified in non-relapse patients in blood (FIGS. 6A and 6B and FIG. 2D). The scRNA-seq exhibited that T cell activation was linked to genes including proliferation, cytotoxic, Interferon response, and cytokine both in the priming and boosting phases in CD4+, CD8+ and CD4/CD8low T cells (FIG. 8). The above results indicate actively response of T cells during the neoantigen immunotherapy. The flow cytometric analysis further conformed the T cells activation with the increase of 4-1BB+ and CD69+ cells in T cells in the blood of non-relapse (FIG. 2E and FIG. 7).

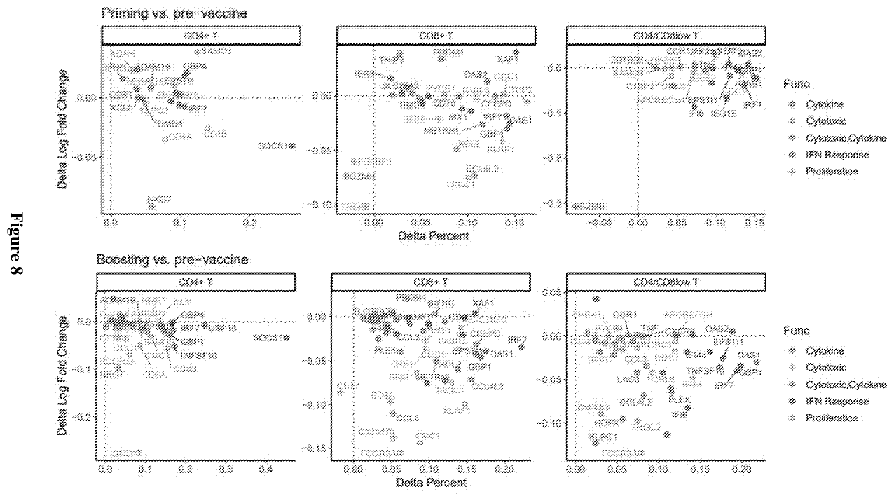

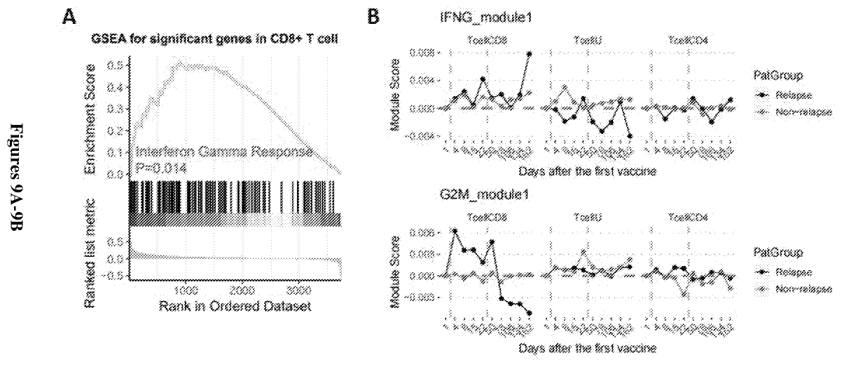

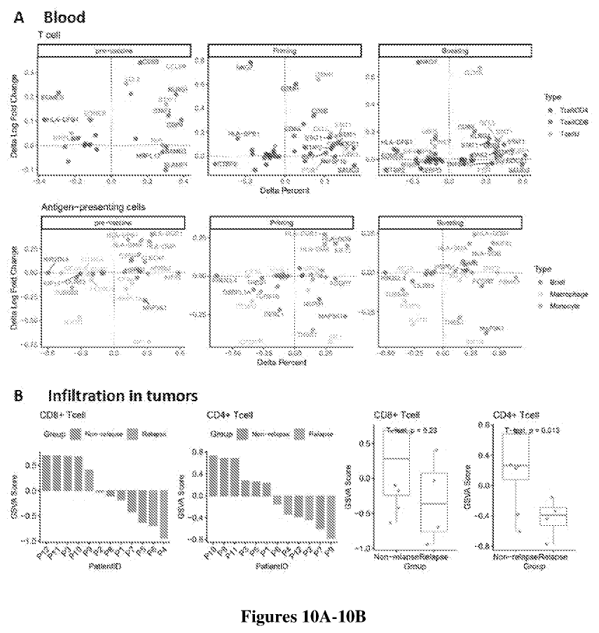

Transcriptional profiling of changes in T cells over the course of vaccination indicated that IFN-γ response and proliferation-related G2M gene signatures were enriched in the either CD4+, CD8+ or the other T cells (more in CD8+) (FIG. 2F and FIG. 9A). By considering non-relapse versus relapse, the activation of the IFN-γ response pathway was also more prominent in the non-relapse group (FIG. 2G) and genes involved in activation of T cells and antigen presentation stood out (FIG. 10A). Similarly, the average expression of IFN-γ and G2M gene modules both significantly increased in T cells during the vaccination (FIG. 2H and FIG. 9B). Moreover, the key downstream gene of IFN-γ response, STAT1, showed the significantly higher expression in the non-relapse than relapse groups in T cells (FIG. 2I). This difference for STAT1 remained in T cells infiltrating in the tumors of the patients (FIG. 10B). The above results indicate the important role of the IFN-γ response pathway in the T cells activation during the neoantigen vaccination.

### 1.2.3 the High Anti-Tumor Activity of Central Memory and Tumor-Reactive T Cells Through Up-Regulating the IFN-γ Response Pathway Genes

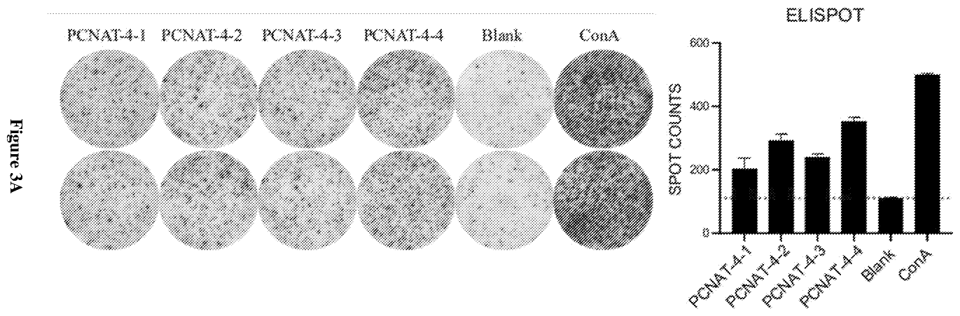

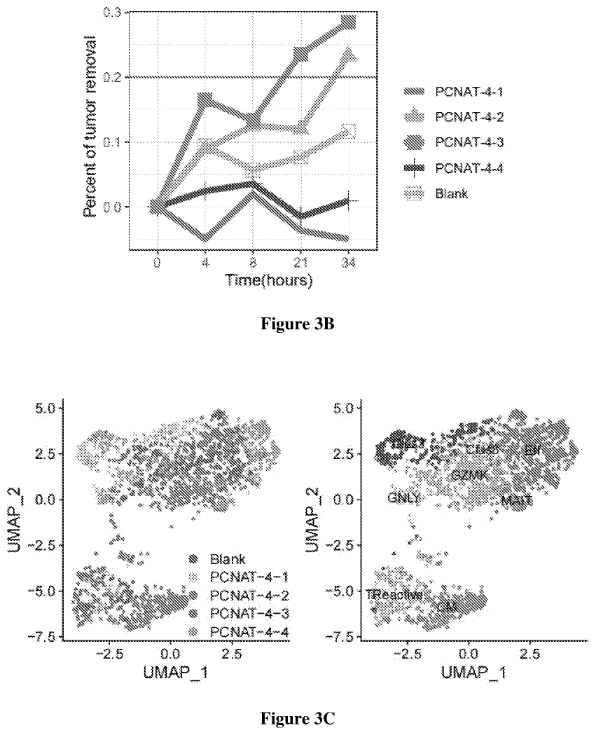

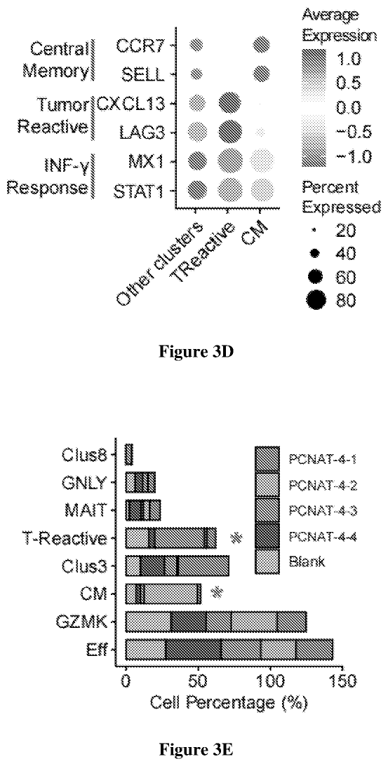

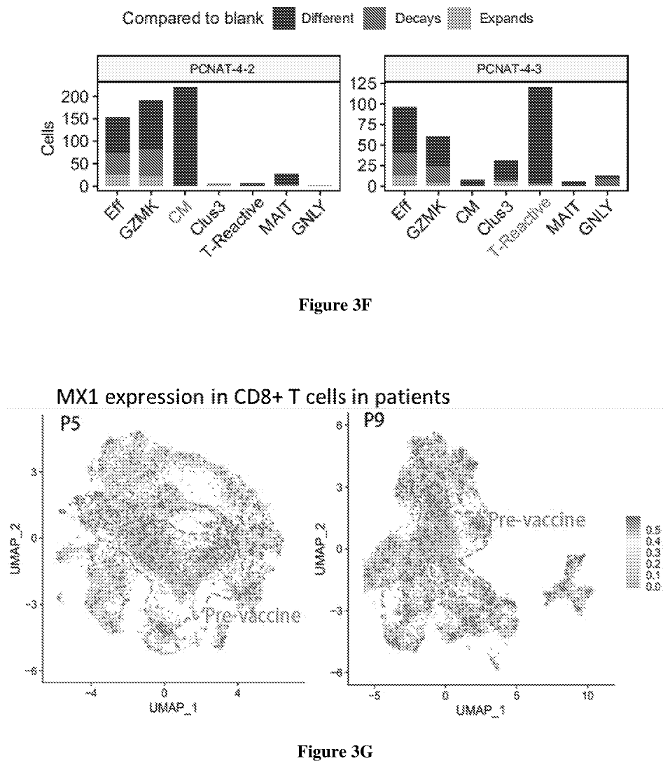

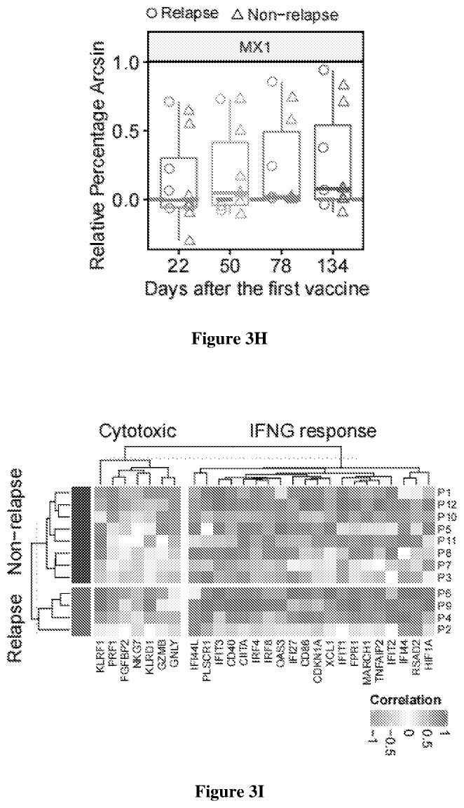

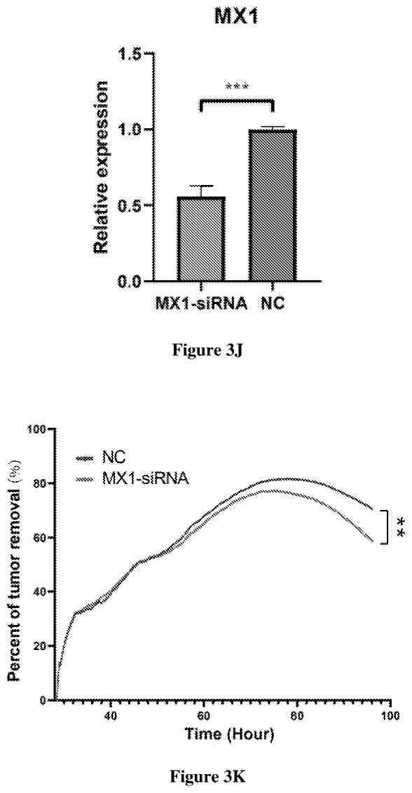

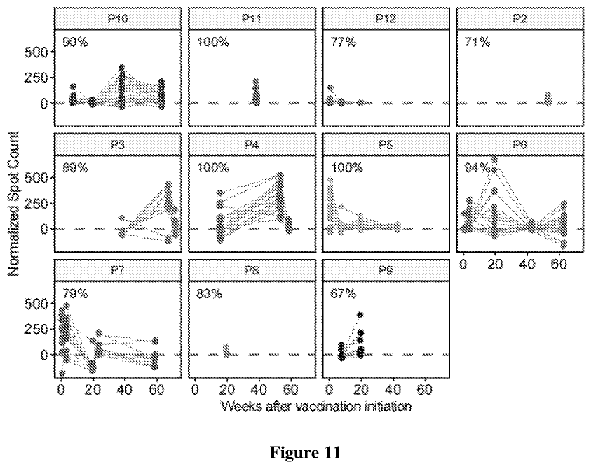

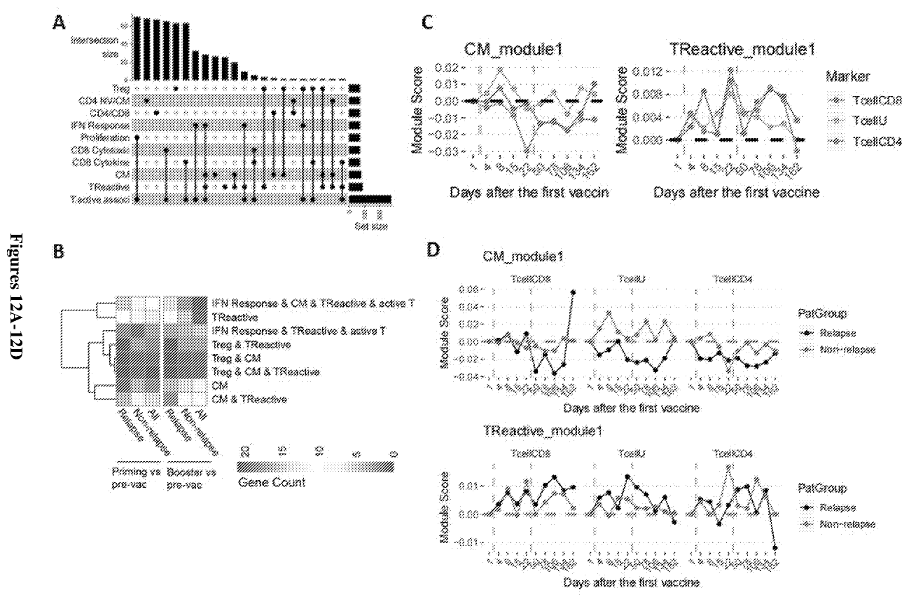

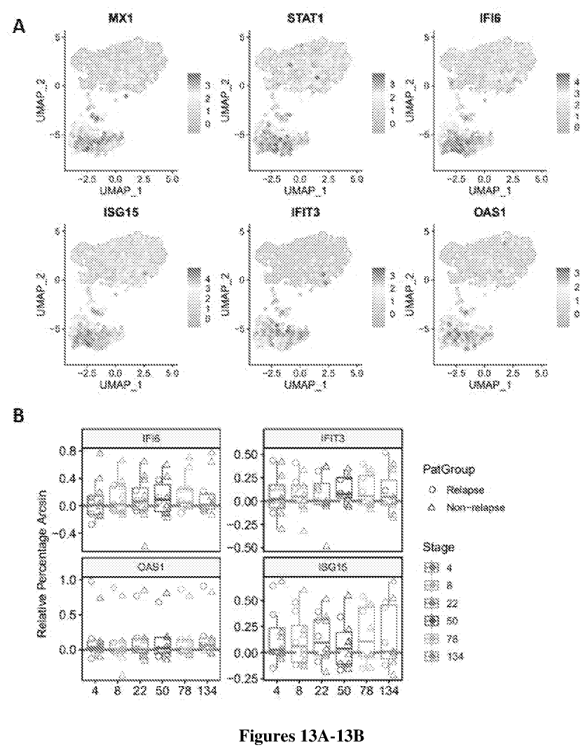

The Elispot assay showed that the most of neoantigen peptides can effectively stimulate the secretion of IFN-γ from patients' PBMC (FIG. 11), including the four neoantigen peptides (PCNAT-4-1˜4) designed for patient P4 (FIG. 3A). However, only PCNAT-4-2 and PCNAT-4-3 exerted their tumor killing function (FIG. 3B). In their stimulation, there were two dominant subtypes of cell in CD8+ T cells, namely Central Memory (CM) and Tumor-reactive (TReactive) cells 7 (FIGS. 3C-3E). Differences in TCR clones of these two subtypes were prominent compared with the blank (FIG. 3F), suggesting amplification of peptide-specific T cells after the stimulation. These two subtypes specifically expressed MX1 and STAT1 (FIG. 3D). Although those subtypes co-expressed shared many IFN-γ response genes (FIG. 12A and FIG. 13A), they seem to be activated at different phases. TReactive subtype was prone to be activated at the priming and maintained during the vaccination while CM subtype was not elevated at the priming in the CD8+ T cells (FIGS. 12B-12D). The marker genes of them including MX1 and other IFN-γ response genes were also significantly up-regulated after the vaccination in the patients' blood (FIGS. 3G and 3H and FIG. 13B). The association between IFN-γ response and tumor killing could also support that our patients obtained anti-tumor ability after administrated neoantigen vaccines. However, the IFN-γ response pathway was not stimulated in the dominant subpopulation of CD4+ and CD4/CD8low T cells (FIG. 14). In the CD4+ T cells, the CXCL13+ subpopulation was the dominant cluster in the stimulation of PCNAT-4-2˜4 (FIGS. 14A and 14B). It has been reported that the CXCL13+ subpopulation in CD4+ T cells were responsible to anti-tumor cytotoxicity in tumor-infiltrating T cells⁷. In the CD4/CD8low T cells, the higher proportion of subpopulation was effector T cells (CXCR6+, KLRB1+ and CTSH+) (FIGS. 14A and 15C). Those markers were also significantly up-regulated after the neoantigen vaccination in the blood of patients (FIGS. 15A and 15B).

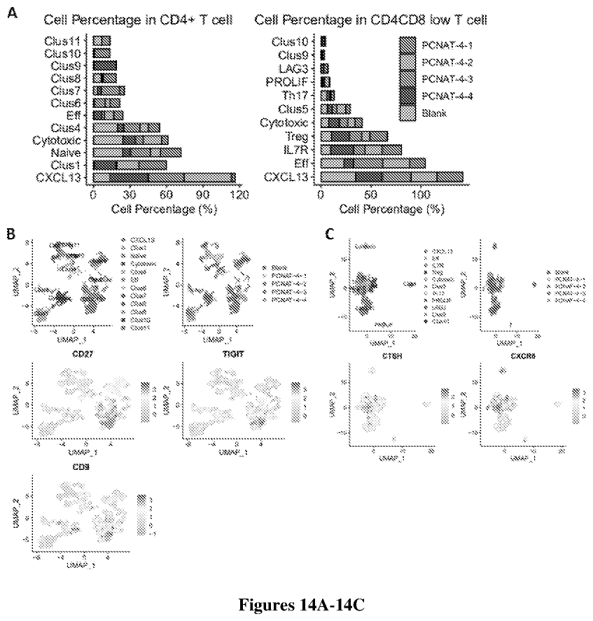

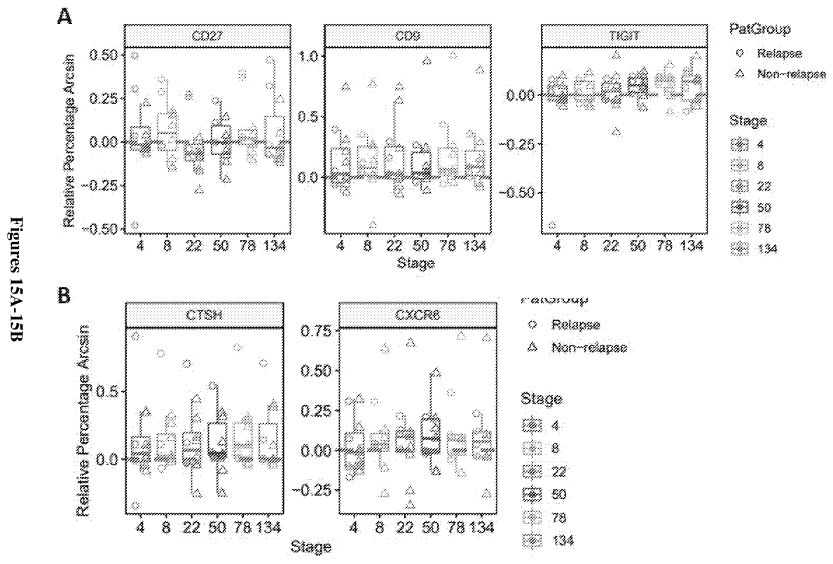

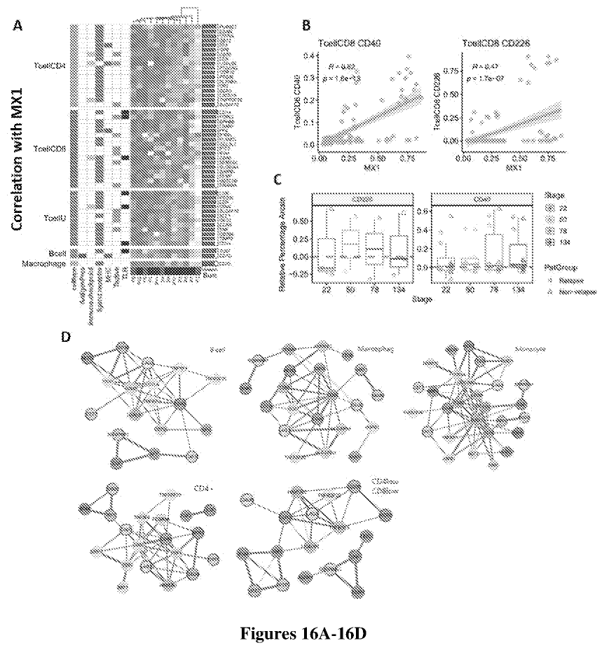

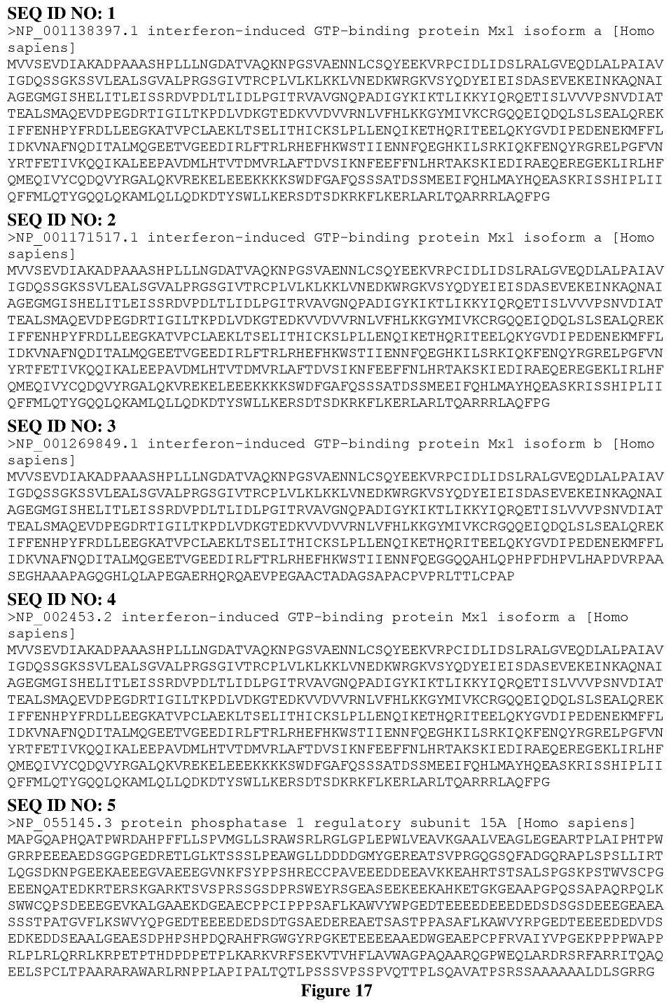

The marker for CM and TReactive cells, MX1, was positively correlated with the genes involved in the cytotoxic function during the vaccine treatment (FIG. 3I). Most of genes that were strong positively correlated with MX1 were related to the activation of T cells (FIG. 16). MX1 depletion remarkably impaired the anti-tumor ability of immune cells in PBMC (FIGS. 3J and 3K).

1.2.4 the Benefit from the Combination of Neoantigen Vaccines and Anti-PD-1 Treatment for Patients with Tumor Relapse Through Activating the Bacterial Stimulus Pathway in CD8+ T Cells

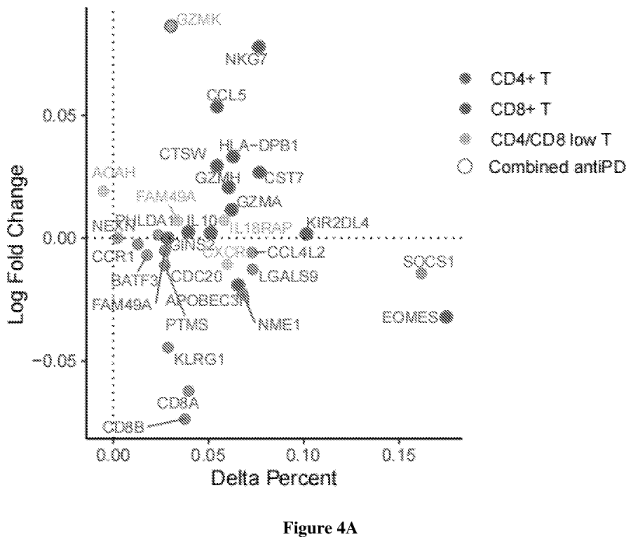

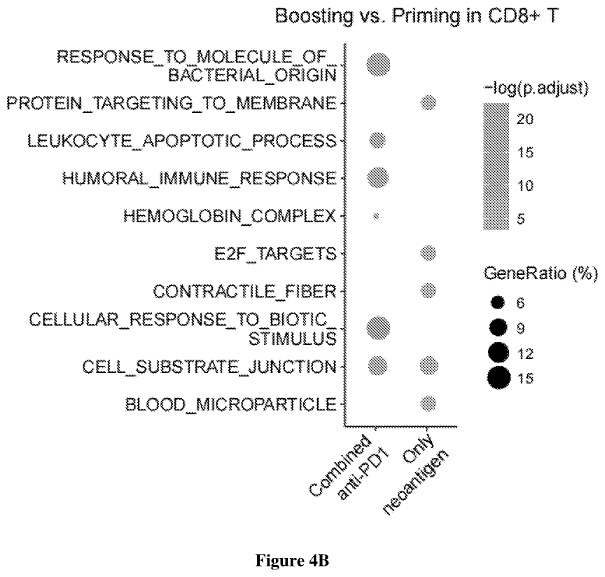

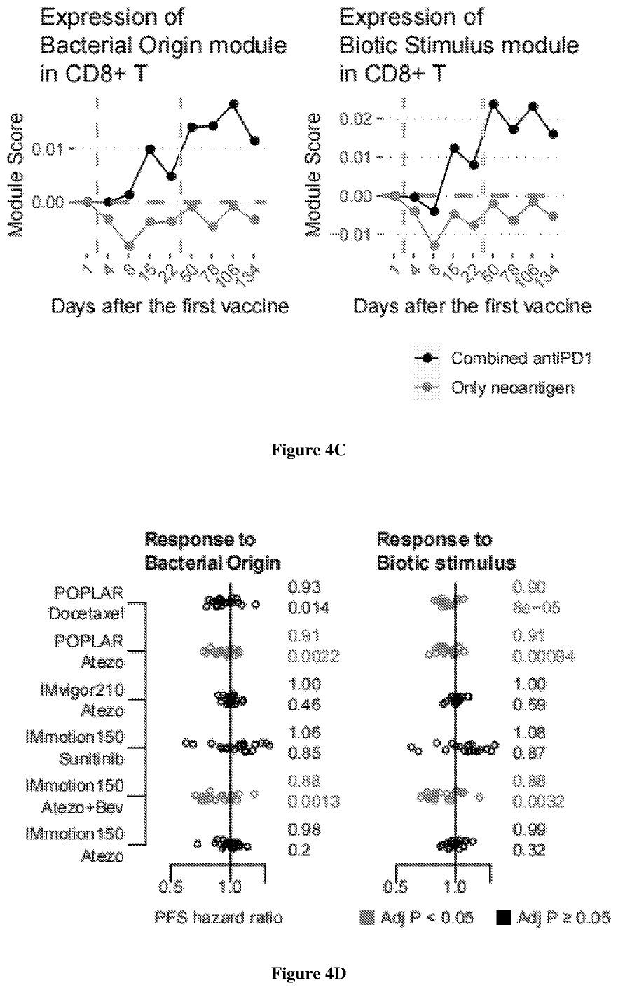

Three patients with tumor relapse before/during the vaccination were treated with adjuvant anti-PD1 in the boosting phase ( ). Combination treatment significantly increased expression of genes related to cytotoxicity and they were enriched in CD8+ T cells (FIG. 4A). Enriched functions of those genes were response to molecule of bacterial origin and cellular response to biotic stimulus pathways (FIG. 4B) and the average expression of these two modules significantly increased only in relapse patients after the combination treatment (FIG. 4C). Although it was reported the patients in response to anti-PD1 benefited from the CD4+ T cells in the peripheral blood 8 and tumor-infiltrating T cells⁷, our result suggested that the major response in T cell was enriched in CD8+ T cells when using the combination of neoantigen vaccines and anti-PD1. The expressions of those genes were positively associated with the progression-free survival in the atezolizumab-containing arm from three clinical trials⁹⁻¹¹ (FIG. 4D). These results might partially explain why relapsed patients' disease can be controlled in our study.

### 1.2.5 Dynamics of Immunologic Responses in Peripheral Blood

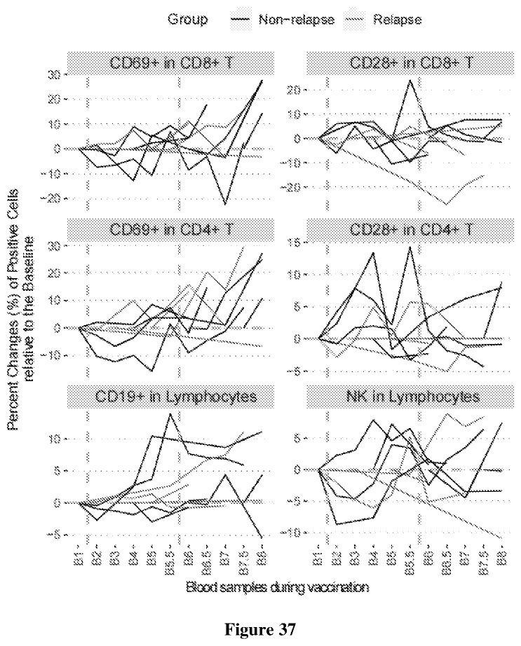

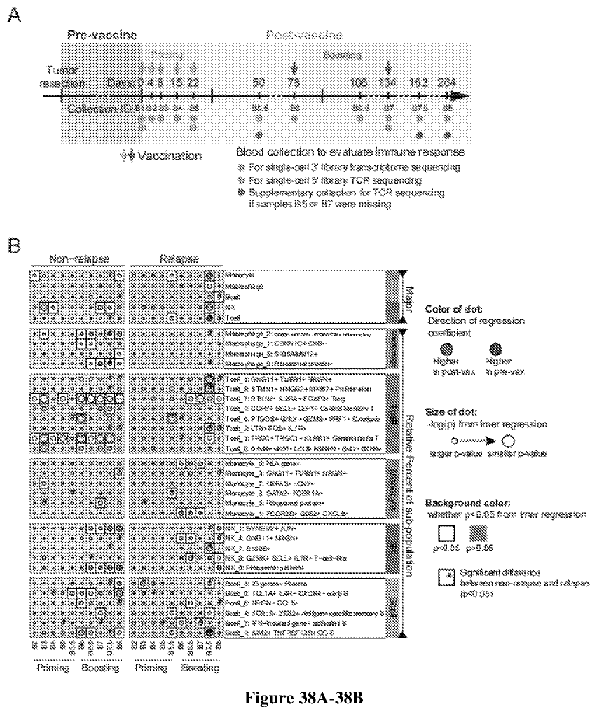

Since immunological parameters are key determinants of efficacy, we also examined the immunologic correlates in the peripheral blood. IFN-γ responses of 96% (177/185) of the individualized neoantigen peptides or pools in the stimulation of PBMCs were detectable by ex vivo ELISpot in all 12 patients. Ten-color flow cytometric analysis of the blood samples showed an increased proportion of CD69+ or CD28+ T and B or NK cells during the vaccination (FIG. 37). Using single cell transcriptome sequencing, we investigated the systemic changes in percent of immune cell types and their subtypes (data below are shown as mean±SE[%] at pre-vaccine to maximum mean±SE at post-vaccine). Compared to pre-vaccine (FIG. 38), we observed significant increases in the inflammation-related macrophage subtype (CD63+ NFKBIA+ APOBEC3A+ 20,21; 12.9±18.3 to 53.4±15.5), Macrophage_1 (CDKN1C+ CKB+; 23.7±10.1 to 43.9±7.2), Tcell_6 cytotoxic T cells (PTGDS+ GNLY+ GZMB+ PRF1+; 2.8±0.3 to 4.8±1.2), central memory T cells (CCR7+ SELL+ LEF1+; 22.1±3.2 to 28.3±4.3) and Bcell_0 (TCL1A+ IL4R+ CXCR4+, which might be early B-cell 22; 34.4±6.2 to 61.9±7.7) in non-relapse patients and significant increases in HLA gene+ monocyte (47.3±2.9 to 73.1±6.9), proliferation-related T cells (STMN1+ HMGB2+ MKI67+; 1.1±0.3 to 11±10.3) and immunoglobulin gene positive B cells (4.9±1.9 to 35±31.6) in relapse patients. Both relapse and non-relapse patients had significant increases of regulatory T cells (RTKN2+ IL2RA+ FOXP3+; 2.7±0.3 to 5.7±0.5 in non-relapse; 3.1±0.4 to 5.1±0.3 in relapse) and significant decreases of T6-T cells (TRDC+ TRGC1+ KLRB1+; 6.5±1.5 to 3.2±0.4 in non-relapse; 9±2.5 to 7.8±4.9 in relapse), antigen-specific memory B cells (FCRL5+ ZEB2+ 23; 11.9±2.8 to 8.6±2.6 in non-relapse; 8.1±0.3 to 3.4±3.4 in relapse) and germinal center B cells (AIM2+ TNFRSF13B+ 24,25; 36.2±5 to 24.8±6.2 in non-relapse; 35.5±6.8 to 8.7±8.7 in relapse).

1.2.6 Identification of Genes Associated with Significant Differential Changes in Immune Response in Peripheral Blood of Pancreatic Cancer Patients Treated with Personalized Neoantigen Vaccines
1.2.6.1 Genes with Significantly Altered Expression Levels During the Priming Phase or Boosting Phase of Vaccination, Compared to the Pre-Vaccination (Table 5)

Genes were selected by adjusted. Pvalue<0.05, PercentChange(%,RelativeToBaseline)>10 and AUC>0.9.

TABLE 5
Genes with significantly altered expression levels during the priming
phase or boosting phase of vaccination, compared to the pre-vaccination

| Gene_{—} | | | PercentChange(%, | | | |
|---|---|---|---|---|---|---|
| name | Cell | Phase | RelativeToBaseline) | Percent.phase(%) | Percent.baseline(%) | Percent.range.phase(%) |
| AC011815.2 | TcellCD8 | Priming | 15.30268 | 18.97834 | 3.675658 | 5.17~25.3 |
| AC011815.2 | TcellCD8 | Boosting | 16.11784 | 19.7935 | 3.675658 | 8.59~26.69 |
| ANKRD29 | TcellCD4 | Boosting | 10.97347 | 11.24181 | 0.268338 | 0.24~37.77 |
| AP3M2 | Monocyte | Boosting | 15.01695 | 17.28933 | 2.272387 | 1.23~68.95 |
| ARL5B | Monocyte | Boosting | 18.99725 | 23.7437 | 4.746449 | 6.5~43.43 |
| ATF4 | Bcell | Priming | 12.04116 | 30.82931 | 18.78815 | 23.81~41.9 |
| ATP5F1B | Bcell | Boosting | 10.85673 | 46.2648 | 35.40807 | 32.7~53.6 |
| ATP5F1B | Monocyte | Boosting | 16.41051 | 43.51306 | 27.10255 | 36.54~54.33 |
| C15orf54 | TcellCD4 | Priming | 11.96527 | 12.2114 | 0.246136 | 0.11~35.95 |
| C15orf54 | TcellCD4 | Boosting | 12.74488 | 12.99102 | 0.246136 | 0.19~31.68 |
| CHCHD2 | Monocyte | Boosting | 18.01593 | 77.25809 | 59.24216 | 62.62~89.92 |
| COL4A3BP | NK | Boosting | 36.10794 | 48.49167 | 12.38373 | 4.84~80.75 |
| COPE | Macrophage | Boosting | 24.3096 | 82.0025 | 57.6929 | 63.94~92.8 |
| COPE | Monocyte | Boosting | 15.40557 | 49.29313 | 33.88755 | 41.57~59.69 |
| CTTNBP2 | Macrophage | Boosting | 17.93018 | 24.71362 | 6.783443 | 13.7~44.27 |
| DDOST | Bcell | Priming | 13.01769 | 31.2867 | 18.26901 | 18.04~48.3 |
| DERL1 | Bcell | Priming | 11.1133 | 24.54877 | 13.43547 | 14.85~36.33 |
| DNPH1 | Bcell | Priming | 10.05609 | 19.00522 | 8.949125 | 13.68~25.08 |
| EGF | TcellCD8 | Priming | 20.00824 | 20.1021 | 0.093866 | 0.26~30.23 |
| EGF | TcellCD8 | Boosting | 20.51555 | 20.60941 | 0.093866 | 7.28~29.36 |
| FERMT3 | NK | Priming | 40.58401 | 80.76042 | 40.17641 | 57.98~95.34 |
| FERMT3 | NK | Boosting | 38.42326 | 78.59968 | 40.17641 | 46.7~96.12 |
| FOSL2 | TcellCD4 | Priming | 15.05711 | 20.01874 | 4.961638 | 10.46~33.49 |
| FOSL2 | TcellCD4 | Boosting | 12.77148 | 17.73311 | 4.961638 | 3.73~29.11 |
| GCH1 | TcellCD4 | Priming | 26.92688 | 32.75041 | 5.823529 | 5.82~69.48 |
| GCH1 | TcellCD8 | Priming | 33.43306 | 39.42794 | 5.99488 | 7.57~80.31 |
| GK | Macrophage | Priming | 13.16868 | 20.28774 | 7.119062 | 14.88~26.06 |
| GK | Macrophage | Boosting | 19.11469 | 26.23375 | 7.119062 | 6.8~53.44 |
| GLA | Monocyte | Boosting | 13.32723 | 19.67405 | 6.346819 | 8.73~44.53 |
| LMAN2 | Bcell | Priming | 15.07443 | 36.2788 | 21.20437 | 24.45~51.08 |
| LRRK1 | Macrophage | Boosting | 15.53785 | 20.88523 | 5.347383 | 9.67~38.17 |
| MAP9 | Bcell | Priming | 18.20469 | 23.76124 | 5.556557 | 5.48~46.96 |
| MAP9 | Bcell | Boosting | 13.72868 | 19.28523 | 5.556557 | 7.59~35.48 |
| MN1 | Monocyte | Boosting | 12.18984 | 12.90195 | 0.712114 | 1.79~27.81 |
| MTDH | Macrophage | Boosting | 21.89294 | 80.82181 | 58.92887 | 68.67~90 |
| MTDH | Monocyte | Boosting | 19.19494 | 57.1951 | 38.00016 | 50.49~64.88 |
| NFIL3 | NK | Boosting | 45.05373 | 63.75578 | 18.70205 | 31.92~84.16 |
| NUAK1 | TcellCD8 | Priming | 16.83026 | 18.46317 | 1.63291 | 2.52~34.85 |
| NUAK1 | TcellCD8 | Boosting | 18.72308 | 20.35599 | 1.63291 | 3.81~32.82 |
| PDE4D | Bcell | Priming | 15.54772 | 24.86794 | 9.320222 | 11.29~34.62 |
| PIM1 | Bcell | Boosting | 41.4424 | 60.4738 | 19.03139 | 23.61~81.49 |
| PRKACA | TcellCD4 | Priming | 21.4698 | 29.8306 | 8.360805 | 6.63~80.64 |
| PRMT1 | Macrophage | Boosting | 11.48025 | 23.51731 | 12.03706 | 12.72~36.67 |
| SERTAD2 | TcellCD4 | Priming | 34.66481 | 40.71115 | 6.046332 | 7.05~82.65 |
| SERTAD2 | TcellCD8 | Priming | 62.71583 | 67.70562 | 4.98979 | 33.88~84.94 |
| SERTAD2 | TcellCD8 | Boosting | 65.96399 | 70.95378 | 4.98979 | 56.21~81.09 |
| SLC16A6 | TcellCD4 | Priming | 12.46152 | 14.1112 | 1.649675 | 2.68~43.21 |
| SLC16A6 | TcellCD4 | Boosting | 10.89199 | 12.54166 | 1.649675 | 3.36~22.25 |
| SYT17 | Macrophage | Boosting | 16.13279 | 23.44766 | 7.314869 | 5.07~37.89 |
| TMED10 | Macrophage | Priming | 12.10987 | 28.48079 | 16.37092 | 21.57~36.94 |
| TMED10 | Macrophage | Boosting | 10.84109 | 27.21201 | 16.37092 | 18.94~35.56 |
| TMEM258 | Bcell | Priming | 15.60784 | 42.31839 | 26.71055 | 34.17~51.2 |
| TRDV1 | TcellCD4 | Boosting | 16.02261 | 20.28409 | 4.261475 | 0.92~34.68 |
| WDFY3 | TcellCD4 | Priming | 13.43445 | 13.73923 | 0.304778 | 3.95~37.16 |
| ZBTB43 | TcellCD4 | Priming | 28.60523 | 35.29939 | 6.694162 | 8.63~85.41 |
| ZDHHC7 | Bcell | Priming | 39.29549 | 51.26929 | 11.97379 | 2.79~83.77 |
| ZDHHC7 | Bcell | Boosting | 38.64275 | 50.61654 | 11.97379 | 20.67~79.03 |

| Gene_{—} | | | | | | |
|---|---|---|---|---|---|---|
| name | Percent.range.baseline(%) | AUC | Threshold | Pvalue | Coeffecient | adjusted.Pvalue |
| AC011815.2 | 0.06~14.19 | 0.958333 | 15.15854 | 4.78E−09 | 0.361721 | 2E−06 |
| AC011815.2 | 0.06~14.19 | 0.958333 | 15.31728 | 2.54E−09 | 0.375872 | 9.27E−07 |
| ANKRD29 | 0.08~0.53 | 0.958333 | 0.63504 | 2.64E−12 | 0.27903 | 1.95E−09 |
| AP3M2 | 1.05~3.22 | 0.956 | 3.277557 | 2.03E−05 | 0.1656 | 0.001092 |
| ARL5B | 0.9~9.07 | 0.971429 | 10.36063 | 0.000777 | 0.26109 | 0.012114 |
| ATF4 | 17.08~21.33 | 1 | 22.57143 | 0.000313 | 0.141605 | 0.015941 |
| ATP5F1B | 28.87~45.56 | 0.925926 | 45.75075 | 0.005151 | 0.124971 | 0.039071 |
| ATP5F1B | 20.69~34.66 | 1 | 35.60152 | 1.35E−05 | 0.190534 | 0.000818 |
| C15orf54 | 0~0.77 | 0.9125 | 0.898872 | 9.8E−07 | 0.320177 | 0.000225 |
| C15orf54 | 0~0.77 | 0.9625 | 0.847246 | 7.31E−07 | 0.331846 | 0.000102 |
| CHCHD2 | 43.72~73.13 | 0.925926 | 74.12439 | 0.000644 | 0.232204 | 0.010787 |
| COL4A3BP | 8.71~14.54 | 0.916667 | 14.90708 | 3.38E−06 | 0.437448 | 0.00032 |
| COPE | 39.3~67.46 | 0.925926 | 71.33175 | 3.22E−05 | 0.267117 | 0.001496 |
| COPE | 24.9~43.56 | 0.925926 | 45.54908 | 0.00111 | 0.183213 | 0.015119 |
| CTTNBP2 | 1.18~17.95 | 0.972222 | 18.97436 | 0.000196 | 0.340547 | 0.004904 |
| DDOST | 14.58~25.11 | 0.916667 | 25.55396 | 0.000657 | 0.16204 | 0.02581 |
| DERL1 | 10.36~18.89 | 0.916667 | 19.97076 | 3.98E−05 | 0.150118 | 0.003733 |
| DNPH1 | 6.35~12 | 1 | 12.83761 | 0.00026 | 0.149083 | 0.014136 |
| EGF | 0.07~0.12 | 1 | 0.189843 | 0.000369 | 0.4104 | 0.017871 |
| EGF | 0.07~0.12 | 1 | 3.702414 | 0.000275 | 0.421949 | 0.006211 |
| FERMT3 | 20.49~78.26 | 0.90625 | 63.55423 | 4.56E−07 | 0.482043 | 0.000117 |
| FERMT3 | 20.49~78.26 | 0.901961 | 65.88727 | 7.56E−06 | 0.437801 | 0.000547 |
| FOSL2 | 0.25~13.34 | 0.958333 | 13.46445 | 0.000149 | 0.249238 | 0.009883 |
| FOSL2 | 0.25~13.34 | 0.942308 | 13.68799 | 0.001574 | 0.211226 | 0.018568 |
| GCH1 | 5.06~6.94 | 0.958333 | 7.22239 | 7.85E−09 | 0.321808 | 3.18E−06 |
| GCH1 | 5.54~6.43 | 1 | 7.002858 | 2.65E−08 | 0.400659 | 9.35E−06 |
| GK | 3.79~15.34 | 0.96875 | 15.54915 | 5.42E−05 | 0.206914 | 0.004748 |
| GK | 3.79~15.34 | 0.90625 | 6.05388 | 1.8E−05 | 0.222228 | 0.000999 |
| GLA | 4.06~9.44 | 0.952381 | 9.59067 | 0.000125 | 0.196184 | 0.0037 |
| LMAN2 | 16.55~29.11 | 0.916667 | 30.41316 | 0.000393 | 0.178077 | 0.018683 |
| LRRK1 | 3.07~7.06 | 1 | 8.360321 | 4.96E−09 | 0.22607 | 1.67E−06 |
| MAP9 | 5.06~6.05 | 0.9375 | 6.321614 | 4.65E−05 | 0.240631 | 0.00423 |
| MAP9 | 5.06~6.05 | 1 | 6.819977 | 0.004891 | 0.177023 | 0.038008 |
| MN1 | 0.2~1.56 | 1 | 1.674855 | 0.005795 | 0.227383 | 0.041917 |
| MTDH | 39.62~68.97 | 0.962963 | 71.67622 | 0.000164 | 0.231458 | 0.004343 |
| MTDH | 27.64~50 | 1 | 50.24631 | 9.12E−05 | 0.218659 | 0.002985 |
| NFIL3 | 7.9~46.15 | 0.931818 | 22.26928 | 4.83E−08 | 0.542398 | 1.26E−05 |
| NUAK1 | 0.77~3.75 | 0.9375 | 1.824599 | 1.81E−13 | 0.3355 | 1.8E−10 |
| NUAK1 | 0.77~3.75 | 1 | 3.780879 | 2.25E−13 | 0.333358 | 2.07E−10 |
| PDE4D | 2.93~15.69 | 0.958333 | 17.98361 | 1.13E−06 | 0.251928 | 0.00025 |
| PIM1 | 11.37~26.69 | 0.9375 | 33.10326 | 6.14E−07 | 0.407535 | 8.88E−05 |
| PRKACA | 1.42~27.17 | 0.916667 | 6.623547 | 0.001256 | 0.209055 | 0.037879 |
| PRMT1 | 9.75~14.44 | 0.962963 | 14.79559 | 0.001524 | 0.152857 | 0.018217 |
| SERTAD2 | 5.82~6.27 | 1 | 6.662071 | 2.42E−09 | 0.413784 | 1.1E−06 |
| SERTAD2 | 3.97~6.01 | 1 | 19.94611 | 5.64E−11 | 0.749591 | 3.48E−08 |
| SERTAD2 | 3.97~6.01 | 1 | 31.11316 | 6.42E−12 | 0.791095 | 4.16E−09 |
| SLC16A6 | 1.09~2.65 | 1 | 2.6662 | 3.88E−05 | 0.228885 | 0.003712 |
| SLC16A6 | 1.09~2.65 | 1 | 3.00702 | 0.000689 | 0.190383 | 0.011216 |
| SYT17 | 1.37~12.32 | 0.952381 | 12.38546 | 0.000152 | 0.226439 | 0.004141 |
| TMED10 | 13.54~21.55 | 1 | 21.56018 | 0.001082 | 0.154889 | 0.034598 |
| TMED10 | 13.54~21.55 | 0.925926 | 22.06618 | 0.003247 | 0.137729 | 0.029501 |
| TMEM258 | 17.61~33.19 | 1 | 33.67594 | 0.00060 | 0.149499 | 0.024559 |
| TRDV1 | 0~20.47 | 0.907692 | 0.741602 | 0.00475 | 0.31014 | 0.037324 |
| WDFY3 | 0.29~0.32 | 1 | 2.134985 | 0.000891 | 0.305407 | 0.030797 |
| ZBTB43 | 3.78~8.92 | 0.979167 | 9.001219 | 3.21E−09 | 0.42554 | 1.43E−06 |
| ZDHHC7 | 6.05~23.63 | 0.916667 | 26.4646 | 5.59E−06 | 0.514059 | 0.000871 |
| ZDHHC7 | 6.05~23.63 | 0.916667 | 26.628 | 6.01E−06 | 0.521797 | 0.000473 |

1.2.6.2 Genes with Significantly Different Expression in Patients with and without Tumor Relapse (Table 6)

Genes were selected by adjusted. Pvalue<0.05, PercentChange(%,Nonrelapse.vs.Relapse)>40 and AUC>0.9.

TABLE 6
Genes with significantly different expression in patients with and without tumor relapse

| Gene_{—} | | | PercentChange(%, | | | |
|---|---|---|---|---|---|---|
| name | Cell | Phase | Nonrelapse.vs.Relapse) | Percent.Nonrelapse(%) | Percent.Relapse(%) | Percent.range.Nonrelapse(%) |
| ABCA13 | Monocyte | Pre- | 40.391 | 41.27517 | 0.884169 | 41.28~41.28 |
| | | vaccine | | | | |
| AC022726.2 | NK | Pre- | 96.10108 | 96.47683 | 0.375759 | 92.95~100 |
| | | vaccine | | | | |
| AC022726.2 | NK | Priming | 57.19124 | 57.51708 | 0.325841 | 54.39~61.5 |
| AC022726.2 | NK | Boosting | 52.18607 | 52.61554 | 0.429479 | 40.89~59.33 |
| AC105094.2 | Bcell | Pre- | 96.88675 | 99.29453 | 2.407785 | 98.59~100 |
| | | vaccine | | | | |
| AC105094.2 | Bcell | Priming | 87.72077 | 99.79893 | 12.07816 | 99.2~100 |
| AC105094.2 | Bcell | Boosting | 80.98152 | 99.38544 | 18.40391 | 98.45~100 |
| AC243829.2 | Bcell | Pre- | 99.38272 | 99.38272 | 0 | 98.77~100 |
| | | vaccine | | | | |
| AC243829.2 | Bcell | Priming | 99.50058 | 99.50058 | 0 | 98.39~100 |
| AC243829.2 | Bcell | Boosting | 98.69427 | 98.74635 | 0.052083 | 96.2~100 |
| AL021807.1 | Bcell | Pre- | 99.35302 | 99.73545 | 0.382434 | 99.47~100 |
| | | vaccine | | | | |
| AL021807.1 | Bcell | Priming | 91.99705 | 92.53397 | 0.536923 | 88.89~96.76 |
| AL021807.1 | Bcell | Boosting | 88.31638 | 88.66536 | 0.348972 | 77.85~96.7 |
| AL391807.1 | Bcell | Pre- | 93.62843 | 93.95218 | 0.323752 | 81.86~100 |
| | | vaccine | | | | |
| AL391807.1 | Bcell | Priming | 91.14354 | 93.36241 | 2.218868 | 66.96~100 |
| AL391807.1 | Bcell | Boosting | 96.65208 | 98.17597 | 1.523889 | 94.77~100 |
| AL662907.3 | TcellCD4 | Pre- | 97.66779 | 97.73243 | 0.064641 | 95.46~100 |
| | | vaccine | | | | |
| APOBEC3C | NK | Priming | 43.40895 | 80.97153 | 37.56258 | 52.35~95.79 |
| ATP1B3 | Monocyte | Pre- | 56.38505 | 56.75336 | 0.368301 | 51.01~62.5 |
| | | vaccine | | | | |
| ATP1B3 | Monocyte | Priming | 42.91369 | 43.07398 | 0.160293 | 35.69~50.15 |
| ATP1B3 | Monocyte | Boosting | 45.284 | 45.3451 | 0.061107 | 33.21~51.13 |
| ATP6V1B2 | Bcell | Priming | 49.81155 | 57.89552 | 8.083975 | 4.02~92.11 |
| ATP6V1B2 | Bcell | Boosting | 54.32738 | 65.00505 | 10.67767 | 14.15~88.12 |
| C1QBP | Bcell | Boosting | 43.08138 | 43.08138 | 0 | 38.98~49.64 |
| CBX5 | Macrophage | Pre- | 66.49309 | 81.58996 | 15.09686 | 81.59~81.59 |
| | | vaccine | | | | |
| CBX5 | Macrophage | Boosting | 43.77394 | 62.40375 | 18.62981 | 47.58~73.6 |
| CCNE2 | Macrophage | Boosting | 65.49028 | 79.64697 | 14.15669 | 53.79~100 |
| CD63 | TcellCD4 | Priming | 87.40899 | 88.76672 | 1.357733 | 77.62~98.16 |
| CEBPA | Macrophage | Boosting | 40.28112 | 40.73122 | 0.450094 | 0~93.99 |
| CHP1 | TcellCD8 | Priming | 46.73653 | 65.92974 | 19.1932 | 51.08~87.09 |
| CHP1 | TcellCD8 | Boosting | 53.14483 | 71.64828 | 18.50345 | 47.44~85.53 |
| CLN8 | Macrophage | Priming | 64.57066 | 72.65072 | 8.080063 | 19.03~91.3 |
| CLN8 | Macrophage | Boosting | 68.47339 | 79.8045 | 11.33112 | 62.9~98.89 |
| CMBL | Bcell | Pre- | 99.74734 | 99.91182 | 0.164474 | 99.82~100 |
| | | vaccine | | | | |
| CMBL | Bcell | Priming | 99.35081 | 99.43614 | 0.085324 | 98.93~100 |
| CMBL | Bcell | Boosting | 95.91621 | 96.28495 | 0.368732 | 86.82~100 |
| CNIH4 | Bcell | Priming | 66.09042 | 74.70358 | 8.613164 | 48.03~93.26 |
| CNIH4 | Bcell | Boosting | 67.69527 | 76.41837 | 8.723097 | 69.6~82.65 |
| COL4A3BP | TcellCD4 | Boosting | 46.06473 | 60.67579 | 14.61106 | 39.53~85.19 |
| CPNE3 | TcellCD4 | Priming | 52.23368 | 70.04425 | 17.81057 | 20.38~90.9 |
| CPNE3 | TcellCD4 | Boosting | 53.92957 | 73.59282 | 19.66325 | 58.64~84.24 |
| DACH1 | NK | Pre- | 94.91658 | 95.07892 | 0.162345 | 85.24~100 |
| | | vaccine | | | | |
| DACH1 | NK | Priming | 94.00085 | 96.43501 | 2.434163 | 86.8~100 |
| DACH1 | NK | Boosting | 92.41794 | 94.3755 | 1.957555 | 90.19~99.59 |
| DNAJC12 | Macrophage | Pre- | 78.20691 | 81.62651 | 3.419596 | 63.25~100 |
| | | vaccine | | | | |
| DNAJC12 | Macrophage | Priming | 89.04369 | 92.44692 | 3.403231 | 83.54~100 |
| DNAJC12 | Macrophage | Boosting | 75.4809 | 79.79297 | 4.312064 | 46.88~92.92 |
| DNAJC3 | Monocyte | Pre- | 83.16712 | 90.25367 | 7.086556 | 90.25~90.25 |
| | | vaccine | | | | |
| DNAJC3 | Monocyte | Boosting | 60.89509 | 70.81373 | 9.918634 | 60.41~83 |
| DOC2B | Bcell | Pre- | 99.45055 | 99.45055 | 0 | 99.45~99.45 |
| | | vaccine | | | | |
| DOC2B | Bcell | Priming | 98.20789 | 98.70131 | 0.493421 | 96.41~100 |
| DOC2B | Bcell | Boosting | 99.23515 | 99.50397 | 0.268817 | 98.94~100 |
| EEA1 | TcellCD4 | Priming | 46.79972 | 58.10452 | 11.30481 | 8.76~85.49 |
| EEA1 | TcellCD4 | Boosting | 56.30989 | 65.03691 | 8.727021 | 44.77~81.92 |
| EEA1 | TcellCD8 | Priming | 49.51294 | 59.3401 | 9.827164 | 9.25~86.51 |
| EEA1 | TcellCD8 | Boosting | 55.46818 | 65.70823 | 10.24006 | 51.01~81.36 |
| ENAM | Monocyte | Pre- | 43.75198 | 43.79172 | 0.039746 | 43.79~43.79 |
| | | vaccine | | | | |
| ERG | NK | Pre- | 99.7124 | 100 | 0.287596 | 100~100 |
| | | vaccine | | | | |
| ERG | NK | Priming | 92.74613 | 93.14054 | 0.394413 | 80.19~100 |
| ERG | NK | Boosting | 97.29172 | 97.42945 | 0.137728 | 85.4~100 |
| FAM43A | Bcell | Priming | 45.57434 | 66.66687 | 21.09253 | 8.36~100 |
| FAM43A | Bcell | Boosting | 56.02853 | 76.83337 | 20.80483 | 34.6~96.09 |
| FBXO43 | Macrophage | Pre- | 81.07227 | 85.98326 | 4.910994 | 85.98~85.98 |
| | | vaccine | | | | |
| FBXO43 | Macrophage | Priming | 95.21661 | 99.87745 | 4.660845 | 99.51~100 |
| FBXO43 | Macrophage | Boosting | 97.14148 | 100 | 2.858515 | 100~100 |
| FIGN | Macrophage | Pre- | 96.7392 | 97.2973 | 0.558101 | 97.3~97.3 |
| | | vaccine | | | | |
| FIGN | Macrophage | Priming | 95.39067 | 96.37858 | 0.987912 | 93.98~100 |
| FIGN | Macrophage | Boosting | 97.40586 | 99.00723 | 1.601368 | 96.93~100 |
| FIGN | Monocyte | Pre- | 95.4586 | 96.97987 | 1.52127 | 96.98~96.98 |
| | | vaccine | | | | |
| FIGN | Monocyte | Priming | 95.50211 | 96.63218 | 1.130072 | 92.65~100 |
| FIGN | Monocyte | Boosting | 94.23131 | 97.47488 | 3.243572 | 90.22~100 |
| GCA | Bcell | Boosting | 47.84331 | 59.24353 | 11.40022 | 15.26~89.28 |
| GNG4 | Monocyte | Priming | 77.85065 | 77.85555 | 0.0049 | 69.62~100 |
| GNG4 | Monocyte | Boosting | 93.26377 | 93.53246 | 0.268682 | 74.13~100 |
| GNLY | Bcell | Boosting | 52.03133 | 66.85169 | 14.82036 | 13.47~99.01 |
| GOLIM4 | Macrophage | Pre- | 72.42311 | 82.21757 | 9.794464 | 82.22~82.22 |
| | | vaccine | | | | |
| GOLIM4 | Macrophage | Boosting | 62.63461 | 77.26313 | 14.62852 | 58.89~88.8 |
| GPM6B | TcellCD4 | Priming | 59.76481 | 60.83639 | 1.071583 | 6.2~86.89 |
| GPM6B | TcellCD4 | Boosting | 62.70722 | 63.82006 | 1.112842 | 30.01~82.21 |
| GPR171 | Macrophage | Boosting | 59.34295 | 65.16553 | 5.822578 | 57.26~76.52 |
| GPRC5D | Macrophage | Pre- | 73.34127 | 91.70279 | 18.36152 | 54.82~100 |
| | | vaccine | | | | |
| GPRC5D | Macrophage | Priming | 87.99223 | 98.07497 | 10.08274 | 84.81~100 |
| GPRC5D | Macrophage | Boosting | 81.88466 | 93.56015 | 11.67548 | 50~100 |
| HHEX | Macrophage | Priming | 42.483 | 48.48021 | 5.997213 | 12~83.54 |
| HHEX | Macrophage | Boosting | 45.60598 | 52.48637 | 6.880387 | 11.76~84.29 |
| HID1 | Bcell | Pre- | 85.98791 | 100 | 14.01209 | 100~100 |
| | | vaccine | | | | |
| HID1 | Bcell | Priming | 77.18721 | 99.34211 | 22.15489 | 97.37~100 |
| HID1 | Bcell | Boosting | 89.42669 | 95.56104 | 6.134354 | 93.02~100 |
| ILF2 | Monocyte | Pre- | 63.40549 | 72.83044 | 9.42495 | 72.83~72.83 |
| | | vaccine | | | | |
| ILF2 | Monocyte | Boosting | 44.03603 | 61.18772 | 17.15169 | 54.78~72.41 |
| IRF2BP2 | NK | Priming | 54.69726 | 64.68104 | 9.98378 | 20.77~90.59 |
| IRF2BP2 | NK | Boosting | 60.50552 | 72.18802 | 11.6825 | 54.6~83.85 |
| KIF22 | Monocyte | Priming | 41.83864 | 52.72321 | 10.88458 | 16.02~73.16 |
| KIF22 | Monocyte | Boosting | 44.41785 | 56.58492 | 12.16707 | 43.53~66.01 |
| LACC1 | Bcell | Priming | 59.75861 | 63.58357 | 3.824956 | 40.38~100 |
| LACC1 | Bcell | Boosting | 56.40865 | 59.30169 | 2.893038 | 32.09~76.22 |
| LIPA | TcellCD4 | Boosting | 51.14612 | 70.37018 | 19.22406 | 35.95~85.09 |
| MANEA | Monocyte | Boosting | 46.84719 | 49.43797 | 2.590786 | 32.39~56.16 |
| MECOM | Macrophage | Priming | 81.4467 | 90.52825 | 9.081541 | 75.41~100 |
| MECOM | Macrophage | Boosting | 72.17237 | 78.35168 | 6.179315 | 63.24~100 |
| MID1IP1 | Bcell | Priming | 53.81266 | 71.39344 | 17.58078 | 17.87~97.47 |
| MID1IP1 | Bcell | Boosting | 50.32638 | 69.78472 | 19.45834 | 44.21~90.16 |
| MX2 | Bcell | Boosting | 40.99385 | 50.93241 | 9.93856 | 6.2~84.08 |
| MYOM2 | NK | Priming | 45.02615 | 60.8552 | 15.82906 | 28.87~96.16 |
| MYOM2 | NK | Boosting | 45.30509 | 63.61692 | 18.31183 | 29.86~93.85 |
| NABP1 | Macrophage | Priming | 46.99533 | 65.23169 | 18.23636 | 48.33~74.24 |
| NABP1 | Macrophage | Boosting | 57.7671 | 73.6499 | 15.8828 | 69.21~81.62 |
| NEFH | Monocyte | Pre- | 88.99503 | 100 | 11.00497 | 100~100 |
| | | vaccine | | | | |
| NFE4 | NK | Pre- | 93.09045 | 100 | 6.909548 | 100~100 |
| | | vaccine | | | | |
| NFE4 | NK | Priming | 90.42847 | 98.07539 | 7.646918 | 87.3~100 |
| NFE4 | NK | Boosting | 76.96331 | 90.57248 | 13.60916 | 75.18~99.94 |
| NLN | Monocyte | Pre- | 55.9809 | 62.08333 | 6.102434 | 62.08~62.08 |
| | | vaccine | | | | |
| OXCT2 | Macrophage | Pre- | 74.17573 | 98.0695 | 23.89377 | 94.21~100 |
| | | vaccine | | | | |
| OXCT2 | Macrophage | Priming | 80.73624 | 92.21657 | 11.48034 | 81.8~100 |
| OXCT2 | Macrophage | Boosting | 76.97117 | 91.20341 | 14.23224 | 83.99~100 |
| P3H2 | Monocyte | Pre- | 72.95229 | 74.01108 | 1.058796 | 65.1~82.5 |
| | | vaccine | | | | |
| P3H2 | Monocyte | Priming | 79.08856 | 82.72692 | 3.638354 | 36.29~100 |
| P3H2 | Monocyte | Boosting | 84.7574 | 87.17314 | 2.415739 | 59.64~100 |
| PI3 | Bcell | Pre- | 72.26702 | 72.26702 | 0 | 0.04~100 |
| | | vaccine | | | | |
| PI3 | Bcell | Priming | 85.77517 | 88.33347 | 2.558302 | 11.02~100 |
| PI3 | Bcell | Boosting | 94.25421 | 94.74138 | 0.48717 | 72.77~100 |
| PIM1 | Bcell | Priming | 50.48055 | 74.14751 | 23.66696 | 61.36~83.66 |
| PLAU | Monocyte | Pre- | 98.01934 | 100 | 1.980663 | 100~100 |
| | | vaccine | | | | |
| PLAU | Monocyte | Priming | 49.27202 | 64.19671 | 14.92469 | 35.13~87.25 |
| PLAU | Monocyte | Boosting | 75.26279 | 81.61375 | 6.350964 | 60.6~91.23 |
| PRSS3 | NK | Pre- | 98.72859 | 99.47472 | 0.746127 | 99.47~99.47 |
| | | vaccine | | | | |
| PRSS3 | NK | Priming | 95.40287 | 98.06912 | 2.666245 | 93.07~100 |
| PRSS3 | NK | Boosting | 94.3796 | 96.26299 | 1.883392 | 94.53~100 |
| PSMA3 | TcellCD4 | Pre- | 62.11745 | 73.19631 | 11.07886 | 73.2~73.2 |
| | | vaccine | | | | |
| PSMA3 | TcellCD4 | Boosting | 55.9765 | 68.76369 | 12.78718 | 52.03~78.91 |
| RAB10 | TcellCD4 | Priming | 54.43719 | 67.08147 | 12.64428 | 9.51~91.55 |
| RAB10 | TcellCD4 | Boosting | 62.33454 | 73.06847 | 10.73393 | 46.41~93.4 |
| RAB20 | TcellCD4 | Boosting | 42.5953 | 44.70686 | 2.111567 | 35.76~48.76 |
| RASGRP2 | Macrophage | Pre- | 46.82354 | 47.03478 | 0.211234 | 38.47~55.6 |
| | | vaccine | | | | |
| RASGRP2 | Macrophage | Priming | 60.06689 | 60.18985 | 0.122962 | 33.36~80.26 |
| RASGRP2 | Macrophage | Boosting | 62.88383 | 62.88383 | 0 | 49.85~82.2 |
| RBMS3 | TcellCD4 | Priming | 61.40912 | 69.95594 | 8.546826 | 59.04~100 |
| RBMS3 | TcellCD4 | Boosting | 65.74807 | 75.7256 | 9.977535 | 57.76~95.29 |
| RPL23 | NK | Pre- | 56.59882 | 56.59882 | 0 | 56.6~56.6 |
| | | vaccine | | | | |
| RPL23 | NK | Priming | 58.72947 | 58.89961 | 0.170136 | 53.87~63.68 |
| RPL23 | NK | Boosting | 56.6829 | 56.79014 | 0.10724 | 54.41~59.05 |
| RPS6KA5 | NK | Pre- | 70.24786 | 77.90261 | 7.65475 | 70.85~84.96 |
| | | vaccine | | | | |
| RPS6KA5 | NK | Boosting | 50.86649 | 68.79586 | 17.92936 | 55.47~84.73 |
| RPS8 | TcellCD4 | Pre- | 74.03179 | 74.03179 | 0 | 66.25~81.96 |
| | | vaccine | | | | |
| RPS8 | TcellCD4 | Priming | 72.43941 | 72.43941 | 0 | 53.73~90.08 |
| RPS8 | TcellCD4 | Boosting | 64.41258 | 64.46399 | 0.051415 | 50.74~77 |
| RPS8 | TcellCD8 | Pre- | 46.10504 | 46.22611 | 0.121065 | 24.33~60.13 |
| | | vaccine | | | | |
| RPS8 | TcellCD8 | Priming | 47.55766 | 47.55766 | 0 | 20.05~62.19 |
| RPS8 | TcellCD8 | Boosting | 40.86011 | 40.91306 | 0.052952 | 17.02~57.44 |
| RUNX1 | TcellCD8 | Priming | 65.27488 | 73.28976 | 8.014875 | 53.62~84.98 |
| RUNX1 | TcellCD8 | Boosting | 65.67337 | 73.22425 | 7.55088 | 58.82~84.27 |
| SAMD3 | TcellCD4 | Priming | 52.65539 | 67.52011 | 14.86472 | 56.04~78.19 |
| SAMD3 | TcellCD4 | Boosting | 59.38169 | 70.52165 | 11.13996 | 59.74~86.64 |
| SEPT11 | Bcell | Pre- | 57.15134 | 63.2967 | 6.145361 | 63.3~63.3 |
| | | vaccine | | | | |
| SEPT11 | Bcell | Boosting | 55.38869 | 60.77732 | 5.38863 | 53.04~66.27 |
| SERTAD2 | TcellCD4 | Priming | 65.00074 | 73.21152 | 8.210774 | 52.81~82.65 |
| SERTAD2 | TcellCD4 | Boosting | 70.16093 | 77.1074 | 6.946463 | 65.89~81.58 |
| SIGLEC6 | Monocyte | Priming | 85.51335 | 94.49019 | 8.976836 | 85.76~100 |
| SIGLEC6 | Monocyte | Boosting | 80.93534 | 95.44228 | 14.50694 | 91.34~99.54 |
| SIT1 | Bcell | Priming | 52.1741 | 64.25739 | 12.08328 | 47.78~81.07 |
| SIT1 | Bcell | Boosting | 69.87031 | 82.1022 | 12.23189 | 63.81~95.24 |
| SOCS1 | TcellCD4 | Priming | 52.12083 | 60.24686 | 8.12603 | 43.62~76.09 |
| SOCS1 | TcellCD4 | Boosting | 61.7899 | 76.43476 | 14.64486 | 70.33~84.3 |
| SSR1 | Monocyte | Boosting | 40.65188 | 60.48985 | 19.83797 | 39.96~81.03 |
| ST6GALNAC1 | Macrophage | Pre- | 95.76618 | 100 | 4.233825 | 100~100 |
| | | vaccine | | | | |
| ST6GALNAC1 | Macrophage | Priming | 91.72337 | 99.60317 | 7.879809 | 98.41~100 |
| ST6GALNAC1 | Macrophage | Boosting | 88.81989 | 95.94957 | 7.129684 | 88.57~100 |
| SYNE2 | Bcell | Pre- | 78.17787 | 91.56118 | 13.38331 | 91.56~91.56 |
| | | vaccine | | | | |
| SYNE2 | Bcell | Priming | 53.57978 | 73.32041 | 19.74063 | 30.62~93.96 |
| SYNE2 | Bcell | Boosting | 69.5806 | 90.24116 | 20.66056 | 87.31~93.17 |
| TRAV16 | Monocyte | Pre- | 73.04872 | 73.14385 | 0.095129 | 27.5~100 |
| | | vaccine | | | | |
| TRAV16 | Monocyte | Priming | 89.52717 | 89.80397 | 0.276802 | 62.13~100 |
| TRAV16 | Monocyte | Boosting | 90.28154 | 90.41082 | 0.129275 | 68.64~99.05 |
| TREM2 | NK | Pre- | 99.95812 | 100 | 0.041876 | 100~100 |
| | | vaccine | | | | |
| TREM2 | NK | Priming | 99.03329 | 99.08344 | 0.05015 | 96.78~100 |
| TREM2 | NK | Boosting | 98.80217 | 98.83574 | 0.033568 | 97.46~100 |
| TXNDC15 | TcellCD4 | Pre- | 65.41154 | 70.4698 | 5.058255 | 70.47~70.47 |
| | | vaccine | | | | |
| TXNDC15 | TcellCD4 | Boosting | 55.28482 | 63.64531 | 8.360491 | 57.04~72.94 |
| TXNDC17 | Macrophage | Priming | 50.34724 | 50.53668 | 0.189435 | 24.06~72.84 |
| TXNDC17 | Macrophage | Boosting | 62.48263 | 62.57219 | 0.089563 | 36.09~92.86 |
| UAP1 | Macrophage | Pre- | 43.98816 | 47.48954 | 3.501382 | 47.49~47.49 |
| | | vaccine | | | | |
| UFM1 | Monocyte | Pre- | 75.81039 | 84.11215 | 8.301757 | 84.11~84.11 |
| | | vaccine | | | | |
| UFM1 | Monocyte | Boosting | 56.39435 | 67.88547 | 11.49112 | 52.91~79.24 |
| UGT2B17 | TcellCD8 | Priming | 85.0632 | 87.02196 | 1.958762 | 61.88~100 |
| UGT2B17 | TcellCD8 | Boosting | 83.62061 | 85.5457 | 1.925093 | 55.14~100 |
| XPNPEP1 | TcellCD8 | Priming | 62.38431 | 66.65648 | 4.272172 | 55.42~82.6 |
| XPNPEP1 | TcellCD8 | Boosting | 60.65513 | 64.43821 | 3.783083 | 47.1~76.6 |
| ZBTB16 | Monocyte | Pre- | 41.26052 | 43.12417 | 1.863646 | 43.12~43.12 |
| | | vaccine | | | | |
| ZNF608 | TcellCD8 | Pre- | 99.79339 | 99.79339 | 0 | 99.59~100 |
| | | vaccine | | | | |
| ZNF608 | TcellCD8 | Priming | 94.78929 | 94.96491 | 0.175626 | 93.07~99.95 |
| ZNF608 | TcellCD8 | Boosting | 96.88765 | 96.99336 | 0.105709 | 88.72~100 |

| Gene_{—} | | | | | | |
|---|---|---|---|---|---|---|
| name | Percent.range.Relapse(%) | AUC | Threshold | Pvalue | Coeffecient | adjusted.Pvalue |
| ABCA13 | 0~2.66 | 1 | 21.9694 | 0.000464 | 0.626825 | 0.028284 |
| AC022726.2 | 0.11~0.69 | 1 | 46.82071 | 0 | 1.377764 | 0 |
| AC022726.2 | 0.06~0.55 | 1 | 27.46687 | 0 | 0.807281 | 0 |
| AC022726.2 | 0.21~0.89 | 1 | 20.89037 | 0 | 0.747449 | 0 |
| AC105094.2 | 0~9.56 | 1 | 54.07231 | 1.11E−10 | 1.425823 | 2.05E−08 |
| AC105094.2 | 0~38.57 | 1 | 68.88113 | 0 | 1.299017 | 0 |
| AC105094.2 | 0~66.96 | 1 | 82.70563 | 2.22E−16 | 1.173148 | 3.51E−14 |
| AC243829.2 | 0~0 | 1 | 49.38272 | 0 | 1.515126 | 0 |
| AC243829.2 | 0~0 | 1 | 49.19571 | 0 | 1.523399 | 0 |
| AC243829.2 | 0~0.16 | 1 | 48.17939 | 0 | 1.464468 | 0 |
| AL021807.1 | 0~1.04 | 1 | 50.25358 | 0 | 1.483219 | 0 |
| AL021807.1 | 0~2.05 | 1 | 45.46834 | 0 | 1.243093 | 0 |
| AL021807.1 | 0~2.65 | 1 | 40.25148 | 0 | 1.218458 | 0 |
| AL391807.1 | 0~0.62 | 1 | 41.23596 | 0 | 1.364604 | 0 |
| AL391807.1 | 0~7.31 | 1 | 37.13402 | 0 | 1.318052 | 0 |
| AL391807.1 | 0~4.84 | 1 | 49.80863 | 0 | 1.358332 | 0 |
| AL662907.3 | 0.06~0.06 | 1 | 47.76475 | 0.000295 | 1.438068 | 0.019464 |
| APOBEC3C | 32.23~41.13 | 1 | 46.7418 | 4.51E−07 | 0.483028 | 3.21E−05 |
| ATP1B3 | 0.31~0.43 | 1 | 25.71734 | 0 | 0.793077 | 0 |
| ATP1B3 | 0~0.31 | 1 | 17.9986 | 0 | 0.681364 | 0 |
| ATP1B3 | 0~0.13 | 1 | 16.67011 | 0 | 0.720795 | 0 |
| ATP6V1B2 | 3.85~17.63 | 0.952381 | 20.43026 | 6.38E−07 | 0.584124 | 4.42E−05 |
| ATP6V1B2 | 3.53~29.17 | 0.97931 | 29.93827 | 2.6E−07 | 0.618851 | 1.7E−05 |
| C1QBP | 0~0 | 1 | 19.48791 | 9.82E−11 | 0.715765 | 1.01E−08 |
| CBX5 | 6.2~35.34 | 1 | 58.46739 | 0.000203 | 0.750122 | 0.013906 |
| CBX5 | 4.46~33.43 | 1 | 40.5063 | 0.001372 | 0.485092 | 0.039426 |
| CCNE2 | 0~40.98 | 1 | 47.38174 | 0.001265 | 0.931839 | 0.037081 |
| CD63 | 0.51~2.27 | 1 | 39.94789 | 0.000663 | 1.149693 | 0.024043 |
| CEBPA | 0~1.97 | 0.90101 | 6.027932 | 0.00085 | 0.621031 | 0.026672 |
| CHP1 | 7.24~36.72 | 1 | 43.90253 | 8.17E−06 | 0.521233 | 0.000483 |
| CHP1 | 4.59~37.17 | 1 | 42.30595 | 7.96E−07 | 0.593479 | 4.89E−05 |
| CLN8 | 1.09~17.66 | 1 | 18.34683 | 3.18E−06 | 0.778916 | 0.000202 |
| CLN8 | 0~24.85 | 1 | 43.87461 | 3.18E−07 | 0.864302 | 2.05E−05 |
| CMBL | 0~0.33 | 1 | 50.07629 | 1.41E−07 | 1.521099 | 1.95E−05 |
| CMBL | 0~0.34 | 1 | 49.63446 | 1.24E−07 | 1.492247 | 9.5E−06 |
| CMBL | 0~1.47 | 1 | 44.14832 | 6.57E−07 | 1.400082 | 4.07E−05 |
| CNIH4 | 5.47~11.95 | 1 | 29.9863 | 0 | 0.770172 | 0 |
| CNIH4 | 5.88~11.8 | 1 | 40.69936 | 0 | 0.768058 | 0 |
| COL4A3BP | 10.42~18.56 | 1 | 29.04522 | 0.000131 | 0.508592 | 0.005223 |
| CPNE3 | 6.79~36.63 | 0.958333 | 46.75657 | 1.85E−07 | 0.581078 | 1.39E−05 |
| CPNE3 | 7.26~35.12 | 1 | 46.87916 | 1.97E−07 | 0.580323 | 1.34E−05 |
| DACH1 | 0.08~0.22 | 1 | 42.729 | 0 | 1.375915 | 0 |
| DACH1 | 0.06~4.96 | 1 | 45.87975 | 0 | 1.307028 | 0 |
| DACH1 | 0.2~5.59 | 1 | 47.8863 | 0 | 1.211876 | 0 |
| DNAJC12 | 0.73~7.97 | 1 | 35.61357 | 2.3E−12 | 1.07098 | 5.06E−10 |
| DNAJC12 | 0~18.21 | 1 | 50.8762 | 0 | 1.190862 | 0 |
| DNAJC12 | 0~14.63 | 1 | 30.75457 | 5.77E−15 | 0.96545 | 8.04E−13 |
| DNAJC3 | 5.27~8.9 | 1 | 49.57835 | 7.59E−07 | 0.985984 | 8.86E−05 |
| DNAJC3 | 6.29~15.29 | 1 | 37.85056 | 1.63E−09 | 0.68822 | 1.42E−07 |
| DOC2B | 0~0 | 1 | 49.72527 | 0 | 1.496603 | 0 |
| DOC2B | 0~1.97 | 1 | 49.18946 | 0 | 1.456162 | 0 |
| DOC2B | 0~0.81 | 1 | 49.87413 | 0 | 1.490965 | 0 |
| EEA1 | 7.52~14.97 | 0.9375 | 16.38715 | 3.21E−05 | 0.525347 | 0.001664 |
| EEA1 | 7.54~10.94 | 1 | 27.85568 | 6.24E−06 | 0.643994 | 0.000345 |
| EEA1 | 7.24~13.59 | 0.958333 | 13.84986 | 2.18E−05 | 0.564076 | 0.001183 |
| EEA1 | 7.81~12.55 | 1 | 31.78074 | 2.99E−05 | 0.625684 | 0.001443 |
| ENAM | 0~0.08 | 1 | 21.93561 | 2.22E−16 | 0.709056 | 6.06E−14 |
| ERG | 0~0.67 | 1 | 50.33501 | 0 | 1.536367 | 0 |
| ERG | 0~1.16 | 1 | 40.6733 | 0 | 1.337054 | 0 |
| ERG | 0~0.3 | 1 | 42.85292 | 0 | 1.452058 | 0 |
| FAM43A | 5.17~51.09 | 0.925 | 51.87595 | 0.000421 | 0.533926 | 0.016269 |
| FAM43A | 5.64~51.96 | 0.980676 | 55.59712 | 2.16E−05 | 0.627265 | 0.001078 |
| FBXO43 | 0~13.87 | 1 | 49.92594 | 7.24E−10 | 1.045264 | 1.3E−07 |
| FBXO43 | 0~16.3 | 1 | 57.90708 | 0 | 1.39221 | 0 |
| FBXO43 | 0~8.01 | 1 | 54.00411 | 0 | 1.447485 | 0 |
| FIGN | 0~2.53 | 1 | 49.91127 | 0 | 1.373565 | 0 |
| FIGN | 0~4.32 | 1 | 49.15422 | 0 | 1.34343 | 0 |
| FIGN | 0~5.95 | 1 | 51.43789 | 0 | 1.416007 | 0 |
| FIGN | 0~7.46 | 1 | 52.219 | 0 | 1.32584 | 0 |
| FIGN | 0~6.4 | 1 | 49.52836 | 0 | 1.344008 | 0 |
| FIGN | 0~10.83 | 1 | 50.5238 | 0 | 1.325654 | 0 |
| GCA | 4.89~22.58 | 0.987879 | 22.74284 | 3.17E−05 | 0.533613 | 0.001523 |
| GNG4 | 0~0.02 | 1 | 34.81911 | 1.8E−05 | 1.136482 | 0.000988 |
| GNG4 | 0~0.81 | 1 | 37.46793 | 2.07E−07 | 1.407432 | 1.39E−05 |
| GNLY | 2.35~44.77 | 0.929293 | 47.1984 | 0.001185 | 0.58501 | 0.035108 |
| GOLIM4 | 6.38~16.07 | 1 | 49.14541 | 1.16E−05 | 0.824067 | 0.001073 |
| GOLIM4 | 8.18~20.85 | 1 | 39.86698 | 8.62E−12 | 0.696482 | 9.67E−10 |
| GPM6B | 0.26~2.22 | 1 | 4.209851 | 0.000112 | 0.802529 | 0.005026 |
| GPM6B | 0.45~1.43 | 1 | 15.72041 | 7.81E-05 | 0.815819 | 0.003348 |
| GPR171 | 5.18~6.92 | 1 | 32.08978 | 0.001309 | 0.698502 | 0.038114 |
| GPRC5D | 17.67~19.05 | 1 | 36.93496 | 8.06E−07 | 0.994071 | 9.28E−05 |
| GPRC5D | 0.05~25.94 | 1 | 55.37549 | 5.99E−12 | 1.226465 | 7.53E−10 |
| GPRC5D | 0~19.93 | 1 | 34.96493 | 2.77E−10 | 1.101441 | 2.7E−08 |
| HHEX | 0~27.88 | 0.957031 | 7.081081 | 2.66E−05 | 0.613543 | 0.001422 |
| HHEX | 0~28.34 | 0.943434 | 33.69556 | 4.38E−06 | 0.671778 | 0.000247 |
| HID1 | 4.91~23.11 | 1 | 61.55556 | 4.37E−11 | 1.208292 | 8.39E−09 |
| HID1 | 1.1~37.56 | 1 | 67.46646 | 0 | 1.080384 | 0 |
| HID1 | 3.31~11.7 | 1 | 52.35612 | 0 | 1.142272 | 0 |
| ILF2 | 7.64~11.21 | 1 | 42.01935 | 0.000177 | 0.711757 | 0.012326 |
| ILF2 | 12.11~26.46 | 1 | 40.62047 | 1.16E−05 | 0.47598 | 0.000611 |
| IRF2BP2 | 5.26~13.67 | 1 | 17.21549 | 5.12E−08 | 0.627287 | 4.23E−06 |
| IRF2BP2 | 5.77~19.65 | 1 | 37.12863 | 1.63E−07 | 0.680356 | 1.12E−05 |
| KIF22 | 8.4~14.84 | 1 | 15.43176 | 0.001046 | 0.472661 | 0.035807 |
| KIF22 | 6.31~19.21 | 1 | 31.3699 | 0.00037 | 0.500994 | 0.012988 |
| LACC1 | 0~6.93 | 1 | 23.65946 | 4.67E−08 | 0.793964 | 3.86E−06 |
| LACC1 | 0~4.42 | 1 | 18.25717 | 1.66E−06 | 0.733809 | 9.82E−05 |
| LIPA | 4.75~34.33 | 1 | 35.14172 | 1.38E−06 | 0.568491 | 8.27E−05 |
| MANEA | 1.85~4.08 | 1 | 18.23557 | 3.37E−05 | 0.619101 | 0.001591 |
| MECOM | 0.28~29.75 | 1 | 52.57731 | 6.88E−07 | 1.101295 | 4.74E−05 |
| MECOM | 0~24.33 | 1 | 43.78223 | 2.29E−05 | 0.934293 | 0.001137 |
| MID1IP1 | 5.58~37.96 | 0.976563 | 43.52356 | 5.63E−06 | 0.620643 | 0.000342 |
| MID1IP1 | 8.06~42.16 | 1 | 43.18369 | 3.27E−05 | 0.575528 | 0.001549 |
| MX2 | 6.56~16.77 | 0.921839 | 16.86009 | 0.000445 | 0.486412 | 0.015192 |
| MYOM2 | 9.5~24.86 | 1 | 26.86512 | 1.67E−08 | 0.501847 | 1.48E−06 |
| MYOM2 | 12.45~28.29 | 1 | 29.0728 | 1.87E−07 | 0.464906 | 1.27E−05 |
| NABP1 | 7.34~32.61 | 1 | 40.47101 | 4.08E−06 | 0.509778 | 0.000255 |
| NABP1 | 7.03~28.04 | 1 | 48.62334 | 4.86E−09 | 0.641265 | 4.14E−07 |
| NEFH | 0.33~18.4 | 1 | 59.20114 | 2.68E−07 | 1.314381 | 3.51E−05 |
| NFE4 | 0~13.82 | 1 | 56.90955 | 2.22E−15 | 1.397548 | 5.75E−13 |
| NFE4 | 0~12.54 | 1 | 49.92012 | 0 | 1.248716 | 0 |
| NFE4 | 12.71~14.12 | 1 | 44.64959 | 1.71E−10 | 0.971463 | 1.7E−08 |
| NLN | 6.1~6.1 | 1 | 34.09288 | 0.00042 | 0.657825 | 0.026006 |
| OXCT2 | 10.13~37.66 | 1 | 65.93336 | 2.69E−08 | 0.988498 | 4.1E−06 |
| OXCT2 | 0.11~28.76 | 1 | 55.28246 | 4.44E−16 | 1.047058 | 7.06E−14 |
| OXCT2 | 0.27~30.1 | 1 | 57.04529 | 2.89E−15 | 0.955567 | 4.11E−13 |
| P3H2 | 0~2.34 | 1 | 33.71877 | 1.09E−06 | 0.960318 | 0.000123 |
| P3H2 | 0~12.43 | 1 | 24.35886 | 1.15E−14 | 1.118676 | 1.73E−12 |
| P3H2 | 0~8.69 | 1 | 34.16496 | 8.88E−16 | 1.172908 | 1.31E−13 |
| PI3 | 0~0 | 1 | 0.019275 | 2.16E−05 | 1.081516 | 0.001919 |
| PI3 | 0~12.96 | 0.992188 | 36.53488 | 3.03E−08 | 1.227085 | 2.6E−06 |
| PI3 | 0~1.39 | 1 | 37.08079 | 9.62E−10 | 1.38061 | 8.68E−08 |
| PIM1 | 18.42~26.72 | 1 | 44.0406 | 0.001444 | 0.536426 | 0.047056 |
| PLAU | 1.37~2.59 | 1 | 51.29573 | 8E−11 | 1.431299 | 1.5E−08 |
| PLAU | 5.82~22.53 | 1 | 28.83003 | 0.00043 | 0.556828 | 0.01653 |
| PLAU | 4.28~8.21 | 1 | 34.40299 | 3.27E−08 | 0.88758 | 2.42E−06 |
| PRSS3 | 0~2.09 | 1 | 50.78426 | 0 | 1.437176 | 0 |
| PRSS3 | 0~9.13 | 1 | 51.09815 | 0 | 1.338912 | 0 |
| PRSS3 | 0~2.92 | 1 | 48.72499 | 0 | 1.277726 | 0 |
| PSMA3 | 9.13~13.03 | 1 | 43.11104 | 0.000332 | 0.688491 | 0.021465 |
| PSMA3 | 6.7~18.99 | 1 | 35.51359 | 2.04E−07 | 0.623109 | 1.37E−05 |
| RAB10 | 8.39~18.33 | 0.9375 | 31.11718 | 2.07E−10 | 0.609938 | 2.32E−08 |
| RAB10 | 8.05~14.24 | 1 | 30.3247 | 3.01E−11 | 0.703878 | 3.23E−09 |
| RAB20 | 1.43~2.68 | 1 | 19.22314 | 2.98E−05 | 0.587357 | 0.00144 |
| RASGRP2 | 0.1~0.32 | 1 | 19.39378 | 2.5E−08 | 0.707464 | 3.85E−06 |
| RASGRP2 | 0~0.32 | 1 | 16.84126 | 6.22E−15 | 0.869714 | 9.53E−13 |
| RASGRP2 | 0~0 | 1 | 24.92355 | 4.44E−16 | 0.907123 | 6.85E−14 |
| RBMS3 | 0.9~21.47 | 1 | 40.25575 | 0.000395 | 0.796328 | 0.01538 |
| RBMS3 | 1.48~21.67 | 1 | 39.71412 | 0.000507 | 0.790711 | 0.016939 |
| RPL23 | 0~0 | 1 | 28.29941 | 0 | 0.851579 | 0 |
| RPL23 | 0~0.3 | 1 | 27.08188 | 0 | 0.839855 | 0 |
| RPL23 | 0~0.19 | 1 | 27.29953 | 0 | 0.826894 | 0 |
| RPS6KA5 | 6.7~9.54 | 1 | 40.19459 | 0.000213 | 0.785437 | 0.014514 |
| RPS6KA5 | 6.04~37.8 | 1 | 46.63532 | 0.000161 | 0.576712 | 0.00626 |
| RPS8 | 0~0 | 1 | 33.12395 | 0 | 1.044254 | 0 |
| RPS8 | 0~0 | 1 | 26.86505 | 0 | 1.023857 | 0 |
| RPS8 | 0~0.1 | 1 | 25.41914 | 0 | 0.920889 | 0 |
| RPS8 | 0.12~0.12 | 1 | 12.22479 | 0.00069 | 0.730394 | 0.039526 |
| RPS8 | 0~0 | 1 | 10.02513 | 0.000331 | 0.757938 | 0.013182 |
| RPS8 | 0~0.16 | 1 | 8.587412 | 0.001112 | 0.689628 | 0.033438 |
| RUNX1 | 2.51~13.79 | 1 | 33.70355 | 0 | 0.755504 | 0 |
| RUNX1 | 2.14~11.08 | 1 | 34.94663 | 0 | 0.762963 | 0 |
| SAMD3 | 10.93~19.77 | 1 | 37.90939 | 6.63E−13 | 0.573028 | 8.99E−11 |
| SAMD3 | 8.03~16.93 | 1 | 38.33854 | 0 | 0.666001 | 0 |
| SEPT11 | 4.28~8.29 | 1 | 35.79343 | 3.77E−06 | 0.671555 | 0.000398 |
| SEPT11 | 2.35~7.42 | 1 | 30.22814 | 4.44E−16 | 0.663635 | 6.85E−14 |
| SERTAD2 | 7.05~9.96 | 1 | 31.38565 | 0 | 0.744429 | 0 |
| SERTAD2 | 5.57~8.22 | 1 | 37.0553 | 0 | 0.808479 | 0 |
| SIGLEC6 | 0~33.54 | 1 | 59.64823 | 2.15E−05 | 1.194101 | 0.001168 |
| SIGLEC6 | 0~46.3 | 1 | 68.82181 | 0.000243 | 1.102413 | 0.008977 |
| SIT1 | 4.1~27.59 | 1 | 37.68199 | 2.22E−15 | 0.594434 | 3.45E−13 |
| SIT1 | 4.9~20.16 | 1 | 41.98578 | 0 | 0.811078 | 0 |
| SOCS1 | 6.43~11.06 | 1 | 27.3433 | 1.46E−12 | 0.604093 | 1.95E−10 |
| SOCS1 | 5.88~20.16 | 1 | 45.24682 | 4.22E−15 | 0.683982 | 5.94E−13 |
| SSR1 | 14.59~25.67 | 1 | 32.81588 | 0.001342 | 0.438069 | 0.038754 |
| ST6GALNAC1 | 0~12.73 | 1 | 56.36605 | 0 | 1.406856 | 0 |
| ST6GALNAC1 | 0~24.25 | 1 | 61.33242 | 0 | 1.321068 | 0 |
| ST6GALNAC1 | 0~12.16 | 1 | 50.3668 | 0 | 1.171837 | 0 |
| SYNE2 | 7.08~22.18 | 1 | 56.87008 | 2.62E−06 | 0.90822 | 0.000283 |
| SYNE2 | 5.58~40.61 | 0.9375 | 23.95205 | 6.02E−09 | 0.615765 | 5.82E−07 |
| SYNE2 | 7.06~29.41 | 1 | 58.3626 | 1.08E−08 | 0.792629 | 8.62E−07 |
| TRAV16 | 0~0.19 | 1 | 13.84513 | 3.94E−07 | 1.104665 | 4.96E−05 |
| TRAV16 | 0~1.03 | 1 | 31.57696 | 2.22E−16 | 1.280029 | 3.55E−14 |
| TRAV16 | 0.1~0.16 | 1 | 34.40338 | 5.59E−13 | 1.240412 | 7.03E−11 |
| TREM2 | 0~0.08 | 1 | 50.04188 | 0 | 1.556324 | 0 |
| TREM2 | 0~0.2 | 1 | 48.49139 | 0 | 1.497752 | 0 |
| TREM2 | 0~0.1 | 1 | 48.77846 | 0 | 1.468753 | 0 |
| TXNDC15 | 4.76~5.36 | 1 | 37.91468 | 0.00026 | 0.769545 | 0.017348 |
| TXNDC15 | 6.13~11.47 | 1 | 34.25663 | 6.1E−08 | 0.634512 | 4.42E−06 |
| TXNDC17 | 0.1~0.34 | 1 | 12.19873 | 4.48E−07 | 0.747633 | 3.2E−05 |
| TXNDC17 | 0~0.27 | 1 | 18.18358 | 8.28E−10 | 0.900156 | 7.64E−08 |
| UAP1 | 1.33~5.6 | 1 | 26.54649 | 5.04E−06 | 0.579315 | 0.000514 |
| UFM1 | 6.59~10.02 | 1 | 47.06402 | 7.25E−05 | 0.870019 | 0.005591 |
| UFM1 | 7.33~17.48 | 1 | 35.19419 | 1.88E−05 | 0.631975 | 0.000954 |
| UGT2B17 | 0~3.06 | 1 | 32.47134 | 8.59E−05 | 1.150121 | 0.00401 |
| UGT2B17 | 0.79~3.53 | 1 | 29.33551 | 0.000102 | 1.125381 | 0.004205 |
| XPNPEP1 | 3.62~4.99 | 1 | 30.20248 | 0 | 0.751365 | 0 |
| XPNPEP1 | 2.59~5.19 | 1 | 26.14538 | 0 | 0.740974 | 0 |
| ZBTB16 | 1.58~2.15 | 1 | 22.63521 | 7.15E−06 | 0.57987 | 0.000705 |
| ZNF608 | 0~0 | 1 | 49.79339 | 0 | 1.538633 | 0 |
| ZNF608 | 0~0.74 | 1 | 46.90031 | 0 | 1.337921 | 0 |
| ZNF608 | 0~0.32 | 1 | 44.52013 | 0 | 1.407232 | 0 |

1.2.6.3 Genes with Significantly Altered Expression after Vaccine Combined with PD1 Treatment (Table 7)

Genes were selected by adjusted. Pvalue<0.05, PercentChange(%,RelativeToPre-antiPD1)>10.

TABLE 7
Genes with significantly altered expression after vaccine combined with PD1 treatment

| Gene_{—} | | | PercentChange(%, | | | |
|---|---|---|---|---|---|---|
| name | Cell | Phase | RelativeToPre) | Percent.phase(%) | Percent.Pre(%) | Percent.range.phase(%) |
| AC092490.1 | Bcell | post- | 12.51008 | 32.52478 | 20.0147 | 0.38~81.21 |
| | | antiPD1 | | | | |
| ALDH1L2 | Monocyte | post- | 12.28871 | 30.49074 | 18.20203 | 0.28~77.26 |
| | | antiPD1 | | | | |
| LILRB5 | TcellCD4 | post- | 14.70517 | 15.91862 | 1.213451 | 0~27.61 |
| | | antiPD1 | | | | |
| PALD1 | Bcell | post- | 23.50248 | 51.47512 | 27.97265 | 0~99.78 |
| | | antiPD1 | | | | |
| PKP2 | Monocyte | post- | 10.13253 | 23.90911 | 13.77657 | 0.27~50.81 |
| | | antiPD1 | | | | |
| TRAV35 | Bcell | post- | 10.29073 | 34.79301 | 24.50228 | 0~100 |
| | | antiPD1 | | | | |

| Gene_{—} | | | | | | |
|---|---|---|---|---|---|---|
| name | Percent.range.Pre(%) | AUC | Threshold | Pvalue | Coeffecient | adjusted.Pvalue |
| AC092490.1 | 0~59.92 | 0.633929 | 13.02203 | 0.000258 | 0.658068 | 0.028856 |
| ALDH1L2 | 0~80.87 | 0.625 | 0.416506 | 5.09E−07 | 0.598652 | 0.000227 |
| LILRB5 | 0~7.13 | 0.75 | 16.39671 | 3.81E−05 | 0.438984 | 0.007166 |
| PALD1 | 0~100 | 0.699519 | 27.78786 | 0.000425 | 0.555102 | 0.04292 |
| PKP2 | 0~47.99 | 0.714286 | 1.271429 | 2E−08 | 0.766312 | 1.3E−05 |
| TRAV35 | 0~100 | 0.641544 | 0.056689 | 0 | 0.55024 | 0 |

### 1.2.7 Expansion of T-Cell Clonotypes During Vaccination

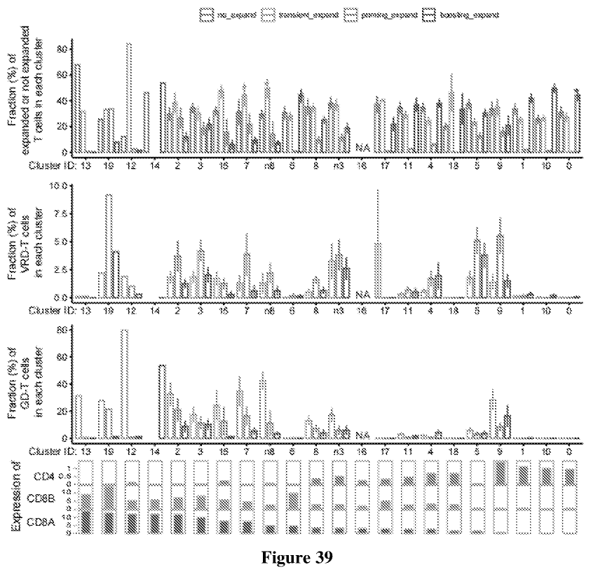

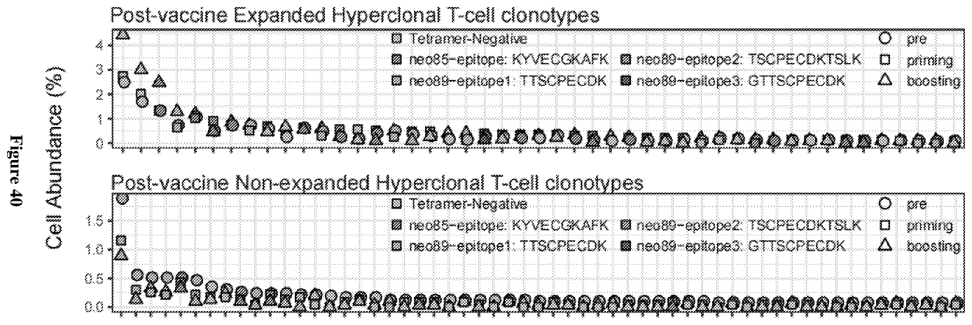

To test the hypothesis that vaccination increased the frequency of specific T-cell clones in blood, we initially performed single-cell TCR sequencing in blood samples from 10 patients (P3˜12; 3 with relapse and 7 without relapse) at pre-vaccination, the end of the priming phase and the boosting phase. After the vaccination, a mean of 67.3% (range, 38.3˜84.6) of detected T-cell clonotypes per patient had an expansion in abundance, but a small number (mean 2.3%, range 0.7˜6.1) were hyper-clonal. Among the expanded hyper-clonal clonotypes, 46.4% (range, 16.7˜80) ones per patient were pre-existing hyper-clonal before vaccination (Guarding T-cell clonotypes [GD-T]) and remaining ones were expanded from non-clonality (Vaccine-related de novo T-cell clonotypes [VRD-T]). We further classified the expanded clones according subtypes and phases (FIG. 39). We observed the CD8+ T cells had higher mean percent of priming-phase-expanded cells while CD8+ memory T cells and CD4+ T cells had higher mean percent of boosting-phase-expanded cells. Both GD-T and VRD-T cells had higher mean percent of priming-phase-expanded cells and were enriched in CD8/4+ cytotoxic T cells. Three patients with relapse (P4, P6, P9) had lower percent of GD-Ts in CD8+ cytotoxic T cells and P4 also had less GD-Ts in CD4+ cytotoxic T cells. P9 had less VRD-Ts in CD8/4+ cytotoxic T cells.

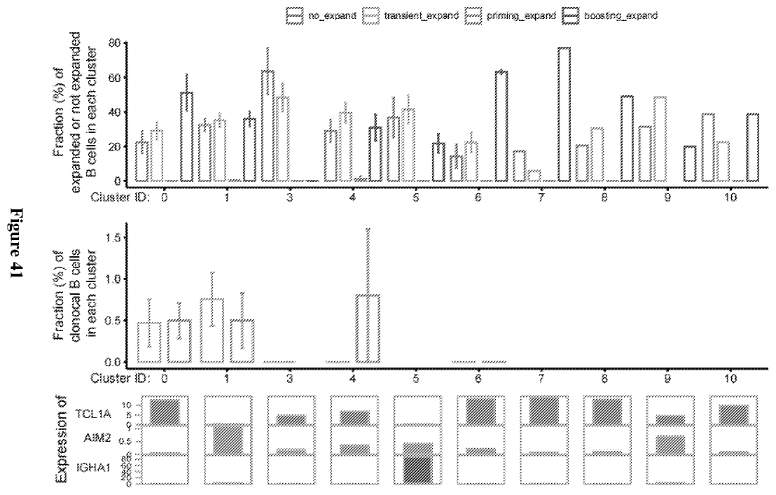

To test whether those expanded T-cell clonotypes after vaccination were directed toward neoantigens, we used the DNA-barcoded neoantigen-peptide-MHC tetramers with subsequent single-cell TCR sequencing to identify neoantigen-specific T-cell clones in blood of patient P6. Among the hyper-clonal T-cell clonotypes, ones with expansion after the vaccination had significantly higher proportion (29.8%, 95% CI, 17.3˜44.9) of neoantigen-85-specific T cells than that (7.0%, 95% CI, 1.9˜17.0) of clones without expansion (P=0.005, by Pearson's Chi-squared test) (FIG. 40).

### 1.2.8 Expansion of B-Cell Clonotypes During Vaccination

To test whether vaccination induced specific B-cell clones in blood, we also performed single-cell BCR sequencing in blood samples from 10 patients (P3˜12). A mean of 70.3% (range, 55.3˜88.4) of all B-cell clonotypes expanded in abundance after vaccination, but only 0.35% (range, 0˜0.83) were clonal. Two relapse patients (P4 and P9) and one non-relapse patient (P11) did not have expanded clonal B cells. These expanded clonal B cells were enriched in AIM2+ CRIP1+ and TCL1A+ IL4R+ FCER2+ early B cells (FIG. 41), which is consistent with the increased percent of TCL1A+ B cells in non-relapse patients seen above.

### 1.3 Discussion

This phase Ib study has shown treating personalized neoantigen peptide-based vaccines following postoperative chemotherapy in 12 patients with resected PDAC was safe. All enrolled patients had good tolerance with only mild adverse effects. A large percentage of patients were alive at postoperative 3 years and more than half of the patients achieved beneficial control of tumor recurrence. The inflammatory macrophage, cytotoxic T, central memory T and TCL1A+ B cells could be increased or clonally expanded systemically by the vaccination.

Due to the advanced stage and aggressive cell biology of PDAC with continuous therapy resistance, there are not enough available treatment options to achieve curative outcomes^{12, 13}. Consequently, clinical trials continue to be largely based on empiric drug combinations. Surgery is the only available option for the long-term cure, which can prolong overall survival by average 10 months¹⁴ and empiric drug combinations are currently the main therapeutic strategy in clinical trials¹⁵. However, patients still suffer the poor long-term survival result from high occurrences of tumor recurrence. Our study demonstrates here that an adjuvant personalized neoantigen vaccine is practicable and capable of immunizing patients to produce beneficial clinical outcomes in favor of enhanced recurrence control for PDAC following surgical resection. Indeed, long-term survivors of pancreatic cancer are prone to possess spontaneous tumor neoantigens of high immunogenicity, suggesting that neoantigen-based immunotherapies could benefit the survival of pancreatic cancer patients¹⁶.

Using the temporal single-cell sequencing at different time points, our study first sought to determine how the functional states of circulating vaccine-induced immune cells dynamically evolved across the course of vaccination. The results suggested that following vaccination, especially at the booster phase, the patients can elicit neoantigen-specific T cell immunity involving IFN-γ response genes (e.g. STAT1 and MX1). Although the major role of IFNs is involved in antiviral immune responses, our data demonstrated the relationship of these molecules to tumor killing and patient prognosis. Indeed, intratumoural stimulation of IFNs or downstream genes correlate with control of virus-unrelated malignancies¹⁷ and IFN-stimulated genes agonist exerted antitumor function in PDAC implanted mouse¹⁸. There exists controversy for the relationship between TCR diversity and the therapeutic response of ICI. Although decreased TCR diversity has been linked to improved clinical outcomes to anti-CTLA4^{19, 20} and anti-PD1 therapy^{21, 22}, anti-CTLA4 treatment increased the diversity of TCR clones in the tumor-specific CD8+ T cells²³ and the TCR diversity in peripheral CD8+ T cells could serve as a prognosis predictor for patients prior to ICI therapy in the non-small cell lung cancer²⁴. In contrast, neoantigen vaccines, due to the subdominant affinity recognized by many other T cells, can broaden breadth and clonal diversity of TCR repertoire^{25, 26}. In our study, the TCR diversity significantly increased after neoantigen vaccination, especially in the booster phase. The genes and amplified TCRs associated identified in this study could be used in the future as detection targets to stratify patients for the susceptibility of neoantigen vaccines, but an expanded patient population is needed to validate the results.

The ratio of responses to anti-PD1 therapy is low²⁷, but in our study, although limited sample size, two patients had the decrease of tumor indicators after the combination of neoantigen and anti-PD1 therapy. As mentioned above, relapsed patients lacked robust T-cell responses before and/or after vaccination, implying the difference in T cells status could explain the poor immune status of these patients during the vaccination. After the combination with anti-PD1 therapy, the relapse patients elicited activation of cytotoxicity genes in CD8+ T cells in addition to those activated by neoantigen vaccines alone in non-relapse patients. It suggests during the neoantigen vaccination the combined anti-PD1 therapy could relieve the patient's immunosuppression, although sometimes the patient's immune cells do not express PD1 or the tumor does not express PDL1. For these patients, post-administration of anti-PD1 could assist the neoantigen vaccines to activate bacterial stimulus pathways in CD8+ T cells that is different from that seen with the neoantigen vaccine alone in non-relapse patients, and different from that seen with anti-PD1 alone in other studies. These data provide the rationale and highlight the potential for further development of the neoantigen vaccines alone and combined with ICI therapies.

### 1.4 Reference

1. Castle J C, Kreiter S, Diekmann J, et al. Exploiting the mutanome for tumor vaccination. Cancer Res 2012; 72(5):1081-91. (Journal Article) (In eng). DOI: 10.1158/0008-5472.Can-11-3722.
2. Hilf N, Kuttruff-Coqui S, Frenzel K, et al. Actively personalized vaccination trial for newly diagnosed glioblastoma. Nature 2019; 565(7738):240-245. (Clinical Trial, Phase I; Journal Article; Research Support, Non-U.S. Gov't) (In eng). DOI: 10.1038/s41586-018-0810-y.
3. Keskin D B, Anandappa A J, Sun J, et al. Neoantigen vaccine generates intratumoral T cell responses in phase Ib glioblastoma trial. Nature 2019; 565(7738):234-239. (Clinical Trial, Phase I; Journal Article; Research Support, N.I.H., Extramural; Research Support, Non-U.S. Gov't) (In eng). DOI: 10.1038/s41586-018-0792-9.
4. Ott P A, Hu Z, Keskin D B, et al. An immunogenic personal neoantigen vaccine for patients with melanoma. Nature 2017; 547(7662):217-221. (Clinical Trial, Phase I; Journal Article; Research Support, N.I.H., Extramural; Research Support, Non-U.S. Gov't) (In eng). DOI: 10.1038/nature22991.
5. Jones R P, Psarelli E E, Jackson R, et al. Patterns of Recurrence After Resection of Pancreatic Ductal Adenocarcinoma: A Secondary Analysis of the ESPAC-4 Randomized Adjuvant Chemotherapy Trial. JAMA Surg 2019; 154(11):1038-1048. DOI: 10.1001/jamasurg.2019.3337.
6. Uesaka K, Boku N, Fukutomi A, et al. Adjuvant chemotherapy of S-1 versus gemcitabine for resected pancreatic cancer: a phase 3, open-label, randomised, non-inferiority trial (JASPAC 01). Lancet 2016; 388(10041):248-57. DOI: 10.1016/S0140-6736(16)30583-9.
7. Oh D Y, Kwek S S, Raju S S, et al. Intratumoral CD4(+) T Cells Mediate Anti-tumor Cytotoxicity in Human Bladder Cancer. Cell 2020; 181(7):1612-1625 e13. DOI: 10.1016/j.cell.2020.05.017.
8. Zhang F, Bai H, Gao R, et al. Dynamics of peripheral T cell clones during PD-1 blockade in non-small cell lung cancer. Cancer Immunol Immunother 2020; 69(12):2599-2611. DOI: 10.1007/s00262-020-02642-4.
9. Fehrenbacher L, Spira A, Ballinger M, et al. Atezolizumab versus docetaxel for patients with previously treated non-small-cell lung cancer (POPLAR): a multicentre, open-label, phase 2 randomised controlled trial. Lancet 2016; 387(10030):1837-46. DOI: 10.1016/S0140-6736(16)00587-0.
10. Mariathasan S, Turley S J, Nickles D, et al. TGFbeta attenuates tumour response to PD-L1 blockade by contributing to exclusion of T cells. Nature 2018; 554(7693):544-548. DOI: 10.1038/nature25501.
11. McDermott D F, Huseni M A, Atkins M B, et al. Clinical activity and molecular correlates of response to atezolizumab alone or in combination with bevacizumab versus sunitinib in renal cell carcinoma. Nat Med 2018; 24(6):749-757. DOI: 10.1038/s41591-018-0053-3.
12. Leroux C, Konstantinidou G. Targeted Therapies for Pancreatic Cancer: Overview of Current Treatments and New Opportunities for Personalized Oncology. Cancers 2021; 13(4):799.
13. Vincent A, Herman J, Schulick R, Hruban R H, Goggins M. Pancreatic cancer. The lancet 2011; 378(9791):607-620.
14. Oberstein P E, Olive K P. Pancreatic cancer: why is it so hard to treat? Therapeutic advances in gastroenterology 2013; 6(4):321-337.
15. Tesfaye A A, Kamgar M, Azmi A, Philip P A. The evolution into personalized therapies in pancreatic ductal adenocarcinoma: challenges and opportunities. Expert Rev Anticancer Ther 2018; 18(2):131-148. DOI: 10.1080/14737140.2018.1417844.
16. Balachandran V P, Luksza M, Zhao I N, et al. Identification of unique neoantigen qualities in long-term survivors of pancreatic cancer. Nature 2017; 551(7681):512-516. DOI: 10.1038/nature24462.
17. Zitvogel L, Galluzzi L, Kepp O, Smyth M J, Kroemer G. Type I interferons in anticancer immunity. Nat Rev Immunol 2015; 15(7):405-14. DOI: 10.1038/nri3845.
18. Vonderhaar E P, Barnekow N S, McAllister D, et al. STING Activated Tumor-Intrinsic Type I Interferon Signaling Promotes CXCR3 Dependent Antitumor Immunity in Pancreatic Cancer. Cell Mol Gastroenterol Hepatol 2021; 12(1):41-58. DOI: 10.1016/j.jcmgh.2021.01.018.
19. Cha E, Klinger M, Hou Y, et al. Improved survival with T cell clonotype stability after anti-CTLA-4 treatment in cancer patients. Sci Transl Med 2014; 6(238):238ra70. DOI: 10.1126/scitranslmed.3008211.
20. Hopkins A C, Yarchoan M, Durham I N, et al. T cell receptor repertoire features associated with survival in immunotherapy-treated pancreatic ductal adenocarcinoma. JCI Insight 2018; 3(13). DOI: 10.1172/jci.insight.122092.
21. Tumeh P C, Harview C L, Yearley J H, et al. PD-1 blockade induces responses by inhibiting adaptive immune resistance. Nature 2014; 515(7528):568-71. DOI: 10.1038/nature13954.
22. Hogan S A, Courtier A, Cheng P F, et al. Peripheral Blood TCR Repertoire Profiling May Facilitate Patient Stratification for Immunotherapy against Melanoma. Cancer Immunol Res 2019; 7(1):77-85. DOI: 10.1158/2326-6066.CIR-18-0136.
23. Kvistborg P, Philips D, Kelderman S, et al. Anti-CTLA-4 therapy broadens the melanoma-reactive CD8+ T cell response. Sci Transl Med 2014; 6(254):254ra128. DOI: 10.1126/scitranslmed.3008918.
24. Han J, Duan J, Bai H, et al. TCR Repertoire Diversity of Peripheral PD-1(+)CD8(+) T Cells Predicts Clinical Outcomes after Immunotherapy in Patients with Non-Small Cell Lung Cancer. Cancer Immunol Res 2020; 8(1):146-154. (Journal Article; Research Support, Non-U.S. Gov't) (In eng). DOI: 10.1158/2326-6066.CIR-19-0398%/(c) 2019 American Association for Cancer Research.
25. Carreno B M, Magrini V, Becker-Hapak M, et al. Cancer immunotherapy. A dendritic cell vaccine increases the breadth and diversity of melanoma neoantigen-specific T cells. Science 2015; 348(6236):803-8. (Clinical Trial, Phase III; Journal Article; Research Support, N.I.H., Extramural; Research Support, Non-U.S. Gov't) (In eng). DOI: 10.1126/science.aaa3828%/Copyright (c) 2015, American Association for the Advancement of Science.
26. Coulie P G, Van den Eynde B J, van der Bruggen P, Boon T. Tumour antigens recognized by T lymphocytes: at the core of cancer immunotherapy. Nat Rev Cancer 2014; 14(2):135-46. (Journal Article; Review) (In eng). DOI: 10.1038/nrc3670.
27. Ott P A, Bang Y J, Piha-Paul S A, et al. T-Cell-Inflamed Gene-Expression Profile, Programmed Death Ligand 1 Expression, and Tumor Mutational Burden Predict Efficacy in Patients Treated With Pembrolizumab Across 20 Cancers: KEYNOTE-028. J Clin Oncol 2019; 37(4):318-327. DOI: 10.1200/JCO.2018.78.2276.
28. Bates, D., et al. (2015). “Fitting Linear Mixed-Effects Models Using lme4.” JOURNAL OF STATISTICAL SOFTWARE 67(1): 1-48.
29. Hothorn, T., et al. (2008). “Simultaneous inference in general parametric models.” Biom J 50(3): 346-363.

### 1.5 Supplementary Methods

### 1.5.1 Clinical Data and Biological Sample

During the vaccine treatment period, adverse events, laboratory values, 12-lead electrocardiogram (ECG), vital signs and physical examination were regularly assessed and graded according to the National Cancer Institute Common Terminology Criteria for Adverse Events (version 5.0) at the first vaccination day and one week after each vaccination. During the follow up period, the above safety assessment was carried out every three months, and radiological examinations were performed every six months. Response Evaluation Criteria in Solid Tumors (RECIST, version 1.1) and the immune-related response criteria (irRC) guideline were used for clinical assessment of disease progression. The data cutoff was October 2021. Blood and serum samples were obtained from study participants throughout treatment. Sample preservation and culture is described in the Methods section in Supplementary Appendix.

### 1.5.2 Manufacturing of Personalized Neoantigen Vaccines

### Next-Generation Sequencing

The personalized neoantigen vaccines were prepared based on the analysis of whole-exome sequencing (WES) and RNA-seq data generated from fresh-frozen tumors obtained at the time of diagnostic resection and whole blood of patients. Whole-exome sequencing (WES) of whole blood and tumor tissue samples, RNA-sequencing of tumor tissue samples were operated by Shanghai Biotecan Medical Inspection Institute. QIAamp DNA Mini Kit (QIAGEN) was used to extract DNA of tumor tissue samples, QIAamp DNA Blood Mini Kit (QIAGEN) was used to extract DNA of whole blood, RNAiso Plus (TAKARA) was used to extract total RNA of tumor tissue samples. DNA library was constructed by SureSelectXT Target Enrichment System for Illumina Paired-End Multiplexed Sequencing Library (Agilent Technologies), RNA library was constructed by NEBNext rRNA Depletion Kit (Human/Mouse/Rat) (E6310) and NEBNext Ultra Directional RNA Library Prep Kit for Illumina (E7420) (NEW ENGLAND BioLabs). Raw data of WES and RNA-Sequencing was generated by Nextseq™ CN 500 System (Illumina).

### Somatic Mutation Calling.

For somatic mutation detection, tumor and matched blood samples from the patients were analyzed for single nucleotide variants. We use BWA-MEM algorithm which is generally recommended for high-quality queries, to map WGS and WES data against human reference genome hg19 with default parameters. We used fastp (version 0.20.0) to make sequencing data quality control. The clean data were aligned to the NCBI Human Reference Genome Build hg19 using Burrows-Wheeler Aligner software (BWA, version 0.7.17). Somatic single nucleotide variations (sSNVs) were detected using Genome Analysis Toolkit (GATK, version 4.1.1.0) and VarScan2 (version). All somatic mutations were annotated using Ensembl Variant Effect Predictor (VEP, version 3.9) to associate the variants with genes, transcripts, potential amino acid sequence changes.

### Transcript Abundance and HLA Calling.

For transcript abundance estimating. RNA-seq data were processed using Aligner HISAT2 (version 2.1.0) for mapping RNA-seq to hg19 reference genome, and StringTie (version 1.3.6) were used to assemble a transcriptome model to estimate transcript abundance.

For HLA calling, four-digit HLA class I alleles (HLA-A, HLA-B, and HLA-C) and class II alleles (HLA-DRB1, HLA-DQB1, HLA-DPB1) were identified by RNA-seq data using Seq2HLA software.

### Identification of Neo-Epitopes for Peptide Design.

For each nonsynonymous mutation identified by targeted NGS from patient, long peptides with 27 amino acids containing the mutated amino acid at position 14 were designed. Peptide binding prediction tools ANN method (version 2.19.2) from Immune Epitope Database (IEDB) were used to predict MHC class I binding of 8- to 11-mer mutant peptides to the patients' HLA-A, HLA-B, and HLA-C alleles. NetMHCII (version 2.17.6) were used to predict MHC class II binding of 15-mer mutant peptides to the patients' HLA-DR, HLA-DQ, HLA-DP. HLA binding affinity score for the respective variants were predicted and screened the best consensus.

### Neo-Epitopes Prioritization and Selection.

The mutated target neo-epitopes per patient required to select and prioritize for peptide preparation. The main principles were applied to rank neo-epitopes score: (1) neoORFs that included predicted binding epitopes; (2) high-affinity binding score (<500 nM) combined with high expression levels of the mutation encoding RNA. (3) high variant allele frequency.

### Manufacturing of Long Peptides, Pooling and Neoantigen Vaccine Preparation.

Neoantigen-derived peptides 27 amino acids in length were synthesized (Sangon Biotech, Shanghai, China) and purified (Qiaoyuan Biotech, Shanghai, China) in Good Manufacturing Practice (GMP) way. A bottle of 300 μg of each peptide was manufactured and cryopreserved at −80° C. Each peptide was tested identity, sterility and endotoxins before clinical use. Each patient's vaccine has four pools (A, B, C, D), with 4-6 distinct peptides of each pools. When the day of vaccination, each pool was added 2 ml 5% glucose injection and was mixed with 0.25 ml of poly-ICLC for a final dose of vaccine that were administered subcutaneously (s.c) on days 1, 4, 8, 15, 22 (priming phase) and weeks 12 and 20 (booster phase). Each of the four vaccine pools were injected into the patient's two arms and inner thighs.

### 1.5.3 Vaccine Administration

The detailed vaccine administration plan is as follows: the right arm, left arm, right thigh and left thigh of the patient are selected as four injection sites and multiple neoantigen peptides designed for each patient will be randomly and evenly distributed to the above four injection sites. If the number of peptides designed for a patient is less than 15, the vaccines will be divided into 2 group for injection on average, so as to avoid the situation that there are too few vaccines in each group. The vaccine was transported to the hospital with dry ice on the day of treatment to ensure the stability of peptide vaccine.

The doses of vaccines and the vaccination interval in this treatment protocol refer to the publication of Catherine J Wu in the Nature, where they demonstrated the feasibility of the neoantigen vaccine therapy and the designed treatment protocol in patients with melanoma. In this clinical study, we took different doses for different patients with a minimum of 2.4 mg and maximum of 7.5 mg. Many clinical trials have shown that adding poly-ICLC as adjuvant can further accelerate the induction of specific immune response to neoantigen vaccine (Sabbatini P, et al. Clin Cancer Res. 2012). The adjuvant dose (1˜1.6 mg) is commonly used in clinical treatments (Okada H, et al. J Clin Oncol. 2011; Rosenfeld M R, et al. Neuro Oncol. 2010). This dose can ensure effective stimulation with less side effects. This project used 0.5 mg poly-ICLC for each vaccine group.

### 1.5.4 Biological Sample Collection, Preservation and Culture

Blood and serum samples were obtained from study participants throughout treatment. Patients PBMCs were isolated by Ficoll density-gradient centrifugation (GE Healthcare) and cryopreserved with 10% DMSO in FBS (Gemini). Cells and serum from patients were first cooled in a gradient in the cryopreservation box to −80° C. and then stored in liquid nitrogen until time of analysis.

Fresh tumor samples were obtained immediately after surgery. A portion of the sample was removed for formalin fixation and paraffin embedding (FFPE). For construction of patient-derived tumor cell lines, samples were minced and digested using the gentleMACS Octo system. After dissociation, the cell suspension was filtered with a 70-μm filter, washed in DMEM medium with FBS, and pelleted by centrifugation at 400 g at 18° C. for 10 min. Cells were then resuspended in DMEM medium with 20% FBS and cultured in 6-well plate. The remainder of the sample were used for generation of the personalized neoantigen vaccine, in which DNA and RNA sequencing were performed when pathology review conformed adequate tumor cellularity.

### 1.5.5 Follow-Up and Pattern of Relapse

The institutional follow-up was jointly completed by department follow-up specialists, and the third-party professional data were provided by LinkDoc Technology Co. Ltd. (Beijing, China). The strategy and definitions of relapse were described as follows. When increased pre-operative levels of CA19-9 were observed, these levels evaluated every three months thereafter. Computed tomography (CT) or magnetic resonance imaging (MRI) was performed every three months for the first two years and every six months for the next three years to screen for relapse. When imaging findings were consistent with relapse, biopsy was performed only rarely, but, MRI and/or fluorodeoxyglucose positron emission tomography (FDG-PET) was carried out if necessary to clarify ambiguous CT findings. Local relapse was defined as relapse in the remnant pancreas or in the operative bed, including the soft tissue along the celiac or superior mesenteric artery, aorta, or around the site of the pancreaticojejunostomy. Distant relapse was stratified into three different categories: “liver-only” and “lung only” for isolated hepatic and pulmonary relapse, respectively, and “other” for relapse occurring in other less frequent locations.

### 1.5.6 Definitions and Statistical Analysis for Survival

Relapse-free survival (RFS) was calculated from the date of pancreatectomy to the date of relapse or last follow-up if relapse did not occur. Overall survival (OS) was defined as the time from pancreatectomy to either death or last follow-up. Log-rank testing was used to test the statistical significance of differences in the curves of the three groups, and the corresponding P-value was obtained. A two-tailed P-value of <0.05 was considered statistically significant. Statistical analysis was performed using SPSS 23.0 software (IBM, Armonk, NY, USA).

To address potential confounders, we modeled the probability of treatment using logistic regression and used the estimated probability as a propensity score. We included relevant baseline variables that might have affected treatment decisions, which included gender, T stage, N stage, differentiation degree and tumor site. Variables were selected on the basis of clinical experience, and the success of balancing distributions between groups. In the propensity-score matched analysis, we used “optimal pair” matching without replacement, and matched vaccinated patients and unvaccinated patients in a 1:1 ratio. We used the “MatchIt” package from R software, version 4.1.0, for the propensity-score matched analysis.

### 1.5.7 IFN-γ ELISPOT Assays

Fresh or thawed cryopreserved PBMCs were cultured in X-VIVO 15 medium (Lonza) supplemented with 10% heat-inactivated FBS and penicillin-streptomycin (100 U/ml, Gibco). For in vitro stimulation and expansion of antigen-specific T cells, PBMCs were stimulated in 96-well round-bottom cell culture plate (Corning) at 2×10≡per well with individual (10 μg/ml) or pooled peptides (each at 2 μg/ml) in the presence of IL-7 (20 ng/ml, R&D Systems). In vitro stimulation was carried out in the presence or absence of anti-HLA-DR (10 μg/ml, clone L243, Biolegend) and anti-HLA-A, B, C (10 μg/ml, clone W6/32, Biolegend), which was added 1 h in advance of addition of peptides. On day 4, IL-2 (20 U/ml, R&D Systems) was added. On day 4, 6, and 10, half-medium with supplementation of cytokines, peptides and blocking antibodies was changed. On day 13, the plate was centrifuged and supernatant was removed. Cells were resuspended in 200 μl medium and counted. IFNγ ELISPOT assays were performed using 96-well MultiScreen Filter Plates (Millipore), coated with 15 μg/ml anti-human IFNγ mAb overnight (1-D1K, Mabtech). Plates were washed with PBS and blocked with X-VIVO medium before addition of pre-stimulated PBMCs. Concanavalin A (5 μg/ml, Sigma-Aldrich) was added as positive control. Plates were rinsed with PBS and then 1 μg/ml anti-human IFNγ mAb (7-B6-1 Biotin, Mabtech) was added, followed by Streptavidin-ALP (Mabtech). After rinsing, BCIP/NBT-plus substrate for ELISpot (Mabtech) was used to develop the immunospots, and spots were imaged and enumerated using Immunospot Analyzer (Cellular Technology Limited). Responses were scored positive if spot-forming cells were more than the Blank control.

### 1.5.8 Neoantigen Peptides Cytotoxicity Assay.

To analyze the killing tumor cell ability of immune cells stimulated by peptides, PBMC from patients were plated at a density of 2*10\hat{ }5 cells per well in a 96-well round-bottom plate and incubated in X-Vivo medium containing 10% FBS, penicillin-streptomycin for 7 days (same as IFN-γ ELISPOT assay). Added individual (10 μg/ml) or pooled peptides (each at 2 μg/ml) in the presence of 20 ng/ml IL-7 (20 ng/ml, R&D Systems). Every 3 days, half-fresh medium with supplementation of 20 U/ml IL-2 and peptides was added to the culture. Target tumor cells were culture in a 6 cm dish and incubated in DMEM medium (Gibco) containing 10% FBS, penicillin and streptomycin. After stimulating PBMC with peptides, labeled tumor cell with fluorescein. Target tumor cell were incubated with CMFDA (Nexcelom, staining alive cells) for 30 minutes at 37° C., 5% CO2 culture environment. Then labeled tumor cells were culture in a 96-well plate pre-cultured with collagen I (Corning) overnight. Stimulated PBMC and labeled tumor cells were co-cultured in a 10:1 ratio in DMEM with 10% FBS and 125×PI (staining death cells). Celigo Image Cytometer (Nexcelom) was used to observe fluorescence intensity of stained alive tumor cells and death cells, which can calculate the killing ratio of PBMCs. We also used another equipment, xCELLigence (Agilent), to observe the real time resistance change of tumor cells while co-culture PBMCs and tumor cells, which also reflect the killing ratio of PBMCs.

### 1.5.9 Single-Cell RNA-Sequencing and T Cell Repertoire Profiling.

We performed 3′ gene expression profiling on the single-cell suspension using the Chromium Single Cell Gene Expression Solution from 10× Genomics according to the manufacturer's instructions. Up to 9,000 cells were loaded onto 10× Genomics cartridge for each sample. Single-cell TCR-seq enriched libraries were prepared using the 10× Single Cell Immune Profiling Solution Kit, according to the manufacturer's instruction. Cell-barcoded 3′ gene expression libraries and scTCR-sequencing libraries were sequenced on an Illumina Nova-PE150 system.

### 1.5.10 Single-Cell Transcriptome Data Generation and Quality Control

Single cell libraries were prepared according to Illumina HiSeqXTen instruments using 150 nt paired-end sequencing. FASTQ files generated from sequencing were processed using the Cell Ranger 3.1.0 pipeline (10× Genomics) with default parameters. The human genome, GRCh38, was used as the reference for reads mapping. Cell Ranger pipeline finally generated Gene-Barcode matrices containing filtered cell barcodes and counts of unique molecular identifiers (UMIs). For each sample, we individually imported the gene-barcode matrix into the Seurat (v3.1.1) R toolkit 1 for quality control and Normalization. Cells (>200 genes per cell, <4000 genes per cell and <20% mitochondrial genes per cell) were selected for downstream analysis of our single cell RNAseq data. Each sample derived from each stage of one patient was inspected as a Seurat object and integrated by patient using FindIntegrationAnchors and IntegrateData function in Seurat package, which is designed for comparative analyses across batches or datasets using the anchor algorithm.

### 1.5.11 Identification of Major Cell Types by Shared Nearest Neighbor (SNN) and Expression of Cell Markers

The Seurat objects integrated by the patients were individually imported for clustering, using the FindNeighbor and FindClusters functions (with the parameter resolution=1) in the Seurat package. Then, the expression of known immune cell markers are used to characterized identities of cell types for each cluster. Specifically, CD68, CSF1R are used for Macrophage; CD14, S100A12 for Monocyte; ITGAM, CD33 for MDSC; CD19, MS4A1, CD79A for B cells; ITGAX, CD83 for DCs; PF4, PPBP for Megakaryocyte; CD247, KLRB1 for NKs; TIMP2, ITGA2B for Platelet; CD3D for T cells. Scaled data of these gene expression obtained from the Seurat object was used to estimate the cell type. Based on these known genes, we scored the gene expression of the given gene (g) of the given cell type (i) for each cluster:

$${Score}_{g} = \{ \begin{matrix}1 , & R \geq 0.8 \\ 0.5 , & 0.5 \leq R < 0.8 \\ 0.25 , & 0.25 \leq R < 0.5\end{matrix}$$

$${Score}_{i} = ( \sum {Score}_{g} ) / n$$

Where R indicated the ratio of scaled expression of marker g (in cell type i)>0 in cells of this cluster. The n indicates the total number of genes for the cell type i.

Further, the type of each cluster was labeled by the cell type with the maximum score comparing across the scores of all cell type. If the maximum score of one cluster got is less than 0.1, the cluster was defined as unknown cells. Finally, the clusters with same labeled were merged. Subsets of T cells were further performed with the same approach, grouped into CD4+, CD8+, CD4+CD8+ and CD4/CD8 low T cells based on the expression of genes CD4 and CD8A.

### 1.5.12 Comparison of Percentage of Cell Types and Gene Expression of Cell Subtypes Across the Treatment and Between Patients

The percentages of the cell types were calculated and normalized gene expression value were obtained in each cell type in patients before and after treatment and at different stages of treatment. The percentage of cell types is the percentage of the each previously defined cell types including B cell, T cell, monocyte, macrophage, etc. in whole cells in one sample. This value was used to estimate the increase or decrease in the relative cell population abundance of main cell types during the course of the immunotherapy. In addition, the gene expression in each cell type is converted into two types of values: 1) The proportion of cells that positively express the gene in the given cell type; 2) The average value of the gene expression in the given cell type. The two types of values were further used to estimate the changes in the relative abundance of cell subpopulation that expressed specific genes and the changes in the expression level of each gene in the given cell types. Positive expression was defined according to the distribution of the normalized expression value of a given gene in the respective patients. The threshold for dividing positive and negative was chosen at the first pit from low to high values, but when there is no pit for a gene in a patient, geometric mean of their expression was defined as the threshold. All pit points in the distribution are determined by the first derivative equal to 0 and the second derivative greater than 0 using the function ‘diff’ in R.

On the one hand, each patient had a time-series data for different stages. On the other hand, patients can be divided into tumor relapse group (P2, P4, P6 and P9) and non-relapse group (P1, P3, P5, P7, P8, P10, P11 and P12), or they can be divided into anti-PD1-treated group (P4, P6 and P9) and non-anti-PD1-treated group (same to the non-relapse group). To be note, when we performed statistics of single-cell data and compared the immune status between the patients, P3 did not show evidence of relapse (including elevated tumor indices or tumor relapse on imaging) until more than one year after the last vaccination and thus the single-cell data of P3 during the vaccination phases were used in the non-relapse group for statistical differences. A linear mixed model (LMM) simultaneously considering multivariate was applied to explore the association between the changes in the proportion of these immune cells or gene expression and the treatment stages or patient groups. LMM was performed using the R package lme4 2. In the model, the response variable (y) could be percentage of cell types, percentage of gene-based positive-expressed cell subpopulation or expression value of a gene. Explanatory variable included patient group and treatment stage as two fixed effects. Individual patient was a random effect accounting for that same patient were measured more than once. In order to gain statistical power, although there were 7˜10 detection time points per patient, we only compared the differences between three phases: months or 1 day before the first vaccination (Pre-vaccine), 1˜22 days (Priming), 50˜162 days (Boosting) after the first vaccination. To test the differences of cell proportion and gene expression respectively among treatment stages and patient groups, we employed the generalized linear hypothesis (glht) function in the R package multcomp 3, to make the following contrasts: 1) the difference between pre-vaccination and priming phase, 2) the difference between pre-vaccination and the boost phase, 3) the difference between the priming and the boost phase, 3) the differences between patient groups in the pre-vaccination phase, 4) in the priming phase and 5) in the boost phase. The resulting p-values were adjusted using the Benjamini-Hochberg procedure. These analyses, test and visualizations were developed in R environment and scripted in in-house R codes. The P-value<0.05 was considered as the significance for all the test. ROC (receiver operating characteristic) curves were used to assess the differences between priming and pre-vaccination or between boosting and pre-vaccination or between non-relapse and relapse patients. AUC (area under the ROC) was used as metric of the differences. These analyses, test and visualizations were developed in R environment and scripted in in-house R codes. The adjusted P-value<0.05 was considered as the significance for all the test.

### 1.5.13 Average Expression of Gene Module Analysis for Data of Single-Cell RNA-Seq

Gene module scores were calculated based on the expression of genes in the given pathway module provided by Seurat package, using the ‘AddModuleScore’ function. This function assigned scores (i.e. the average expression) for each cell. The scores were contrasted for the differences among different phases by using the LMM method.

### 1.5.14 Data Analysis for Single-Cell RNA-Seq in Cytotoxicity Assay

Raw sequencing data was also individually processed using Cell Ranger 3.1.0 pipeline with default parameters and the output was imported into the Seurat to filter the low-quality cell (>200 genes per cell, <5000 genes per cell and <15% mitochondrial genes per cell) and do normalization. All the single neo-epitope-stimulated samples and one blank control sample were merged using the function IntegrateData. The cytotoxicity marker was identified by respectively comparing the neo-epitope-stimulated samples to the blank control using the function FindMarkers with the option min.pct=0.01, log fc.threshold=0.2, test.use=‘wilcox’.

### 1.5.15 Single-Cell T Cell Receptor V(D)J Clonotypes (TCR) Data Generation and Analysis

TCR data for each sample was processed using Cell Ranger 3.1.0 pipeline (‘cellranger vdj’ command) with default parameters using human reference genome GRCh38. For each sample, Cell Ranger generated an output file, filtered_contig_annotations.csv, containing TCR α-chain and β-chain CDR3 nucleotide sequences for single cells that were identified by barcodes. In order to compare the TCR sequences across patients and treatment stages, we merged the separate output of samples using the procedure and code script from Thomas D. Wu et al 4. Cell Ranger also annotated the TCR V(D)J genes for each clonotype, so the usage of TCR genes were also counted according the merged result. As single cell TCR-seq was done using the Chromium Single Cell 5′ Library, its counterpart for gene expression was also sequenced and cells were labeled same barcodes. The cells simultaneously contained TCR clonotypes and expressed gene CD3D were regarded as T cells and they were categorized into CD4+, CD8+ and CD4-low CD8-low T cells based on the expression of CD4 and CD8A using the same approach descripted in the single-cell transcriptome data.

### 1.5.16 TCR Clones Prediction in Tumors of Patients

The sequences of TCR clones infiltrating tumors were predicted using MiXCR software 5. The typical analysis workflow processing the RNA-sequencing data was applied for the tumors of patients. The α-chain and β-chain CDR3 nucleotide sequences derived from the tumor's sequences of each patient were obtained and the sequences were matched to the TCR clones of T cells in blood by software blastn (with the option −evalue 0.01−num_alignments 1). TCR clones that can be matched were considered as occurrence together in blood and tumor tissues.

### 1.5.17 Assessment of the Diversity of Usage of TCR Genes

Shannon diversity index was employed to assess the diversity of TCRs. The shannon index value for each cell was calculated by the equation as:

$$shannon index = - {\sum }_{i} {p}_{i} * ln {p}_{i}$$

where pi indicates the relative value of TCR gene i divided by summation of total V(D)J genes.

For the 3′ single-cell transcriptome data, the TCR V(D)J gene expression value that Seurat had normalized was used. For the 5′ single-cell TCR-seq data, the count of V(D)J genes subjected to the all the TCR clones in each cell was used.

1.5.18 Knock Down of MX1 in PBMCs and qPCR Assay

PBMCs were cultured in X-VIVO 15 medium (Lonza) supplemented with 10% Fetal Bovine Serum (Gemini), Penicillin-Streptomycin (Gibco), Antibiotic-Antimycotic (Gibco), 20 ng/ml recombinant human IL-7 (R&D), 50 U/ml recombinant human IL-2 (R&D) for one week. Then, TransIT-TKO kit (Mirus) was used to transient transfect MX1 siRNA into PBMCs. After 3 days of culture at 37° C., PBMCs were collected. A part of PBMCs co-incubated with tumor cells to test the killing effect of PBMCs after siRNA interference. The other part of PBMCs was extracted RNA with AllPrep DNA/RNA Kits (QIAGEN) and reverse transcription to cDNA with PrimeScript RT reagent Kit (Takara), and then used NovoStart SYBR qPCR SuperMix Plus (Novoprotein) performs qPCR to verify the efficiency of siRNA interference. The relative expressions of MX1 gene at the mRNA level was calculated by the 2-ΔΔCt method. GAPDH was used as a housekeeping gene. The siRNA and primer sequences were as follows:

siRNA,
5′-GCTTTGTGAATTACAGGACAT-3′;
MX1,
5′-GTTTCCGAAGTGGACATCGCA-3′ (Forward);
5′-CTGCACAGGTTGTTCTCAGC-3′ (Reverse);
GAPDH,
5′-GGAGCGAGATCCCTCCAAAAT-3′ (Forward);
5′-GGCTGTTGTCATACTTCTCATGG-3′ (Reverse).

### 1.6 Supplementary References

1. Stuart T, Butler A, Hoffman P, et al. Comprehensive Integration of Single-Cell Data. Cell 2019; 177(7):1888-1902.e21. (Journal Article; Research Support, N.I.H., Extramural; Research Support, Non-U.S. Gov't; Research Support, U.S. Gov't, Non-P.H.S.) (In eng). DOI: 10.1016/j.cell.2019.05.031%/Copyright (c) 2019 Elsevier Inc. All rights reserved.
2. Bates D, Maechler M, Bolker B M, Walker S C. Fitting Linear Mixed-Effects Models Using lme4. JOURNAL OF STATISTICAL SOFTWARE 2015; 67(1):1-48. (Article) (In English). DOI: 10.18637/jss.v067.i01%/JOURNAL STATISTICAL SOFTWARE.
3. Hothorn T, Bretz F, Westfall P. Simultaneous inference in general parametric models. Biom J 2008; 50(3):346-63. (Journal Article; Research Support, Non-U.S. Gov't; Review) (In eng). DOI: 10.1002/bimj.200810425%/Copyright 2008 WILEY-VCH Verlag GmbH & Co. KGaA, Weinheim.
4. Wu T D, Madireddi S, de Almeida P E, et al. Peripheral T cell expansion predicts tumour infiltration and clinical response. Nature 2020; 579(7798):274-278. (Journal Article) (In eng). DOI: 10.1038/s41586-020-2056-8.
5. Bolotin D A, Poslavsky S, Davydov A N, et al. Antigen receptor repertoire profiling from RNA-seq data. Nat Biotechnol 2017; 35(10):908-911. (Journal Article; Research Support, Non-U.S. Gov't) (In eng). DOI: 10.1038/nbt.3979.

### Example 2

Nowadays, cancer treatment and cancer therapy has been more and more concentrated on targeting tumors precisely without damaging normal tissues. In this process, immunotherapy is more and more widely used, and tumor immune microenvironment (TIME) is very important for this kind of cancer treatment [5]. Some kinds of lymphocytes are crucial in anti-tumor immune activities, including cytotoxic T cells, which can identify the tumor cells and perform cytotoxic functions precisely [6, 7], and natural killer cells (NK cells), which can kill tumor cells in the absence of antigen presentation [8-10]. Some myeloid cells are also important in the tumor immune environment, including dendritic cells (DCs) and tumor associated macrophages (TAMs) [11, 12]. Therefore, to understand the roles of different cells in the TIME and genes and proteins that can affect their functions is very important for immunotherapy in the cancer treatment.

Protein Phosphatase 1 Regulatory Subunit 15A (PPP1R15A) is an important gene in mammal cells, including in human and mouse cells. PPP1R15A is also known as Growth Arrest And DNA Damage-Inducible Protein (GADD34), which plays an important role in apoptosis and can be induced when DNA damage happens [13]. Further research shows that PPP1R15A can bind to the catalytic subunit protein phosphatase 1 (PP1c) and promote the dephosphorylation of eukaryotic translation initiation factor 2α (eIF2α) [14, 15]. eIF-2α plays a very important role in the integrated stress response (ISR) in cells [16], thus the phosphorylation and dephosphorylation process of eIF-2α can be crucial for the ISR process. As an evolutionarily conserved process, ISR can coupled to both UPR and HSR activation [17, 18], and activated by both ER and cytosol lumen [19], which make this process crucial for cells, tissues, and organisms to adapt to variable environment and maintain homeostasis [20].

ISR functions through the regulation to the ternary complex (TC) [20], and eIF2 is an important compartment of the TC. eIF2 is composed of three subunits, eIF2α, eIF2β and eIF2θ. In the four units, the phosphorylation on serine 51 of the eIF2α is the most important process [21]. When eIF2α is phosphorylated, the global protein synthesis will reduced and the translation of activating transcription factor 4 (ATF4) is activated [22], which benefits to the cell survival and recovery. On the contrary, the dephosphorylation of eIF2α will make the cell recover normal protein synthesis process [23, 24]. The ISR outcome and levels are determined by the levels of phosphorylation of eIF2α and the activity of ATF4 [25, 26]. ATF4 can function as complex with other kinds of bZIP transcription factors [27], including the C/EBP homologous protein (CHOP) [28], which is more commonly seen in cell biological process. The ATF4-CHOP complex plays an important role in the mammalian autophagy process, including the induction of autophagy and activation of autophagy-related genes [28, 29]. The target genes of ATF4-CHOP complex include ATF3, PPP1R15A, TRIB3, etc [30]. Many autophagy genes also can be upregulated, including ATG3, ATG5, ATG7, ATG10, ATG12, ATG16, BECN1, GABARAP, GABARAPL2, MAP1LC3B, and SQSTM1 [29].

Based on the fact that eIF2, especially eIF2α, is crucial to the ISR process, the regulation of the phosphorylation level of eIF2α can be important. The dephosphorylation of eIF2α is performed by two phosphatase complexes, the PPP1R15A-PP1c complex and PPP1R15B-PP1c complex. PPP1R15B, also known as constitutive repressor of eIF2α phosphorylation (CReP), can repress the constitutive eIF2α-directed phosphatase activity, thus activate the ISR process in a steady way [31]. PPP1R15A, on the other hand, is used as a feedback way to antagonize the relative strength of ISR activation [23]. Because of the different function types of PPP1R15A and PPP1R15B, the selectively suppression of the function of PPP1R15A could be safer, and the inhibition of the function of PPP1R15B could be lethal [26]. Therefore, inhibitors of PPP1R15A can be used as regulators of the ISR process in cells.

The ISR can have multiple influences on cellular biological processes, and affect the function and homeostasis in mammalian bodies. Several researches have proved that the ISR functions in cognitive and neurodegenerative disorders [32-34], metabolic disorders [35, 36], cancers [37, 38], and can also have important influence on mammalian immunity. Existing studies have shown that the ISR can affect the innate immune response [39, 40], and also the secretion of some kinds of cytokines, including IL1IP and IL6 [41, 42]. These influences may highly dependent on the phosphorylation and dephosphorylation of the eIF2 complex [43], especially the eIF2α subunit, and on the other hand, the activation of ATF4 is also important in the related process [44].

Sephin1 is a selective inhibitor of a holophosphastase. It can selectively bind to PPP1R15A, thus inhibit the formulation of PPP1R15A-PPlc complex. Because of its high selectivity, it will not bind to PPP1R15B, which made it safer for animals [45]. Sephin1 has been reported to have the ability to restore motor function and rescue myelin deficits in mouse models [45]. However, in our research, we found that the injection of Sephin1 can inhibit the immune system functions in C57BL/6 mice, and in the mouse group injected with Sephin1, the tumor growth rate was much higher than the control group. This result indicates that the function of PPP1R15A can be important for the anti-tumor immune activities. By single-cell sequencing, we found that the influence of Sephin1 to mouse tumor microenvironment was complicated, and the immune suppressing effect can exist in multiple immune cell types.

### 2.1 Materials and Methods

### 2.1.1 Reagent Preparation

Sephin1 solution was prepared before the injection. 50 mg Sephin1 (APExBIO, A8708-50) was firstly dissolved by 625 μl DMSO, and then dissolved in 12.5 ml PBS. The final solution contained 4 mg/ml Sephin1 and 5% DMSO. This solution would be used for mouse injection. The same volume of DMSO solution with the same percentage (5% DMSO in PBS) was used in the control group.

### 2.1.2 Mice Injection

6-8-week-old C57BL/6 mice were used in this experiment. Mice were firstly separated into two groups, control group and Sephin1 group. The Sephin1 group were injected intraperitoneally with 100 μl Sephin1 solution prepared in the first step, and the control group were injected with equal 5% DMSO-PBS solution. Both groups were injected three times a week, and the injection lasted two weeks. All mice were subcutaneously inoculated with 3×10⁵ B16F1 cells the next week after the injection completed. About one week after the injection, the tumor volumes were measured and analyzed. The tumor tissues were collected after two-week development for further experiment. In addition, the growth rate of a mouse triple-negative breast cancer cell line, 4T1, was measured in 8-week-old female BALB/c mice (eight mice in each group). After two weeks of injection of DMSO or Sephin1, each BALB/c mouse was subcutaneously inoculated with 10⁶ 4T1 cells. Tumor volume was then measured every 2-3 days.

### 2.1.3 Generation of Mouse PBMCs

The peripheral blood samples were collected from mouse eyes. Each sample was firstly mixed with 200 μl EDTA, and then mixed with PBS in equal volume. Equal volume of Ficoll-Paque PREMIUM (Amersham/GE, 17544602) was and the blood-EDTA-PBS solution were added into an 15 ml centrifuge tube and centrifuged with 400 g, 20 min. The peripheral blood mononuclear cells (PBMCs) were collected, and the erythrocytes were removed with ACK lysing buffer (ThermoFisher, A1049201). The lysed cells were filtered with 30 μm MACS SmarterStrainer (Miltenyi/MACS, 130-110-915) and washed by PBS for 1-2 times, and resuspended with PBS in proper volume. The cells were stained with AO/PI (Nexcelom Bioscience, CS2-0106-5 mL) and calculated using Cellometer K2 (Nexcelom Bioscience).

### 2.1.4 Tumor Tissue Processing

The tumor tissues were collected and cut into small pieces (about 1-2 mm). Then we dissolved the tumor tissue with the mouse tumor dissociation kit (Miltenyi/MACS, 130-096-730) following the standard procedure. After that, the cell suspension were filtered with 30 μm MACS SmarterStrainer. Then we performed either FACS analysis or FACS sorting procedure (sorting for CD45+ and living cells).

### 2.1.5 FACS Sorting and Analysis

The FACS sorting procedure were performed before the single-cell library construction of immune cells in tumor samples. The tumor cell suspensions were firstly incubated with mouse CD45 antibody (BioLegend, 157607) for 30 minutes, and then incubated with PI (Nexcelom Bioscience, CS1-0109-5 mL). Cells were sorting with the BD SORP FACSAria machine.

FACS analysis were performed on the tumor tissues. After getting the single-cell suspension, each sample was firstly stimulated by Cell Activation Cocktail with Brefeldin A (BioLegend, 423303) with the concentration of about 5×10⁶ cells/mL, and the volume ratio of the cocktail and the cell suspension was 1:500. After 4 hour stimulation at 37° C., the cells were centrifuged with 400 g, 7 min, and the supernatant was discarded, and the FACS antibodies were incubated with the samples. The cells were first incubated with mouse surface antibodies, including CD45 (Thermo Fisher, 12-0451-83; BioLegend, 103105; BioLegend, 157607), CD3E (BD Pharmingen, 553064), CD4 (BioLegend, 100548), CD8A (Thermo Fisher, 25-0081-81), NK1.1 (Thermo Fisher, 48-5941-80), FOXP3 (BioLegend, 126419), PD-1 (Thermo Fisher, 17-9985-82), CD11b (BioLegend, 101215), F4/80 (BioLegend, 123125), TCRβ (BioLegend, 109205) and reagents from a LIVE/DEAD Viability Kit (ThermoFisher, L34994/L34963) for 30 min. After that, the cells were centrifuged with 1500 rpm, 5 min, and washed by PBS once. Cytofix/Cytoperm Kit (554714, BD Pharmingen) were then used for cell fixation and permeabilization. Then the cells were washed and incubated with mouse IFNG antibody (BioLegend, 505806) for 30 min. After that, the cells were washed and resuspended by BD Perm/Wash buffer from the Cytofix/Cytoperm Kit. The prepared cell suspensions were analyzed on the CytoFLEX LX machine from Beckman Coulter.

### 2.1.6 Library Construction and Sequencing of Single-Cell RNA-Seq

Twelve samples were used for single-cell library construction in all. Firstly, after two-week injection of DMSO/Sephin1, we selected two mice in each group randomly and collected all four PBMC samples. Secondly, after two weeks of the B16F1 cell injection, we also selected four mice from the two groups, and collected four PBMC samples and four tumor immune samples (CD45+ cells separated from tumor tissues by FACS) from them.

The cell suspension samples we got in the last procedure were used for single-cell library construction. We performed the single cell immune profiling following the standard procedure from 10× Genomics. The library construction kit we used including Chromium Next GEM Single Cell 5′ Library & Gel Bead Kit v1.1 (16 rxns, PN-1000165), Chromium Single Cell 5′ Library Construction Kit (16 rxns, PN-1000020), Chromium Single Cell V(D)J Enrichment Kit (Mouse T cell, 96 rxns, PN-1000071), Chromium Next GEM Chip G Single Cell Kit (48 rxns, PN-1000120) and Single Index Kit T Set A (96 rxns, PN-1000213). After the single-cell library construction, all samples were sequenced with the Illumina NovaSeq PE150 platform. We got 12 5′ gene expression libraries and 12 matched TCR enriched libraries in all. All the gene expression libraries were sequenced with the data size of 80 G each, and the TCR enriched libraries were 10 G.

2.1.7 Single-Cell Data Processing and Integration with TCR Enrichment Data

Firstly, all 12 expression sequencing data and 12 TCR enrichment sequencing data, we firstly analyzed the data using Cell Ranger (version 6.0.0) software from 10× Genomics. The single-cell expression data was then imported into R and integrated with Seurat (version 3.2.3). To minimum the information loss and filter out low quality and duplicated cells at the same time, genes that were expressed in at least 2 cells were kept, and cells with genes more than 100 and less than 4000 were kept. The cells were also filtered by the percentage of mitochondria genes, and cells that were discrete in the violin plot were filtered out. The filtered cells were then integrated, normalized, scaled and clustered with Seurat. Cell type annotation was made using classic immune cell markers.

The filtered TCR contig matrix were analyzed and integrated using scRepertoire (version 1.2.1) [62] and then integrated with the gene expression data. The integration process was scripted and performed on python (version 2.7.5) and R (version 3.6.3) platforms. Cells between the TCR enrichment data and expression data were matched according to their specific barcode sequence. Plots of different TCR types were made by ggplot2 (version 3.3.5).

### 2.1.8 Regulation Gene Set Analysis Performed by SCENIC

SCENIC [63] analysis was also performed for analyzing the activity of important transcription factors and their related genes. Firstly, 5000 cells from all 12 samples were randomly selected to identify the co-expression network with higher activities using GENIE3 (version 1.8.0). After that, SCENIC analysis was performed on all cells and regulons were filtered from the com-expression network. Then we calculate the activities of different regulons from different sample types and cell types.

### 2.1.9 Analysis of Differentiate Gene Patterns in Different Clusters

Differentially expressed gene in different clusters or samples were identified by FindMarkers package from Seurat. The differentially expressed genes were then used to perform enrichment analysis, including GSVA and GSEA, which were completed with R package GSVA (version 1.30.0) and fgsea (version 1.8.0). Besides, the AddModuleScore package from Seurat was also used to analyze the expression activities of genes involved in important pathways related to anti-tumor immunity. All the processed were completed on python (version 2.7.5) and R (version 3.6.3) platforms. Plots were made with ggplot2 (version 3.3.5), ggpubr (version 0.4.0), pheatmap (version 1.0.12) and ComplexHeatmap (2.8.0) packages in R.

### 2.1.10 Cell-Cell Communication Analysis

Cell-cell communication analysis were completed mainly with the R package CellChat (version 1.0.0) [64]. Five cell types that played important roles in the antitumor immunity were chosen for communication analysis. The communication numbers and strengths between the normal and Sephin1 groups in different tissues were calculated and compared.

### 2.1.11 In Vitro Analysis of the Effect of Sephin1 on Mouse CD8+ T Cells

A round-bottom 96-well plate was first prepared by incubation with 100 μl PBS supplemented with 1 μg/mL anti-mouse CD3F (BioXCell, BE0001-1-5MG) and anti-mouse CD28 (BioXCell, BE0015-1-5MG) the day before CD8+ T-cell isolation. The supernatant was discarded before use.

CD8+ T cells were isolated from the spleen tissue of adult male C57BL/6 mice with a MojoSort Mouse CD8 T Cell Isolation Kit (BioLegend, 480035) according to the standard protocol. The isolated CD8+ T cells were first incubated with reagents from a CFSE Cell Division Tracker Kit (BioLegend, 423801) according to the standard protocol and then resuspended in RPMI 1640 medium (Gibco, 11875093) supplemented with 10% FBS (Biological Industries, 04-001-1A) and 1% Pen Strep (Gibco, 15140122). Then, 20 ng/mL mouse IL2 (Novoprotein, CK24) and IL7 (Novoprotein, CC73) were added, and the concentration of cells was 10⁶/mL.

The isolated CD8+ T cells were then incubated in the precoated 96-well plates for 72 hours. After that, the cells were collected and stained with a LIVE/DEAD Fixable Violet Dead Cell Stain Kit (Invitrogen, L34963), PerCP/Cyanine 5.5-conjugated anti-mouse CD8a antibody (BioLegend, 100733) and PE-conjugated anti-mouse IFN-γ antibody (BioLegend, 163503) as described above. The prepared cell suspensions were analyzed on a CytoFLEX LX from Beckman Coulter.

### 2.1.12 Immunofluorescence Analysis

Immunofluorescence analysis was performed with mouse antibodies of F4/80 (Servicebio, GB11027), CD44 (Servicebio, GB112054), FN1 (Servicebio, GB112093) and SPP1 (Servicebio, GB11500). The tumor tissue was firstly fixed with paraformaldehyde, and embedded in paraffin, and then used for immunofluorescence staining.

### 2.1.13 Statistical Analysis

All statistical analyses were conducted using GraphPad Prism 7 (La Jolla, CA, USA) or R (version 3.6.3). Differences between 2 groups were calculated by either unpaired two-tailed Student's t-test (figures without statistical method annotation) or other suited statistical methods (annotated in the figures). Statistical parameters are shown as: *: p<0.05, **: p<0.01, ***: p<0.001, ****: p<0.0001.

### 2.2 Results

2.2.1 Sephin1 Accelerates Tumor Progression with ISR Activation and Immune Response Suppression.

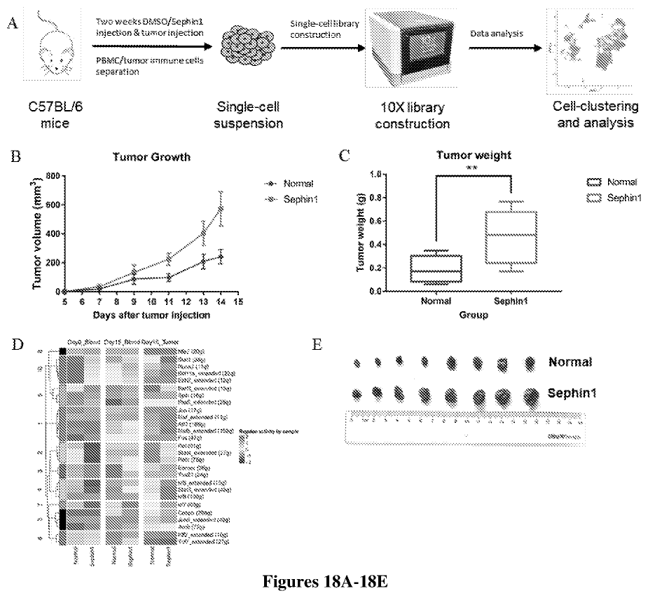

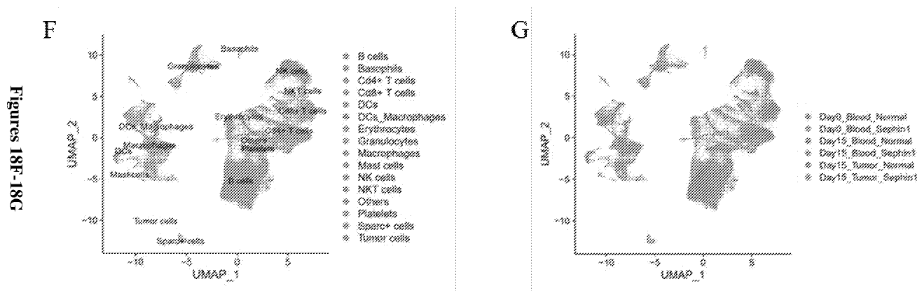

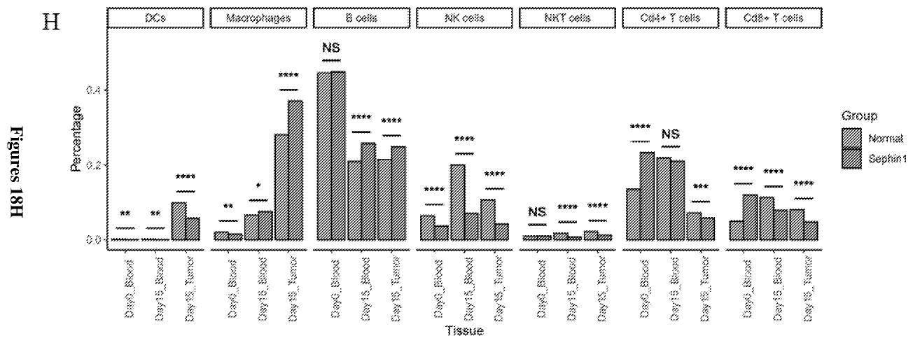

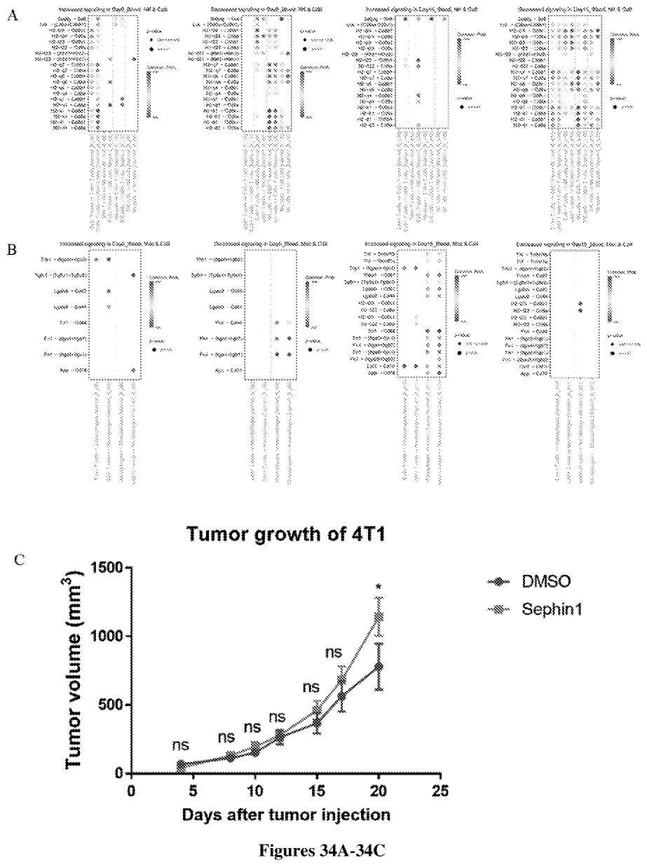

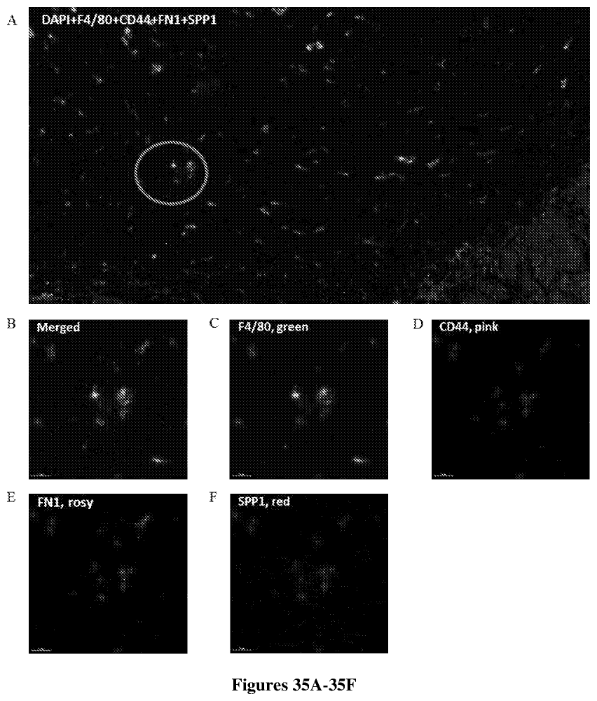

Three types of samples including PBMCs harvested on Day 0 (the day of tumor cell injection) and Day 15 (15 days after tumor cell injection) and immune cells isolated from tumor tissues harvested on Day 15 were collected for single-cell sequencing (FIG. 18A). Tumor growth curves showed that the tumor growth rate in the Sephin1 group was significantly higher than that in the normal group (FIG. 18B). Tumor tissues taken from both groups on Day 15 demonstrated that the tumor volume and weight in the Sephin1 group were also significantly higher than those in the normal group (FIGS. 18C and 18E). Apart from these findings, we repeated the in vivo Sephin1 experimental procedure with female BALB/c mice implanted with the mouse triple-negative breast cancer cell line 4T1. The tumor growth rate of 4T1 cells in the Sephin1 group was also higher than that in the normal group, indicating that the suppressive effect of Sephin1 on antitumor immunity may be common among different tumor types. However, the result was not as significant as that for the B16F1 cell line in male C57BL/6 mice (FIG. 34C), implying that the mechanism underlying the difference between the two cell lines needs to be further explored.

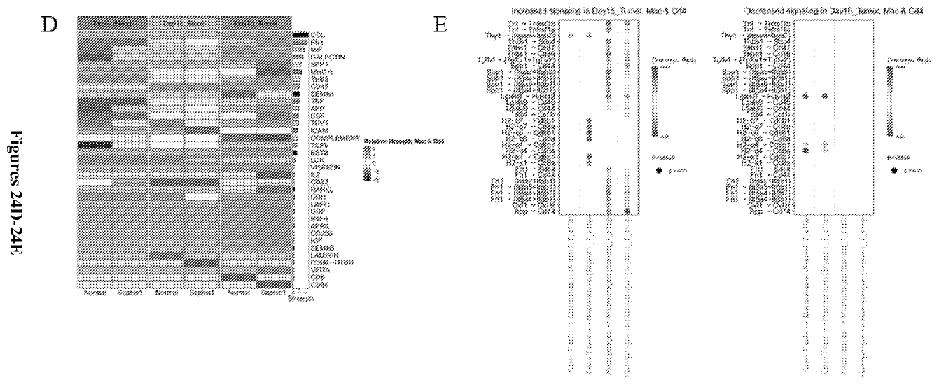

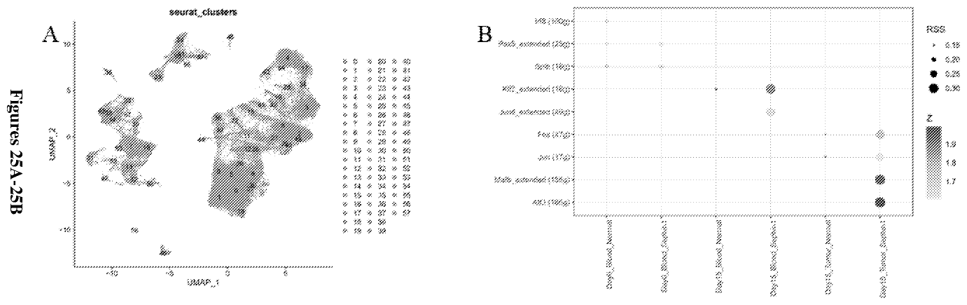

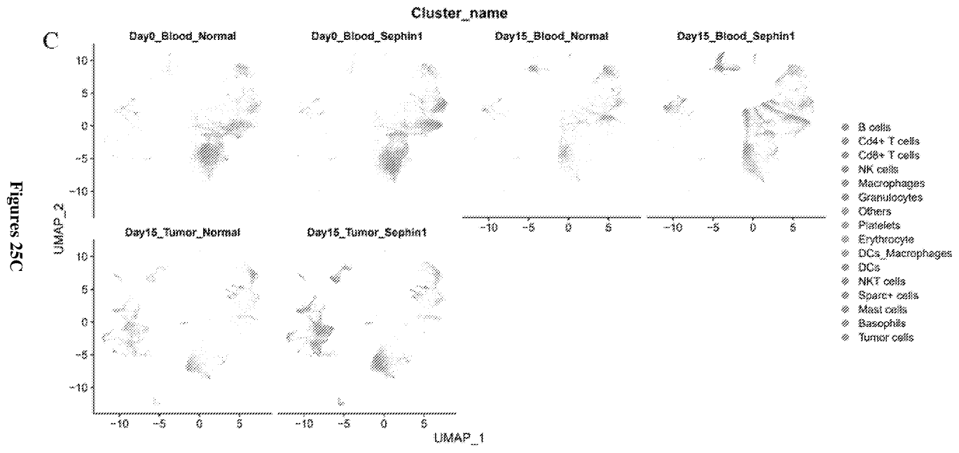

Raw single-cell sequencing data were first analyzed using CellRanger software developed by 10× Genomics and then merged, filtered and clustered with Seurat. After quality control, 68531 cells from 12 samples were included. The 12 samples were analyzed by sample type, and the samples included the normal and Sephin1 samples of blood collected on Day 0 and Day 15 and tumor immune cell samples collected on Day 15, for a total of 6 sample types. SCENIC analysis was performed and used to compare the samples to analyze regulon activity. A regulon is a coexpression module with significant motif enrichment of a certain upstream regulator, and higher regulon activity reveals higher cis-regulatory activity [63]. Twenty-seven regulons were identified in all six sample types, and the regulons were separated into 10 clusters by K-means based on their activity profiles (FIG. 18D). The Atf3 regulon, which includes genes involved in the ISR, such as Atf4 and Atf3, and related regulatory genes, had higher activity levels in the Sephin1 group blood samples collected on Day 0 and Day 15 and tumor samples collected on Day 15. Regulons in the same cluster as Atf3, including Jun, Maf_extended, Mafb_extended and Fos regulons, had activity profiles to that of the Atf3 regulon. However, regulons related to immune cell activities and differentiation, such as Stat1-, Stat3- and Stat4-related regulons, were downregulated in the Sephin1 group. In addition, RSS analysis also indicated that the Atf3 regulon was the most specific regulon in the Day 15 tumor samples (FIG. 25B).

### 2.2.2 Sephin1 Causes Suppression of Antitumor Immunity Mediated by Multiple Immune Cell Types

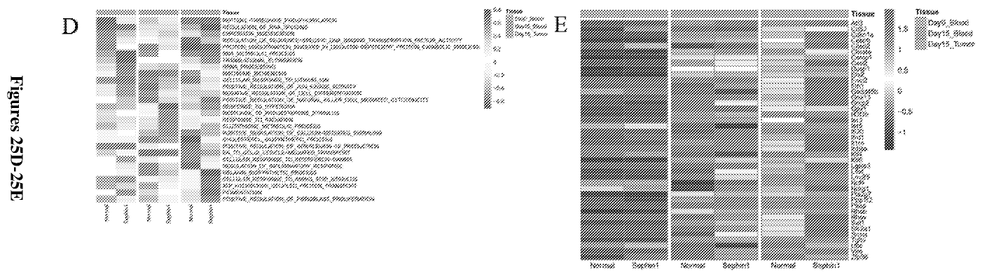

To further explore the modulatory effect of Sephin1 on the immune microenvironment, we analyzed the compositional changes in the broad categories of immune cells in the peripheral blood and tumors. Cells from all 12 samples were first separated into 6 sample types (FIGS. 18G and 25C) and then preprocessed, integrated and clustered with Seurat, and a total of 58 clusters were identified (FIG. 25A). These clusters were classified as different cell types based on classic cell markers, and all cells were separated into 16 types, including 5 lymphocyte cell types (NK cells, NKT cells, Cd8+ T cells, Cd4+ T cells and B cells) and 6 myeloid cell types (Basophils, Granulocytes, DCs_Macrophages, Macrophages, DCs and Mast cells; FIG. 18F). The marker gene expression profiles of different cell types are also shown (FIG. 26A). In the UMAP graph grouped by sample (FIG. 18G), we saw that the clustering between samples did not have a significant batch effect, which demonstrated that the sequencing data were comparable among different samples. The percentages of different cell types were calculated and analyzed (FIGS. 18H and 26B), and different samples showed different cell type distribution profiles. The cell types related to antitumor immune activities were more likely to be reduced in the Sephin1 group, especially in the tumor microenvironment on Day 15, including Cd8+ T cells, NKT cells, NK cells, and DCs. However, the distribution of macrophages was more likely to be enriched in the Sephin1 group. Subtype analysis was further performed on these important immune cell types.

GSVA analysis was also performed to compare the different sample types (FIG. 25D). In the tumor samples, genes expression level related to the melanin biosynthetic process, the cellular response to amino acid stimulus and ATP hydrolysis-coupled proton transport was upregulated in the Sephin1 group, which indicated higher ISR levels.

### 2.2.3 Sephin1 can Lead to Significantly Reduction of the Percentage of Anti-Tumor Lymphocytes

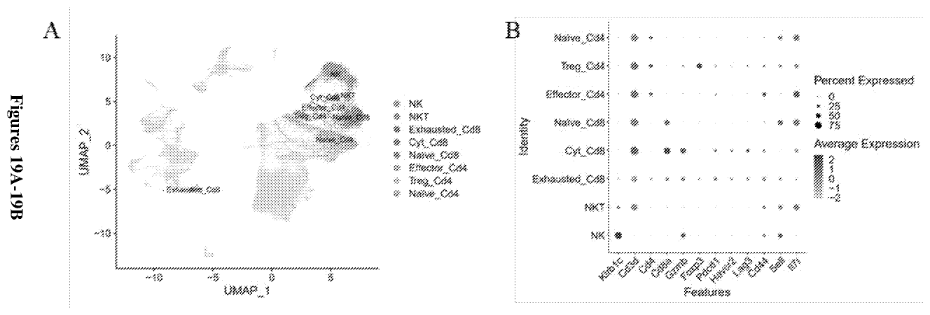

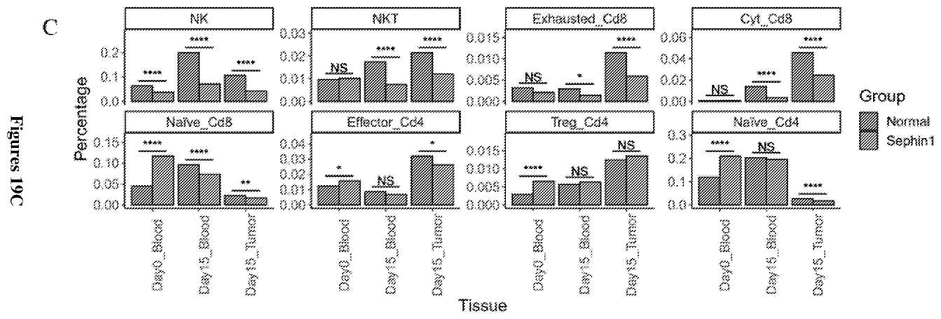

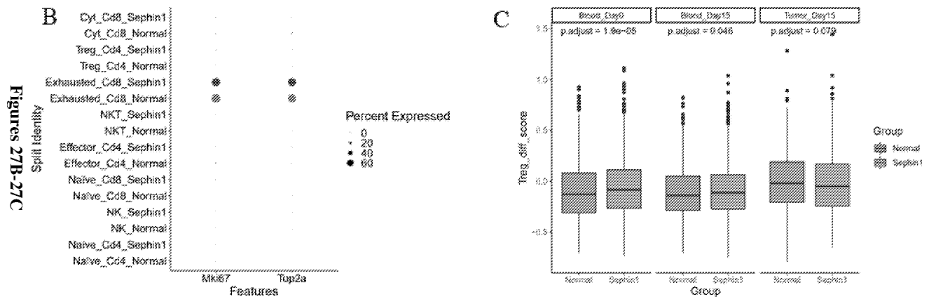

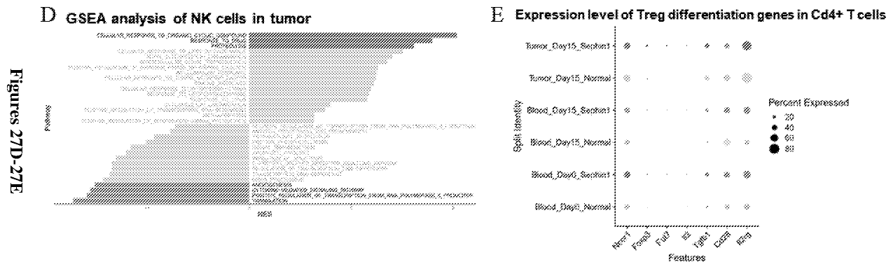

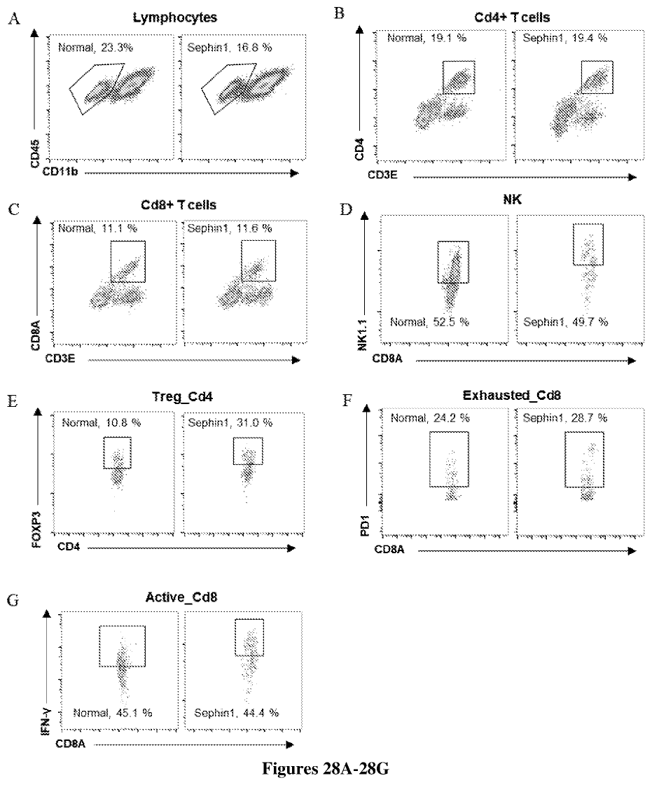

Considering the functional diversity of immune cells, we wondered which lymphocyte type plays the most crucial role in Sephin1-induced immunosuppression. We focused on several key antitumor immune cell types and analyzed their transcriptional profiles. B cells showed a similar distribution and expression patterns between the normal and Sephin1 groups and thus were excluded from further analysis. The remaining four lymphocyte cell types were further annotated as 8 subtypes. In contrast to NK cells and NKT cells, Cd4+ T cells were annotated into three subgroups: effector T cells, regulatory T cells and naïve T cells. Additionally, Cd8+ T cells were annotated into three subgroups: exhausted T cells, cytotoxic T cells and naïve T cells (FIG. 19A). Different markers were used for cell annotation (FIG. 19B). Compared with the blood samples collected on Day 0, the blood and tumor samples collected on day 15 showed more remarkable alterations in immune cell compositions induced by Sephin1 (FIG. 19C). The percentages of NK cells, NKT cells, exhausted Cd8+ T cells and cytotoxic Cd8+ T cells were all reduced significantly in the blood and tumor samples collected on Day 15 from the Sephin1 group. However, the change was not significant in the Day 0 blood samples. The percentage of naïve_Cd8+ T cells was slightly reduced in Day 15 samples but increased in Day 0 samples. The number of regulatory T cells was slightly increased in Day 15 samples but not as high as in Day 0 samples. FACS was used to verify these results, and the percentage of antitumor-related immune cell types in all immune cells were calculated and compared between the normal and Sephin1 group in the tumor microenvironment. The percentage of Cd4+ and Cd8+ T cells, NK cells and active Cd8+ T cells were all significantly downregulated in the Sephin1 group, while the regular Cd4+ T cells and exhausted Cd8+ T cells didn't vary significantly between the two groups (FIG. 19D, and FIGS. 28A-28G). We observed that in the FACS analysis results, the percentages of exhausted and activated Cd8+ T cells and NK cells were significantly reduced, but the percentage of regulatory T cells was increased.

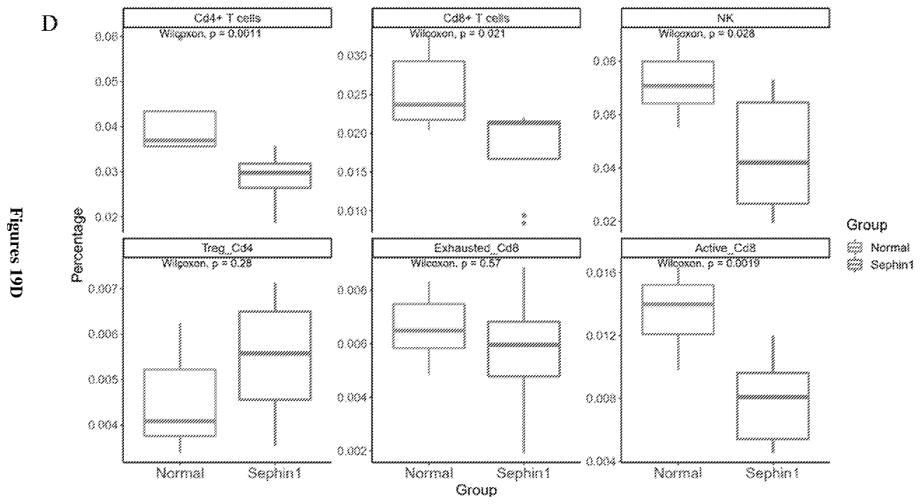

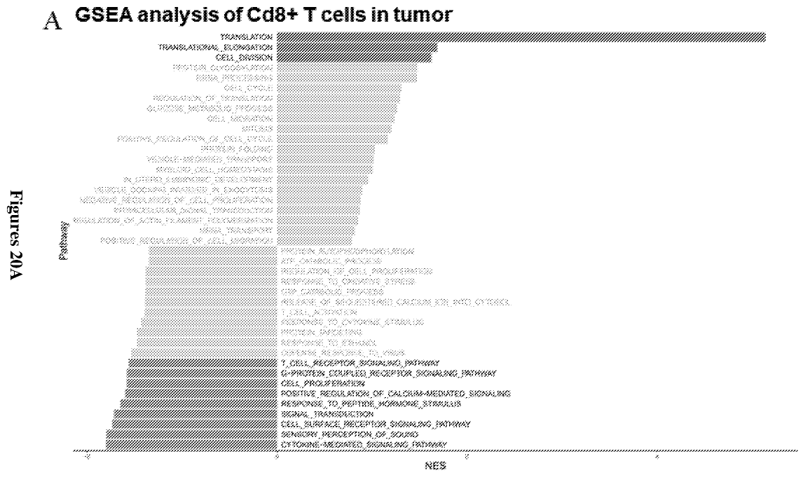

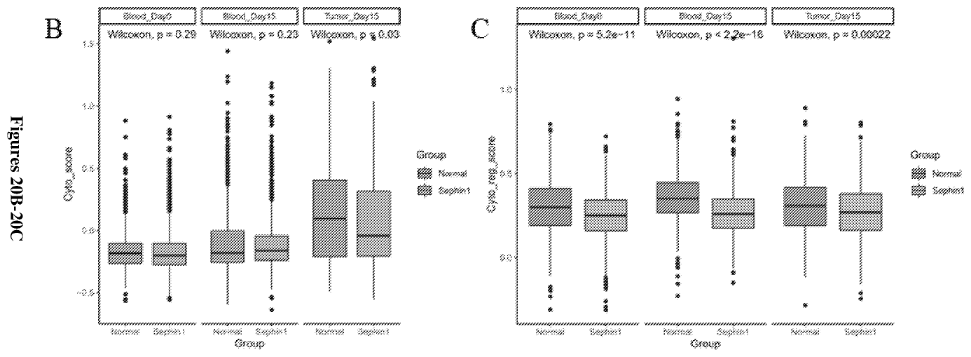

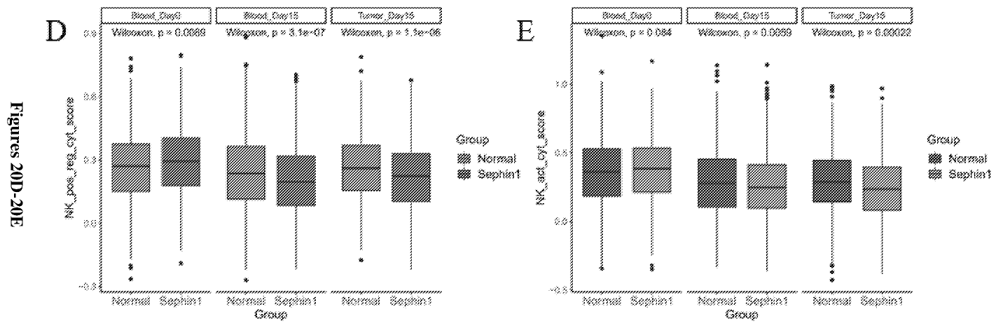

Apart from the composition change, Sephin1 also affected the expression levels in lymphocytes. To determine the effect of Sephin1 on the expression patterns of different lymphocyte subtypes, we analyzed differential expression levels between the normal and Sephin1 groups. In Cd8+ T cells, we found that several important pathways related to antitumor immunity were significantly downregulated in the Sephin1 group (FIG. 20A). The significantly downregulated pathways included cytokine-mediated signaling, cell surface receptor signaling, signal transduction, response to peptide hormone stimulus, positive regulation of calcium-mediated signaling, cell proliferation, G-protein coupled receptor signaling and the T cell receptor signaling pathway. The downregulation of these pathways indicated that the function of Cd8+ T cells was suppressed significantly. The significantly upregulated pathways in the Sephin1 group included pathways related to translation, translational elongation and cell division, which indicated the loss of feedback regulatory function in the translational process.

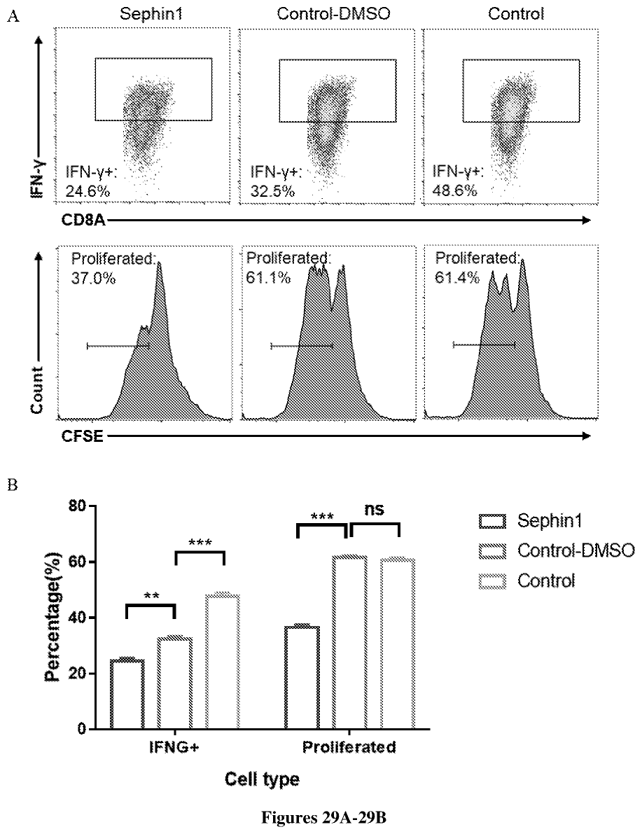

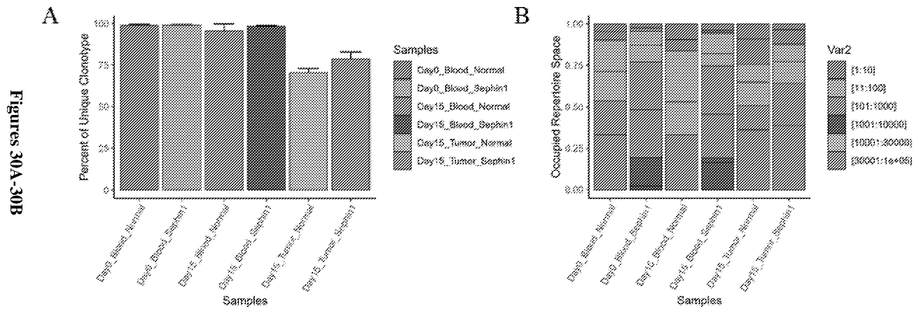

To identify the specific pathways most affected by Sephin1, we calculated the expression score of Cd8+ T cells using the Seurat package AddModuleScore. Genes involved in T-cell cytotoxicity and positive regulation of T-cell cytotoxicity from Gene Ontology was analyzed. Genes involved in T-cell cytotoxicity had significantly lower expression scores in the Day 15 tumor microenvironment samples but not the Day 0 or Day 15 blood samples from the Sephin1 group (FIG. 20B). However, the expression levels of genes related to the positive regulation of T-cell cytotoxicity were significantly downregulated in all three sample types (FIG. 20C). These results showed that the activity of Cd8+ T cells was inhibited by Sephin1, especially in the tumor microenvironment. In vitro analysis of Cd8+ T cells comparing the control and Sephin1 groups also showed that the percentage of cells expressing IFN-γ in the Sephin1 group was significantly lower than that in the control group, which indicated that the Sephin1 group had lower Cd8+ T-cell activity in vitro (FIGS. 29A and 29B).

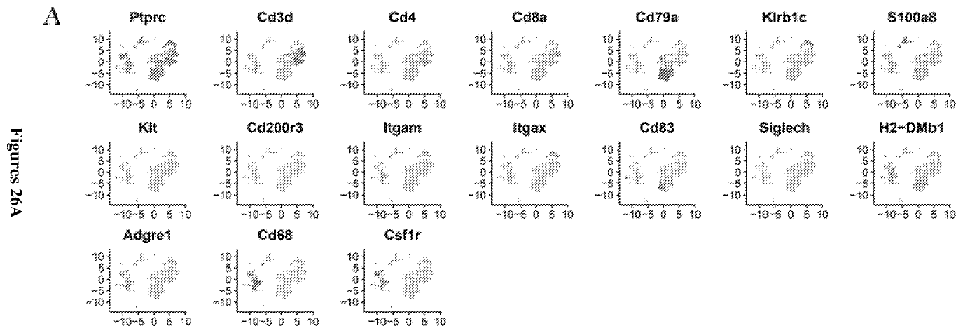

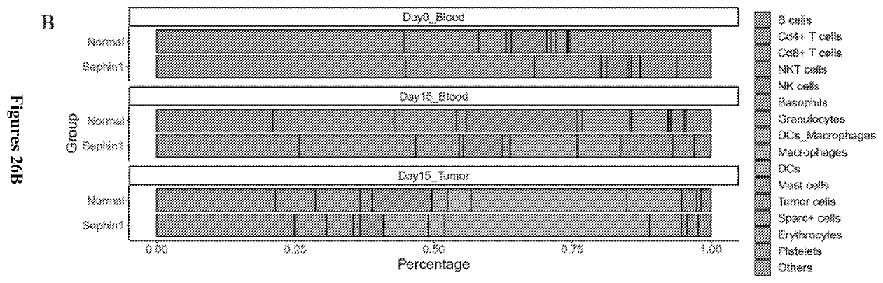

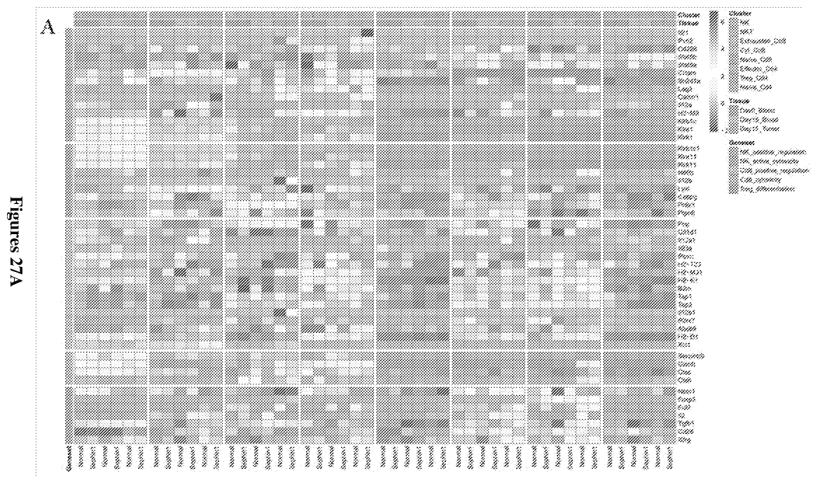

The scores of genes related to NK-cell positive regulation (FIG. 20D) and activity (FIG. 20E) from Gene Ontology was also calculated, and they exhibited similar patterns. GSEA of NK cells also demonstrated that genes related to NK-cell activity, including genes involved in the cytokine-mediated signaling pathway and induction of apoptosis, were downregulated in the tumor Sephin1 group (FIG. 27D). The Treg differentiation score was also calculated for regulatory T cells, and the score was significantly upregulated in PBMCs in the Sephin1 group but downregulated in tumor samples (FIG. 27C). The gene expression levels of these gene sets were also calculated and compared among different clusters in different sample types (FIG. 27A). The expression levels of NK-cell positive regulation- and activity-related genes were downregulated in both the blood and tumor microenvironment on Day 15 but slightly upregulated in the blood on Day 0. We also performed SCENIC analysis of all lymphocyte subtypes (FIG. 20F). The activity of the Atf3 regulon was upregulated in exhausted Cd8+ T cells, cytotoxic Cd8+ T cells, NK cells and NKT cells but downregulated in regulatory T cells. These results indicated that the inhibitory effect of Sephin1 might vary among different cell types but have similar immunoinhibitory functions. The PI3K-related regulons showed similar patterns in these cell types, and the activity scores of PI3K-related regulons were all downregulated in both cytotoxic T cells and regulatory T cells.

2.2.4 TCR Analysis Reveals that Specific T Cell Proliferation could be Inhibited by Sephin1

By analyzing CFSE-stained Cd8+ T cells in vitro, we found that the Sephin1 group had a significantly lower proliferative ability than the control group (FIGS. 29A and 29B). Although the overall expression of proliferation-related genes was not different between the two groups, the clonal expansion of T cells in tumor was inhibited in the Sephin1 group based on single-cell TCR analysis. TCRs were separated into four types: hyperexpanded, large, medium and small based on the described methods.

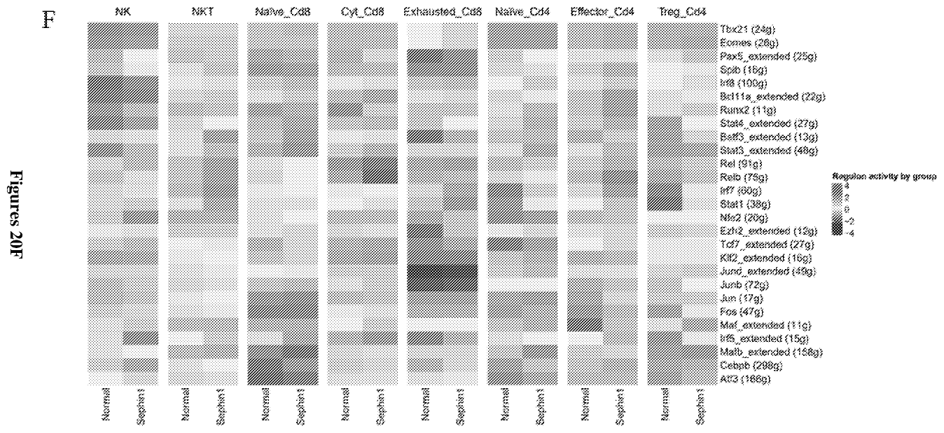

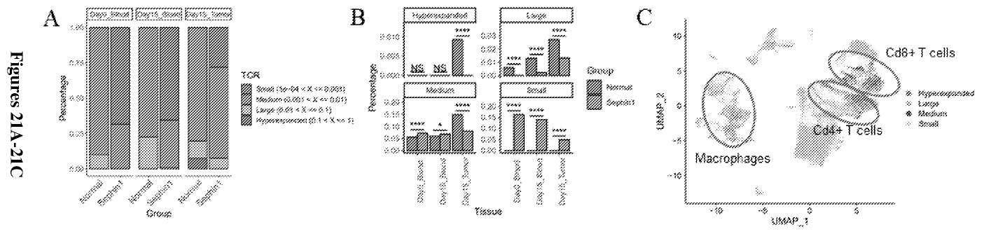

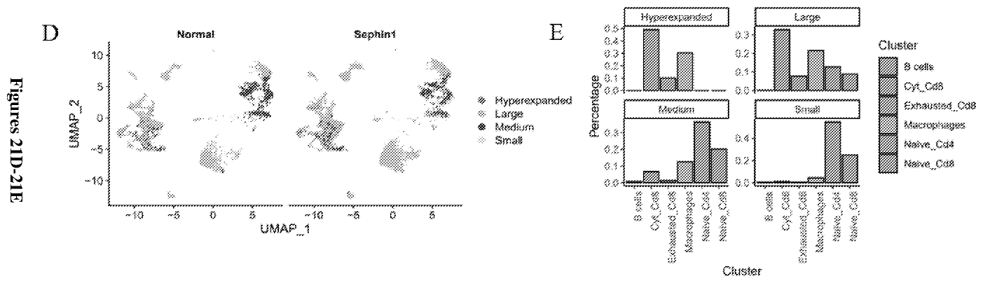

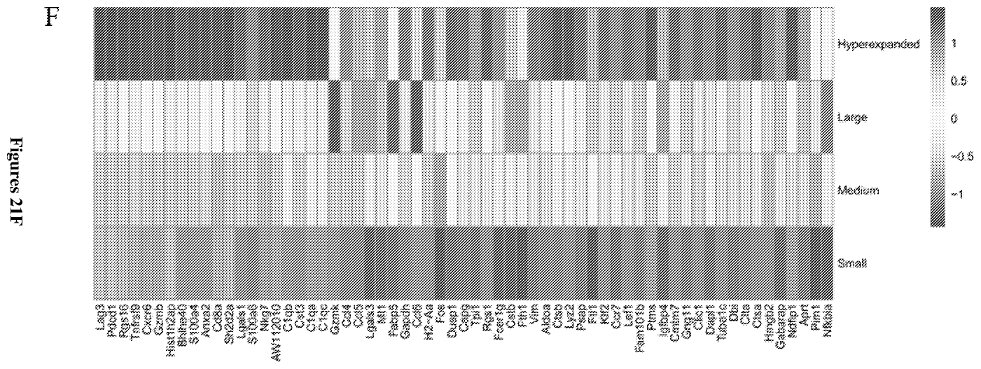

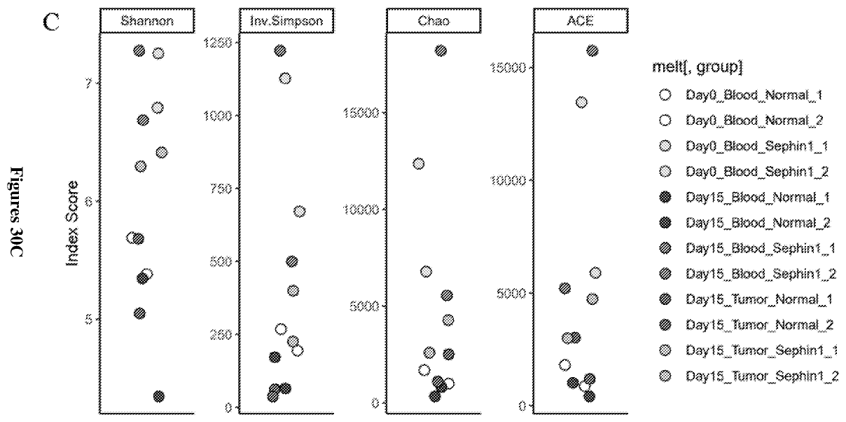

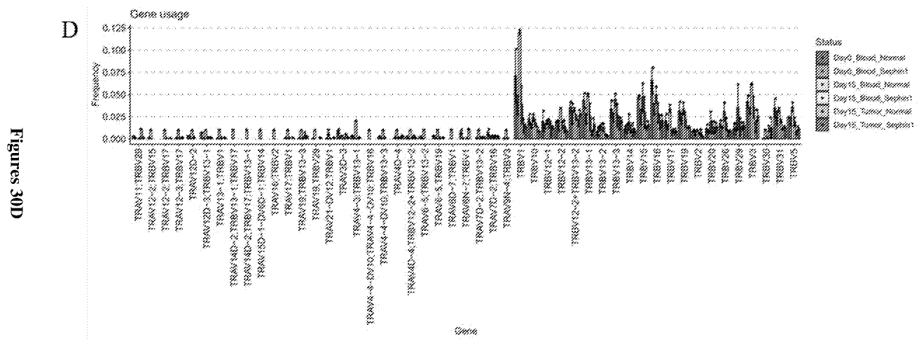

First, we calculated the percentages of different categories of clonotypes in one sample (FIG. 21A). We found that for all three sample types, the samples in the Sephin1 group had a lower percentage of highly expanded clonotypes. In the blood samples collected on Day 0 or Day 15, the percentages of TCR types in the large and medium categories were significantly lower in the Sephin1 group. In the Day 15 tumor samples, the hyperexpanded, large and medium TCR types were also significantly downregulated in the Sephin1 samples. Apart from the clonotype percentages, the percentage of cells belonging to each TCR type showed a similar pattern. The percentages of cells belonging to the hyperexpanded and large types were downregulated in the Sephin1 samples for all three sample types, while the percentage of the small type was upregulated (FIG. 21B). The ranking of different TCR clonotypes showed similar results. Clonotypes were ranked according to their clone number proportion within the complete TCR repertoire of one sample, and the percentages of clonotypes with higher ranks were decreased in all three sample types (FIG. 30B).

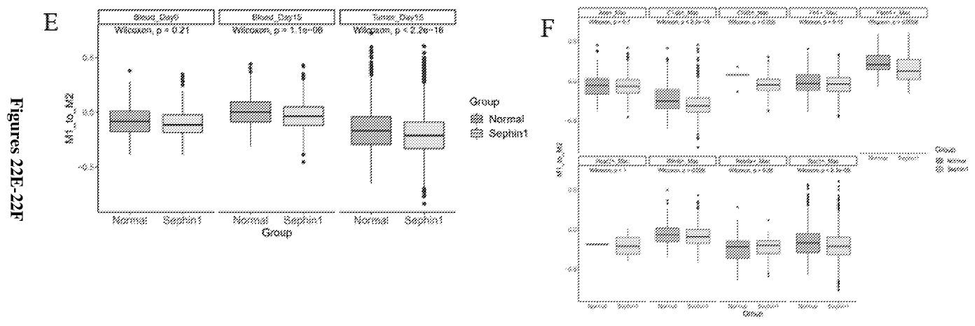

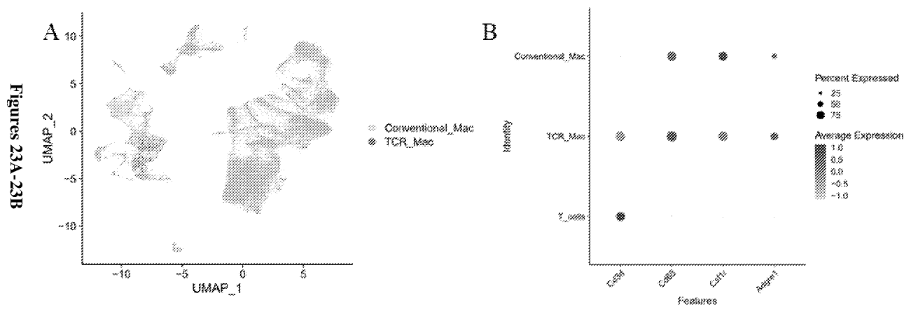

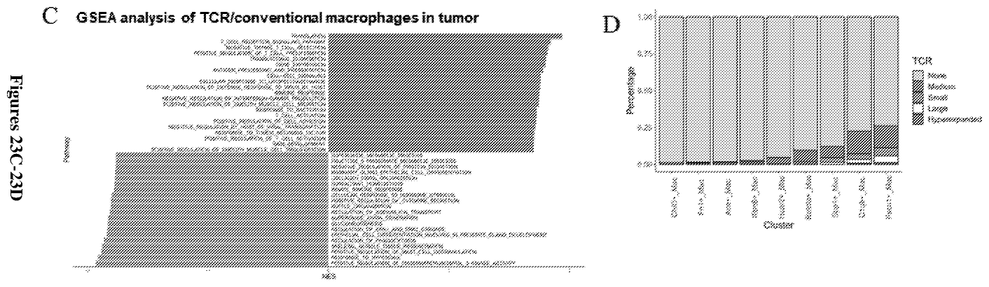

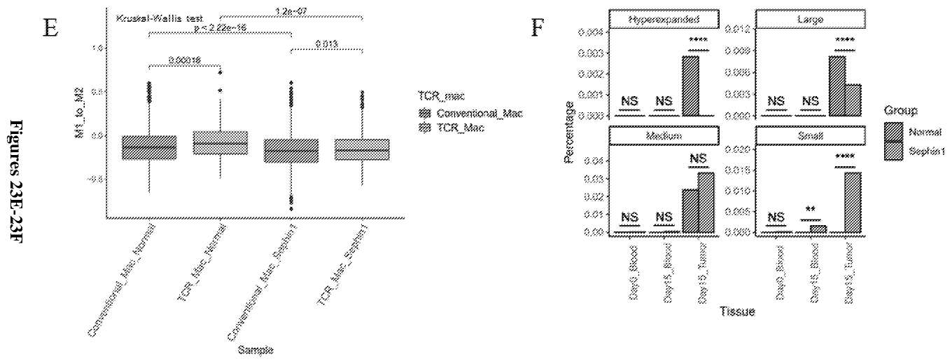

Further analysis found that although TCR+ cells were mainly distributed in T cells, there was also a large number of TCR+ cells found in the macrophage population, and a comparatively high proportion of macrophages had a hyperexpanded- or large-clonal TCR types (FIGS. 21C and 21E). In addition, the percentage of highly expanded TCR types, namely, a hyperexpanded or large TCR type, was also higher in the normal group than in the Sephin1 group for both T cells and macrophages (FIGS. 21B, 21D, and 23F).

To determine the expression patterns of different TCR types, especially highly expanded TCR types, we analyzed the differentially expressed genes in different TCR types. The hyperexpanded type had much higher expression levels of cytotoxicity-related genes, such as Gzmb and Gzmk (FIG. 21F). GSVA was performed according to the expression levels of the differentially expressed genes of each TCR type, and the results showed that the highly expanded TCR cell types had higher metabolic activities. Pathways such as the cholesterol biosynthetic process and tricarboxylic acid cycle were found to be highly expressed in the hyperexpanded type. Immune-related pathways, such as the cellular response to hypoxia pathway and the antigen processing and presentation of an exogenous peptide antigen via MHC class II pathway, were activated in the large type (FIG. 21G). Immunity-related and cell-killing-related pathways were also upregulated in the hyperexpanded type, such as the inflammatory response and positive regulation of natural killer cell chemotaxis pathways (FIG. 21H).

### 2.2.5 Macrophages in the Sephin1 Group are More Likely to be in an M2-Polarized State

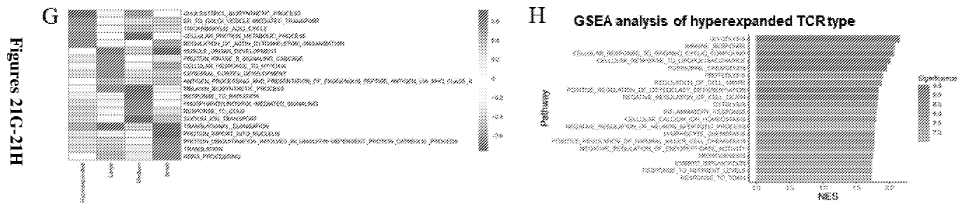

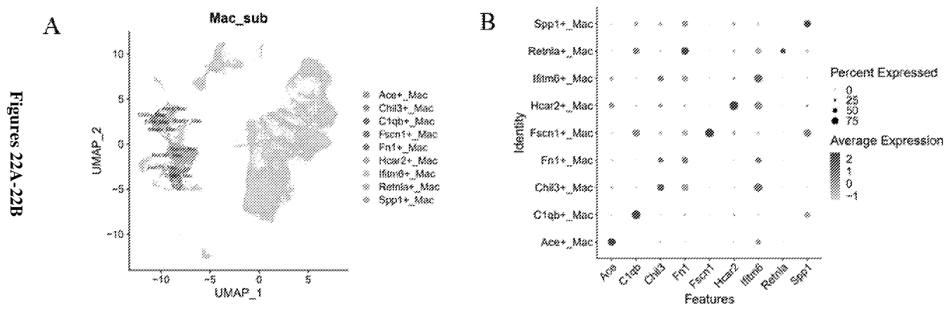

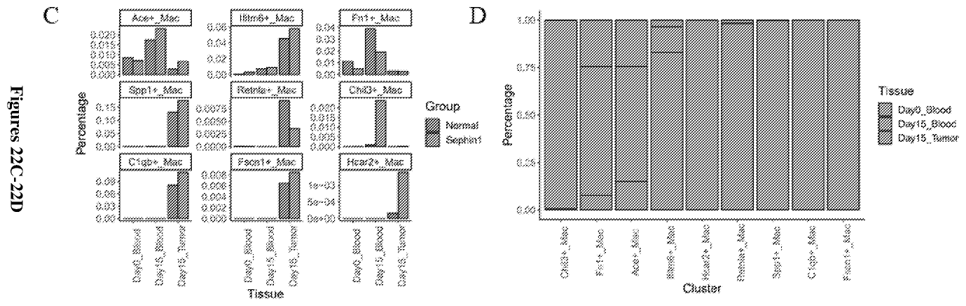

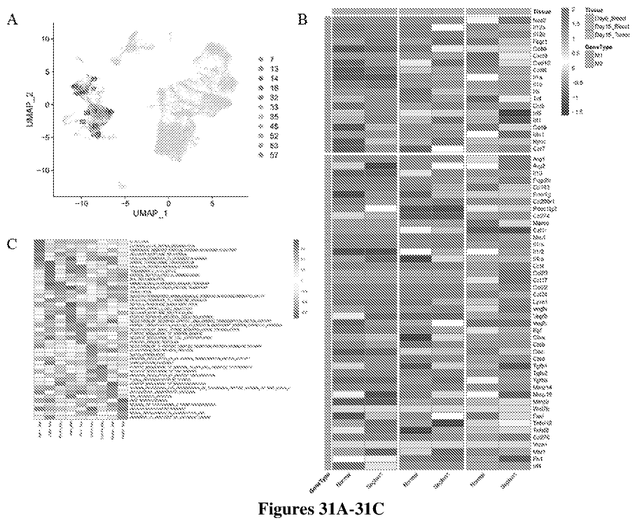

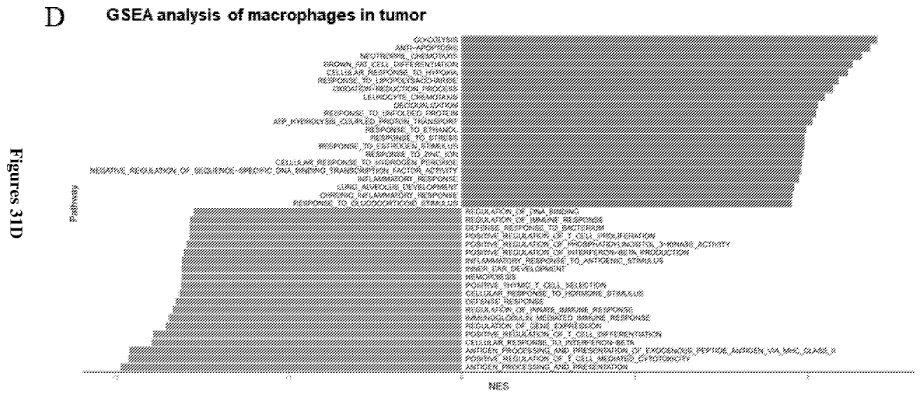

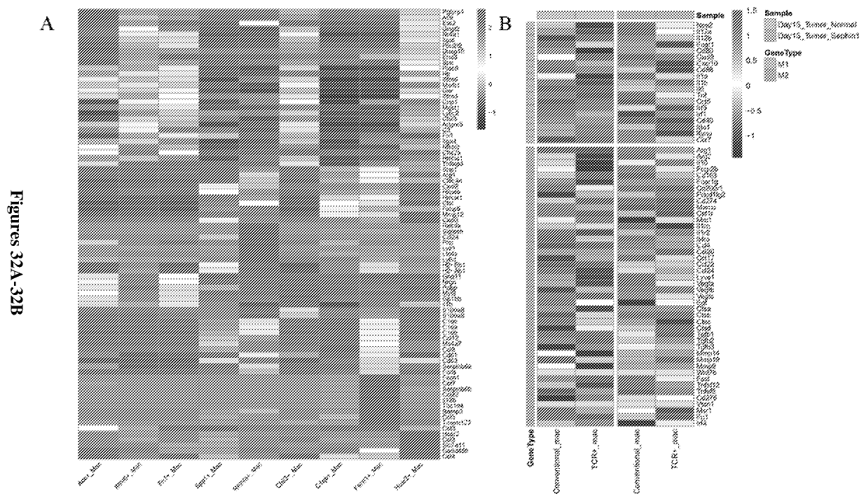

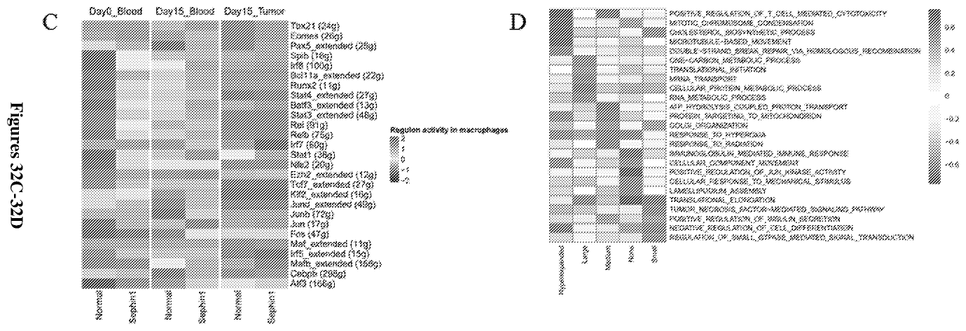

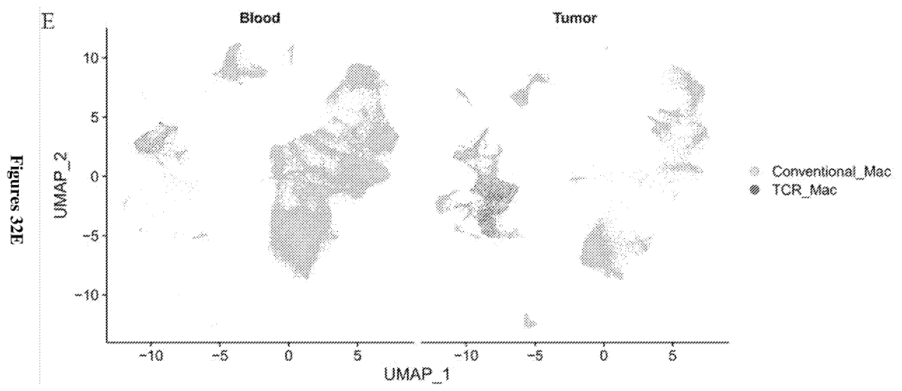

In addition to antitumor lymphocytes, macrophages also play key roles in the tumor microenvironment. Thus, we also analyzed the characteristics and functions of macrophages in different samples. Macrophages in all merged samples were divided into 11 clusters with Seurat (FIG. 31A), and we annotated them into 9 subtypes named based on their specific marker genes (FIGS. 22A and 22B). Chil3+, Fn1+ and Ace+ macrophages mainly existed in the blood. Ifitm6+, Hcar2+, Retnla+, Spp1+, C1qb+ and Fscn1+ macrophages mainly existed in tumor tissues (FIG. 22D). There were two macrophage subgroups that mainly existed in the Sephin1 group: Chil3+ macrophages, which mainly existed in the Day 15 blood samples, and Hcar2+ macrophages, which mainly existed in Day 15 tumor samples (FIG. 22C). GSVA of different macrophage subtypes showed that the highly expressed genes in the Chil3+ group were enriched in the regulation of G-protein coupled receptor protein signaling and negative regulation of NF-κB pathways (FIGS. 31C and 32A).

There are two macrophage polarization states, M1 and M2. Typically, M1 macrophages produce type I proinflammatory cytokines and have antitumorigenic functions, while M2 macrophages produce type II cytokines and have protumorigenic functions [65, 66]. We then analyzed the expression levels of genes related to the M1- and M2-polarized states [67] by calculating the gene expression scores for M1 and M2 polarization with AddModuleScore followed by the M1_to_M2 score determined by subtracting the M2 score from the M1 score. The higher the M1_to_M2 score was, the more the cells were polarized toward the M1 state. We found that in the blood and tumor tissues collected on Day 15, the macrophages in the Sephin1 group were significantly polarized toward the M2 state compared with those in the normal group, but the variation in the Day 0 blood samples was not significant (FIGS. 22E and 31B). We then analyzed the M1- and M2-polarized states of different macrophage subtypes. Except for two subtypes with too few cells (Retnla+ macrophages and Hcar2+ macrophages), we found that the subtypes mainly existing in tumor tissues were more tended to M2 polarization, including the Spp1+, Clqb+, Ifitm6+ and Facn1+ macrophages (FIG. 22F).

GSEA was also performed on macrophages in tumor tissue to compare the Sephin1 and normal groups. We selected the 20 most upregulated and downregulated pathways between the two groups based on the normalized enrichment score (NES) (FIG. 31D). We found that pathways related to T-cell activities, antigen processing and presentation were downregulated in the Sephin1 group. In contrast, the pathway related to the ISR process such as the cellular response to hypoxia pathway was upregulated in the Sephin1 group. In addition, SCENIC analysis of macrophages also indicated that the ISR-related regulon, i.e., the Atf3 regulon, had higher activity in the Sephin1 group (FIG. 32C).

2.2.6 the Macrophage Subtype with TCR Expression May have Important Functions in Antitumor Immunity

By TCR analysis, we found that TCRs existed not only in T cells but also in macrophages and that the percentage of TCR+ macrophages for all three TCR types was as high as approximately 0.3 (hyperexpanded type, FIG. 21E). In addition, GSEA comparing macrophages between the normal and Sephin1 groups indicated that pathways related to T-cell activities were affected by Sephin1 (FIG. 31D). These results indicated that macrophages with TCR sequences might have important functions in the immune system and antitumor procedures. Research on CD3+ macrophages has found that this cell type can produce proinflammatory cytokines [68], yet their role in antitumor immunity is still unknown. In our research, we found that Cd3+ macrophages, especially TCR+ macrophages, may had important functions in antitumor immune activities. TCR+ macrophages existed in both the blood and tumor tissues but were more enriched in the tumor microenvironment (FIGS. 23A and 32E). Approximately 13.7% of the macrophages in tumors were TCR+, but only approximately 0.5% of the macrophages in the blood were TCR+. Marker genes for both macrophages and T cells were highly expressed by this cell type, including Cd3d for T cells and Cd68, Csf1r and Adgre1 for macrophages (FIG. 23B). GSEA comparing TCR+ macrophages and conventional macrophages indicated that the genes upregulated in TCR+ macrophages were more enriched in pathways related to T-cell activation and regulation and that the downregulated genes were enriched in pathways related to the innate immune response, cytokine secretion and other related pathways (FIG. 23C). The hyperexpanded and large TCR types mainly existed in C1qb+ and especially Fscn1+ macrophages, which mainly existed in tumor tissues (FIG. 23D). GSVA of TCR+ macrophages was also performed by TCR type (FIG. 32D). In the hyperexpanded macrophage group, the most upregulated genes were enriched in the positive regulation of the T-cell-mediated cytotoxicity pathway, which was similar to cytotoxic T-cell functions. This result also indicated that TCR+ macrophages might perform antitumor functions in both macrophage-like and T-cell-like ways.

We also calculated the M1_to_M2 scores of both conventional macrophages and TCR+ macrophages with AddModuleScore (FIG. 23E). TCR+ macrophages had significantly higher M1_to_M2 scores in the normal group, implying that these macrophages were more likely to have antitumor functions. The differences between the two macrophage types were less significant in the Sephin1 group, but both cell types were more polarized toward the M2 state in the Sephin1 group. These results showed that TCR+ macrophages were likely to have antitumor functions mediated through both macrophage- and T-cell-related pathways and that these functions could be inhibited by Sephin1.

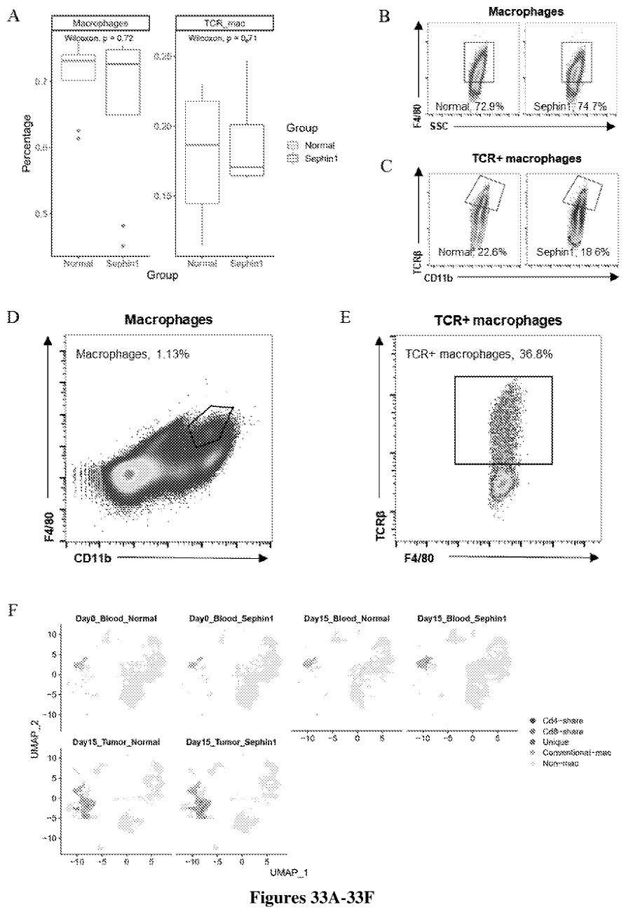

Distribution analysis of the different types of TCR+ macrophages in different tissue types showed that the percentages of the hyperexpanded and large types of macrophages were significantly downregulated in the Sephin1 group, while the percentages of the medium and small types of macrophages were upregulated, which was consistent with the overall TCR+ cell patterns (FIG. 23F). In addition, we also calculated the percentage of TCR+ macrophages with shared TCR sequences with Cd4+ (Cd4-share) and Cd8+ T cells (Cd8-share) in the tumor tissue. It turns out that in the Sephin1 group, the Cd8-share macrophages was significantly fewer than the normal group (FIGS. 23G and 23H), which indicated that the TCR+ macrophages may got the TCR sequences through interaction with T cells. The existence of TCR+ macrophages was also proved by FACS results in the tumor microenvironment, and there was no significantly difference between the normal and Sephin1 group (FIGS. 33A-33C). In addition, we also found that TCR+ macrophages existed in the normal mouse spleen tissue (FIGS. 33D and 33E).

### 2.2.7 Sephin1 Suppresses Antitumor Immunity in Cell-Cell Communication Level

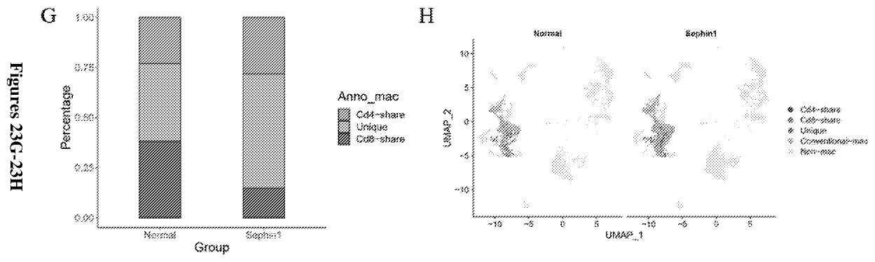

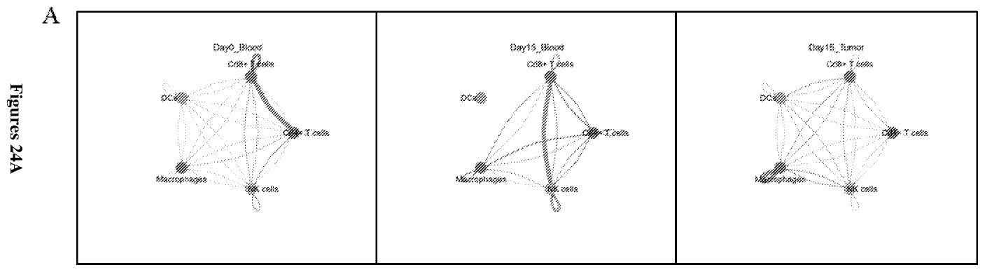

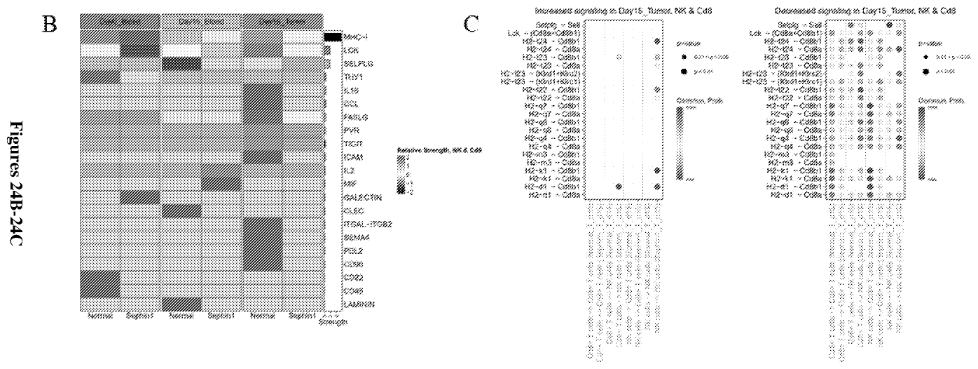

In order to find the differentially expressed genes and communication strengths between the normal and Sephin1 group, we calculated the cell-cell communication score and strength between different samples by CellChat [64]. In the tumor tissue, most communication strengths were downregulated in the Sephin1 group, except the communications of macrophages-macrophages and macrophages-Cd4+ T cells. In all three tissue types, communications between Cd8+ T cells and NK cells were all downregulated in the Sephin1 group, which indicated that these cell-cell communications may have more important functions (FIG. 24A).

By analyzing the differentially expressed ligand-receptor pairs between the normal and Sephin1 groups between Cd8+ T cells and NK cells, we found that these pairs were mostly enriched in MHC-I related pathways (FIG. 24B). Besides, the strengths of ligand-receptor pairs in MHC-I pathway were all downregulated in the Sephin1 group of all three tissue types. This result indicated that the influence of Sephin1 to the antitumor immunity was enriched in MHC-I related pathways. Apart from this, two other pathways with relatively high overall strength also showed reduced tendency in the Sephin1 group, including LCK pathway and SELPLG pathway (FIG. 24B). In the tumor microenvironment, Selplg-Sell and Lck-(Cd8a+Cd8b1) pairs were included in the significantly decreased pairs in Sephin1 group compared to normal (FIG. 24C).

On the contrary, the communication strength of macrophages-Cd4+ T cells and macrophages-macrophages was upregulated in the Sephin1 group. Therefore, we also analyzed the differentially expressed communication pathways between these two cell types (FIG. 24D). Pathways with high expression levels and also significantly upregulated in the Sephin1 group included FN1, GALECTIN, SPP1, MHC-I, THBS, TGFb, APP, THY1, TNF and CSF, in which many were related to antitumor immunity suppression. We further analyzed the ligand-receptor pairs in these pathways (FIGS. 24E and 34B). Although MHC-I pathway was upregulated in the Sephin1 group of tumor tissue by overall strength (FIG. 24D), the number of upregulated ligand-receptor pairs was less than downregulated ones (FIG. 24E). In addition, most up-regulated pairs were enriched in the macrophage-macrophage interaction, which was also verified by immunofluorescence results (FN1-CD44 & SPP1-CD44 pairs. FIGS. 35A-35F). Furthermore, in the tumor tissue, the upregulated ligand-receptor pairs mostly existed in the macrophage-macrophage communication including Thbs1, Spp1, Lgals9 and Csf related pairs, which were highly related to immunity suppression.

### 2.3 Discussion

Sephin1 is a selective inhibitor of PPP1R15A, and can inhibit dephosphorylation of eIF2α by inhibiting the formulation of the PPP1R15A-PP1c complex [31], eIF2α is a key component of the integrated stress response process (ISR), which can be induced by both extrinsic factors and intrinsic cellular stresses, including oncogene activation [16, 69, 70]. Usage of Sephin1 in mammals can lead to a promotion of ISR activity, thus used as a potential treatment in neuron, motor and proteostasis related diseases [45, 71, 72]. In our study, we found that the usage of Sephin1 in mice can lead to antitumor immunity suppression, which is most likely to be achieved by ISR process by single-cell expression analysis. In the C56BL/6 mice injected subcutaneously with B16F1 cells, the tumor growth rate in the Sephin1 group was significantly higher than that in the normal group, which indicated a possible relationship between the ISR process and antitumor immune activities. SCENIC analysis of the single-cell data for all immune cells between the normal and Sephin1 groups showed that all three sample types showed higher activities of the Atf3 regulon, which includes core genes related to the ISR, and other related regulons in the Sephin1 group, which indicated a higher ISR level. However, regulons related to immune cell activities were downregulated in the Sephin1 group, which indicated the induction of an immunosuppressive effect by Sephin1. To fully understand the suppressive effect on antitumor immune activities mediated by different kinds of immune cell types, we analyzed the expression and distribution patterns of different cell types in different tissues.

Lymphocytes that are important for antitumor immunity were more likely to be affected by Sephin1 injection. NK cells, NKT cells and Cd8+ T cells were all significantly reduced among the immune cells in tumor tissue in the Sephin1 group, while regulatory T cells were more enriched. In addition, as key antitumor cell types in innate and adaptive immune systems [73, 74], Cd8+ T cells and NK cells also exhibited lower expression and cell-killing activities in the Sephin1 group. As for NKT cells, previous studies have shown that depending on the cell type, NKT cells can either suppress (type I NKT cells) or promote (type II NKT cells) tumor development [75, 76]; thus, the effects of the reduction in NKT cells may be controversial. Additionally, the enrichment of regulatory T cells in the Sephin1 group also indicated suppression of antitumor immunity [77]. SCENIC analysis also indicated that Atf3 regulon activity in the Sephin1 group in tumor tissue, was higher in antitumor cell types such as NK cells, NKT cells, and Cd8+ T cells, but lower in the suppressive T-cell type, regulatory Cd4+ T cells [78], which also indicated that the antitumor suppression effects of Sephin1.

By analyzing the TCR clonotype distribution, we found that tumor-specific T-cell proliferation was also suppressed by Sephin1 injection. The TCR sequencing analysis indicated that highly expanded TCR clonotypes were significantly decreased in the Sephin1 group in terms of the numbers of both clonotypes and clones. Highly expanded TCR clonotypes were more enriched in cytotoxic Cd8+ T cells and macrophages and had higher expression of genes related to cytotoxicity-related pathways, which indicated that these cells were important for tumor-specific identification and cell killing. Additionally, clonotypes with a lower clone number were more enriched in naïve T cells.

Macrophages can also exert important antitumor immune activities. Macrophages can have a tendency to polarize toward the M1 or M2 state [79, 80] but exist along a continuum and cannot be distinctly separated into the M1 or M2 type [67, 81]. Previous studies have demonstrated that M1 macrophages are proinflammatory, while M2 macrophages are anti-inflammatory [82]. In the tumor microenvironment, M1-like macrophages are more likely to have antitumor functions, while M2 macrophages have the opposite impact [83]. In our experiment, by evaluating the polarization tendency through evaluation of a series of M1- and M2-related genes, we found that in the Sephin1 group, macrophages tended to exhibit an M2-polarized state, which was more likely to promote tumor development. In addition, macrophage subtypes in the tumor microenvironment were more deeply affected by Sephin1 than those in the blood, indicating that Sephin1 had stronger influence on tumor-associated macrophages.

Previous studies have shown that CD3+TCRαβ+ macrophages can produce proinflammatory cytokines and have import functions in infection-related biological process [68], and this kind of macrophages may generated through the trogocytosis between macrophages and T cells [84, 85]. However, the traditional trogocytosis theory only included the exchange of membrane and membrane-associated proteins. In our study, the single cell sequencing data was at mRNA level, which indicated that a large number of macrophages in the tumor microenvironment contained the mRNAs of TCRs and other T-cell related genes. This phenomenon indicated that the interaction between APC and T cells may not be limited to the cell surface, but also involve a deeper level of substance exchange. In addition, clonotype analysis of TCR+ macrophages indicated that macrophages with higher TCR frequency were more likely to be suppressed in the Sephin1 group. Besides, TCR+ macrophages tended to undergo M1 polarization more than conventional macrophages and were also more enriched in the tumor microenvironment. These results all indicated that TCR+ macrophages could play vital roles in both T-cell- and macrophage-related pathways. The suppressive effect of Sephin1 on this cell type was also more significant than that on conventional macrophages.

Based on these results, we further analyzed the cell-cell communication between Cd8+ T cells, Cd4+ T cells, NK cells, macrophages and DCs, and analyzed cell pairs that had similar patterns between all three tissue types. In the cell-cell communications that were downregulated in the Sephin1 group, MHC-I, LCK and SELPLG pathways were significantly downregulated and also had relatively high communication strengths. The SELPLG pathway is known with cell-cell adhesion function, and may have functions in antitumor immunity [86, 87]. MHC-I and LCK pathways have important functions in antigen presenting and also associated with each other. LCK is known as inducing initial TCR-triggering event [88]. FN1, GALECTIN, SPP1, THBS, TGFb, APP, THY1, TNF and CSF pathways were upregulated in the Sephin1 group. Most of these pathways were antitumor suppressive. GALECTIN can lead to T cell inhibition by Lgals9-Havcr2 interaction [89]. SPP1 can facilitate immune escape in tumor tissues [90]. THBS1 can limit antitumor immunity by CD47-dependent regulation of innate and adaptive immune cells [91]. CSF1/CSF1R pathway can lead to inhibition to T-cell checkpoint immunity [92]. TNF is an important pathway in cell apoptosis, which is also highly related to ISR process, can also trigger the death signaling in immune cells [93]. TGFb is known as an important markers of M2 macrophages, which is highly related to pro-tumor effects [94]. APP and THY1 pathways may also have important functions in antitumor immunity, however research on these two pathways about antitumor immunity is still lack.

In conclusion, the injection of Sephin1 could lead to the suppression of antitumor immunity during the development of implanted B16F1 tumors. This finding was also verified in another model using 4T1 tumor cells. As a selective inhibitor of PPP1R15A, Sephin1 can inhibit the binding of PPP1R15A to the PPP1R15A-PP1c complex and promote the integrated stress response in mice. From our results, we inferred that PPP1R15A and other ISR-related genes and their protein products could be important potential targets in tumor immunotherapy. The ISR is also an important pathway related to the immune response in mammals. A novel macrophage subtype was identified to be highly associated with Sephin1 treatment and to play a crucial role in antitumor immunity, suggesting a potential mechanism by which Sephin1 exerts its protumorigenic effect. Furthermore, cell-cell communication analysis also proved that the antitumor-related immunity interactions were suppressed by Sephin1 in mouse blood and tumor microenvironment. In a word, PPP1R15A and its related ISR play a key role in the immune system, especially antitumor immunity, and can be used as a new target for tumor immunotherapy. The inhibitor Sephin1 also has the potential for immunity related diseases, such as autoimmune disease [95, 96].

### 2.4 References

1. Castle, J. C., et al., Exploiting the mutanome for tumor vaccination. Cancer Res, 2012. 72(5): p. 1081-91.
2. Hilf, N., et al., Actively personalized vaccination trial for newly diagnosed glioblastoma. Nature, 2019. 565(7738): p. 240-245.
3. Keskin, D. B., et al., Neoantigen vaccine generates intratumoral T cell responses in phase Ib glioblastoma trial. Nature, 2019. 565(7738): p. 234-239.
4. Ott, P. A., et al., An immunogenic personal neoantigen vaccine for patients with melanoma. Nature, 2017. 547(7662): p. 217-221.
5. Binnewies, M., et al., Understanding the tumor immune microenvironment (TIME) for effective therapy. Nat Med, 2018. 24(5): p. 541-550.
6. van der Leun, A. M., D. S. Thommen, and T. N. Schumacher, CD8(+) T cell states in human cancer: insights from single-cell analysis. Nat Rev Cancer, 2020. 20(4): p. 218-232.
7. Mahmoud, S. M., et al., Tumor-infiltrating CD8+ lymphocytes predict clinical outcome in breast cancer. J Clin Oncol, 2011. 29(15): p. 1949-55.
8. Joyce, J. A. and J. W. Pollard, Microenvironmental regulation of metastasis. Nat Rev Cancer, 2009. 9(4): p. 239-52.
9. Fridman, W. H., et al., The immune contexture in human tumours: impact on clinical outcome. Nat Rev Cancer, 2012. 12(4): p. 298-306.
10. Vivier, E., et al., Functions of natural killer cells. Nat Immunol, 2008. 9(5): p. 503-10.
11. Palucka, K. and J. Banchereau, Cancer immunotherapy via dendritic cells. Nat Rev Cancer, 2012. 12(4): p. 265-77.
12. Qian, B. Z. and J. W. Pollard, Macrophage diversity enhances tumor progression and metastasis. Cell, 2010. 141(1): p. 39-51.
13. Hollander, M. C., et al., Mammalian GADD34, an apoptosis-and DNA damage-inducible gene. J Biol Chem, 1997. 272(21): p. 13731-7.
14. Brush, M. H., D. C. Weiser, and S. Shenolikar, Growth arrest and DNA damage-inducible protein GADD34 targets protein phosphatase 1 alpha to the endoplasmic reticulum and promotes dephosphorylation of the alpha subunit of eukaryotic translation initiation factor 2. Mol Cell Biol, 2003. 23(4): p. 1292-303.
15. Connor, J. H., et al., Growth arrest and DNA damage-inducible protein GADD34 assembles a novel signaling complex containing protein phosphatase 1 and inhibitor 1. Mol Cell Biol, 2001. 21(20): p. 6841-50.
16. Pakos-Zebrucka, K., et al., The integrated stress response. EMBO Rep, 2016. 17(10): p. 1374-1395.
17. Scheper, G. C., et al., Inactivation of eIF2B and phosphorylation of PHAS-I in heat-shocked rat hepatoma cells. J Biol Chem, 1997. 272(43): p. 26850-6.
18. Harding, H. P., Y Zhang, and D. Ron, Protein translation and folding are coupled by an endoplasmic-reticulum-resident kinase. Nature, 1999. 397(6716): p. 271-4.
19. Harding, H. P., et al., An integrated stress response regulates amino acid metabolism and resistance to oxidative stress. Mol Cell, 2003. 11(3): p. 619-33.
20. Costa-Mattioli, M. and P. Walter, The integrated stress response: From mechanism to disease. Science, 2020. 368(6489).
21. Ron, D., Translational control in the endoplasmic reticulum stress response. J Clin Invest, 2002. 110(10): p. 1383-8.
22. Lu, P. D., H. P. Harding, and D. Ron, Translation reinitiation at alternative open reading frames regulates gene expression in an integrated stress response. J Cell Biol, 2004. 167(1): p. 27-33.
23. Novoa, I., et al., Feedback inhibition of the unfolded protein response by GADD34-mediated dephosphorylation of eIF2alpha. J Cell Biol, 2001. 153(5): p. 1011-22.
24. Novoa, I., et al., Stress-induced gene expression requires programmed recovery from translational repression. Embo j, 2003. 22(5): p. 1180-7.
25. Dey, S., et al., Both transcriptional regulation and translational control of ATF4 are central to the integrated stress response. J Biol Chem, 2010. 285(43): p. 33165-33174.
26. Scheuner, D., et al., Double-stranded RNA-dependent protein kinase phosphorylation of the alpha-subunit of eukaryotic translation initiation factor 2 mediates apoptosis. J Biol Chem, 2006. 281(30): p. 21458-21468.
27. Ameri, K. and A. L. Harris, Activating transcription factor 4. Int J Biochem Cell Biol, 2008. 40(1): p. 14-21.
28. Malhi, H. and R. J. Kaufman, Endoplasmic reticulum stress in liver disease. J Hepatol, 2011. 54(4): p. 795-809.
29. B'Chir, W., et al., The eIF2α/ATF4 pathway is essential for stress-induced autophagy gene expression. Nucleic Acids Res, 2013. 41(16): p. 7683-99.
30. Han, J., et al., ER-stress-induced transcriptional regulation increases protein synthesis leading to cell death. Nat Cell Biol, 2013. 15(5): p. 481-90.
31. Jousse, C., et al., Inhibition of a constitutive translation initiation factor 2alpha phosphatase, CReP, promotes survival of stressed cells. J Cell Biol, 2003. 163(4): p. 767-75.
32. Sharma, V., et al., Local Inhibition of PERK Enhances Memory and Reverses Age-Related Deterioration of Cognitive and Neuronal Properties. J Neurosci, 2018. 38(3): p. 648-658.
33. Sen, T., et al., Activation of PERK Elicits Memory Impairment through Inactivation of CREB and Downregulation of PSD95 After Traumatic Brain Injury. J Neurosci, 2017. 37(24): p. 5900-5911.
34. Ma, T., et al., Suppression of eIF2α kinases alleviates Alzheimer's disease-related plasticity and memory deficits. Nat Neurosci, 2013. 16(9): p. 1299-305.
35. Delépine, M., et al., EIF2AK3, encoding translation initiation factor 2-alpha kinase 3, is mutated inpatients with Wolcott-Rallison syndrome. Nat Genet, 2000. 25(4): p. 406-9.
36. Harding, H. P., et al., Diabetes mellitus and exocrine pancreatic dysfunction in perk−/− mice reveals a role for translational control in secretory cell survival. Mol Cell, 2001. 7(6): p. 1153-63.
37. Robichaud, N., et al., Translational Control in Cancer. Cold Spring Harb Perspect Biol, 2019. 11(7).
38. Chu, J., et al., Translation Initiation Factors: Reprogramming Protein Synthesis in Cancer. Trends Cell Biol, 2016. 26(12): p. 918-933.
39. Cláudio, N., et al., Mapping the crossroads of immune activation and cellular stress response pathways. Embo j, 2013. 32(9): p. 1214-24.
40. Pulendran, B., The varieties of immunological experience: of pathogens, stress, and dendritic cells. Annu Rev Immunol, 2015. 33: p. 563-606.
41. Iwasaki, Y, et al., Activating transcription factor 4 links metabolic stress to interleukin-6 expression in macrophages. Diabetes, 2014. 63(1): p. 152-61.
42. Abdel-Nour, M., et al., The heme-regulated inhibitor is a cytosolic sensor of protein misfolding that controls innate immune signaling. Science, 2019. 365(6448).
43. Lu, B., et al., Novel role of PKR in inflammasome activation and HMGB1 release. Nature, 2012. 488(7413): p. 670-4.
44. D'Osualdo, A., et al., Transcription Factor ATF4 Induces NLRP1 Inflammasome Expression during Endoplasmic Reticulum Stress. PLoS One, 2015. 10(6): p. e0130635.
45. Das, I., et al., Preventing proteostasis diseases by selective inhibition of a phosphatase regulatory subunit. Science, 2015. 348(6231): p. 239-42.
46. Leroux, C. and G. Konstantinidou, Targeted Therapies for Pancreatic Cancer: Overview of Current Treatments and New Opportunities for Personalized Oncology. Cancers, 2021. 13(4): p. 799.
47. Vincent, A., et al., Pancreatic cancer. The lancet, 2011. 378(9791): p. 607-620.
48. Oberstein, P. E. and K. P. Olive, Pancreatic cancer: why is it so hard to treat? Therapeutic advances in gastroenterology, 2013. 6(4): p. 321-337.
49. Tesfaye, A. A., et al., The evolution into personalized therapies in pancreatic ductal adenocarcinoma: challenges and opportunities. Expert Rev Anticancer Ther, 2018. 18(2): p. 131-148.
50. Balachandran, V P., et al., Identification of unique neoantigen qualities in long-term survivors of pancreatic cancer. Nature, 2017. 551(7681): p. 512-516.
51. Zitvogel, L., et al., Type I interferons in anticancer immunity. Nat Rev Immunol, 2015. 15(7): p. 405-14.
52. Vonderhaar, E. P., et al., STING Activated Tumor-Intrinsic Type I Interferon Signaling Promotes CXCR3 Dependent Antitumor Immunity in Pancreatic Cancer. Cell Mol Gastroenterol Hepatol, 2021. 12(1): p. 41-58.
53. Cha, E., et al., Improved survival with T cell clonotype stability after anti-CTLA-4 treatment in cancer patients. Sci Transl Med, 2014. 6(238): p. 238ra70.
54. Hopkins, A. C., et al., T cell receptor repertoire features associated with survival in immunotherapy-treated pancreatic ductal adenocarcinoma. JCI Insight, 2018. 3(13).
55. Tumeh, P. C., et al., PD-1 blockade induces responses by inhibiting adaptive immune resistance. Nature, 2014. 515(7528): p. 568-71.
56. Hogan, S. A., et al., Peripheral Blood TCR Repertoire Profiling May Facilitate Patient Stratification for Immunotherapy against Melanoma. Cancer Immunol Res, 2019. 7(1): p. 77-85.
57. Kvistborg, P., et al., Anti-CTLA-4 therapy broadens the melanoma-reactive CD8+ T cell response. Sci Transl Med, 2014. 6(254): p. 254ra128.
58. Han, J., et al., TCR Repertoire Diversity of Peripheral PD-1(+)CD8(+) T Cells Predicts Clinical Outcomes after Immunotherapy in Patients with Non-Small Cell Lung Cancer. Cancer Immunol Res, 2020. 8(1): p. 146-154.
59. Carreno, B. M., et al., Cancer immunotherapy. A dendritic cell vaccine increases the breadth and diversity of melanoma neoantigen-specific T cells. Science, 2015. 348(6236): p. 803-8.
60. Coulie, P. G., et al., Tumour antigens recognized by T lymphocytes: at the core of cancer immunotherapy. Nat Rev Cancer, 2014. 14(2): p. 135-46.
61. Ott, P. A., et al., T-Cell-Inflamed Gene-Expression Profile, Programmed Death Ligand 1 Expression, and Tumor Mutational Burden Predict Efficacy in Patients Treated With Pembrolizumab Across 20 Cancers: KEYNOTE-028. J Clin Oncol, 2019. 37(4): p. 318-327.
62. Borcherding, N., N. L. Bormann, and G. Kraus, scRepertoire: An R-based toolkit for single-cell immune receptor analysis. F1000Res, 2020. 9: p. 47.
63. Aibar, S., et al., SCENIC: single-cell regulatory network inference and clustering. Nat Methods, 2017. 14(11): p. 1083-1086.
64. Jin, S., et al., Inference and analysis of cell-cell communication using CellChat. Nat Commun, 2021. 12(1): p. 1088.
65. Biswas, S. K. and A. Mantovani, Macrophage plasticity and interaction with lymphocyte subsets: cancer as a paradigm. Nat Immunol, 2010. 11(10): p. 889-96.
66. Quail, D. F. and J. A. Joyce, Microenvironmental regulation of tumor progression and metastasis. Nat Med, 2013. 19(11): p. 1423-37.
67. Azizi, E., et al., Single-Cell Map of Diverse Immune Phenotypes in the Breast Tumor Microenvironment. Cell, 2018. 174(5): p. 1293-1308.e36.
68. Rodriguez-Cruz, A., et al., CD3(+) Macrophages Deliver Proinflammatory Cytokines by a CD3- and Transmembrane TNF-Dependent Pathway and Are Increased at the BCG-Infection Site. Front Immunol, 2019. 10: p. 2550.
69. Tameire, F., et al., ATF4 couples MYC-dependent translational activity to bioenergetic demands during tumour progression. Nat Cell Biol, 2019. 21(7): p. 889-899.
70. Clementi, E., et al., Persistent DNA damage triggers activation of the integrated stress response to promote cell survival under nutrient restriction. BMC Biol, 2020. 18(1): p. 36.
71. Chen, Y, et al., Sephin1, which prolongs the integrated stress response, is a promising therapeutic for multiple sclerosis. Brain, 2019. 142(2): p. 344-361.
72. Ruiz, A., et al., Sephin1 Protects Neurons against Excitotoxicity Independently of the Integrated Stress Response. Int J Mol Sci, 2020. 21(17).
73. Myers, J. A. and J. S. Miller, Exploring the NK cell platform for cancer immunotherapy. Nat Rev Clin Oncol, 2021. 18(2): p. 85-100.
74. Farhood, B., M. Najafi, and K. Mortezaee, CD8(+) cytotoxic T lymphocytes in cancer immunotherapy: A review. J Cell Physiol, 2019. 234(6): p. 8509-8521.
75. Terabe, M. and J. A. Berzofsky, Tissue-Specific Roles of NKT Cells in Tumor Immunity. Front Immunol, 2018. 9: p. 1838.
76. Tiwary, S., J. A. Berzofsky, and M. Terabe, Altered Lipid Tumor Environment and Its Potential Effects on NKT Cell Function in Tumor Immunity. Front Immunol, 2019. 10: p. 2187.
77. Wing, J. B., A. Tanaka, and S. Sakaguchi, Human FOXP3(+) Regulatory T Cell Heterogeneity and Function in Autoimmunity and Cancer. Immunity, 2019. 50(2): p. 302-316.
78. Bos, P. D., et al., Transient regulatory T cell ablation deters oncogene-driven breast cancer and enhances radiotherapy. J Exp Med, 2013. 210(11): p. 2435-66.
79. Mantovani, A., et al., The chemokine system in diverse forms of macrophage activation and polarization. Trends Immunol, 2004. 25(12): p. 677-86.
80. Mills, C. D., et al., M-1/M-2 macrophages and the Th1 Th2 paradigm. J Immunol, 2000. 164(12): p. 6166-73.
81. Braun, D. A., et al., Progressive immune dysfunction with advancing disease stage in renal cell carcinoma. Cancer Cell, 2021. 39(5): p. 632-648.e8.
82. Shapouri-Moghaddam, A., et al., Macrophage plasticity, polarization, and function in health and disease. J Cell Physiol, 2018. 233(9): p. 6425-6440.
83. Wu, K., et al., Redefining Tumor-Associated Macrophage Subpopulations and Functions in the Tumor Microenvironment. Front Immunol, 2020. 11: p. 1731.
84. Miyake, K. and H. Karasuyama, The Role of Trogocytosis in the Modulation of Immune Cell Functions. Cells, 2021. 10(5).
85. Li, G., et al., T cell antigen discovery via trogocytosis. Nat Methods, 2019. 16(2): p. 183-190.
86. Tu, L., et al., L-selectin binds to P-selectin glycoprotein ligand-1 on leukocytes: interactions between the lectin, epidermal growth factor, and consensus repeat domains of the selectins determine ligand binding specificity. J Immunol, 1996. 157(9): p. 3995-4004.
87. Spertini, O., et al., P-selectin glycoprotein ligand 1 is a ligand for L-selectin on neutrophils, monocytes, and CD34+ hematopoietic progenitor cells. J Cell Biol, 1996. 135(2): p. 523-31.
88. Casas, J., et al., Ligand-engaged TCR is triggered by Lck not associated with CD8 coreceptor. Nat Commun, 2014. 5: p. 5624.
89. Anderson, A. C., N. Joller, and V K. Kuchroo, Lag-3, Tim-3, and TIGIT. Co-inhibitory Receptors with Specialized Functions in Immune Regulation. Immunity, 2016. 44(5): p. 989-1004.
90. Zhang, Y, et al., Upregulation of PD-L1 by SPP1 mediates macrophage polarization and facilitates immune escape in lung adenocarcinoma. Exp Cell Res, 2017. 359(2): p. 449-457.
91. Kaur, S., et al., Functions of Thrombospondin-1 in the Tumor Microenvironment. Int J Mol Sci, 2021. 22(9).
92. Zhu, Y, et al., CSF1/CSF1R blockade reprograms tumor-infiltrating macrophages and improves response to T-cell checkpoint immunotherapy in pancreatic cancer models. Cancer Res, 2014. 74(18): p. 5057-69.
93. Xia, S., et al., Higher-order assemblies in immune signaling: supramolecular complexes and phase separation. Protein Cell, 2021. 12(9): p. 680-694.
94. Fernandes, T. L., et al., Macrophage: A Potential Target on Cartilage Regeneration. Front Immunol, 2020. 11: p. 111.
95. Stojanovich, L., Stress and autoimmunity. Autoimmun Rev, 2010. 9(5): p. A271-6.
96. Stojanovich, L. and D. Marisavljevich, Stress as a trigger of autoimmune disease. Autoimmun Rev, 2008. 7(3): p. 209-13.

1-166. (canceled)

167. A MX1 agonist for use in
(a) enhancing cell-mediated immunity in a subject in need thereof, preferably wherein the cell-mediated immunity is T cell-mediated immunity;
(b) stimulating and/or expanding T cells in a subject in need thereof;
(c) potentiating immunogenicity of an immunogenic composition in a subject, preferably wherein the immunogenic composition is a vaccine or a composition for CAR-T treatment, preferably wherein the vaccine is a tumor vaccine; optionally
wherein the subject is suffering from a condition that would benefit from upregulation of immune response, preferably wherein the condition is tumor, infectious disease, cardiovascular disease or inflammatory disease, preferably wherein the condition is tumor or infectious disease; optionally,
wherein the subject is receiving a therapy whose efficacy can be potentiated by enhanced cell-mediated immunity, preferably wherein the therapy is an anti-tumor therapy or anti-infectious therapy, preferably wherein the anti-tumor therapy is selected from the group consisting of chemotherapy, targeted therapy, immunotherapy, cell therapy (e.g. CAR-T therapy), and tumor vaccine;
(d) treating a condition that would benefit from upregulation of immune response in a subject in need thereof, preferably wherein the condition is tumor, infectious disease, cardiovascular disease or inflammatory disease, preferably wherein the condition is tumor or infectious disease; optionally
wherein the MX1 agonist is administered in combination with a therapy that treats the condition, preferably wherein the therapy is an anti-tumor therapy or anti-infectious therapy, preferably wherein the anti-tumor therapy is selected from the group consisting of chemotherapy, targeted therapy, immunotherapy, cell therapy (e.g. CAR-T therapy), tumor vaccine and CAR-T therapy; optionally
wherein the MX1 agonist is selected from the group consisting of full-length mRNA of MX1, and small molecule compounds of DMXAA, ADU-S100, 2′,3′-cGAMP sodium and cGAMP;
(e) promoting clonal expansion of T cells, preferably wherein the T cells are memory T cells;
(f) promoting T cell activation or promoting cytotoxicity of T cells, preferably wherein the T cells are cultured with the MX1 agonist in combination with a second agent for stimulating the T cells; optionally
wherein the MX1 agonist is selected from the group consisting of full-length mRNA of MX1, and small molecule compounds of DMXAA, ADU-S100, 2′,3′-cGAMP sodium and cGAMP;

168. A composition comprising the T cells prepared using the MX1 agonist of claim 167(e) for use in (a) treating a condition that would benefit from upregulation of immune response in a subject in need thereof.

169. A method of evaluating activation state, or activity or cytotoxicity of T cells, comprising detecting expression level of MX1 in the T cells, wherein an increase in the expression level of MX1 relative to a control T cell is indicative of cytotoxicity of the T cells, preferably wherein the T cells are CAR-T cells, or TCR-T cells, preferably wherein the control T cell is CD8+ T cell.

170. A method of preparing a population of T cells, wherein the population of T cells is for cell therapy, or the population of T cells is a second population of active T cells converted from a first population of inactive T cells, wherein when the population of T cells is for cell therapy, the method comprises:
(i) a) identifying the population of T cells having increased expression level of MX1 relative to a control T cell; and
b) selectively enriching the identified T cells for cell therapy; or
(ii) a) identifying the population of T cells as inactive if it does not show an increase in the expression level of MX1 relative to a control T cell; and
b) treating the identified inactive population of T cells with an effective amount of a MX1 agonist under suitable conditions, thereby obtaining a population of activated T cells; optionally
wherein the MX1 agonist is selected from the group consisting of full-length mRNA of MX1, and small molecule compounds of DMXAA, ADU-S100, 2′,3′-cGAMP sodium and cGAMP; or
wherein when the population of T cells is the second population of active T cells, the method comprises:
(a) providing the first population of inactive T cells, wherein the inactive T cells do not show an increase in the expression level of MX1 relative to a control T cell;
(b) treating the first population of inactive T cells with an effective amount of a MX1 agonist under suitable conditions; and
(c) detecting expression level of MX1 in the population of T cells obtained in step (b), wherein an increase in the expression level of MX1 relative to a control T cell is indicative of successful conversion to the second population of active T cells; optionally
wherein the control T cell is CD8+ T cell.

171. A composition comprising the population of T cells prepared or converted by a method of claim 169 for use in treating a condition that would benefit from upregulation of immune response in a subject in need thereof.

172. A MX1 antagonist for use in
(a) reducing cell-mediated immunity in a subject in need thereof; preferably wherein the cell-mediated immunity is T cell-mediated immunity;
(b) deactivating T cells in a subject in need thereof; optionally
wherein the subject has been determined to have T cells having an increase in the expression level of MX1 relative to a control T cell; optionally
wherein the subject is suffering from a condition characterized in excessive cell-mediated immunity;
(c) treating a condition that would benefit from downregulation of immune response in a subject in need thereof; optionally
wherein the condition is an inflammatory disease, an autoimmune disease, an allergic disease, or a T cell cancer; optionally
wherein the condition is where the subject has received or is contemplated to receive an allogeneic or xenogeneic transplant; optionally
wherein the MX1 antagonist is selected from the group consisting of CCCP and H-151.

173. A method of
(i) assessing responsiveness of a subject to a tumor neoantigen vaccine, wherein
(I) the method comprises:
a) determining expression level of one or more genes in one or more immune cells from a sample obtained from the subject; wherein the one or more genes are selected from the group consisting of: AC011815.2, ANKRD29, AP3M2, ARL5B, ATF4, ATP5F1B, C15orf54, CHCHD2, COL4A3BP, COPE, CTTNBP2, DDOST, DERL1, DNPH1, EGF, FERMT3, FOSL2, GCH1, GK, GLA, LMAN2, LRRK1, MAP9, MN1, MTDH, NFIL3, NUAK1, PDE4D, PIM1, PRKACA, PRMT1, SERTAD2, SLC16A6, SYT17, TMED10, TMEM258, TRDV1, WDFY3, ZBTB43 and ZDHHC7, or are any combination thereof;
b) comparing the expression level of the one or more genes determined in step a) with a reference level to determine difference from the reference level; and
c) Assessing the responsiveness of the subject to the tumor neoantigen vaccine based on the difference determined in step b); preferably
wherein the one or more immune cells are selected from the group consisting of: CD8+ T cells, CD4+ T cells, monocytes, B cells, NK cells and macrophages; optionally
wherein the one or more immune cells are CD8+ T cells and the one or more genes are selected from the group consisting of: AC011815.2, EGF, GCH1, NUAK1 and SERTAD2, or are any combination thereof; optionally
wherein the one or more immune cells are CD4+ T cells and the one or more genes are selected from the group consisting of: ANKRD29, C15orf54, FOSL2, GCH1, PRKACA, SERTAD2, SLC16A6, TRDV1, WDFY3 and ZBTB43, or are any combination thereof; optionally
wherein the one or more immune cells are monocytes and the one or more genes are selected from the group consisting of: AP3M2, ARL5B, ATP5F1B, CHCHD2, COPE, GLA, MN1 and MTDH, or are any combination thereof; optionally
wherein the one or more immune cells are B cells and the one or more genes are selected from the group consisting of: ATF4, ATP5F1B, DDOST, DERL1, DNPH1, LMAN2, MAP9, PDE4D, PIM1, TMEM258 and ZDHHC7, or are any combination thereof; optionally
wherein the one or more immune cells are NK cells and the one or more genes are selected from the group consisting of: COL4A3BP, FERMT3 and NFIL3, or are any combination thereof; optionally
wherein the one or more immune cells are macrophages and the one or more genes are selected from the group consisting of: COPE, CTTNBP2, GK, LRRK1, MTDH, PRMT1, SYT17 and TMED10, or are any combination thereof; or
(II) wherein during priming phase, the method comprises:
a) determining expression level of one or more genes in one or more immune cells from a sample obtained from the subject following the at least one priming dose of tumor neoantigen vaccine; wherein the one or more genes are selected from the group consisting of: AC011815.2, ATF4, C15orf54, DDOST, DERL1, DNPH1, EGF, FERMT3, FOSL2, GCH1, GK, LMAN2, MAP9, NUAK1, PDE4D, PRKACA, SERTAD2, SLC16A6, TMED10, TMEM258, WDFY3, ZBTB43 and ZDHHC7, or are any combination thereof;
b) comparing the expression level of the one or more genes determined in step a) with a reference level to determine difference from the reference level; and
c) Assessing the responsiveness of the subject to the at least one priming dose of tumor neoantigen vaccine, based on the difference determined in step b); optionally
wherein the one or more immune cells are selected from the group consisting of: CD8+ T cells, CD4+ T cells, monocytes, B cells, NK cells and macrophages; optionally
wherein the one or more immune cells are selected from the group consisting of: CD8+ T cells, CD4+ T cells, B cells, NK cells and macrophages; optionally
wherein the one or more immune cells are CD8+ T cells and the one or more genes are selected from the group consisting of: AC011815.2, EGF, GCH1, NUAK1 and SERTAD2, or are any combination thereof; optionally
wherein the one or more immune cells are CD4+ T cells and the one or more genes are selected from the group consisting of: C15orf54, FOSL2, GCH1, PRKACA, SERTAD2, SLC16A6, WDFY3 and ZBTB43, or are any combination thereof; optionally
wherein the one or more immune cells are B cells and the one or more genes are selected from the group consisting of: ATF4, DDOST, DERL1, DNPH1, LMAN2, MAP9, PDE4D, TMEM258 and ZDHHC7, or are any combination thereof; optionally
wherein the one or more immune cells are NK cells and the gene is FERMT3; optionally
wherein the one or more immune cells are macrophages and the one or more genes are selected from the group consisting of: GK and TMED10, or are any combination thereof; or
(III) wherein during boosting phase, the method comprises:
a) determining expression level of one or more genes in one or more immune cells from a sample obtained from the subject during the boosting phase; wherein the one or more genes are selected from the group consisting of: AC011815.2, ANKRD29, AP3M2, ARL5B, ATP5F1B, C15orf54, CHCHD2, COL4A3BP, COPE, CTTNBP2, EGF, FERMT3, FOSL2, GK, GLA, LRRK1, MAP9, MN1, MTDH, NFIL3, NUAK1, PIM1, PRMT1, SERTAD2, SLC16A6, SYT17, TMED10, TRDV1 and ZDHHC7, or are any combination thereof;
b) comparing the expression level of the one or more genes determined in step a) with a reference expression level to determine difference from the reference level; and
c) Assessing the responsiveness of the subject to the at least one boosting dose of tumor neoantigen vaccine, based on the difference determined in step b); optionally
wherein the one or more immune cells are selected from the group consisting of: CD8+ T cells, CD4+ T cells, monocytes, B cells, NK cells and macrophages; optionally
wherein the one or more immune cells are CD8+ T cells and the one or more genes are selected from the group consisting of: AC011815.2, EGF, NUAK1 and SERTAD2, or are any combination thereof; optionally
wherein the one or more immune cells are CD4+ T cells and the one or more genes are selected from the group consisting of: ANKRD29, C15orf54, FOSL2, SLC16A6 and TRDV1, or are any combination thereof; optionally
wherein the one or more immune cells are monocytes and the one or more genes are selected from the group consisting of: AP3M2, ARL5B, ATP5F1B, CHCHD2, COPE, GLA, MN1 and MTDH, or are any combination thereof; optionally
wherein the one or more immune cells are B cells and the one or more genes are selected from the group consisting of: ATP5F1B, MAP9, PIM1 and ZDHHC7, or are any combination thereof; optionally
wherein the one or more immune cells are NK cells and the one or more genes are selected from the group consisting of: COL4A3BP, FERMT3 and NFIL3, or are any combination thereof; optionally
wherein the one or more immune cells are macrophages and the one or more genes are selected from the group consisting of: COPE, CTTNBP2, GK, LRRK1, MTDH, PRMT1, SYT17 and TMED10, or are any combination thereof; optionally
wherein the expression level of a given gene is represented by percentage of a given type of immune cells that express the given gene; optionally
wherein the reference expression level is the expression level of the one or more genes in the one or more immune cells from a sample obtained from the subject before receiving first dose of the tumor neoantigen vaccine; optionally
wherein the subject is determined as having a good response to the tumor neoantigen vaccine when the difference reaches or exceeds a predetermined threshold, or the subject is determined as having an insufficient response to the tumor neoantigen vaccine when the difference is below a predetermined threshold; or
(ii) predicting the risk of tumor relapse in a subject before or after receiving at least one dose of tumor neoantigen vaccine, comprising:
a) determining expression level of one or more genes in one or more immune cells from a sample obtained from the subject; wherein the one or more genes are selected from the group consisting of: ABCA13, AC022726.2, AC105094.2, AC243829.2, AL021807.1, AL391807.1, AL662907.3, APOBEC3C, ATP1B3, ATP6V1B2, C1QBP, CBX5, CCNE2, CD63, CEBPA, CHP1, CLN8, CMBL, CNIH4, COL4A3BP, CPNE3, DACH1, DNAJC12, DNAJC3, DOC2B, EEA1, ENAM, ERG, FAM43A, FBXO43, FIGN, GCA, GNG4, GNLY, GOLIM4, GPM6B, GPR171, GPRC5D, HHEX, HID1, ILF2, IRF2BP2, KIF22, LACC1, LIPA, MANEA, MECOM, MID1IP1, MX2, MYOM2, NABP1, NEFH, NFE4, NLN, OXCT2, P3H2, PI3, PIM1, PLAU, PRSS3, PSMA3, RAB10, RAB20, RASGRP2, RBMS3, RPL23, RPS6KA5, RPS8, RUNX1, SAMD3, 11-Sep, SERTAD2, SIGLEC6, SIT1, SOCS1, SSR1, ST6GALNAC1, SYNE2, TRAV16, TREM2, TXNDC15, TXNDC17, UAP1, UFM1, UGT2B17, XPNPEP1, ZBTB16, ZNF608 and STAT1, or are any combination thereof;
b) comparing the expression level of the one or more genes determined in step a) with a reference level to determine difference from the reference level; and
c) Assessing the risk of tumor relapse in the subject based on the difference determined in step b); optionally
wherein the one or more genes are selected from the group consisting of: AC022726.2, AC105094.2, AC243829.2, AL021807.1, AL391807.1, ATP1B3, CMBL, DACH1, DNAJC12, DOC2B, EEA1, ERG, FBXO43, FIGN, GPRC5D, HID1, NFE4, OXCT2, P3H2, PI3, PLAU, PRSS3, RASGRP2, RPL23, RPS8, ST6GALNAC1, SYNE2, TRAV16, TREM2 and ZNF608, or are any combination thereof; optionally
wherein the expression level of the one or more genes are determined in one or more immune cells from a sample obtained from the subject after receiving the at least one dose of tumor neoantigen vaccine, wherein the one or more genes are selected from the group consisting of: AC022726.2, AC105094.2, AC243829.2, AL021807.1, AL391807.1, ATP1B3, ATP6V1B2, CHP1, CLN8, CMBL, CNIH4, CPNE3, DACH1, DNAJC12, DOC2B, EEA1, ERG, FAM43A, FBXO43, FIGN, GNG4, GPM6B, GPRC5D, HHEX, HID1, IRF2BP2, KIF22, LACC1, MECOM, MID1IP1, MYOM2, NABP1, NFE4, OXCT2, P3H2, PI3, PLAU, PRSS3, RAB10, RASGRP2, RBMS3, RPL23, RPS8, RUNX1, SAMD3, SERTAD2, SIGLEC6, SIT1, SOCS1, ST6GALNAC1, SYNE2, TRAV16, TREM2, TXNDC17, UGT2B17, XPNPEP1 and ZNF608, or are any combination thereof; optionally
wherein the expression level of the one or more genes are determined in one or more immune cells from a sample obtained from the subject before receiving the at least one dose of tumor neoantigen vaccine, wherein the one or more genes are selected from the group consisting of: ABCA13, AC022726.2, AC105094.2, AC243829.2, AL021807.1, AL391807.1, AL662907.3, ATP1B3, CBX5, CMBL, DACH1, DNAJC12, DNAJC3, DOC2B, ENAM, ERG, FBXO43, FIGN, GOLIM4, GPRC5D, HID1, ILF2, NEFH, NFE4, NLN, OXCT2, P3H2, PI3, PLAU, PRSS3, PSMA3, RASGRP2, RPL23, RPS6KA5, RPS8, 11-Sep, ST6GALNAC1, SYNE2, TRAV16, TREM2, TXNDC15, UAP1, UFM1, ZBTB16 and ZNF608, or are any combination thereof; optionally
wherein the one or more immune cells are selected from the group consisting of: CD8+ T cells, CD4+ T cells, monocytes, B cells, NK cells and macrophages; optionally
wherein the one or more immune cells are CD8+ T cells and the one or more genes are selected from the group consisting of: CHP1, EEA1, RPS8, RUNX1, UGT2B17, XPNPEP1, ZNF608 and STAT1, or are any combination thereof; optionally
wherein the one or more immune cells are CD4+ T cells and the one or more genes are selected from the group consisting of: AL662907.3, CD63, COL4A3BP, CPNE3, EEA1, GPM6B, LIPA, PSMA3, RAB10, RAB20, RBMS3, RPS8, SAMD3, SERTAD2, SOCS1, TXNDC15 and STAT1, or are any combination thereof; optionally
wherein the one or more immune cells are monocytes and the one or more genes are selected from the group consisting of: ABCA13, ATP1B3, DNAJC3, ENAM, FIGN, GNG4, ILF2, KIF22, MANEA, NEFH, NLN, P3H2, PLAU, SIGLEC6, SSR1, TRAV16, UFM1 and ZBTB16, or are any combination thereof; optionally
wherein the one or more immune cells are B cells and the one or more genes are selected from the group consisting of: AC105094.2, AC243829.2, AL021807.1, AL391807.1, ATP6V1B2, C1QBP, CMBL, CNIH4, DOC2B, FAM43A, GCA, GNLY, HID1, LACC1, MID1IP1, MX2, PI3, PIM1, 11-Sep, SIT1 and SYNE2, or are any combination thereof; optionally
wherein the one or more immune cells are NK cells and the one or more genes are selected from the group consisting of: AC022726.2, APOBEC3C, DACH1, ERG, IRF2BP2, MYOM2, NFE4, PRSS3, RPL23, RPS6KA5 and TREM2, or are any combination thereof; optionally
wherein the one or more immune cells are macrophages and the one or more genes are selected from the group consisting of: CBX5, CCNE2, CEBPA, CLN8, DNAJC12, FBXO43, FIGN, GOLIM4, GPR171, GPRC5D, HHEX, MECOM, NABP1, OXCT2, RASGRP2, ST6GALNAC1, TXNDC17 and UAP1, or are any combination thereof; optionally
wherein the expression level of a given gene is represented by percentage of a given type of immune cells that express the given gene; optionally
wherein the reference expression level is expression level of the corresponding gene in the corresponding immune cells that is representative for a relapse subject; optionally
wherein the reference expression level is a standard or average expression level determined from a representative population of relapse subjects; optionally
wherein the subject is determined as having low risk of tumor relapse when the difference reaches or exceeds a predetermined threshold, or the subject is determined as having high risk of tumor relapse when the difference is below a predetermined threshold; optionally
wherein the reference expression level is expression level of the corresponding gene in the corresponding immune cells that is representative for a non-relapse subject; optionally
wherein the reference expression level is a standard or average expression level determined from a representative population of non-relapse subjects; optionally
wherein the subject is determined as having high risk of tumor relapse when the difference reaches or exceeds a predetermined threshold, or the subject is determined as having low risk of tumor relapse when the difference is below a predetermined threshold; or
(iii) assessing therapeutic efficacy in a subject having been treated with an anti-tumor therapy in combination with at least one dose of tumor neoantigen vaccine, comprising:
a) determining the level of one or more genes in one or more immune cells from a sample obtained from the subject after the treatment; wherein the one or more genes are selected from the group consisting of: AC092490.1, ALDH1L2, LILRB5, PALD1, PKP2 and TRAV35, or are any combination thereof;
b) comparing the level of the one or more genes determined in step a) with a reference level to determine difference from the reference level; and
c) Assessing the therapeutic efficacy in the subject based on the difference determined in step b); optionally
wherein the one or more immune cells are selected from the group consisting of: CD8+ T cells, CD4+ T cells, monocytes, B cells, NK cells and macrophages; optionally
wherein the one or more immune cells are CD4+ T cells and the one or more genes is LILRB5; optionally
wherein the one or more immune cells are monocytes and the one or more genes are selected from the group consisting of: ALDH1L2 and PKP2, or are any combination thereof; optionally
wherein the one or more immune cells are B cells and the one or more genes are selected from the group consisting of: AC092490.1, PALD1 and TRAV35, or are any combination thereof; optionally
wherein the expression level of a given gene is represented by percentage of a given type of immune cells that express the given gene; optionally
wherein the reference expression level is expression level of the corresponding gene in the corresponding immune cells from a sample obtained from the subject before receiving the anti-tumor therapy; optionally
wherein the anti-tumor therapy comprises a PD-1 antagonist; optionally
wherein the subject has shown tumor relapse after tumor neoantigen vaccination;
wherein the subject has received tumor resection surgery before receiving first dose of the tumor neoantigen vaccine, optionally the subject had no chemotherapy before the resection surgery; optionally
wherein tumor tissue, adjacent tissue and/or a peripheral blood sample of the subject have been analysed to identify one or more tumor-specific mutations in the subject, preferably wherein the tumor neoantigen vaccine is prepared based on the identified tumor-specific mutations; optionally
wherein the subject has been diagnosed to have pancreatic cancer, optionally pancreatic ductal adenocarcinoma; optionally
wherein the sample comprises or is derived from peripheral blood mononuclear cells (PBMCs), a blood sample, or tumor infiltrating immune cells; optionally
wherein the level of the one or more genes is measured via an amplification assay, a hybridization assay, sequencing methods (e.g. single-cell sequencing), or an immunoassay (e.g. flow cytometry or immunohistochemistry).

174. A kit for
(i) assessing responsiveness of a subject to a tumor neoantigen vaccine, comprising one or more reagents for detecting expression level of one or more genes in one or more immune cells from a sample obtained from the subject; wherein the one or more genes are selected from the group consisting of: AC011815.2, ANKRD29, AP3M2, ARL5B, ATF4, ATP5F1B, C15orf54, CHCHD2, COL4A3BP, COPE, CTTNBP2, DDOST, DERL1, DNPH1, EGF, FERMT3, FOSL2, GCH1, GK, GLA, LMAN2, LRRK1, MAP9, MN1, MTDH, NFIL3, NUAK1, PDE4D, PIM1, PRKACA, PRMT1, SERTAD2, SLC16A6, SYT17, TMED10, TMEM258, TRDV1, WDFY3, ZBTB43 and ZDHHC7, or are any combination thereof; or
(ii) assessing responsiveness of a subject to at least one priming dose of tumor neoantigen vaccine, comprising one or more reagents for detecting expression level of one or more genes in one or more immune cells from a sample obtained from the subject following the at least one priming dose of tumor neoantigen vaccine; wherein the one or more genes are selected from the group consisting of: AC011815.2, ATF4, C15orf54, DDOST, DERL1, DNPH1, EGF, FERMT3, FOSL2, GCH1, GK, LMAN2, MAP9, NUAK1, PDE4D, PRKACA, SERTAD2, SLC16A6, TMED10, TMEM258, WDFY3, ZBTB43 and ZDHHC7, or are any combination thereof; or
(iii) assessing responsiveness of a subject to at least one boosting dose of tumor neoantigen vaccine, comprising one or more reagents for detecting expression level of one or more genes in one or more immune cells from a sample obtained from the subject during the boosting phase; wherein the one or more genes are selected from the group consisting of: AC011815.2, ANKRD29, AP3M2, ARL5B, ATP5F1B, C15orf54, CHCHD2, COL4A3BP, COPE, CTTNBP2, EGF, FERMT3, FOSL2, GK, GLA, LRRK1, MAP9, MN1, MTDH, NFIL3, NUAK1, PIM1, PRMT1, SERTAD2, SLC16A6, SYT17, TMED10, TRDV1 and ZDHHC7, or are any combination thereof; or
(iv) predicting the risk of tumor relapse in a subject before or after receiving at least one dose of tumor neoantigen vaccine, comprising one or more reagents for detecting expression level of one or more genes in one or more immune cells from a sample obtained from the subject; wherein the one or more genes are selected from the group consisting of: ABCA13, AC022726.2, AC105094.2, AC243829.2, AL021807.1, AL391807.1, AL662907.3, APOBEC3C, ATP1B3, ATP6V1B2, C1QBP, CBX5, CCNE2, CD63, CEBPA, CHP1, CLN8, CMBL, CNIH4, COL4A3BP, CPNE3, DACH1, DNAJC12, DNAJC3, DOC2B, EEA1, ENAM, ERG, FAM43A, FBXO43, FIGN, GCA, GNG4, GNLY, GOLIM4, GPM6B, GPR171, GPRC5D, HHEX, HID1, ILF2, IRF2BP2, KIF22, LACC1, LIPA, MANEA, MECOM, MID1IP1, MX2, MYOM2, NABP1, NEFH, NFE4, NLN, OXCT2, P3H2, PI3, PIM1, PLAU, PRSS3, PSMA3, RAB10, RAB20, RASGRP2, RBMS3, RPL23, RPS6KA5, RPS8, RUNX1, SAMD3, 11-Sep, SERTAD2, SIGLEC6, SIT1, SOCS1, SSR1, ST6GALNAC1, SYNE2, TRAV16, TREM2, TXNDC15, TXNDC17, UAP1, UFM1, UGT2B17, XPNPEP1, ZBTB16, ZNF608 and STAT1, or are any combination thereof; or
(v) assessing therapeutic efficacy in a subject having been treated with an anti-tumor therapy in combination with at least one dose of tumor neoantigen vaccine, comprising one or more reagents for detecting the level of one or more genes in one or more immune cells from a sample obtained from the subject after the treatment; wherein the one or more genes are selected from the group consisting of: AC092490.1, ALDH1L2, LILRB5, PALD1, PKP2 and TRAV35, or are any combination thereof.

175. A PPP1R15A agonist for use in
(a) enhancing cell-mediated immunity in a subject in need thereof, preferably wherein the cell-mediated immunity is T cell-mediated immunity
(b) stimulating and/or expanding T cells in a subject in need thereof
(c) potentiating immunogenicity of an immunogenic composition in a subject, preferably wherein the immunogenic composition is a vaccine or a composition for CAR-T treatment, preferably wherein the vaccine is a tumor vaccine; optionally
wherein the subject is suffering from a condition that would benefit from upregulation of immune response; optionally
wherein the subject is determined to have reduced expression level of PPP1R15A; optionally
wherein the condition is tumor, infectious disease, cardiovascular disease or inflammatory disease, preferably wherein the condition is tumor or infectious disease, preferably wherein the tumor is selected from the group consisting of brain cancer, renal cell carcinoma, ovarian cancer, gastric cancer, bladder cancer, breast cancer, ovarian cancer, prostate cancer, colon cancer, lung cancer, squamous cell carcinoma of head and neck, colorectal cancer, melanoma, and myeloma; optionally
wherein the subject is receiving a therapy whose efficacy can be potentiated by enhanced cell-mediated immunity, preferably wherein the therapy is an anti-tumor therapy or anti-infectious therapy, preferably wherein the anti-tumor therapy is selected from the group consisting of chemotherapy, targeted therapy, immunotherapy, cell therapy (e.g. CAR-T therapy), and tumor vaccine;
(d) treating a condition that would benefit from upregulation of immune response in a subject in need thereof, preferably wherein the condition is tumor, infectious disease, cardiovascular disease or inflammatory disease, preferably wherein the condition is tumor or infectious disease, preferably wherein the tumor is selected from the group consisting of brain cancer, renal cell carcinoma, ovarian cancer, gastric cancer, bladder cancer, breast cancer, ovarian cancer, prostate cancer, colon cancer, lung cancer, squamous cell carcinoma of head and neck, colorectal cancer, melanoma, and myeloma; optionally
wherein the subject is diagnosed as having reduced expression level of PPP1R15A, optionally
wherein the PPP1R15A agonist is administered in combination with a therapy that treats the condition, preferably wherein the therapy is an anti-tumor therapy or anti-infectious therapy, preferably wherein the anti-tumor therapy is selected from the group consisting of chemotherapy, targeted therapy, immunotherapy, cell therapy (e.g. CAR-T therapy), tumor vaccine and CAR-T therapy;
(e) promoting clonal expansion of T cells, preferably wherein the T cells are memory T cells; and/or
(f) promoting T cell activation or promoting cytotoxicity of T cells, preferably wherein the T cells are cultured with the PPP1R15A agonist in combination with a second agent for stimulating the T cells; optionally
wherein the PPP1R15A agonist is selected from the group consisting of full-length mRNA of PPP1R15A, and a synthetic cannabinoid (WIN 55,212-2 mesylate [WIN]).

176. A composition comprising the T cells prepared using the PPP1R15A agonist of claim 175(e) for use in treating a condition that would benefit from upregulation of immune response in a subject in need thereof.

177. A method of evaluating activation state, or activity or cytotoxicity of T cells, comprising detecting expression level of PPP1R15A in the T cells, wherein an increase in the expression level of PPP1R15A relative to a control T cell is indicative of cytotoxicity of the T cells, preferably wherein the T cells are CAR-T cells, or TCR-T cells; optionally
wherein the control T cell is CD8+ T cell; optionally
wherein the PPP1R15A agonist is selected from the group consisting of full-length mRNA of PPP1R15A, and a synthetic cannabinoid (WIN 55,212-2 mesylate [WIN]).

178. A method of preparing a population of T cells, wherein the population of T cells is for cell therapy, or the population of T cells is a second population of active T cells converted from a first population of inactive T cells, wherein when the population of T cells is for cell therapy, the method comprises:
(i) a) identifying the population of T cells having increased expression level of PPP1R15A relative to a control T cell; and
b) selectively enriching the identified T cells for cell therapy; or
(ii) a) identifying the population of T cells as inactive if it does not show an increase in the expression level of PPP1R15A relative to a control T cell; and
b) treating the identified inactive population of T cells with an effective amount of a PPP1R15A agonist under suitable conditions, thereby obtaining a population of activated T cells; or
wherein when the population of T cells is the second population of active T cells, the method comprises:
(a) providing the first population of inactive T cells, wherein the inactive T cells do not show an increase in the expression level of PPP1R15A relative to a control T cell;
(b) treating the first population of inactive T cells with an effective amount of a PPP1R15A agonist under suitable conditions; and
(c) detecting expression level of PPP1R15A in the population of T cells obtained in step (b), wherein an increase in the expression level of PPP1R15A relative to a control T cell is indicative of successful conversion to the second population of active T cells; optionally
wherein the control T cell is CD8+ T cell; optionally
wherein the PPP1R15A agonist is selected from the group consisting of full-length mRNA of PPP1R15A, and a synthetic cannabinoid (WIN 55,212-2 mesylate [WIN]).

179. A composition comprising the population of T cells prepared or converted by a method of claim 177.

180. A method of treating a condition that would benefit from upregulation of immune response in a subject in need thereof, comprising administering to the subject the population of T cells prepared or converted by a method of claim 177.

181. A PPP1R15A antagonist for use in
(a) reducing cell-mediated immunity in a subject in need thereof; preferably wherein the cell-mediated immunity is T cell-mediated immunity;
(b) deactivating T cells in a subject in need thereof; optionally
wherein the subject has been determined to have T cells having an increase in the expression level of PPP1R15A relative to a control T cell; optionally
wherein the subject is suffering from a condition characterized in excessive cell-mediated immunity;
(c) A method of treating a condition that would benefit from downregulation of immune response in a subject in need thereof, comprising administering to the subject an effective amount of a PPP1R15A antagonist; optionally
wherein the condition is an autoimmune disease, organ or tissue graft rejection, a graft-versus-host disease, an inflammatory disease, an infectious disease, or a cancer; optionally
wherein the subject is diagnosed as having increased expression level of PPP1R15A; optionally
wherein the PPP1R15A antagonist is selected from the group consisting of Guanabenz and Sephin1.

182. A composition comprising the population of T cells prepared or converted by a method of claim 170 for use in treating a condition that would benefit from upregulation of immune response in a subject in need thereof.

183. A composition comprising the T cells prepared using the PPP1R15A agonist of claim 175(f) for use in treating a condition that would benefit from upregulation of immune response in a subject in need thereof.

184. A composition comprising the population of T cells prepared or converted by a method of claim 178.

185. A method of treating a condition that would benefit from upregulation of immune response in a subject in need thereof, comprising administering to the subject the population of T cells prepared or converted by a method of claim 178.

186. A composition comprising the T cells prepared using the MX1 agonist of claim 167(f) for use in
(a) treating a condition that would benefit from upregulation of immune response in a subject in need thereof.


SEQUENCE LISTING (5 sequences)

SEQ ID NO: 1 - protein, 662 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKVNAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 2 - protein, 662 aa, Homo sapiens; identical to SEQ ID NO: 1

SEQ ID NO: 3 - protein, 508 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKVNAFNQDITALMQGEETVGEEDIRLFTRLRHEFHKWSTIIENNFQEGGQQAHLQPHPFDHPVLHAPDVRPAASEGHAAAPAGQGHLQLAPEGAERHQRQAEVPEGAACTADAGSAPACPVPRLTTLCPAP

SEQ ID NO: 4 - protein, 662 aa, Homo sapiens; identical to SEQ ID NO: 1

SEQ ID NO: 5 - protein, 674 aa, Homo sapiens
MAPGQAPHQATPWRDAHPFFLLSPVMGLLSRAWSRLRGLGPLEPWLVEAVKGAALVEAGLEGEARTPLAIPHTPWGRRPEEEAEDSGGPGEDRETLGLKTSSSLPEAWGLLDDDDGMYGEREATSVPRGQGSQFADGQRAPLSPSLLIRTLQGSDKNPGEEKAEEEGVAEEEGVNKFSYPPSHRECCPAVEEEDDEEAVKKEAHRTSTSALSPGSKPSTWVSCPGEEENQATEDKRTERSKGARKTSVSPRSSGSDPRSWEYRSGEASEEKEEKAHKETGKGEAAPGPQSSAPAQRPQLKSWWCQPSDEEEGEVKALGAAEKDGEAECPPCIPPPSAFLKAWVYWPGEDTEEEEDEEEDEDSDSGSDEEEGEAEASSSTPATGVFLKSWVYQPGEDTEEEEDEDSDTGSAEDEREAETSASTPPASAFLKAWVYRPGEDTEEEEDEDVDSEDKEDDSEAALGEAESDPHPSHPDQRAHFRGWGYRPGKETEEEEAAEDWGEAEPCPFRVAIYVPGEKPPPPWAPPRLPLRLQRRLKRPETPTHDPDPETPLKARKVRFSEKVTVHFLAVWAGPAQAARQGPWEQLARDRSRFARRITQAQEELSPCLTPAARARAWARLRNPPLAPIPALTQTLPSSSVPSSPVQTTPLSQAVATPSRSSAAAAAALDLSGRRG